(19) United States
(12) Patent Application Publication
(10) Pub. No.: US 2013/0324503 A1
(43) Pub. Date: Dec. 5, 2013

(54) Method of identifying and treating a person having a predisposition to or afflicted with Parkinson disease

(71) Applicants: Haydeh Payami, Slingerlands, NY (US); Taye H. Hamza, Albany, NY (US); Stewart A. Factor, Atlanta, GA (US); John Nutt, Portland, OR (US); Cyrus Zabetian, Seattle, WA (US); Honglei Chen, Cary, NC (US); Erin Hill Burns, Niskayuna, NY (US)

(72) Inventors: Haydeh Payami, Slingerlands, NY (US); Taye H. Hamza, Albany, NY (US); Stewart A. Factor, Atlanta, GA (US); John Nutt, Portland, OR (US); Cyrus Zabetian, Seattle, WA (US); Honglei Chen, Cary, NC (US); Erin Hill Burns, Niskayuna, NY (US)

(73) Assignees: Health Research Inc., Menands, NY (US); Emory University School of Medicine, Atlanta, GA (US); National Institute of Environmental Health Sciences, Research Triangle Park, NC (US); Veterans Affairs Puget Sound Health Care System, Seattle, WA (US); Oregon Health & Science University, Portland, OR (US)

(21) Appl. No.: 13/574,000

(22) Filed: Oct. 19, 2012

Related U.S. Application Data
(63) Continuation-in-part of application No. PCT/US2011/000702, filed on Apr. 19, 2011.
(60) Provisional application No. 61/325,582, filed on Apr. 19, 2010.
(60) Provisional application No. 61/327,508, filed on Apr. 23, 2010.
(60) Provisional application No. 61/361,675, filed on Jul. 6, 2010.
(60) Provisional application No. 61/441,912, filed on Feb. 11, 2011.

(51) Int. Cl. C12Q 1/68; A61K 31/465; A61K 31/522; A61K 45/06

(52) U.S. Cl. CPC C12Q 1/6883; A61K 45/06; A61K 31/465; A61K 31/522
USPC 514/165; 514/343; 514/263.34; 514/570; 514/420; 514/682; 514/605; 514/413; 514/569; 514/567; 514/378; 514/406; 544/274; 546/279.4; 435/6.11

(57) ABSTRACT

The present invention relates to methods of treatment for Parkinson Disease (PD) in a person by identifying gene variants which may indicate a more favorable response to specific medicaments, thereby allowing for personalized or individualized treatment. The present invention relates to a method of screening for a genetic predisposition to PD in a person. The present invention is also directed to a method of testing a person for the presence of particular gene variants, wherein the presence of a gene variant indicates a higher predisposition to PD, and the absence of a gene variant indicates a lower predisposition to PD, compared to a control sample. The present invention further relates to methods and kits for treating, or inhibiting the development of, PD in a person. The present invention is also directed to a method of identifying the heritage of an individual based on the genetic profile of the individual.

### INCORPORATION BY REFERENCE

This application is a continuation-in-part application of international patent application Serial No. PCT/US11/00702 filed Apr. 19, 2011* (*USPTO is correcting—petition pending), which published as PCT Publication No. 2011/133215 on Oct. 27, 2011, which claims priority to U.S. provisional patent application Ser. Nos. 61/325,582; 61/327,508; 61/361,675 and 61/441,912 filed Apr. 19, 2010, Apr. 23, 2010, Jul. 6, 2010 and Feb. 11, 2011.

### FEDERAL FUNDING LEGEND

This invention was made with government support under NS036960 and NS067469 awarded by the NIH. The government has certain rights in the invention.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

This patent application contains lengthy table sections. Copies of the Tables have been submitted in triplicate in compact disc form (i.e., “Copy 1”, Copy 2″ and “Copy 3”) and are hereby incorporated herein by reference, and may be employed in the practice of the invention. Each compact disc, created Mar. 17, 2013 contains the following files: (1) Table A .txt, 1,957,888 bytes, (2) Table A Legend .txt, 8,192 bytes, (3) Table B .txt, 49,152 bytes, (4) Table C .txt, 35,368,960 bytes and (5) Table D .txt, 1,429,504 bytes.

LENGTHY TABLES
The patent application contains a lengthy table section. A copy of the table is available in electronic form from the USPTO web site (http://seqdata.uspto.gov/?pageRequest=docDetail&DocID=US20130324503A1). An electronic copy of the table will also be available from the USPTO upon request and payment of the fee set forth in 37 CFR 1.19(b)(3).

### FIELD OF THE INVENTION

The present invention is directed to methods and kits for identifying and treating a person having a predisposition to Parkinson disease (“PD”) to prevent the development of disease, to identify persons with PD whose genotype will determine if they respond well or poorly to each treatment so that drug efficacy can be maximized and the side effects minimized for each person individually, and for using novel genes identified for PD as targets for drug development. In particular, for example the invention is directed, but not limited, to methods for identifying a person having polymorphic gene variants in the HLA region or in different genetic loci, for example, and optionally treating the person, on a genotype-specific manner, with one or more NSAIDs; and using the interaction of GRIN2A genotype and caffeine intake and/or the interaction of SV2C genotype and nicotine to formulate individualized ideal caffeine dose for minimizing risk of developing PD, and using the HLA, GRIN2A and SV2C genes and other genes identified as a target to develop novel drugs. In sum, invention provides at least two applications: (a) use of genetic markers for pharmacogenomics i.e., individualized or personalized treatments for prevention and treatment of PD by understanding the correlation between genes and their environment (b) use of discovered genes as targets for drug development.

### BACKGROUND OF THE INVENTION

Parkinson disease (or Parkinson's disease) (PD) is a progressive movement disorder that affects millions of individuals worldwide [Factor, S. A., W. J. Weiner, and eds., Parkinson's Disease: Diagnosis and Clinical Management. Second ed. 2008, New York, N.Y.: Demos Medical Publishing, Inc.]. The clinical features include rest tremor, muscle rigidity and bradykinesia, which are linked to dopamine depletion in the corpus striatum, resulting from neuronal loss in the substantia nigra. PD is the second most common neurodegenerative disease after Alzheimer's disease (AD). The prevalence of PD is expected to double in the next 20 years and continue to rise with the aging of the population. Presently, there is neither a cure nor an effective treatment that can prevent the disease or delay its onset. Current therapies are symptomatic. Drug-induced complications and progression of disease cause increasing disability. Clinical neuroprotective trials have not been successful. A key feature of PD is that Dopamine-producing neurons in the substantia nigra selectively degenerate, resulting in a drastic reduction in the brain dopamine levels. Dopamine is a neurotransmitter and can impact many functions including voluntary movement, cognition, mood, behavior and sleep, all of which are altered in PD. About 80% of PD patients progress to develop dementia or hallucinations in addition to becoming physically disabled.

Afflicting 2% of population over the age of 65, and many who are younger, PD is the second most common neurodegenerative disease, only after Alzheimer disease. Prevalence of PD is expected to rise with our expanding lifespan and the growing population of elderly.

Currently, there is no prevention or cure. Symptomatic treatment is available, but progression of disease and complications of therapy cause increasing disability. Clinical neuroprotective treatment trials have not succeeded. Current treatments are directed towards dopamine replacement. While they help with early motor difficulties, they do not slow the progression of the disease and are associated with several late complications. To date, none of the clinical trials for neuroprotective treatments of PD have succeeded. The inability so far to account for genetic differences that affect drug response has been a hindrance to treatment trials.

Genetics research has revolutionized PD research. Up until mid 1990, PD was thought to be a purely environmental disorder with no genetic component. In the last 15 years, several disease causing genes have been identified [Polymeropoulos et al. (1997) Science 276, 2045-7, Kitada et al. (1998) Nature 392, 605-608, Leroy et al. (1998) Nature 395, 451-452, Valente et al. (2004) Science 304, 1158-60, Bonifati et al. (2003) Science 299, 256-9, Le et al. (2003) Nat Genet 33, 85-9, Paisan-Ruiz et al. (2004) Neuron 44, 595-600 and Zimprich et al. (2004) Neuron 44, 601-7]. Subsequently, transgenic animal models were developed, diagnostic gene tests came to market, and each gene provided a new insight into the disease mechanisms. This unprecedented progress was the result of discovering the genes that cause the Mendelian forms of PD. Identification of genes that are involved in the more common and typical forms of PD (henceforth, PD) may have an even greater impact on advancing the field.

Several lines of evidence point to a strong genetic component in PD. Many candidate genes have been examined [pdgene website] at least two of which have been firmly associated with a risk for developing PD: α-synuclein (SNCA) polymorphisms [Kay et al. (2008) Am J Med Genet B Neuropsychiatr Genet 147B, 1222-30 and Maraganore et al. (2006) JAMA 296, 661-70] and microtubule associated protein tau (MAPT) haplotypes [Healy et al. (2004) J Neurol Neurosurg Psychiatry 75, 962-5 and Zabetian et al. (2007) Ann Neurol 62, 137-144]. Four complex segregation analyses have been conducted; all favored mixed genetic models to a purely environmental model [Zareparsi et al. (1998) Am J Med Genet 80, 410-417, Maher et al. (2002) Am J Med Genet 109, 191, McDonnell et al. (2006) Ann Neurol 59, 788-95 and Moilanen et al. (2001) Hum Genet 108, 184-9]. Heritability was estimated at 0.45 [Moilanen et al. (2001) Hum Genet 108, 184-9], indicating presence of both genetic and environmental components. Familial aggregation studies have consistently found increased risk to first degree relatives of patients [Payami et al. (1994) Ann Neurol 36, 659-661, Autere et al. (2000) J Neurol Neurosurg Psychiatry 69, 107-9, Bonifati et al. (1995) Can J Neurol Sci 22, 272-279, De Michele et al. (1996) Mov Disord 11, 17-23, Kurz et al. (2003). Eur J Neurol 10, 159-63, Marder et al. (2003) Ann Neurol 54, 507-13, Marder et al. (1996) Neurology 47, 155-160, Payami et al. (2002) Arch Neurol 59, 848-50, Uitti et al. (1997) Can J Neurol Sci 24, 127-132, Vieregge et al. (1995) Ann Neurol 37, 685, Zorzon et al. (2002) Acta Neurol Scand 105, 77-82 and Rocca et al. (2004) Ann Neurol 56, 495-502]. A meta analysis of 29 studies placed the relative risk (RR) of PD for first degree relatives of patients at 2.9 (p<10⁻¹³) [Thacker et al. (2008) Mov Disord 23, 1174-83]. Tracing 600,000 people over 11 centuries for familial clustering of PD in Iceland, individuals who had developed PD were found to be significantly more related to each other than subjects in matched groups of controls, and RR was estimated at 6.7 for siblings and 3.2 for offspring of patients [Sveinbjornsdottir et al. (2000) N Engl J Med 343, 1765-1770]. Three twin studies were published in the last decade. Two using twin registries found no evidence for heritability of late onset PD. The third study, using imaging and clinical follow up, found combined concordance for subclinical and clinical PD to be significantly higher in monozygotic than dizygotic twins (75% vs. 25%).

Aspects of the present invention relate to genes that may influence drug response and may allow for new treatments for PD. Specifically, methods of treatments that otherwise would not reach the threshold of efficacy required for regulatory approval as a PD drug or medicament, will show high efficacy in individuals preselected by genotype as being high responders, and the “average” efficacy will not be diluted by inclusion of individuals who would not be expected to respond to the drug. This in turn will enable formulation and use of appropriate treatments for appropriate patient groups permitting individualized and personalized treatment.

Environment epidemiological studies suggest that smoking cigarettes (henceforth smoking) and drinking caffeinated coffee (henceforth coffee) are associated with reduced risk of developing PD, and elements of rural living and exposure to pesticides, herbicides and toxins are associated with increased risk [Tanner, C., Etiology: the role of environmental and genetics. In: Factor S A & Weiner W J, eds., in Parkinson's Disease: Diagnosis and Clinical Management. 2002, Demos: New York. p. 265-280 and Mayeux (2003) Annu Rev Neurosci 26, 81-104]. While toxins and the elements of rural living remain elusive, the inverse associations with smoking and coffee have been widely replicated and firmly established [Hernan et al. (2002) Ann Neurol 52, 276-84 and Powers et al. (2008) Mov Disord 23, 88-95]. The association with smoking has been verified in twin studies [Tanner et al. (2002) Neurology 58, 581-8], sib pairs [Scott et al. (2005) Neurology 64, 442-7], in a population whose risk for PD was increased by professional pesticide exposure [Galanaud et al. (2005) Mov Disord 20, 181-9], and in second-hand smokers who are only passively exposed [Mellick et al. (2006) Neurology 67, 179-80].

Experimental work in animal models and cell culture has shown that caffeine and nicotine are neuroprotective. In mouse and primate MPTP models, nicotine and cigarette-smoke decrease striatal dopamine loss [Can et al. (1990) Neuropharmacology 29, 311-4, Shahi et al. (1991) Neurosci Lett 127, 247-50, Janson et al. (1992) Clin Investig 70, 232-238 and Quik (2004) Trends Neurosci 27, 561-8], and caffeine reduces dopaminergic (DA) neuron toxicity [Chen et al. (2001) J Neurosci 21, RC143]. In 6-hydroxy dopamine treated rats, pretreatment with caffeine doses comparable to human doses protects against parkinsonian-like behaviors and substantia nigra neuron loss [Joghataie et al. (2004) Parkinsonism Relat Disord 10, 465-8]. Nicotine treatment can partially protect against paraquat-induced nigrostriatal damage in mice [Khwaja et al. (2007) J Neurochem 100, 180-90]. Nicotine may protect DA neurons via an anti-inflammatory mechanism mediated by the modulation of microglial activation [Park et al. (2007) Eur J Neurosci 26, 79-89]. Monoamine oxidase inhibition [Shahi et al. (1991) Neurosci Lett 127, 247-50 and Castagnoli et al. (2001) Chem Res Toxicol 14, 523-7], and/or cytochrome P450 induction [Shahi et al. (1991) Neurosci Lett 127, 247-50], are thought to be important.

In the brain, nicotine binds to nicotinic acetylcholine receptors with high affinity and enhances vesicular release of dopamine [Turner, J Neurosci. 2004 Dec. 15; 24(50):11328-36]. Dopamine depletion is a hallmark of PD. A growing body of work has implicated altered synaptic transmitter release in the pathogenesis of PD [Esposito, Dev Neurobiol. 2012 January; 72(1):134-44.]. Synaptic vesicle proteins SV2A/SV2B/SV2C are integral membrane components of synaptic vesicles and have been implicated in storage and release of neurotransmitters [Feany, Cell. 1992 Sep. 4; 70(5):861-7.; Dardou, Brain Res. 2011 Jan. 7; 1367:130-45]. A recent study has shown that modest changes in SV2 expression, in either direction, can have a significant impact on synaptic function [Nowack, PLoS One. 2011; 6(12)]. SV2C is densely expressed in dopaminergic neurons in substantia nigra [Janz, Neuroscience. 1999; 94(4):1279-90]. The connection between nicotinic enhancement of vesicular dopamine release and altered neurotransmission may be due to changes in expression of SV2C gene. Aspects of the genetic basis of the variable effects of smoking/nicotine in Parkinson's disease, including a genome-wide search that indicated protective benefit from smoking/nicotine depends on SV2C genotype, has been referred to in Hill-Burns E M et al. A genetic basis for the variable effect of smoking/nicotine on Parkinson's disease, The Pharmacogenomics Journal, (2 Oct. 2012). Administration of nicotine may not be considered or approved for PD treatment, despite being potentially safe and effective for prevention and treatment in some individuals, unless individuals who can benefit from it can be distinguished from those who do not.

Caffeine is a non-selective competitive adenosine A2A receptor antagonist; a family of compounds with neuroprotective properties [Chen et al. (2001) J Neurosci 21, RC143 and 56]. A2A receptor antagonists, such as Istradefylline have been tested for PD in clinical trials and shown promising results with fewer complications than current therapies [Factor (2008) Neurotherapeutics 5, 164-80]. A 29-site clinical trial and a 40-site clinical trial with Istradefylline recently completed reported significant and clinically meaningful reduction in “off” time without increased troublesome dyskinesia [LeWitt et al. (2008) Ann Neurol 63, 295-302 and Stacy et al. (2008) Neurology 70, 2233-40]. Further studies relate to caffeinated-coffee consumption protecting some people from developing PD, although not all benefit equally. In Hamza T H, Chen H, Hill-Burns E M, Rhodes S L, Montimurro J, Kay D M, et al. Genome-Wide Gene-Environment Study Identifies Glutamate Receptor Gene GRIN2A as a Parkinson's Disease Modifier Gene via Interaction with Coffee. PLoS Genet 2011; 7(8): e1002237 (incorporated by reference in its entirety) a genome-wide search indicated that variations in the glutamate-receptor gene GRIN2A may modulate the risk of developing PD in heavy coffee drinkers. Administration of caffeine/A2ARA may not be considered or approved for PD treatment, despite being potentially safe and effective for prevention and treatment in some people, unless people who can benefit from it can be distinguished from those who do not.

Furthermore, Neuro-inflammation and humoral immunity are involved in PD pathogenesis. Studies have indicated that there may be elevated expression of HLA-DR antigens in the brain and cerebrospinal fluid of PD patients. It is postulated that the chronic immune activation and neuro-inflammation is in response to an initial trigger, possibly related to alpha-synuclein accumulation, and produce neurotoxins and oxidative damage that could kill neurons. An inflammatory pathogenesis for PD was first proposed in 1980's by McGeer. Whether inflammation was the cause or consequence of PD remained a question until the discovery of a genetic association between PD and major histocompatibility gene (HLA). The validation of the existence of a genetic etiology for PD in the HLA region firmly grounded, at the DNA level the long held notion that the immune system and inflammation play a significant role in etiology of PD and is described in Hamza et al, Common genetic variation in the HLA region is associated with late-onset sporadic Parkinson's disease, Nat Genet. 2010 September; 42(9): 781-785, the disclosure of which is incorporated herein in its entirety. From a therapeutic perspective, immunomodulatory drugs, including NSAIDS, aimed at down regulating inflammatory processes are considered strong candidates for Parkinson disease treatment. Vaccination aimed at neutralizing neuro-immune dysfunction was recently shown to attenuate neurodegeneration in a PD model. From a prevention perspective, NSAID use, in particular ibuprofen, has been shown to associate with reduced risk of developing PD in humans. Aspects of the invention relate to the administration of over-the-counter and prescription NSAIDS separately to PD individuals in need thereof that may also possess different gene variants associated with a favorable response to each type of administration.

Most cases of PD are thought to result from interactions between genes and environmental factors. There is support of the long-held notion that PD susceptibility is determined by cumulative and synergistic effects of genes and environmental factors [McCulloch et al. (2008) Hum Genet 123, 257-65].

Previously a number of genome-wide association and interaction studies were aimed at deciphering genetics of common disorders as genome-wide linkage studies were for Mendelian disorders [Hirschhorn et al. (2005) Nat Rev Genet 6, 95-108]. Genome-wide association studies (GWAS) are in full force with successful results emerging for a wide range of common and complex disorders [Estivill et al. (2007) PLoS Genet 3, e190 and Topol, E. J., S. S. Murray, and K. A. Frazer (2007) Jama 298, 218-21]. Three GWAS have been published for PD [Maraganore et al. (2005) Am J Hum Genet 77, 685-93, Fung et al. (2006) Lancet Neurol 5, 911-6 and Pankratz et al. (2009) Hum Genet 124, 593-605]. None has revealed novel reproducible associations [Elbaz et al. (2006) Lancet Neurol 5, 917-23], nor did a meta-analysis identify any robust associations [Evangelou et al. (2007) PLoS ONE 2, e196].

The individual genetic effects found for other complex disorders are small, with odds ratios ranging from 1.1 to 1.7 [Topol et al. (2007) Jama 298, 218-21]. The associations found to date account for only a fraction of genetic variability in each disorder. For example, more than ten loci identified for type 2 diabetes account for <5% of the genetic variability in that disorder [Frayling(2007) Nat Rev Genet 8, 657-62].

Over the past ten years, numerous neurodegenerative diseases have been modeled in Drosophila and these models have been used successfully to identify disease pathways and drug targets [Bilen et al. (2005) Annu Rev Genet 39, 153-71, Cauchi et al. (2006) Neurodegener Dis 3, 338-56 and Marsh et al. (2006) Neuron 52, 169-78]. Six genetic and two environmental models of parkinsonism have been developed in flies. In three genetic models, over-expression of SNCA, mutant human PRKN, or mutant human LRRK2 results in age dependent loss of DA neurons and motor abnormalities [Auluck et al. (2002) Science 295, 865-8, Feany et al. (2000) Nature 404, 394-8, Liu et al. (2008) Proc Natl Acad Sci USA 105, 2693-8, Sang et al. (2007) J Neurosci 27, 981-92 and Trinh et al. (2008) J Neurosci 28, 465-72]. In the case of LRRK2, there is modest reduction in lifespan. In the PRKN, DJ-1 and Pink-1 models, the endogenous gene is knocked out or down; remarkably all three mutants show increased sensitivity to oxidative stress, and both Pink-1 and PRKN models have severe mitochondrial pathology [Greene et al. (2005) Hum Mol Genet 14, 799-811, Greene et al. (2003) Proc Natl Acad Sci USA 100, 4078-83, Menzies et al. (2005) Curr Biol 15, 1578-82, Meulener et al. (2005) Curr Biol 15, 1572-7 and Wang et al. (2006) Proc Natl Acad Sci USA 103, 13520-5]. Anti-oxidants and/or genetic manipulation that increase resistance to oxidative stress are uniformly beneficial and can reduce or prevent mitochondrial pathology, muscle degeneration and loss of DA neurons, and increase lifespan [Meulener et al. (2005) Curr Biol 15, 1572-7, Bier (2006) Proc Natl Acad Sci USA 103, 13269-70, Pesah et al. (2004) Development 131, 2183-94 and Whitworth et al. (2005) Proc Natl Acad Sci USA 102, 8024-9]. Recent genetic epistasis studies indicate the three genes operate in a common pathway that senses and responds to oxidative stress [Bier (2006) Proc Natl Acad Sci USA 103, 13269-70, Clark et al. (2006) Nature 441, 1162-6 and Yang et al. (2006) Proc Natl Acad Sci USA 103, 10793-8]. Parkin and Pink 1 are in a common pathway that controls mitochondrial dynamics. All these genetic mutations show an interaction with the environmental toxicant, paraquat [Meulener et al. (2005) Curr Biol 15, 1572-7, Wang et al. (2006) Proc Natl Acad Sci USA 103, 13520-5 and Pesah et al. (2004) Development 131, 2183-94], which has been associated with development of PD in humans [Dinis-Oliveira et al. (2006) Neurotoxicology 27, 1110-22, Costello et al. (2009) Am J Epidemiol 169, 919-26, Firestone et al. (2005) Arch Neurol 62, 91-5 and Liou et al. (1997) Neurology 48, 1583-8]. The paraquat fly model of parkinsonism is created by feeding paraquat to flies, which results in greatly shortened lifespan, selective and progressive loss of DA neurons, and motor abnormalities. Thus, in this model many of the phenotypic hallmarks of the human parkinsonism are recapitulated, and the significantly shortened life span provides a phenotype that is amenable to high through put screening.

Citation or identification of any document in this application is not an admission that such document is available as prior art to the present invention.

### SUMMARY OF THE INVENTION

The present invention is directed to a method of treating or inhibiting the development of Parkinson's Disease (PD) in a person in need thereof wherein said method may comprise providing individualized or personalized treatment which may comprise (a) analyzing DNA from a blood, saliva or tissue sample obtained from the person; (b) determining from said analyzing the presence of a polymorphic site in a genetic locus, whereby the presence of the polymorphic site identifies a gene variant that allows or increases the neuroprotective effect of (i) nicotine (ii) caffeine or (iii) NSAID consumption; and (c) if the polymorphic site is present and (i) the presence of the polymorphic site identifies a gene variant that allows or increases the neuroprotective effect of nicotine, then administering to the person a medicament comprising nicotine or a nicotine analog, or (ii) the presence of the polymorphic site identifies a gene variant that allows or increases the neuroprotective effect of caffeine, then administering to the person a medicament comprising caffeine or a caffeine analog, or (iii) the presence of the polymorphic site identifies a gene variant that allows or increases the neuroprotective effect of NSAID consumption, then administering to the person a medicament comprising one or more NSAID or NSAID analogs; wherein the nicotine or nicotine analog, the caffeine or caffeine analog or NSAID or NSAID analog is administered in an effective Parkinson's disease treating amount or an amount effective for inhibiting the development of PD.

In an embodiment of the invention, the analyzing DNA step may comprise (i) obtaining a blood, saliva or tissue sample from the person, (ii) isolating DNA from the blood, saliva or tissue sample, and (iii) genotyping the DNA.

Another embodiment of the invention may relate to NSAIDs selected from the group consisting of: ibuprofen, aspirin, indomethacin, nabumetone, nimesulide, selective cyclooxygenase (COX) inhibitors, licofelone, Anaprox (naproxen), Arthrotec (diclofenac sodium), Bextra (valdecoxib), Cataflam (diclofenac potassium), Celebrex (celecoxib), Clinoril (sulindac), Dolobid (diflunisal), EC-naprosyn (naproxen), Feldene (piroxicam), Indocin (indomethacin), Mobic (meloxicam), Motrin (ibuprofen), Naprelan (naproxen controlled release), Naprosyn (naproxen), Ponstel (mefenamic acid), Relafen (nabumetone), Toradol (ketorolac tromethamine), Trilisate (choline magnesium salicylate), Vioxx (rofecoxib) and Voltaren (diclofenac sodium).

In a still further embodiment of the invention the caffeine analog may be selected from the group consisting of Theophylline, 1-Proparagyl 3,7-Dimethyl Xanthine, 7-Proparagyl 1,3-Dimethyl Xanthine, 3-Proparagyl 1,7-Dimethyl Xanthine, 1,3,7-Triproparagyl Xanthine, IBMX, 1,3,7-Tripropyl Xanthine, 7-Benzyl-IBMX, 1-Propyl 3,7-Dimethyl Xanthine, 1,3-Dipropyl 7-Methyl Xanthine, 1,3-Dipropyl 7-Proparagyl Xanthine, 3,7-Dimethyl 1-Propyl Xanthine and 7-allyl 1,3-dimethyl Xanthine. In a preferred embodiment, the caffeine analog may be a adenosine A2A receptor antagonist.

In yet a further embodiment of the invention, the nicotine analog may be selected from the group consisting of N-succinyl-6-amino-(+/−)-nicotine, 6-(sigma-aminocapramido)-(+/−)-nicotine, O-succinyl-3′-hydroxymethyl-nicotine and 3′-(hydroxymethyl)-nicotine hemisuccinate.

Particular embodiments of the invention relate to a polymorphic site which may comprise a single nucleotide polymorphism (SNP).

In an advantageous embodiment the genetic locus may be GRIN2A locus (encodes NMDA Glutamate receptor subunit), the SNP may comprise rs4998386 having a thymine (T) in heterozygous or homozygous state and the administering may be of caffeine or a caffeine analog.

In a further advantageous embodiment the genetic locus may be SV2C locus (encodes synaptic vesicle protein), the SNP may comprise rs30196 having a cytosine (C) in heterozygous or homozygous state or rs10214163 having a thymine (T) in heterozygous or homozygous state and the administering may be of nicotine or a nicotine analog.

In other aspects of the invention, the SNP may comprise rs2338971 having a cytosine (C) in homozygous state or rs2072029 having a thymine (T) in heterozygous or homozygous state and the administering may be of a NSAID.

In yet another advantageous embodiment of the invention, the genetic locus may be LAMA4 locus (encodes laminin subunit alpha 4), the SNP comprises rs2072029 having a thymine (T) in heterozygous or homozygous state and the administering is of ibuprofen.

Certain aspects of the invention relate to a polymorphic site which may comprise a proxy SNP.

In one embodiment the genetic locus may comprise a GRIN2A locus, the proxy SNP may have at least 50% correlation with rs4998386 having a thymine (T) in heterozygous or homozygous state and the administering may be of caffeine or a caffeine analog.

In a second embodiment the genetic locus may comprise a SV2C locus, the proxy SNP may have at least 50% correlation with rs30196 having a cytosine (C) in heterozygous or homozygous state or at least 50% correlation with rs10214163 having a thymine (T) in heterozygous or homozygous state and the administering may be of nicotine or a nicotine analog.

In a third embodiment the proxy SNP may have at least 50% correlation with rs2338971 having a cytosine (C) in homozygous state or has at least 50% correlation with rs2072029 having a thymine (T) in heterozygous or homozygous state and the administering may be of a NSAID.

In a further embodiment, the genetic locus may comprise a LAMA4 locus, the proxy SNP may have at least 50% correlation with rs2072029 having a thymine (T) in heterozygous or homozygous state and the administering may be of ibuprofen.

Further embodiments of the invention relate to medicaments which may further comprise levodopa, a dopamine agonist, a catechol O-methyltransferase (COMT) inhibitor, a monoamine oxidase B (MAO B) inhibitor, an anticholinergic agent, an antiviral agent or a combination thereof.

Embodiments of the invention may also relate to kits which may comprise: (a) a medicament comprising one or more NSAIDs, caffeine or caffeine analogs, nicotine or nicotine analogs, or combinations thereof; and (b) instructions for performing the method of the invention. The kit may further comprise levodopa, a dopamine agonist, a catechol O-methyltransferase (COMT) inhibitor, a monoamine oxidase B (MAO B) inhibitor, an anticholinergic agent, an antiviral agent or a combination thereof and instructions for administration thereof.

The present invention is also directed to a method of identifying in a person a genetic predisposition to PD, wherein the method may comprise: (a) obtaining a blood sample or saliva or another tissue from the patient; (b) analyzing the DNA from the blood sample or other tissue for the presence or absence of a polymorphic site in a gene listed in Tables A, B, C or D, whereby the presence of the polymorphic site identifies a gene variant; wherein the presence of the gene variant may indicate a higher predisposition to PD, and the absence of the gene variant may indicate a lower predisposition to PD, compared to a control sample. The method may also comprise (a) obtaining a blood sample from the patient; (b) analyzing the DNA from the blood sample for the presence or absence of a polymorphic site in a gene listed in Tables A, B, C or D, whereby the presence of the polymorphic site may identify a gene variant; wherein the presence of the gene variant may indicate a better response to protective effect of caffeine or other bioactive ingredients of coffee, or an analogue (iii) the presence of the gene variant may indicate a better response to protective effect of nicotine or other bioactive ingredients of cigarettes, or an analogue (iii) the presence of the gene variant may indicate a better response to protective effect of NSAIDs, (iv) the presence of the gene variant may indicate a better response to symptomatic treatment with adenosine related drugs (such as, but not limited to, a2A antagonists), (v) the presence of the gene variant may indicate a better response to treatment with glutamate-related drugs (such as, but not limited to, NMDA receptor antagonists).

The present invention is also directed to a method of testing a blood sample of a person for the presence of a polymorphic site, wherein said method may comprise: (a) obtaining a blood sample from the patient; (b) analyzing the DNA from the blood sample for the presence or absence of a polymorphic site in a gene listed in Tables A, B, C or D, whereby the presence of the polymorphic site may identify a gene variant; wherein the presence of the gene variant may indicate a higher predisposition to PD, and the absence of the gene variant may indicate a lower predisposition to PD, compared to a control sample.

In one embodiment of the invention, the gene variant may be associated with an increased risk or a decreased risk of developing PD. In another embodiment, the gene variant may interact with and increase or decrease a neuroprotective effect from nicotine/smoking, caffeine/coffee and NSAID consumption.

In one embodiment of the invention, the gene variant may be related to disease progression, for example, to the development of dementia and/or hallucination in patients having or at risk of developing PD.

In one embodiment of the invention, the gene variants may be used as targets to develop treatments for PD (e.g., drug discovery). In another embodiment, the presence of particular gene variants in a patient or a patient's genetic profile may be used to assess and refine drug safety and efficacy (e.g., pharmacogenomics). In yet another embodiment, the presence of particular gene variants in a patient or a patient's genetic profile may be used to prescribe custom drug therapies for treating or inhibiting the development of PD in the patient.

The present invention is further directed to a method of treating, or inhibiting the development of, PD in a person, which may comprise: (a) determining in a person the presence or absence of a polymorphic site in a gene listed in Tables A, B, C or D, whereby the presence of the polymorphic site may identify a gene variant, wherein the presence of the gene variant may indicate a higher predisposition to PD, and the absence of the gene variant may indicate a lower predisposition to PD, compared to a control sample; and (b) administering to the person having the gene variant a therapeutically effective amount of a medicament comprising one or more non-steroidal anti-inflammatory drugs (NSAIDs), caffeine or caffeine analogs, and nicotine or nicotine analogs, wherein the medicament may treat, or inhibit the development of, PD. In one embodiment of the invention, the drug may be chosen based on the particular gene variant in the patient. For example, a person having a GRIN2A gene variant may be given caffeine or a caffeine analog, such as Istradefylline or xanthine, to treat or prevent the development of PD in the patient. Similarly, a person having a MAPK10 or PPIAL3 gene variant may also be given caffeine or a caffeine analog to treat or prevent the development of PD. In another embodiment, a person having a SV2C gene variant may also be given nicotine or a nicotine analog to treat or prevent the development of PD.

In one embodiment of the invention, the medicament may further comprise but is not limited to levodopa, dopamine agonists, catechol O-methyltransferase (COMT) inhibitors, monoamine oxidase B (MAO B) inhibitors, anticholinergic agents, antiviral agents or a combination thereof.

In one embodiment of the invention, PD may be sporadic PD or late-onset PD.

In one embodiment of the invention, the polymorphic site may be a single nucleotide polymorphism (SNP). In another embodiment of the invention, the SNP may be at a position listed in Tables A, B, C or D. In one embodiment, the SNP may be at a position within about 500 kb upstream or within about 500 kb downstream from a position listed in Tables A, B, C or D. In another embodiment, the SNP may be at a position within about 50 kb upstream or within about 50 kb downstream from a position listed in Tables A, B, C or D. In yet another embodiment, the SNP may be at a position within about 1 kb upstream or within about 1 kb downstream from a position listed in Tables A, B, C or D. In still another embodiment, the SNP may be within about 100 bp upstream or within about 100 bp downstream from a position listed in Tables A, B, C or D. In regions of the genome where linkage disequilibrium structure is complex, HLA is the prime example, the SNP may be with several megabases from the SNP in the position listed in Tables A, B, C or D.

In one embodiment of the invention, the gene may be on chromosome 6. In another embodiment, the gene may be HLA. HLA may be HLA-DR, HLA-DRA, or HLA-DRB. In one embodiment, the SNP may be on intron-1 of the HLA gene. In still another embodiment, the SNP may be at position 32517508 on chromosome 6, within the HLA gene. In still a further embodiment, the gene or SNP position may be selected from the genes and/or positions listed in Table A. In another embodiment, the gene or SNP position may be selected from the genes and/or positions listed in Table B. In yet another embodiment, the gene or SNP position may be selected from the genes and/or positions listed in Table C. In still another embodiment, the gene or SNP position may be selected from the genes and/or positions listed in Table D.

In one embodiment of the invention, the method may further comprise determining whether the person smokes cigarettes, consumes caffeinated coffee, and/or uses NSAIDs, wherein a predisposition to developing PD may be associated, inversely, with cigarette smoking, caffeinated coffee consumption and NSAID use. The person may be identified via a questionnaire, personal interview, medical history analysis, or other means typically used to identify patients who will participate in clinical trials.

Any one of the methods of the present invention may comprise: (i) obtaining a blood, saliva or tissue sample from the patient, (ii) isolating DNA from the blood sample, and (iii) genotyping the DNA, all of which are well-known in the art and described in detail below.

In one embodiment of the invention, the one or more NSAID may in both conjugated and unconjugated form. Suitable NSAIDs include, but are not limited to, aspirin, indomethacin, ibuprofen, fenoprofen, licofelone, naproxen, mefanamic acid, mefclofenamic acid, nabumetone, nimesulide, selective cyclooxygenase (COX) inhibitors, sulindac, tolmetin, etc.

In one embodiment of the invention, the medicament may be administered orally, intravenously or topically, or by any other means deemed appropriate by an attending physician.

The present invention is also directed to a kit which may comprise: (a) a medicament comprising one or more NSAIDs, caffeine or caffeine analog, nicotine or nicotine analogs, or a combination thereof; and (b) optionally instructions for administering the medicament to a person having a genetic predisposition to PD, identified by the methods disclosed herein. The medicament may comprise a pharmaceutically acceptable excipient or carrier. The medicament may further comprise levodopa, dopamine agonists, catechol O-methyltransferase (COMT) inhibitors, monoamine oxidase B (MAO B) inhibitors, anticholinergic agents, antiviral agents or a combination thereof.

The present invention is also directed to a method for identifying the heritage of an individual, comprising: (a) obtaining a blood, saliva or tissue sample from the individual; (b) analyzing the DNA from the blood sample for the presence or absence of a one or more genetic markers; (c) assigning a genetic profile to the individual based on the presence or absence of the one or more genetic markers; (d) correlating the genetic profile with a geographic location and ethnic heritage; and (e) identifying the heritage of an individual based on the correlation. The individual may be of Jewish heritage. The geographic location may be but is not limited to Eastern Europe, France, Great Britain, Germany-Austria, Holland, Ireland, Italy, Russia or Scandinavia.

The present invention is further directed to a method of identifying in a person a genetic predisposition to or protection against an HLA-associated disease, such as an autoimmune disease, and infectious disease, or a cancer. HLA-associated diseases may include, but are not limited to, primary hemochromatosis, ankylosing spondylitis, inflammatory bowel, reiter's disease, psoriatic arthritis, system lupus erythematosus, rheumatoid arthritis, Graves' disease, celiac sprue, multiple sclerosis, hay fever, Goodpasture's syndrome, Sjögren's syndrome, pernicious anemia, Hashimoto's thyroiditis, narcolepsy, lyme disease, pemphigus vulgaris, Type 1 diabetes mellitus, acute uveitis, and psoriasis. See R. T. D. Oliver, The HLA system and immunological defense against cancer: a review, Journal of the Royal Society of Medicine, Vol. 71, January 1978. pg. 50-54, incorporated herein by reference. HLA-associated diseases may also include breast cancer. See Leong, et al. HLA-A and breast cancer in West Peninsular Malaysia, Medical Oncology, published online Jan. 13, 2010, herein incorporated by reference.

Accordingly, it is an object of the invention to not encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. §112, first paragraph) or the EPO (Article 83 of the EPC), such that Applicants reserve the right and hereby disclose a disclaimer of any previously described product, process of making the product, or method of using the product.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings, in which:

FIG. 1 depicts sample call rates.

FIG. 2 depicts genotype calls for HLA-DRA rs3129882.

FIG. 3A-B depicts genome-wide association P-values, wherein (a) the Manhattan plot shows the P-values for association of 811,597 SNPs with PD, and (b) the quantile-quantile plot depicts the distribution of expected P-values for no disease association (black line) vs. the observed P-values in NeuroGenetics Research Consortium (NGRC) dataset. The observed are plotted once for all 811,597 SNPs tested (red), once excluding SNCA and MAPT regions (green), and again excluding SNCA, MAPT and HLA-DR regions (blue). λ=Genomic inflation factor.

FIG. 4A-C depicts top hits in HLA-DR region and linkage disequilibrium among them.

FIG. 5 depicts odds ratios for the combination of SNCA, MAPT and HLA-DR.

FIG. 6A-F depicts moving average plots (MAPs), wherein (a) two theoretical models are shown for contrast to actual data, (b) HLA-DRA rs3129882 G allele frequency begins to rise in patients at around age 50 and remains consistently higher in patients than controls to advanced ages, (c) MAPT rs199533 T allele shows a uniformly lower frequency in patients than controls, (d) SNCA rs356220 T allele and (e) GAK rs11248051 T allele are elevated in PD across the age spectrum, and (f) rs823128 identified as a new risk factor for PD in the Japanese (PARK16) is not associated with PD in the NGRC Caucasian dataset in any age range.

FIG. 7A-D depicts Principal Component Analysis (PCA) and self-reported ancestry, wherein (a) NGRC samples (blue) are plotted against HapMap Caucasian Population (purple), HapMap Chinese-Japanese population (red) and HapMap Yoruba African population (green), (b) PCA is repeated without HapMap samples (PC1 and PC2 separate two clusters; cases (red) and controls (blue) are distributed in both clusters), (c) subjects who reported as Ashkenazi Jewish are plotted in red and those who did not specify and therefore may or may not be Jewish are shown in blue, and (d) subjects whose paternal and maternal ancestors immigrated to the US from the same European country are designated by the same color.

FIG. 8 depicts localization of chromosomal regions responsible for significant principle component (PC1). To pinpoint the genomic regions that gave rise to PC1, linear regression was used to test association of SNPs with PC1 and the P-values were displayed in a Manhattan plot.

FIG. 9 depicts linkage disequilibrium (LD) between PC1- and PD-associated variants within HLA. The haploview diagram shows the level of LD (r2) between the top six variants from HLA that showed the strongest association with PC1 (columns 1-6) and rs3129882 which are the PD-associated variants in HLA-DRA (column 7). PC1-associated HLA variants are ˜200 kb way from HLA-DRA. There was low-level LD between PC1-associated variants and rs3129882 (r2=0.03-0.08).

FIG. 10 depicts LD between DRA and DRB.

FIG. 11A-B depicts a Manhattan Plot and QQ plot of genome-wide SNP+SNP*coffee interaction test. Applicants tested 811,597 SNPs in combination with coffee consumption, for association with PD. The model was [SNP+SNP*coffee] test with 2 df, adjusted for sex, age, PC1 and PC2. Additive model is shown here, Dominant and Recessive are in FIG. 12. Dominant and Additive models yielded similar results for top hits (see Table 10). Panel A depicts the Manhattan plot, demonstrating the spike on chromosome 16 corresponding to 12 GRIN2A SNPs. Panel B is the QQ plot where the observed P values (red line) are plotted against expected P values under no association (straight black line). The plots were made first by including all SNPs (red), then excluding SNCA, HLA and MAPT (green) and finally by excluding GRIN2A (blue). Two points in the plot are noteworthy, a deviation around P<10⁻³ and a larger deviation at the extreme<10⁻⁵. The deviation around 10⁻³ is due to the known PD genes, once SNCA, MAPT and HLA are removed it disappears (green line). The extreme deviation at <10⁻⁵ is primarily due to GRIN2A. When GRIN2A is removed, shown in blue line, much of the deviation at the extreme is abolished.

FIG. 12A depicts a Manhattan Plot of GWAIS. Applicants tested 811,597 SNPs in combination with coffee consumption for association with PD. The model was [SNP+SNP*coffee] test with 2 df, adjusted for sex, age, PC1 and PC2. Dominant and Additive models yielded similar results for the top hits (see Table 10 of Example 3). Additive model is shown in FIG. 11; Dominant and Recessive are shown here.

FIG. 12B depicts GWAS in heavy- and light-coffee drinkers. The P values in stratified GWAS are for SNP main effect on PD risk, adjusted for sex, age, PC1 and PC2. Dominant and Additive models yielded similar results for top hits (see Table 11 in Example 3). Additive is shown in FIG. 14 of main text; Dominant is shown here.

FIG. 13A-C depicts a map of GRIN2A. Panel (A): Chromosomal location and gene structure of GRIN2A. Numbers 1-14 denote exons. Panels (B) and (C): Linkage disequilibrium (LD) map of all the genotyped SNPs that are located in the GRIN2A gene or within 50 kb upstream or downstream of the gene. LD is measured as r2 (shades of grey) in panel (b) and as D′ (shades of red) in panel (c). The intensity of the color depicts strength of LD and the numbers in the grids are the values of r2 and D′ in percentage.

FIG. 14A-D depicts GWAS in heavy- and in light-coffee drinkers. The P values in stratified GWAS are for SNP main effect on PD risk, adjusted for sex, age, PC1 and PC2. Additive model is shown here; see FIG. 12B for Dominant model. Dominant and additive models yielded similar results for top hits (see Table 11). Panel A depicts GWAS in heavy-coffee drinkers with GRIN2A achieving lowest P value. Panel B is the QQ plot for heavy-coffee drinkers where the observed P values (red line) are plotted against expected P values under no association (straight black line). The plots were made first by including all SNPs (red), then excluding SNCA, HLA and MAPT (green) and finally by excluding GRIN2A (blue). Unlike the QQ plot for GWAIS, the effects of SNCA, HLA and MAPT are unnoticeable. The only deviation is seen at the extreme<10⁻⁵ which is primarily due to GRIN2A. When GRIN2A is removed, shown in blue line, much of the deviation at the extreme is abolished. Panel C and Panel D are GWAS and QQ plot for light-coffee drinkers, which display no clear signal.

FIG. 15A-B depicts LD among the PD-associated SNPs. SNPs marked in red boxes were genotyped and achieved P<10⁻⁵ in either 2 df GWAIS or GWAS in heavy-coffee drinkers. SNPs not in red boxes were imputed and achieved P≦5×10⁻⁸ in either 2 df GWAIS or GWAS in heavy-coffee drinkers. Panels (A) r2, (B) D′.

FIG. 16A-B depicts LD among the PD-associated and HD-associated SNPs. SNPs marked in red boxes were genotyped and achieved P<10⁻⁵ in either 2 df GWAIS or GWAS in heavy-coffee drinkers with PD. SNPs not in boxes were imputed and achieved P≦5×10⁻⁸ in either 2 df GWAIS or GWAS in heavy-coffee drinkers. SNPs in blue boxes are reported as being associated with HD. Panels (A) r2, (B) D′.

FIG. 17 depicts Drosophila: Effects of paraquat and nicotine on survival. Nicotine improved survival of paraquat-treated flies in a dose-dependent manner. Each treatment combination was started with 420 flies. Survival Curves were plotted using Kaplan Meier survival analysis, and differences between survival curves were calculated using log rank statistics)(P=4×10⁻³⁰.

FIG. 18 depicts Drosophila: Effects of paraquat and nicotine on gene expression. CG14691 gene expression was increased significantly (P=5×10⁻⁸) in response to paraquat and restored to normal with co-treatment with nicotine (paraquat-nicotine interaction P=2×10⁻¹¹).

FIGS. 19A-B depict Human: Genome-wide SNP*smoking interaction study. (A) Manhattan plot of −log₁₀(P) values for interaction tested between 810 234 genotyped SNPs and smoking. The red data point is a two-SNP interaction test at 5′ of SV2C. (B) Quantile-quantile plot of SNP*smoking interaction P values. Black: full data. Red: excluding SV2C region.

FIG. 20 depicts Human: Linkage disequilibrium in SV2C region. Genotyped SNPs that achieved P_{Interaction}<10⁻³ for SNP*smoking interaction in the SV2C region were tested for LD; the numbers in the grid represent the correlation (r²) between each pair of SNPs. Although there appear to be three haploblocks, the SNP in the far-right block (rs 183766 shown in grey box) did not have an effect independent of the other blocks. Signals from the other two haploblocks (rs30196 and rs10214163 shown in black boxes) appeared to be independent and additive, as indicated by persistent significance of one when conditioned on the more significant one. In line with this evidence for independent effects, joint consideration of genotypes at rs30196 and rs10214163 improved significance level for interaction with smoking to P=9×10⁻⁸.

FIG. 21A-B depicts Drosophila: Pilot experiment with paraquat and nicotine. Each treatment dosing combination started with 180 flies. (A) Kaplan Meier survival analysis, (B) Mean and standard error of survival.

FIG. 22A-B depicts Drosophila: Large-scale survival experiment. Each treatment dosing combination started with 420 flies. Dead flies were counted daily and the surviving flies were transferred to a fresh, pre-warmed (25° C.) vial every other day. Survival curves were plotted using Kaplan Meier survival analysis.

FIGS. 23A-D depict Drosophila: Gene expression data quality measures. Panels (A), (B), (C): Density plots of log(intensity) of All probes, perfect match (PM) probes; and mismatch (MM) probes on all arrays. (D): RNA degradation plot. The sample shown in red was excluded; it was one of the triplicates for paraquat-only treatment.

### DETAILED DESCRIPTION

PD is not one disease, and one therapy will not work for all. One object of this invention is to genetically sub-type individuals and assess drugs on appropriate genetic backgrounds. In particular, the instant invention is directed to the identification of genetic markers for identifying appropriate patient populations for clinical trials such that patients with particular gene variants are predicted to respond most effectively to particular types of pharmacological agents, as well as to the development of new drug targets for personalized treatments of PD. The application of personalized or individualized treatment in a clinical setting will maximize drug efficacy and safety for each patient or person in need thereof by administering the appropriate drug in view of the preselection based on genetic background.

Methods that may be used to treat PD use a variety of approaches such as drug treatments aimed to increase the level of dopamine, surgical treatments, gene therapy, stem cells etc. (Parkinsons.org.uk website, U.S. Pat. Nos. 4,824,860; 8,257,929). The present invention is an improvement on such treatments and can be employed in conjunction or addition therewith.

The invention is based, in part, on a genome-wide association study (GWAS) with 2,000 PD patients with 1,986 control subjects from NGRC. The study on which the instant invention is based, revealed a significant 23-56% dose-dependent reduction in PD risk for smokers, and 25-43% for caffeinated coffee consumers. While the present invention is directed to identifying individuals having a genetic basis of nicotine and caffeine associated PD risk reduction, it is not limited to nicotine and caffeine associations.

In one study on which the instant invention is based, known genetic associations with SNCA and MAPT were confirmed and a novel association with HLA-DR (P_{NGRC}=70.5×10⁻⁹) was detected, which was replicated in two independent datasets (P_{Replication1+2}=1.1×10⁻³, P_{Replication1+2+NGRC}=2.2×10⁻¹⁰). The association was robust to population structure; uniform across genetic and environmental risk strata; and strong in sporadic PD (P=1.2×10⁻¹⁰), late-onset PD (P=5.3×10⁻⁹) and men (P=4.5×10⁻⁸). The inventors designate the new PD locus PARK18.

“Parkinson disease” also refers to “Parkinson's disease”.

As used herein “diagnosis” or “identifying a patient having” refers to a process of determining if an individual is afflicted with, or has a genetic predisposition to develop, PD.

The terms “treat,” treating,” “treatment,” and the like refer to reducing or ameliorating PD or symptoms associated therewith. It will be appreciated that, although not precluded, treating PD or the risk of developing PD does not require that the disease or the risk be completely eliminated.

As used herein, “inhibiting the development of,” “reducing the risk of,” “prevent,” “preventing,” and the like refer to reducing the probability of developing PD in a patient who may not have PD, but may have a genetic predisposition to developing PD. As used herein, “at risk,” “susceptible to,” or “having a genetic predisposition to,” refers to having a propensity to develop PD. For example, a patient having a genetic mutation in a gene associated with PD has increased risk (e.g., “higher predisposition”) of developing the disease relative to a control subject having a “lower predisposition” (e.g., a patient without a genetic mutation in a gene associated with PD).

As used herein, “reduces,” “reducing,” “inhibit,” or “inhibiting,” may mean a negative alteration of at least 10%, 15%, 25%, 50%, 75%, or 100%.

As used herein, “increases” or “increasing” may mean a positive alteration of at least 10%, 15%, 25%, 50%, 75%, or 100%.

A “therapeutically effective amount” refers to the amount of a compound required to improve, inhibit, or ameliorate a condition of a patient, or a symptom of a disease, in a clinically relevant manner. Any improvement in the patient is considered sufficient to achieve treatment. A sufficient amount of an active compound used to practice the present invention for the treatment of PD varies depending upon the manner of administration, the age, body weight, genotype, and general health of the patient. Ultimately, the prescribers or researchers will decide the appropriate amount and dosage regimen. Such determinations are routine to one of ordinary skill in the art.

As mentioned previously, neuro-inflammation and humoral immunity are involved in PD pathogenesis. From a therapeutic perspective, immunomodulatory drugs, including NSAIDS, aimed at down regulating inflammatory processes have are considered strong candidates for Parkinson disease treatment. From a prevention perspective, NSAID use, in particular ibuprofen, has been shown to associate with reduced risk of developing PD in humans. Aspects of the invention relate to the administration of over-the-counter and prescription NSAIDS separately to PD individuals in need thereof that may also possess different gene variants associated with a favorable response to each type of administration. This is reasonable because the underlying conditions for which prescription and over-the-counter NSAIDs are used for are often different.

According to the present invention, the dose of NSAID administered to a patient in need thereof may be, but is not limited to a range of about 5 mg to about 5000 mg daily. The NSAID may be administered in multiple doses, and may be determined by the attending physician.

The NSAIDs of the instant invention may include, but is not limited to ibuprofen, aspirin, naproxen, indomethacin, nabumetone, nimesulide, selective cyclooxygenase (COX) inhibitors, or licofelone.

Non-steroidal anti-inflammatory drugs (NSAIDs or NAIDs), sometimes also referred to as non-steroidal anti-inflammatory agents/analgesics (NSAIAs) or non-steroidal anti-inflammatory medicines (NSAIMs), are drugs with analgesic, antipyretic and, in higher doses, anti-inflammatory effects. NSAIDs can reduce pain, fever and inflammation. The term “non-steroidal” is used to distinguish these drugs from steroids, which have a similar cicosanoid-depressing, anti-inflammatory effect. As analgesics, NSAIDs are non-narcotic. NSAIDs include but are not limited to diclofenac, etodolac, indomethacin, sulindac, tolmetin, nabumetone, piroxicam, acetaminophen, fenoprofen, flurbiprofen, ibuprofen, ketoprofen, naproxen, oxaprozin, aspirin, choline magnesium trisalicylate, diflunisal, meclofenamic acid, mefenamic acid, phenylbutazone, fluocinolone acetonide, prednisolone, prednisolone tertiary-butylacetate, dexamethasone, or prodrugs or active metabolites thereof. The most prominent members of this group of drugs are aspirin, ibuprofen, and naproxen. Paracetamol (acetaminophen) has negligible anti-inflammatory activity, and is not an NSAID.

Most NSAIDs act as non-selective inhibitors of the enzyme cyclooxygenase, inhibiting both the cyclooxygenase-1 (COX-1) and cyclooxygenase-2 (COX-2) isoenzymes. Cyclooxygenase catalyzes the formation of prostaglandins and thromboxane from arachidonic acid. Prostaglandins act as messengers in the process of inflammation. NSAIDs are usually indicated for the treatment of acute or chronic conditions where pain and inflammation are present. COX2 inhibitors include but are not limited to rofecoxib (VIOXX™, or 4-[4-(methylsulfonyl)phenyl]-3-phenyl-2(5H)-furanone), celecoxib (CELEBREX™, or 445-(4-methylphenyl)-3-(trifluoromethyl)-1H-pyrazol-1-yl]benzenesulfonam-ide), and valdecoxib (BEXTRA™, and 4-(5-methyl-3-phenyl-4-isoxazolyl)benzenesulfonamide).

NSAIDs are generally indicated for the symptomatic relief of conditions including but not limited to rheumatoid arthritis, osteoarthritis, inflammatory arthropathies, acute gout, dysmenorrhoea, metastatic bone pain, headache and migraine, postoperative pain, mild-to-moderate pain due to inflammation and tissue injury, pyrexia (fever), ileus, and renal colic.

The NSAID may be a COX-2 inhibitor such as celecoxib, rofecoxib, meloxicam, piroxicam, valdecoxib, parecoxib, etoricoxib, CS-502, JTE-522, L-745,337 or NS398. Alternatively, the NSAID may be aspirin, acetaminophen, ibuprofen, flurbiprofen, ketoprofen, naproxen, oxaprozin, etodolac, indomethacin, ketorolac, lornoxicam, nabumetone, or diclofenac. Some embodiments of the invention may relate to NSAIDs that include but are not limited to a NSAID selected from the group consisting of: ibuprofen, aspirin, indomethacin, nabumetone, nimesulide, selective cyclooxygenase (COX) inhibitos, licofelone, Anaprox (naproxen), Arthrotec (diclofenac sodium), Bextra (valdecoxib), Cataflam (diclofenac potassium), Celebrex (celecoxib), Clinoril (sulindac), Dolobid (diflunisal), EC-naprosyn (naproxen), Feldene (piroxicam), Indocin (indomethacin), Mobic (meloxicam), Motrin (ibuprofen), Naprelan (naproxen controlled release), Naprosyn (naproxen), Ponstel (mefenamic acid), Relafen (nabumetone), Toradol (ketorolac tromethamine), Trilisate (choline magnesium salicylate), Vioxx (rofecoxib) and Voltaren (diclofenac sodium) The most preferred NSAID is in an amount of between 50 mg and 1500 mg, and more preferably, in an amount of between 200 mg and 600 mg. It will be understood that, for the purposes of the present invention, reference to an acid inhibitor, NSAID, or analgesic agent will include all of the common forms of these compounds and, in particular, their pharmaceutically acceptable salts. The amounts of NSAIDs which are therapeutically effective may be lower in the current invention than otherwise found in practice due to potential positive kinetic interaction and NSAID absorption in the presence of an acid inhibitor.

The term “unit dosage form” refers to a single entity for drug administration (see, e.g., U.S. Pat. No. 6,926,907). For example, a single tablet or capsule combining both an acid inhibitor and an NSAID would be a unit dosage form. A unit dosage form of the present invention preferably provides for coordinated drug release, in a way that elevates gastric pH and reduces the deleterious effects of the NSAID on the gastroduodenal mucosa, i.e., the acid inhibitor is released first and the release of NSAID is delayed until after the pH in the GI tract has risen. In a preferred embodiment, the unit dosage form is a multilayer tablet, having an outer layer comprising the acid inhibitor and an inner core which comprises the NSAID. In the most preferred form, coordinated delivery is accomplished by having the inner core surrounded by a polymeric barrier coating that does not dissolve unless the surrounding medium is at a pH of at least 3.5, preferably at least 4 and more preferably, at least 5. Alternatively, a barrier coating may be employed which controls the release of NSAID by time, as opposed to pH, with the rate adjusted so that NSAID is not released until after the pH of the gastrointestinal tract has risen to at least 3.5, preferably at least 4, and more preferably at least 5. Thus, a time-release formulation may be used to prevent the gastric presence of NSAID until mucosal tissue is no longer exposed to the damage enhancing effect of very low pH.

The invention includes methods of treating a patient for pain, inflammation and/or other conditions by administering the pharmaceutical compositions described above, being particularly useful in patients with osteoarthritis or rheumatoid arthritis. Other conditions that may be treated include, but are not limited to: all form of headache, including migraine headache; acute musculoskeletal pain; ankylosing spondylitis; dysmenorrhoea; myalgias; and neuralgias. More preferred embodiments of the invention relate to administering pharmaceutical compounds comprising a NSAID which by suppressing inflammation alleviate specific PD symptoms response that may include but are not limited to death of dopamine-generating cells in the substantia nigra, rest tremors, shaking, rigidity, bradykinesia, slowness of movement, difficulty with walking and gait, cognitive and behavioral problems, dementia, sensory, sleep and emotional problems and postural instability.

In a more general sense, the invention includes methods of treatment by orally administering an acid inhibitor at a dose effective to raise a patient's gastric pH to at least 3.5, preferably to at least 4 or and more preferably to at least 5. The patient is also administered an NSAID, for example in a coordinated dosage form, that has been coated in a polymer that only dissolves at a pH of least 3.5, preferably at least 4 and, more preferably, 5 or greater or which dissolves at a rate that is slow enough to prevent NSAID release until after the pH has been raised. When acid inhibitor and NSAID are administered in separate doses, e.g., in two separate tablets, they should be given concomitantly (i.e., so that their biological effects overlap) and may be given concurrently, i.e., NSAID is given within one hour after the acid inhibitor. Preferably, the acid inhibitor is an H2 blocker and, in the most preferred embodiment, it is famotidine at a dosage of between 5 mg and 100 mg. Any of the NSAIDs described above may be used in the method but naproxen at a dosage of between 200 and 600 mg is most preferred. It is expected that the inhibitor and analgesic will be typically delivered as part of a single unit dosage form which provides for the coordinated release of therapeutic agents. The most preferred dosage form is a multilayer tablet having an outer layer comprising an H2 blocker and an inner core comprising an NSAID.

See, e.g., U.S. Pat. Nos. 8,062,656; RE41,151; 7,111,346; 6,511,966; 6,429,223 and 6,355,666 for further disclosure regarding NSAIDs for the treatment of PD.

As mentioned previously, caffeine is a non-selective competitive adenosine A2A receptor antagonist; a family of compounds with neuroprotective properties [Chen et al. (2001) J Neurosci 21, RC143 and 56]. A2A receptor antagonists, such as Istradefylline have been tested for PD in clinical trials and shown promising results with fewer complications than current therapies [Factor (2008) Neurotherapeutics 5, 164-80]. Further studies relate to caffeinated-coffee consumption protecting some people from developing PD, although not all benefit equally. In Hamza T H, Chen H, Hill-Burns E M, Rhodes S L, Montimurro J, Kay D M, et al. Genome-Wide Gene-Environment Study Identifies Glutamate Receptor Gene GRIN2A as a Parkinson's Disease Modifier Gene via Interaction with Coffee. PLoS Genet 2011; 7(8): e1002237 (incorporated by reference in its entirety) a genome-wide search indicated that variations in the glutamate-receptor gene GRIN2A may modulate the risk of developing PD in heavy coffee drinkers. Administration of caffeine/A2ARA may not be considered or approved for PD treatment, despite being potentially safe and effective for prevention and treatment in some people, unless people who can benefit from it can be distinguished from those who do not.

The caffeine, caffeine analogs, nicotine or nicotine analogs of the present invention may be administered in dosages deemed appropriate by the attending physician.

Caffeine has a long history for use in the treatment of headaches. The term “caffeine” as used herein is intended to encompass not only caffeine as the anhydrous powder, but any salt or derivative of caffeine or any compounded mixture thereof, which is nontoxic and pharmaceutically acceptable. By itself, caffeine has little or no analgesic effect. However, caffeine will enhance, or potentiate, the analgesic activity of acetaminophen. In human trials, only a dose greater than 50 mg of caffeine will significantly potentiate acetaminophen or NSAID analgesia. Caffeine potentiation of acetaminophen analgesia is likely due to a change in the pharmacokinetics of acetaminophen.

Pharmaceutically effective combinations can typically contain between about 50 and about 200 mg of caffeine. The preferred pharmaceutically effective combinations contain between about 50 and about 150 mg of caffeine. Higher doses of caffeine, up to about 1000 mg, may be employed if tolerated by the patient.

Caffeine analogs and/or derivatives such as those disclosed in U.S. Pat. Nos. 8,137,984; 8,029,770; 7,569,396; 6,069,132; 5,763,625 may be contemplated for the present invention. Embodiments of the invention may relate to caffeine analogs that include but are not limited to Theophylline, 1-Proparagyl 3,7-Dimethyl Xanthine, 7-Proparagyl 1,3-Dimethyl Xanthine, 3-Proparagyl 1,7-Dimethyl Xanthine, 1,3,7-Triproparagyl Xanthine, IBMX, 1,3,7-Tripropyl Xanthine, 7-Benzyl-IBMX, 1-Propyl 3,7-Dimethyl Xanthine, 1,3-Dipropyl 7-Methyl Xanthine, 1,3-Dipropyl 7-Proparagyl Xanthine, 3,7-Dimethyl 1-Propyl Xanthine and 7-allyl 1,3-dimethyl Xanthine.

As mentioned previously, Nicotine treatment can partially protect against paraquat-induced nigrostriatal damage in mice [Khwaja et al. (2007) J Neurochem 100, 180-90]. Nicotine may protect DA neurons via an anti-inflammatory mechanism mediated by the modulation of microglial activation [Park et al. (2007) Eur J Neurosci 26, 79-89]. Monoamine oxidase inhibition [Shahi et al. (1991) Neurosci Lett 127, 247-50 and Castagnoli et al. (2001) Chem Res Toxicol 14, 523-7], and/or cytochrome P450 induction [Shahi et al. (1991) Neurosci Lett 127, 247-50], are thought to be important. Aspects of the genetic basis of the variable effects of smoking/nicotine in Parkinson's disease has been referred to in Hill-Burns E M et al. A genetic basis for the variable effect of smoking/nicotine on Parkinson's disease. The Pharmacogenomics Journal, (2 Oct. 2012). Administration of nicotine may not be considered or approved for PD treatment, despite being potentially safe and effective for prevention and treatment in some individuals, unless individuals who can benefit from it can be distinguished from those who do not.

Nicotine, the primary alkaloid in tobacco products binds stereo-selectively to nicotinic-cholinergic receptors on autonomic ganglia, the adrenal medulla, neuromuscular junctions and in the brain. Nicotine exerts two effects, a stimulant effect exerted at the locus ceruleus and a reward effect in the limbic system. Intravenous administration of nicotine causes release of acetylcholine, norepinephrine, dopamine, serotonin, vasopressin, beta-endorphin and ACTH. Nicotine is a highly addictive substance. Nicotine also induces peripheral vasoconstriction, tachycardia and elevated blood pressure. Nicotine inhalers and patches are used to treat smoking withdrawal syndrome. Nicotine is classified as a stimulant of autonomic ganglia. Nicotine, or 1-methyl-2-(3-pyridyl)pyrrolidone, is an oily colorless or pale yellow liquid with a pyridine odor, a molecular weight of about 162, an octanol:water partition coefficient (log P) of about 1.2, a dissociation constant (pKa) of about 3.1, a solubility in water of about and a melting point of approximately −79 C. Nicotine is miscible with water below 60 C. (See monograph of nicotine in Clarke's Analysis of Drugs and Poisons, Pharmaceutical Press 2005, the entire content of which is herein incorporated as reference). Nicotine is readily absorbed from the gastro-intestinal tract, the buccal mucosa, the respiratory tract, and intact skin, and widely distributed throughout the tissues. Nicotine undergoes extensive first-pass metabolism when administered orally, thus reducing the bioavailability. Oral bioavailability of nicotine is about 30%.

Nicotine numerous commercial uses include utilities such as a fumigant, an insecticide and the like. It is therapeutically valuable in the treatment of the smoking withdrawal syndrome. Nicotine has also been found therapeutically valuable in the treatment of other conditions involving release of dopamine such as attention deficit hyperactive disorder (ADHD), attention deficit disorder (ADD), Tourette's syndrome, schizophrenia, Alzheimer's disease, Parkinson's disease, anxiety and depression (see, e.g., U.S. Pat. Nos. 6,911,475; 6,479,076; 6,034,079, 5,278,176); in the therapeutic angiogenesis and vasculogenesis (see, e.g., U.S. Pat. No. 6,417,205); in the treatment of inflammatory bowel disease (see, e.g., U.S. Pat. No. 6,166,044)

The phrase “therapeutically effective amount” as used herein refers to a nontoxic but sufficient amount of a drug, agent, or compound to provide a desired therapeutic effect, for example, one or more doses of nicotine that will be effective in relieving symptoms of smoking cessation, inflammatory bowel disease, neurological disorder (e.g., anxiety, depression, schizophrenia, Alzheimer's Disease, Parkinson's Disease, Restless Legs Syndrome, Tourette's Syndrome, Chronic Tic Disorder, Essential Tremor, and Attention Deficit Hyperactivity Disorder).

The term “nicotine compound” as used herein refers to any of the conventional nicotine compounds, including nicotine, nicotine free base, pharmaceutically acceptable salts thereof, as well as mixtures of free base and salt forms. One example of a pharmaceutically acceptable salt of nicotine is the hydrogen tartrate salt (or, whose systematic name is pyridine, 3-(1-methyl-2-pyrrolidinyl)-, (S)—, (R—(R*,R*))-2,3-dihydroxybutanedioate (1:2), or nicotine, tartrate (1:2), or nicotine dihydrogen ditartrate).

Methods for production of nicotine derivatives and analogues are well known in the art. See, e.g., U.S. Pat. Nos. 4,590,278; 4,321,387; 4,452,984; 4,442,292; and 4,332,945.

Additional nicotine receptor agonists of interest include, but are not necessarily limited to, naturally occurring plant alkaloids (e.g., lobeline, lobeline derivatives, and the like), which plant-derived compounds can be provided in a herbal preparation (e.g., in the form of dried tobacco leaves, in a poultice, in a botanical preparation, etc.), in isolated form (e.g., separated or partially separated from the materials that naturally accompany it), or in a substantially purified form. Other nicotine receptor agonists include choline esterase inhibitors (acetylcholinesterase inhibitors) (e.g., inhibitors that increase local concentration of acetylcholine), derivatives of epibatidine that specifically bind the neuronal type of nicotinic receptors (with reduced binding to the muscarinic receptor) and having reduced deleterious side-effects (e.g., Epidoxidine, ABT-154, ABT-418, ABT-594; Abbott Laboratories (Damaj et al. (1998) J. Pharmacol Exp. Ther. 284:1058-65, describing several analogs of epibatidine of equal potency but with high specificity to the neuronal type of nicotinic receptors). Further nicotine receptor agonists of interest include, but are not necessarily limited to, N-methylcarbamyl and N-methylthi-O-carbamyl esters of choline (e.g., trimethylaminoethanol) (Abood et al. (1988) Pharmacol. Biochem. Behay. 30:403-8); acetylcholine (an endogenous ligand for the nicotine receptor); and the like.

Another example of a pharmaceutically acceptable salt of nicotine is nicotine, tartrate, hydrate (1:2:2) (synonym: nicotine bitartrate dihydrate). The molecular weight of nicotine hydrogen tartrate is approximately 498. As used herein after, the phrase “nicotine bitartrate dihydrate” and “nicotine hydrogen tartrate” are totally interchangeable.

The phrase “nicotine free base equivalent” (nicotine FBE) as used herein typically refers to the actual amount of the nicotine molecule in a formulation, that is, independent of the amount of the associated salt forming compound that is present in a nicotine salt. The phrase nicotine free base equivalent may be used to provide ease of comparison between formulations made using nicotine free base or any of a number of nicotine salts to show the amount of active ingredient (e.g., nicotine) that is present in the formulation. For example, free base nicotine has a molecular weight of approximately 162. Nicotine hydrogen tartrate has a molecular weight of approximately 462 of which approximately 300 of the molecular weight is attributed to tartaric acid. The molecular weight ratio of nicotine hydrogen tartrate to free base nicotine is 2.85. Accordingly, when nicotine hydrogen tartrate is present in a formulation at 4.28 weight percent this corresponds to a nicotine free base equivalent of 1.50 weight percent (4.28/2.85=1.50). Nicotine bitartrate dihydrate has a molecular weight of approximately 498 of which approximately 336 of the molecular weight is attributed to tartaric acid and water. The molecular weight ratio of nicotine bitartrate dihydrate to free base nicotine is 3.07. Accordingly, when nicotine bitartrate dihydrate is present in a formulation at 4.6 weight percent this corresponds to a nicotine free base equivalent of 1.50 weight percent (4.6/3.07=1.50).

The term “nicotine pharmaceutically acceptable salts” as used herein refers to formation of salts with acceptable salt formers such as, but not limited to, hydrochloride, sulphate, tosylate, mesylate, napsylate, besylate, maleate, phosphate, salicylate, tartrate, lactate, citrate, benzoate, succinate, acetate, pivalate, oxalate, picrate, phthalate, etc. As used herein, “nicotine pharmaceutically acceptable salts” can designate anhydrous salts or hydrated salts. Nicotine hydrated salts can be mono hydrated salts or polyhydrated salts. The term “nicotine pharmaceutically acceptable salts” as used herein also refers to formation of salts with polymers such as, but not limited to, methacrylic acid polymers, polyvinylpyrrolidone, polyvinyl alcohol, cyclodextrins, etc.

Nicotine therapy has been used to treat a variety of diseases and disorders of the central nervous system. Some specific conditions/disease states responsive to treatment with nicotine include, but are not limited to, anxiety, depression, schizophrenia, Alzheimer's Disease, Parkinson's Disease, Restless Legs Syndrome, Tourette's Syndrome, Chronic Tic Disorder, Essential Tremor, and Attention Deficit Hyperactivity Disorder, irritable bowel syndrome. See, e.g., U.S. Pat. No. 7,387,788.

The nicotine compositions of the present invention may be self-applied by a subject in need of treatment or the composition may be applied by a care-giver or health care professional. The compositions may be applied in single daily doses, multiple daily doses, or divided doses. Transdermal delivery of nicotine, as described herein, provides a number of advantages relative to oral dosing, including, but not limited to, continuous delivery which provides for steady-state blood levels of the nicotine, avoidance of the first-pass effect, and substantial avoidance of gastrointestinal and many other side effects. The likelihood of patient acceptance may also be much improved particularly among populations that have difficulty swallowing or chewing, for example, some elderly subjects and pediatrics. In view of the aforementioned, skin irritation arising from use of the non-occlusive compositions of the present invention is likely to be non-existent or minimal; however, evaluation of the degree of skin irritation caused by the nicotine formulations of the present invention may be tested in standard animal models.

Ease of application of the non-occlusive compositions of the present invention, for example, gel formulations comprising nicotine hydrogen tartrate, provides several advantages relative to oral administration of nicotine. For example, when the subject in need of treatment cannot self-medicate (e.g., young children or the infirmed) transdermal delivery avoids forcing subjects to take and swallow a pill or chew a gum. Further, transdermal application of the non occlusive compositions of the present invention assures correct dosing, versus a pill that may be inappropriately chewed (for example, when the pill is a time-release formulation), spit out, and/or regurgitated. Dose escalation or titration is particularly facilitated by a nicotine non occlusive transdermal gel in that larger doses may be administered by increasing the area of application to the skin while keeping the concentration of the formulation fixed.

In one embodiment of the present invention, up to about 5 grams of a gel formulation, having an amount of nicotine free base equivalents between about 0.5 and about 5 weight percent, is applied daily to a skin surface area of between about 50 to about 1000 cm². In another embodiment, up to about 5 grams of a gel formulation, having an amount of nicotine free base equivalents of about 1.5 weight percent, is applied daily to a skin surface area of between about 70 to about 1000 cm².

In one embodiment of the present invention, experiments performed in support of the present invention have provided good in vitro/in vivo correlation based on bioavailability of nicotine in the compositions of the present invention. These results are intended for illustration purposes only and to provide a general basis for in vitro/in vivo comparison, thus they should not be considered limiting. As a first example, in vitro/in vivo correlation based on bioavailability of formulations presented in the Examples hereinafter may be evaluated as follows. In vitro data can be extrapolated to in vivo conditions in order to evaluate the gel dose for bioequivalence to occlusive nicotine transdermal absorption. NICORETTE patches are transdermal delivery systems for topical application of nicotine base, available in sizes of 30, 20 and 10 cm² each containing 0.83 mg/cm² of nicotine, releasing 15 mg, 10 mg and 5 mg respectively over 16 hours (see, for example, NICORETTE®. Prescribing Information, GlaxoSmithKline, Middlesex UK). Considering the difference of transdermal in vitro bioavailability of formulation 7.2 in comparison with the intermediate dose of the 10 mg/16 hours NICORETTE patch, formulation 7.2 would be bioequivalent to the 10 mg/16 hours NICORETTE patch if about 3.7 g of the formulation 7.2 gel is applied over about 660 cm² of skin surface, for an intermediate gel loading of 5.6 mg/cm². This corresponds to a daily dose of 170 mg nicotine hydrogen tartrate (equivalent to about 60 mg free base).

Theoretical evaluations of transdermal nicotine delivery using exemplary compositions of the present invention have shown the feasibility to achieve therapeutic levels, for example, application of 0.5-10 g of gel at 4.6% nicotine hydrogen tartrate (equivalent to 1.5% nicotine free base) over 50-1000 cm² skin surface theoretically provides similar plasma levels as occlusive transdermal dose of NICORETTE patches.

Nicotine analogs are known in the art (see, e.g., Cerny et al., Onkologie, 25: 406-411 (2002); Lindblom et al., Respiration, 69: 254-260 (2002); de Villiers et al., Respiration, 69: 247-253 (2002); Tuncok et al., Exp. Clin. Psychopharmacol., 9: 228-234 (2001); Hieda et al., Int. J. Immunopharmacol., 22: 809-819 (2000); Pentel et al., Pharmacol. Biochem. Behay., 65: 191-198 (2000); Isomura et al., J. Org. Chem., 66: 4115-4121 (2001); and Meijler et al., J. Am. Chem. Soc., 125: 7164-7165 (2003). For example, embodiments of the invention may include nicotine analog that include but are not limited to N-succinyl-6-amino-(+/−)-nicotine (Castro et al., Biochem. Biophys. Res. Commun., 67: 583-589 (1975)), 6-(sigma-aminocapramido)-(+/−)-nicotine (Noguchi et al., Biochem. Biophys. Res. Comm., 83: 83-86 (1978)), O-succinyl-3′-hydroxymethyl-nicotine (Langone et al., Biochemistry, 12: 5025-5030 (1973); and Meth. Enzymol., 84: 628-640 (1982)) and 3′-(hydroxymethyl)-nicotine hemisuccinate (Langone et al., supra, Abad et al., Anal. Chem., 65: 3227-3231 (1993)). Nicotine analogs and/or derivatives such as those disclosed in U.S. Pat. Nos. 8,252,321; 7,888,512; 7,875,724; 7,820,826; 7,671,209; 7,553,968; 7,531,555; 7,501,520; 7,435,749; 7,387,788; 7,361,768; 7,341,745; 7,304,160; 7,179,917; 7,132,545; 7,112,678; 7,087,575; 7,067,672; 6,995,265; 6,911,475; 6,846,817; 6,596,740; 6,503,922; 6,479,076; 6,344,222; 6,238,689; 6,211,194; 6,177,451; 6,034,079; 5,929,034; 5,922,679; 5,780,433; 5,278,176; 6,232,082; 6,932,971 and 5,069,904 may be contemplated for the present invention.

Dopamine agonists of the present invention may include but are not limited to pramipexole (Mirapex®), ropinirole (Requip®), rotigotine (Neupro®), and apomorphine (Apokyn®). COMT inhibitors may include but are not limited to tolcapone (Tasmar®) and entacapone (Comtan®). MAO B inhibitors may include but are not limited to selegiline (Eldepryl®) and rasagiline (Azilect®). Anticholinergics may include but are not limited to trihexyphenidyl and benzotropine (Cogentin®). Antivirals may include but are not limited to amantadine (Symmetrel®).

The NSAIDs, caffeine or caffeine analog, nicotine or nicotine analogs, or a combinations thereof may be administered singly or in combination with levodopa, dopamine agonists, catechol O-methyltransferase (COMT) inhibitors, monoamine oxidase B (MAO B) inhibitors, anticholinergics, antivirals or a combination thereof.

The present invention also contemplates administering a cyclic GMP-specific phosphodiesterase inhibitor (see, e.g., U.S. Pat. No. 6,492,371).

As used herein an “analog” refers to a drug having a structure and function related to that of another drug but whose chemical and biological properties may be different. Analogs of the present invention include congeners. A “congener” as used herein refers to a substance generated or synthesized by essentially the same synthetic chemical reactions and the same procedures used for another substance. For example, caffeine is an adenosine A2A receptor antagonist, and istradefylline similarly functions as an adenosine A2A receptor antagonist, thus istradefylline is considered herein as a caffeine analog. As used herein “rs number” is the reference number for a SNP in Tables A, B, C or D that may be associated with PD.

“Gene symbol” refers to the gene in which a SNP is located.

“Chr” refers to the chromosome on which a SNP is located.

“Position” refers to the base-pair position on the chromosome where a SNP is located.

As used herein, “SNPs” may include approximately 1 million SNPs on the Illumina HumanOmni-Quad array that were tested for association with PD.

As used herein, “CNV” refers copy number variation including deletions and duplications that are commonly found across the genome.

As used herein “OR” refers to odds ratio and “P” refers to p value for the selected SNPs in Tables A, B, C or D. The chosen SNPs were selected based on p value, density of significant SNPs in the region, and odds ratio.

“High coffee” refers to subjects (PD patients and controls) that were stratified by their coffee consumption.

“Low coffee” refers to low coffee drinking PD patients vs. low coffee drinking controls.

“Coffee interaction” refers to a test wherein all subjects were included regardless of coffee consumption, testing whether the association of coffee with PD and SNP with PD are independent (if not, coffee and SNP are interacting in predisposing/protecting against PD).

“Ever smoke” refers to subjects that were stratified by smoking: smokers with PD vs. smoker controls.

“Never smoke” refers to non-smokers with PD vs. non-smoker controls.

“Smoke interaction” refers to a test wherein all subjects were included regardless of smoking status, testing whether the association of smoking with PD and SNP with PD are independent (if not, smoking and SNP are interacting).

Ever OTC” refers to PD patients who have used over the counter non-steroidal anti-inflammatory drugs vs. controls who have used over the counter non-steroidal anti-inflammatory drugs.

“Never OTC” refers to PD patients who have never used over the counter non-steroidal anti-inflammatory drugs vs. controls who have never used over the counter non-steroidal anti-inflammatory drugs.

“OTC interaction” refers to a test wherein all subjects were included regardless of OTC non-steroidal anti-inflammatory drug use, testing whether the association of OTC with PD and SNP with PD are independent (if not, OTC and SNP are interacting).

“Ever Rx” refers to PD patients who have used prescription non-steroidal anti-inflammatory drugs vs. controls who have used prescription non-steroidal anti-inflammatory drugs.

“Never Rx” refers to PD patients who have never used prescription non-steroidal anti-inflammatory drugs vs. controls who have never used prescription non-steroidal anti-inflammatory drugs.

“Rx interaction” refers to a test wherein all subjects were included regardless of prescription non-steroidal anti-inflammatory drug use, testing whether the association of Rx with PD and SNP with PD are independent (if not, Rx and SNP are interacting).

“Male” may refer to men with PD vs. men controls.

“Female” may refer to women with PD vs. women controls.

“Gender interaction” refers to a test wherein all subjects were included regardless of gender, testing whether the association of gender with PD and SNP with PD are independent (if not, gender and SNP are interacting).

“Early-onset” refers to PD patients with early-onset PD vs. all controls.

Late-onset” refers to PD patients with late-onset PD vs. all controls.

“Familial” refers to PD patient having a relative with PD.

“Sporadic” refers to PD patients having no known relative with PD.

As used herein “administering” or “applying” refers to a method of giving a dosage of a drug to a patient. The medicaments utilized in the methods of the invention can be administered by a route selected from, without limitation, inhalation, ocular, parenteral, dermal, transdermal, buccal, rectal, sublingual, perilingual, nasal, topical administration and oral administration. Parenteral administration includes intravenous, intraperitoneal, subcutaneous, and intramuscular administration. The preferred method of administration can vary depending on various factors, e.g., the components of the medicament being administered and the severity of the condition being treated.

Administration of compounds or medicaments in controlled release formulations is useful where the compound, either alone or in combination, has (i) a narrow therapeutic index (e.g., the difference between the plasma concentration leading to harmful side effects or toxic reactions and the plasma concentration leading to a therapeutic effect is small; generally, the therapeutic index, TI, is defined as the ratio of median lethal dose (LD₅₀) to median effective dose (ED₅₀)); (ii) a narrow absorption window in the gastro-intestinal tract; or (iii) a short biological half-life, so that frequent dosing during a day is required in order to sustain the plasma level at a therapeutic level.

Appropriate dosages of compounds used in the methods of the invention depend on several factors, including the administration method, the severity of the PD, and the age, weight, and health of the patient to be treated. Additionally, pharmacogenomic (the effect of genotype on the pharmacokinetic, pharmacodynamic or efficacy profile of a therapeutic) information about a particular patient may affect dosage used.

Continuous daily dosing with compounds used in the methods of the invention may not be required. A therapeutic regimen may require cycles, during which time a drug is not administered, or therapy may be provided on an as-needed basis.

Medicaments according to the invention may be formulated to release the active compound substantially immediately upon administration or at any predetermined time period after administration, using controlled release formulations.

Many strategies can be pursued to obtain controlled release in which the rate of release outweighs the rate of metabolism of the therapeutic compound. For example, controlled release can be obtained by the appropriate selection of formulation parameters and ingredients, including, e.g., appropriate controlled release medicaments and coatings. Suitable formulations are known to those of skill in the art. Examples include single or multiple unit tablet or capsule formulations, oil solutions, suspensions, emulsions, microcapsules, microspheres, nanoparticles, patches, and liposomes.

Formulations for oral use include tablets containing the active ingredient in a mixture 20 with non-toxic pharmaceutically acceptable excipients. These excipients may be, for example, inert diluents or fillers (e.g., sucrose and sorbitol), lubricating agents, glidants, and antiadhesives (e.g., magnesium stearate, zinc stearate, stearic acid, silicas, hydrogenated vegetable oils, or talc).

Formulations for oral use may also be provided as chewable tablets, or as hard gelatin capsules wherein the active ingredient is mixed with an inert solid diluent, or as soft gelatin capsules wherein the active ingredient is mixed with water or an oil medium.

As described above, the compound or medicament in question may be administered orally in the form of tablets, capsules, elixirs or syrups, or rectally in the form of suppositories. Parenteral administration of a compound is suitably performed, for example, in the form of saline solutions or with the compound incorporated into liposomes. In cases where the compound in itself is not sufficiently soluble to be dissolved, a solubilizer such as ethanol can be applied.

The dosage of any of the chemical compounds used in the methods of the invention can readily be determined by one skilled in the art. Desirably, the dosage of any of the chemical compounds used in the methods of the invention will be sufficient to ameliorate a symptom of PD in the patient.

As used herein “patient” refers to any human being receiving or who may receive medical treatment.

A “polymorphic site” refers to a polynucleotide that differs from another polynucleotide by one or more single nucleotide changes.

A “single nucleotide polymorphism” or “SNP” refers to polynucleotide that differs from another polynucleotide by a single nucleotide exchange. For example, without limitation, exchanging one A for one C, G, or T in the entire sequence of polynucleotide constitutes a SNP. Of course, it is possible to have more than one SNP in a particular polynucleotide. For example, at one locus in a polynucleotide, a C may be exchanged for a T, at another locus a G may be exchanged for an A, and so on. When referring to SNPs, the polynucleotide is most often DNA. Preferred embodiments of the invention relate to the SNPs which include but are not limited to rs4998386, rs30196, rs10214163 rs 2338971 and rs2072029.

A “proxy” or “proxy SNP” refers to any marker on the DNA, such as a SNP or a longer stretch of DNA showing variability in sequence or structure, that shows a correlation of 50% or higher with a reference SNP or longer stretch of DNA. Preferred embodiments of the invention relate to proxies or proxy SNPs which include but are not limited to proxies or proxy SNPS showing a correlation of 50% or higher with rs4998386, rs30196, rs10214163 rs 2338971 and rs2072029. Proxy SNPs may be identified using web based search programs that include but are not limited to SNAP Proxy Search, GLIDERS and programs in the GENEPI toolbox. In embodiments of the invention the proxies or proxy SNPs may have a correlation to a reference SNP or stretch of DNA or share a sequence identity that is at least 50%, 55%, 60%, 65%, 705, 75%, 80%, 85%, 90%, 95%, or 99%.

Therapy or treatment according to the invention may be performed alone or in conjunction with another therapy, and may be provided at home, the doctor's office, a clinic, a hospital's outpatient department, or a hospital. Treatment generally begins at a hospital so that the doctor can observe the therapy's effects closely and make any adjustments that are needed. The duration of the therapy depends on the age and condition of the patient, the stage of the patient's PD, and how the patient responds to the treatment. Additionally, a person having a greater risk of developing PD (e.g., a person who is genetically predisposed) may receive prophylactic treatment to inhibit or delay symptoms of the disease.

Other non-genetic methods of diagnosing patients as having or being at risk of having PD are well-known in the art and may be used with the instant invention. For example, the presence of one or more of the following symptoms may be used as part of a PD diagnosis: trembling, e.g., an involuntary, rhythmic tremor of one arm or one leg; muscular rigidity, stiffness, or discomfort; general slowness in any of the activities of daily living, e g, akinesia or bradykinesia; difficulty with walking, balance, or posture; alteration in handwriting; emotional changes; memory loss; speech problems; and difficulty sleeping. Review of a patient's symptoms, activity, medications, concurrent medical problems, or possible toxic exposures can be useful in making a PD diagnosis. In addition, a patient may be tested for the presence or absence of other genetic mutations that can indicate an increased likelihood of having PD. For example, the presence of one or more specific mutations or polymorphisms in the NURR1, alpha-synuclein, parkin, MAPT, DJ-1, PINK1, SNCA, NAT2, or LRRK2 genes may be used to diagnose a patient as having or being at risk of having PD. See, e.g., U.S. Patent Application Publication Nos. 2003-0119026 and 2005-0186591; Bonifati, Minerva Med. 96:175-186, 2005; and Cookson et al., Curr. Opin. Neurol. 18:706-711, 2005, each of which is hereby incorporated by reference.

The medicaments of the invention are prepared in a manner known to those skilled in the art, for example, by means of conventional dissolving, lyophilizing, mixing, granulating or confectioning processes. Methods well known in the art for making formulations are found, for example, in Remington: The Science and Practice of Pharmacy, 20th ed., ed. A. R. Gennaro, 2000, Lippincott Williams & Wilkins, Philadelphia, and Encyclopedia of Pharmaceutical Technology, eds. J. Swarbrick and J. C. Boylan, 1988-1999, Marcel Dekker, New York.

Administration of medicaments of the invention may be by any suitable means that results in a compound concentration that is effective for treating or inhibiting (e.g., by delaying) the development of PD. The compound is admixed with a suitable carrier substance, e.g., a pharmaceutically acceptable excipient that preserves the therapeutic properties of the compound with which it is administered. One exemplary pharmaceutically acceptable excipient is physiological saline. The suitable carrier substance is generally present in an amount of 1-95% by weight of the total weight of the medicament. The medicament may be provided in a dosage form that is suitable for oral, rectal, intravenous, intramuscular, subcutaneous, inhalation, nasal, topical or transdermal, vaginal, or ophthalmic administration. Thus, the medicament may be in form of, e.g., tablets, capsules, pills, powders, granulates, suspensions, emulsions, solutions, gels including hydrogels, pastes, ointments, creams, plasters, drenches, delivery devices, suppositories, enemas, injectables, implants, sprays, or aerosols.

In order to determine the genotype of a patient according to the methods of the present invention, it may be necessary to obtain a sample of genomic DNA from that patient. That sample of genomic DNA may be obtained from a sample of tissue or cells taken from that patient.

The tissue sample may comprise but is not limited to hair (including roots), skin, buccal swabs, blood, or saliva. The tissue sample may be marked with an identifying number or other indicia that relates the sample to the individual patient from which the sample was taken. The identity of the sample advantageously remains constant throughout the methods of the invention thereby guaranteeing the integrity and continuity of the sample during extraction and analysis. Alternatively, the indicia may be changed in a regular fashion that ensures that the data, and any other associated data, can be related back to the patient from whom the data was obtained. The amount/size of sample required is known to those skilled in the art.

Generally, the tissue sample may be placed in a container that is labeled using a numbering system bearing a code corresponding to the patient. Accordingly, the genotype of a particular patient is easily traceable.

In one embodiment of the invention, a sampling device and/or container may be supplied to the physician. The sampling device advantageously takes a consistent and reproducible sample from individual patients while simultaneously avoiding any cross-contamination of tissue. Accordingly, the size and volume of sample tissues derived from individual patients would be consistent.

According to the present invention, a sample of DNA is obtained from the tissue sample of the patient of interest. Whatever source of cells or tissue is used, a sufficient amount of cells must be obtained to provide a sufficient amount of DNA for analysis. This amount will be known or readily determinable by those skilled in the art.

DNA is isolated from the tissue/cells by techniques known to those skilled in the art (see, e.g., U.S. Pat. Nos. 6,548,256 and 5,989,431, Hirota et al., Jinrui Idengaku Zasshi. September 1989; 34(3):217-23 and John et al., Nucleic Acids Res. Jan. 25, 1991; 19(2):408; the disclosures of which are incorporated by reference in their entireties). For example, high molecular weight DNA may be purified from cells or tissue using proteinase K extraction and ethanol precipitation. DNA may be extracted from a patient specimen using any other suitable methods known in the art.

It is an object of the present invention to determine the genotype of a given patient of interest by analyzing the DNA from the patent, in order to identify a patient carrying specific alleles of the SNPs of the invention that are associated with developing PD.

There are many methods known in the art for determining the genotype of a patient and for identifying or analyzing whether a given DNA sample contains a particular SNP. Any method for determining genotype can be used for determining genotypes in the present invention. Such methods include, but are not limited to, amplimer sequencing, DNA sequencing, fluorescence spectroscopy, fluorescence resonance energy transfer (or “FRET”)-based hybridization analysis, high throughput screening, mass spectroscopy, nucleic acid hybridization, polymerase chain reaction (PCR), RFLP analysis and size chromatography (e.g., capillary or gel chromatography), all of which are well known to one of skill in the art. In particular, methods for determining nucleotide polymorphisms, particularly single nucleotide polymorphisms, are described in U.S. Pat. Nos. 6,514,700; 6,503,710; 6,468,742; 6,448,407; 6,410,231; 6,383,756; 6,358,679; 6,322,980; 6,316,230; and 6,287,766 and reviewed by Chen and Sullivan, Pharmacogenomics J 2003; 3(2):77-96, the disclosures of which are incorporated by reference in their entireties.

In one embodiment, the presence or absence of the SNPs of the present invention is determined by sequencing the region of the genomic DNA sample that spans the polymorphic locus. Many methods of sequencing genomic DNA are known in the art, and any such method can be used, see for example Sambrook et al., Molecular Cloning; A Laboratory Manual 2d ed. (1989). For example, as described below, a DNA fragment spanning the location of the SNP of interest can be amplified using the polymerase chain reaction or some other cyclic polymerase mediated amplification reaction. The amplified region of DNA can then be sequenced using any method known in the art. Advantageously, the nucleic acid sequencing is by automated methods (reviewed by Meldrum, Genome Res. September 2000; 10(9):1288-303, the disclosure of which is incorporated by reference in its entirety), for example using a Beckman CEQ 8000 Genetic Analysis System (Beckman Coulter Instruments, Inc.). Methods for sequencing nucleic acids include, but are not limited to, automated fluorescent DNA sequencing (see, e.g., Watts & MacBeath, Methods Mol Biol. 2001; 167:153-70 and MacBeath et al., Methods Mol Biol. 2001; 167:119-52), capillary electrophoresis (see, e.g., Bosserhoff et al., Comb Chem High Throughput Screen. December 2000; 3(6):455-66), DNA sequencing chips (see, e.g., Jain, Pharmacogenomics. August 2000; 1(3):289-307), mass spectrometry (see, e.g., Yates, Trends Genet. January 2000; 16(1):5-8), pyrosequencing (see, e.g., Ronaghi, Genome Res. January 2001; 11(1):3-11), and ultrathin-layer gel electrophoresis (see, e.g., Guttman & Ronai, Electrophoresis. December 2000; 21 (18):3952-64), the disclosures of which are hereby incorporated by reference in their entireties. The sequencing can also be done by any commercial company. Examples of such companies include, but are not limited to, the University of Georgia Molecular Genetics Instrumentation Facility (Athens, Ga.) or SeqWright DNA Technologies Services (Houston, Tex.).

In certain embodiments of the present invention, the detection of a given SNP can be performed using cyclic polymerase-mediated amplification methods. Any one of the methods known in the art for amplification of DNA may be used, such as for example, the polymerase chain reaction (PCR), the ligase chain reaction (LCR) (Barany, F., Proc. Natl. Acad. Sci. (U.S.A.) 88:189-193 (1991)), the strand displacement assay (SDA), or the oligonucleotide ligation assay (“OLA”) (Landegren, U. et al., Science 241:1077-1080 (1988)). Nickerson, D. A. et al. have described a nucleic acid detection assay that combines attributes of PCR and OLA (Nickerson, D. A. et al., Proc. Natl. Acad. Sci. (U.S.A.) 87:8923-8927 (1990)). Other known nucleic acid amplification procedures, such as transcription-based amplification systems (Malek, L. T. et al., U.S. Pat. No. 5,130,238; Davey, C. et al., European Patent Application 329,822; Schuster et al., U.S. Pat. No. 5,169,766; Miller, H. I. et al., PCT Application WO89/06700; Kwoh, D. et al., Proc. Natl. Acad. Sci. (U.S.A.) 86:1173 (1989); Gingeras, T. R. et al., PCT Application WO88/10315)), or isothermal amplification methods (Walker, G. T. et al., Proc. Natl. Acad. Sci. (U.S.A.) 89:392-396 (1992)) may also be used.

The most advantageous method of amplifying DNA fragments containing the SNPs of the invention employs PCR (see e.g., U.S. Pat. Nos. 4,965,188; 5,066,584; 5,338,671; 5,348,853; 5,364,790; 5,374,553; 5,403,707; 5,405,774; 5,418,149; 5,451,512; 5,470,724; 5,487,993; 5,523,225; 5,527,510; 5,567,583; 5,567,809; 5,587,287; 5,597,910; 5,602,011; 5,622,820; 5,658,764; 5,674,679; 5,674,738; 5,681,741; 5,702,901; 5,710,381; 5,733,751; 5,741,640; 5,741,676; 5,753,467; 5,756,285; 5,776,686; 5,811,295; 5,817,797; 5,827,657; 5,869,249; 5,935,522; 6,001,645; 6,015,534; 6,015,666; 6,033,854; 6,043,028; 6,077,664; 6,090,553; 6,168,918; 6,174,668; 6,174,670; 6,200,747; 6,225,093; 6,232,079; 6,261,431; 6,287,769; 6,306,593; 6,440,668; 6,468,743; 6,485,909; 6,511,805; 6,544,782; 6,566,067; 6,569,627; 6,613,560; 6,613,560 and 6,632,645; the disclosures of which are incorporated by reference in their entireties), using primer pairs that are capable of hybridizing to the proximal sequences that define or flank a polymorphic site in its double-stranded form.

To perform a cyclic polymerase mediated amplification reaction according to the present invention, the primers are hybridized or annealed to opposite strands of the target DNA, the temperature is then raised to permit the thermostable DNA polymerase to extend the primers and thus replicate the specific segment of DNA spanning the region between the two primers. Then the reaction is thermocycled so that at each cycle the amount of DNA representing the sequences between the two primers is doubled, and specific amplification of gene DNA sequences, if present, results.

Any of a variety of polymerases can be used in the present invention. For thermocyclic reactions, the polymerases are thermostable polymerases such as Taq, KlenTaq, Stoffel Fragment, Deep Vent, Tth, Pfu, Vent, and UlTma, each of which are readily available from commercial sources. For non-thermocyclic reactions, and in certain thermocyclic reactions, the polymerase will often be one of many polymerases commonly used in the field, and commercially available, such as DNA pol 1, Klenow fragment, T7 DNA polymerase, and T4 DNA polymerase. Guidance for the use of such polymerases can readily be found in product literature and in general molecular biology guides.

Typically, the annealing of the primers to the target DNA sequence is carried out for about 2 minutes at about 37-55° C., extension of the primer sequence by the polymerase enzyme (such as Taq polymerase) in the presence of nucleoside triphosphates is carried out for about 3 minutes at about 70-75° C., and the denaturing step to release the extended primer is carried out for about 1 minute at about 90-95° C. However, these parameters can be varied, and one of skill in the art would readily know how to adjust the temperature and time parameters of the reaction to achieve the desired results. For example, cycles may be as short as 10, 8, 6, 5, 4.5, 4, 2, 1, 0.5 minutes or less.

Also, “two temperature” techniques can be used where the annealing and extension steps may both be carried out at the same temperature, typically between about 60-65° C., thus reducing the length of each amplification cycle and resulting in a shorter assay time.

Typically, the reactions described herein are repeated until a detectable amount of product is generated. Often, such detectable amounts of product are between about 10 ng and about 100 ng, although larger quantities, e.g. 200 ng, 500 ng, 1 mg or more can also, of course, be detected. In terms of concentration, the amount of detectable product can be from about 0.01 pmol, 0.1 pmol, 1 pmol, 10 pmol, or more. Thus, the number of cycles of the reaction that are performed can be varied, the more cycles are performed, the more amplified product is produced. In certain embodiments, the reaction comprises 2, 5, 10, 15, 20, 30, 40, 50, or more cycles.

For example, the PCR reaction may be carried out using about 25-50 μl samples containing about 0.01 to 1.0 ng of template amplification sequence, about 10 to 100 pmol of each generic primer, about 1.5 units of Taq DNA polymerase (Promega Corp.), about 0.2 mM dDATP, about 0.2 mM dCTP, about 0.2 mM dGTP, about 0.2 mM dTTP, about 15 mM MgCl.sub.2, about 10 mM Tris-HCl (pH 9.0), about 50 mM KCl, about 1 μg/ml gelatin, and about 10 ill/μl Triton X-100 (Saiki, 1988).

Those of skill in the art are aware of the variety of nucleotides available for use in the cyclic polymerase mediated reactions. Typically, the nucleotides will consist at least in part of deoxynucleotide triphosphates (dNTPs), which are readily commercially available. Parameters for optimal use of dNTPs are also known to those of skill, and are described in the literature. In addition, a large number of nucleotide derivatives are known to those of skill and can be used in the present reaction. Such derivatives include fluorescently labeled nucleotides, allowing the detection of the product including such labeled nucleotides, as described below. Also included in this group are nucleotides that allow the sequencing of nucleic acids including such nucleotides, such as chain-terminating nucleotides, dideoxynucleotides and boronated nuclease-resistant nucleotides. Commercial kits containing the reagents most typically used for these methods of DNA sequencing are available and widely used. Other nucleotide analogs include nucleotides with bromo-, iodo-, or other modifying groups, which affect numerous properties of resulting nucleic acids including their antigenicity, their replicatability, their melting temperatures, their binding properties, etc. In addition, certain nucleotides include reactive side groups, such as sulfhydryl groups, amino groups, N-hydroxysuccinimidyl groups, that allow the further modification of nucleic acids comprising them.

The present invention provides oligonucleotides that can be used as primers to amplify specific nucleic acid sequences of a gene in cyclic polymerase-mediated amplification reactions, such as PCR reactions. These primers are useful in detecting the SNPs in a gene. In certain embodiments, these primers consist of oligonucleotide fragments. Such fragments should be of sufficient length to enable specific annealing or hybridization to the nucleic acid sample. The sequences typically will be about 8 to about 44 nucleotides in length, but may be longer. Longer sequences, e.g., from about 14 to about 50, are advantageous for certain embodiments.

In embodiments where it is desired to amplify a fragment of DNA comprising the SNPs, primers having contiguous stretches of about 8, 9, 10, 11, 12, 13, 14, 15, 16, 17, 18, 19, 20, 21, 22, 23, or 24 nucleotides from a gene sequence are contemplated.

Although various different lengths of primers can be used, and the exact location of the stretch of contiguous nucleotides in a gene used to make the primer can vary, it is important that the sequences to which the forward and reverse primers anneal are located on either side of the particular nucleotide position that is substituted in the SNP to be amplified.

The methods may employ primers located on either side of, and not overlapping with, the SNP in order to amplify a fragment of DNA that includes the nucleotide position at which the SNP is located. Such methods require additional steps, such as sequencing of the fragment, or hybridization of allele specific probes to the fragment, in order to determine the genotype at the polymorphic site. However, in some embodiments of the present invention, the amplification method is itself a method for determining the genotype of the polymorphic site, as for example, in “allele-specific PCR”. In allele-specific PCR, primer pairs are chosen such that amplification itself is dependent upon the input template nucleic acid containing the polymorphism of interest. In such embodiments, primer pairs are chosen such that at least one primer spans the actual nucleotide position of the SNP and is therefore an allele-specific oligonucleotide primer. Typically, the primers contain a single allele-specific nucleotide at the 3′ terminus preceded by bases that are complementary to the gene of interest. The PCR reaction conditions are adjusted such that amplification by a DNA polymerase proceeds from matched 3′-primer termini, but does not proceed where a mismatch occurs. Allele specific PCR can be performed in the presence of two different allele-specific primers, one specific for each allele, where each primer is labeled with a different dye, for example one allele specific primer may be labeled with a green dye (e.g. fluorescein) and the other allele specific primer labeled with a red dye (e.g. sulforhodamine). Following amplification, the products are analyzed for green and red fluorescence. The aim is for one homozygous genotype to yield green fluorescence only, the other homozygous genotype to give red fluorescence only, and the heterozygous genotype to give mixed red and green fluorescence.

Methods for performing allele specific PCR are well known in the art, and any such methods may be used. For example suitable methods are taught in Myakishev et al. Genome Research, vol 1, p 163-169 (2001), Alexander et al. Mol Biotechnol. vol 28(3), p 171-174 (2004), and Ruano et al. Nucleic Acids Res. vol 17(20), p 8392 (1989), the contents of which are incorporated by reference. In some embodiments of the present invention, allele-specific primers are chosen so that amplification creates a restriction site, facilitating identification of a polymorphic site. To perform, allele specific PCR the reaction conditions must be carefully adjusted such that the allele specific primer will only bind to one allele and not the alternative allele, for example, in some embodiments the conditions are adjusted so that the primers will only bind where there is a 100% match between the primer sequence and the DNA, and will not bind if there is a single nucleotide mismatch.

In certain embodiments of the present invention, the detection of a given SNP can be performed using oligonucleotide probes that bind or hybridize to the DNA. The present invention provides oligonucleotide probes to detect SNPs in a gene.

In certain embodiments, these probes consist of oligonucleotide fragments. Such fragments should be of sufficient length to provide specific hybridization to the nucleic acid sample. The sequences typically will be about 8 to about 50 nucleotides, but may be longer. Nucleic acid probes having contiguous stretches of about 8, 9, 10, 11, 12, 13, 14, 15, 16, 17, 18, 19, 20, 21, 22, 23, or 24 nucleotides from a sequence selected from a gene sequence are contemplated.

Although various different lengths of probes can be used, and the precise location of the stretch of contiguous nucleotides in a gene from which the probe sequence is derived can vary, the probe sequence must span the particular nucleotide position that is substituted in the particular SNP to be detected.

These probes will be useful in a variety of hybridization embodiments, such as Southern blotting, Northern blotting, and hybridization disruption analysis. Also the probes of the invention can be used to detect SNPs in amplified sequences, such as amplified PCR products generated using the primers described above. For example, in one embodiment a target nucleic acid is first amplified, such as by PCR or strand displacement amplification (SDA), and the amplified double stranded DNA product is then denatured and hybridized with a probe.

In other embodiments double stranded DNA (amplified or not) is denatured and hybridized with a probe of the present invention and then the hybridization complex is subjected to destabilizing or disrupting conditions. By determining the level of disruption energy required wherein the probe has different disruption energy for one allele as compared to another allele, the genotype of a gene at a polymorphic locus can be determined. In one example, there can be lower disruption energy, e.g., melting temperature, for an allele that harbors a cytosine residue at a polymorphic locus, and a higher required energy for an allele with a thymine residue at that polymorphic locus. This can be achieved where the probe has 100% homology with one allele (a perfectly matched probe), but has a single mismatch with the alternative allele. Since the perfectly matched probe is bound more tightly to the target DNA than the mismatched probe, it requires more energy to cause the hybridized probe to dissociate.

In one embodiment the destabilizing conditions comprise an elevation of temperature. The higher the temperature, the greater the degree of destabilization. In another embodiment, the destabilizing conditions comprise subjecting the hybridization complex to a temperature gradient, whereby, as the temperature is increased, the degree of destabilization increases. In an alternative embodiment, the destabilizing conditions comprise treatment with a destabilizing compound, or a gradient comprising increasing amounts of such a compound. Suitable destabilizing compounds include, but are not limited to, salts and urea. Methods of destabilizing or denaturing hybridization complexes are well known in the art, and any such method may be used in accordance with the present invention. For example, methods of destabilizing or denaturing hybridization complexes are taught by Sambrook et al., Molecular Cloning; A Laboratory Manual 2d ed. (1989).

For optimal detection of single-base pair mismatches, it is preferable that there is about a 1° C. to about a 10° C. difference in melting temperature of the probe DNA complex when bound to one allele as opposed to the alternative allele at the polymorphic site. Thus, when the temperature is raised above the melting temperature of a probe:DNA duplex corresponding to one of the alleles, that probe will disassociate.

In one embodiment of the above method, a second (“anchor”) probe can be used. Generally, the anchor probe is not specific to either allele, but hybridizes regardless of what nucleotide is present at the polymorphic locus. The anchor probe does not affect the disruption energy required to disassociate the hybridization complex but, instead, contains a complementary label for using with the first (“sensor”) probe, for example for use in fluorescence resonance energy transfer or “FRET”. A sensor probe acquires energy from the anchor probe once conditions are adequate for hybridization between the target DNA and the anchor and sensor probes. Once hybridization occurs, the anchor probe transfers its florescence energy to the sensor probe, which only will emit a specific wavelength after it has acquired the energy from the anchor probe. Detection of the SNP occurs as the temperature is raised at a predetermined rate, and a reading is acquired from the florescent light emitted. If there is a single base mismatch of the probe and target DNA caused by the presence of the alternative polymorphic nucleotide (i.e. the SNP) the sensor probe will dissociate sooner, or at a lower temperature, since the homology between the genomic DNA and the sensor probe will be less than that of genomic DNA that does not harbor the altered nucleotide or SNP. Thus, there will be a loss of fluorescence that can be detected. Where the probe is designed to bind to the wild-type sequence, the dissociation of the probe from the DNA (i.e. the “melting”) will occur at a lower temperature if the SNP is present, since the stability of the binding of the probe to the SNP is slightly less than for the wild-type sequence. This occurs, obviously, on both chromosomes at the same time, thus yielding either a reading of two identical melting temperatures for a homozygote, or a reading of two different melting temperatures for the heterozygote. For example, where a probe is designed to have the sequence of the C-containing allele of the UASMS 1 polymorphism, the probe will dissociate or melt at a lower temperature in DNA samples from individuals that harbor two copies of the polymorphic T-containing allele, than in individuals that harbor two copies of the C-containing allele.

In other embodiments, two different “allele-specific probes” can be used for analysis of a SNP, a first allele-specific probe for detection of one allele, and a second allele-specific probe for the detection of the alternative allele. In another embodiment the different alleles of the polymorphism may be detected using two different allele-specific probes.

Whichever probe sequences and hybridization methods are used, one skilled in the art can readily determine suitable hybridization conditions, such as temperature and chemical conditions. Such hybridization methods are well known in the art. For example, for applications requiring high selectivity, one will typically desire to employ relatively stringent conditions for the hybridization reactions, e.g., one will select relatively low salt and/or high temperature conditions, such as provided by about 0.02 M to about 0.10 M NaCl at temperatures of about 50°C to about 70° C. Such high stringency conditions tolerate little, if any, mismatch between the probe and the template or target strand, and are particularly suitable for detecting specific SNPs according to the present invention. It is generally appreciated that conditions can be rendered more stringent by the addition of increasing amounts of formamide. Other variations in hybridization reaction conditions are well known in the art (see for example, Sambrook et al., Molecular Cloning; A Laboratory Manual 2d ed. (1989)).

In addition to the SNPs described above, it will be appreciated by those skilled in the art that other DNA sequence polymorphisms of a gene may exist. Such natural allelic variations can typically result in about 1-5% variance in the nucleotide sequence of the gene. It is possible that other polymorphic loci may also exist within this fragment. In addition to naturally-occurring allelic variants of the nucleotide sequence, the skilled artisan will further appreciate that changes can be introduced by mutation into the nucleotide sequence of the nucleotide sequences described herein. Any and all such additional nucleotide variations are intended to be within the scope of the invention. Thus, for example a probe according to the present invention may be designed to bind to a sequence of a gene containing not only the SNPs described herein, but also other SNPs that may occur within the same region.

Moreover, nucleic acid molecules that differ from the sequences of the primers and probes disclosed herein, are intended to be within the scope of the invention. Nucleic acid sequences that are complementary to these sequences, or that are hybridizable to the sequences described herein under conditions of standard or stringent hybridization, and also analogs and derivatives are also intended to be within the scope of the invention. Advantageously, such variations will differ from the sequences described herein by only a small number of nucleotides, for example by 1, 2, or 3 nucleotides.

Nucleic acid molecules corresponding to natural allelic variants, homologues (i.e., nucleic acids derived from other species), or other related sequences (e.g., paralogs) of the sequences described herein can be isolated based on their homology to the nucleic acids disclosed herein, for example by performing standard or stringent hybridization reactions using all or a portion of the sequences of the invention as probes. Such methods for nucleic acid hybridization and cloning are well known in the art.

Similarly, a nucleic acid molecule of the invention may include only a fragment of the specific sequences described. Fragments provided herein are defined as sequences of at least 6 (contiguous) nucleic acids, a length sufficient to allow for specific hybridization of nucleic acid primers or probes, and are at most some portion less than a full-length sequence. Fragments may be derived from any contiguous portion of a nucleic acid sequence of choice. Derivatives and analogs may be full length or other than full length, if the derivative or analog contains a modified nucleic acid or amino acid, as described below.

Derivatives, analogs, homologues, and variants of the nucleic acids of the invention include, but are not limited to, molecules comprising regions that are substantially homologous to the nucleic acids of the invention, in various embodiments, by at least about 70%, 80%, 85%, 90%, 95%, 96%, 97%, 98%, or even 99% identity over a nucleic acid sequence of identical size or when compared to an aligned sequence in which the alignment is done by a computer homology program known in the art.

The primers and probes described herein may be readily prepared by, for example, directly synthesizing the fragment by chemical means or by introducing selected sequences into recombinant vectors for recombinant production. Methods for making a vector or recombinants or plasmid for amplification of the fragment either in vivo or in vitro can be any desired method, e.g., a method which is by or analogous to the methods disclosed in, or disclosed in documents cited in: U.S. Pat. Nos. 4,603,112; 4,769,330; 4,394,448; 4,722,848; 4,745,051; 4,769,331; 4,945,050; 5,494,807; 5,514,375; 5,744,140; 5,744,141; 5,756,103; 5,762,938; 5,766,599; 5,990,091; 5,174,993; 5,505,941; 5,338,693; 5,494,807; 5,591,639; 5,589,466; 5,677,178; 5,591,439; 5,552,143; 5,580,859; 6,130,066; 6,004,777; 6,130,066; 6,497,883; 6,464,984; 6,451,770; 6,391,314; 6,387,376; 6,376,473; 6,368,603; 6,348,196; 6,306,400; 6,228,846; 6,221,362; 6,217,883; 6,207,166; 6,207,165; 6,159,477; 6,153,199; 6,090,393; 6,074,649; 6,045,803; 6,033,670; 6,485,729; 6,103,526; 6,224,882; 6,312,682; 6,348,450; and 6,312,683; U.S. patent application Ser. No. 920,197, filed Oct. 16, 1986; WO 90/01543; WO91/11525; WO 94/16716; WO 96/39491; WO 98/33510; EP 265785; EP 0 370 573; Andreansky et al., Proc. Natl. Acad. Sci. USA 1996; 93:11313-11318; Ballay et al., EMBO J. 1993; 4:3861-65; Feigner et al., J. Biol. Chem. 1994; 269:2550-2561; Frolov et al., Proc. Natl. Acad. Sci. USA 1996; 93:11371-11377; Graham, Tibtech 1990; 8:85-87; Grunhaus et al., Sem. Virol. 1992; 3:237-52; Ju et al., Diabetologia 1998; 41:736-739; Kitson et al., J. Virol. 1991; 65:3068-3075; McClements et al., Proc. Natl. Acad. Sci. USA 1996; 93:11414-11420; Moss, Proc. Natl. Acad. Sci. USA 1996; 93:11341-11348; Paoletti, Proc. Natl. Acad. Sci. USA 1996; 93:11349-11353; Pennock et al., Mol. Cell. Biol. 1984; 4:399-406; Richardson (Ed), Methods in Molecular Biology 1995; 39, “Baculovirus Expression Protocols,” Humana Press Inc.; Smith et al. (1983) Mol. Cell. Biol. 1983; 3:2156-2165; Robertson et al., Proc. Natl. Acad. Sci. USA 1996; 93:11334-11340; Robinson et al., Sem. Immunol. 1997; 9:271; and Roizman, Proc. Natl. Acad. Sci. USA 1996; 93:11307-11312.

Oligonucleotide sequences used as primers or probes according to the present invention may be labeled with a detectable moiety. As used herein the term “sensors” refers to such primers or probes labeled with a detectable moiety. Various labeling moieties are known in the art. Said moiety may be, for example, a radiolabel (e.g., 3H, 125I, 35S, 14C, 32P, etc.), detectable enzyme (e.g. horse radish peroxidase (HRP), alkaline phosphatase etc.), a fluorescent dye (e.g., fluorescein isothiocyanate, Texas red, rhodamine, Cy3, Cy5, Bodipy, Bodipy Far Red, Lucifer Yellow, Bodipy 630/650-X, Bodipy R6G-X and 5-CR 6G, and the like), a colorimetric label such as colloidal gold or colored glass or plastic (e.g. polystyrene, polypropylene, latex, etc.), beads, or any other moiety capable of generating a detectable signal such as a colorimetric, fluorescent, chemiluminescent or electrochemiluminescent (ECL) signal.

Primers or probes may be labeled directly or indirectly with a detectable moiety, or synthesized to incorporate the detectable moiety. In one embodiment, a detectable label is incorporated into a nucleic acid during at least one cycle of a cyclic polymerase-mediated amplification reaction. For example, polymerases can be used to incorporate fluorescent nucleotides during the course of polymerase-mediated amplification reactions. Alternatively, fluorescent nucleotides may be incorporated during synthesis of nucleic acid primers or probes. To label an oligonucleotide with the fluorescent dye, one of conventionally-known labeling methods can be used (Nature Biotechnology, 14, 303-308, 1996; Applied and Environmental Microbiology, 63, 1143-1147, 1997; Nucleic Acids Research, 24, 4532-4535, 1996). An advantageous probe is one labeled with a fluorescent dye at the 3′ or 5′ end and containing G or C as the base at the labeled end. If the 5′ end is labeled and the 3′ end is not labeled, the OH group on the C atom at the 3′-position of the 3′ end ribose or deoxyribose may be modified with a phosphate group or the like although no limitation is imposed in this respect.

Spectroscopic, photochemical, biochemical, immunochemical, electrical, optical or chemical means can be used to detect such labels. The detection device and method may include, but is not limited to, optical imaging, electronic imaging, imaging with a CCD camera, integrated optical imaging, and mass spectrometry. Further, the amount of labeled or unlabeled probe bound to the target may be quantified. Such quantification may include statistical analysis. In other embodiments the detection may be via conductivity differences between concordant and discordant sites, by quenching, by fluorescence perturbation analysis, or by electron transport between donor and acceptor molecules.

In yet another embodiment, detection may be via energy transfer between molecules in the hybridization complexes in PCR or hybridization reactions, such as by fluorescence energy transfer (FET) or fluorescence resonance energy transfer (FRET). In FET and FRET methods, one or more nucleic acid probes are labeled with fluorescent molecules, one of which is able to act as an energy donor and the other of which is an energy acceptor molecule. These are sometimes known as a reporter molecule and a quencher molecule respectively. The donor molecule is excited with a specific wavelength of light for which it will normally exhibit a fluorescence emission wavelength. The acceptor molecule is also excited at this wavelength such that it can accept the emission energy of the donor molecule by a variety of distance-dependent energy transfer mechanisms. Generally the acceptor molecule accepts the emission energy of the donor molecule when they are in close proximity (e.g. on the same, or a neighboring molecule). FET and FRET techniques are well known in the art, and can be readily used to detect the SNPs of the present invention. See for example U.S. Pat. Nos. 5,668,648, 5,707,804, 5,728,528, 5,853,992, and 5,869,255 (for a description of FRET dyes), Tyagi et al. Nature Biotech. vol. 14, p 303-8 (1996), and Tyagi et al., Nature Biotech. vol 16, p 49-53 (1998) (for a description of molecular beacons for FET), and Mergny et al. Nucleic Acid Res. vol 22, p 920-928, (1994) and Wolf et al. PNAS vol 85, p 8790-94 (1988) (for general descriptions and methods fir FET and FRET), each of which is hereby incorporated by reference.

The oligonucleotide primers and probes of the present invention have commercial applications in diagnostic kits for the detection of the gene SNPs in patients. A test kit according to the invention may comprise any of the oligonucleotide primers or probes according to the invention. Such a test kit may additionally comprise one or more reagents for use in cyclic polymerase mediated amplification reactions, such as DNA polymerases, nucleotides (dNTPs), buffers, and the like. An SNP detection kit may also include, a lysing buffer for lysing cells contained in the specimen. In a particular advantageous embodiment, a diagnostic for the present invention may comprise testing for any of the genes in Tables A, B, C and/or D. In a particularly advantageous embodiment, the diagnostic may comprise testing for any of the genes and SNPs in Table A. In another particularly advantageous embodiment, the diagnostic may comprise testing for any of the genes and SNPs in Table B. In still another particularly advantageous embodiment, the diagnostic may comprise testing for any of the genes and SNPs in Table C. In yet another particularly advantageous embodiment, the diagnostic may comprise testing for any of the genes and SNPs in Table D.

A test kit according to the invention may comprise a pair of oligonucleotide primers according to the invention and a probe comprising an oligonucleotide according to the invention. In some embodiments such a kit will contain two allele specific oligonucleotide probes. Advantageously, the kit further comprises additional means, such as reagents, for detecting or measuring the binding or the primers and probes of the present invention, and also ideally a positive and negative control.

The present invention further encompasses probes according to the present invention that are immobilized on a solid or flexible support, such as paper, nylon or other type of membrane, filter, chip, glass slide, microchips, microbeads, or any other such matrix, all of which are within the scope of this invention. The probe of this form is now called a “DNA chip”. These DNA chips can be used for analyzing the SNPs of the present invention. The present invention further encompasses arrays or microarrays of nucleic acid molecules that are based on one or more of the sequences described herein. As used herein “arrays” or “microarrays” refers to an array of distinct polynucleotides or oligonucleotides synthesized on a solid or flexible support, such as paper, nylon or other type of membrane, filter, chip, glass slide, or any other suitable solid support. In one embodiment, the microarray is prepared and used according to the methods and devices described in U.S. Pat. Nos. 5,446,603; 5,545,531; 5,807,522; 5,837,832; 5,874,219; 6,114,122; 6,238,910; 6,365,418; 6,410,229; 6,420,114; 6,432,696; 6,475,808 and 6,489,159 and PCT Publication No. WO 01/45843 A2, the disclosures of which are incorporated by reference in their entireties.

For the purposes of the present invention, sequence identity or homology is determined by comparing the sequences when aligned so as to maximize overlap and identity while minimizing sequence gaps. In particular, sequence identity may be determined using any of a number of mathematical algorithms. A nonlimiting example of a mathematical algorithm used for comparison of two sequences is the algorithm of Karlin & Altschul, Proc. Natl. Acad. Sci. USA 1990; 87: 2264-2268, modified as in Karlin & Altschul, Proc. Natl. Acad. Sci. USA 1993; 90: 5873-5877.

Another example of a mathematical algorithm used for comparison of sequences is the algorithm of Myers & Miller, CABIOS 1988; 4: 11-17. Such an algorithm is incorporated into the ALIGN program (version 2.0) which is part of the GCG sequence alignment software package. When utilizing the ALIGN program for comparing amino acid sequences, a PAM 120 weight residue table, a gap length penalty of 12, and a gap penalty of 4 can be used. Yet another useful algorithm for identifying regions of local sequence similarity and alignment is the FASTA algorithm as described in Pearson & Lipman, Proc. Natl. Acad. Sci. USA 1988; 85: 2444-2448.

Advantageous for use according to the present invention is the WU-BLAST (Washington University BLAST) version 2.0 software. WU-BLAST version 2.0 executable programs for several UNIX platforms can be downloaded from the FTP site for Blast at the Washington University in St. Louis website. This program is based on WU-BLAST version 1.4, which in turn is based on the public domain NCBI-BLAST version 1.4 (Altschul & Gish, 1996, Local alignment statistics, Doolittle ed., Methods in Enzymology 266: 460-480; Altschul et al., Journal of Molecular Biology 1990; 215: 403-410; Gish & States, 1993; Nature Genetics 3: 266-272; Karlin & Altschul, 1993; Proc. Natl. Acad. Sci. USA 90: 5873-5877; all of which are incorporated by reference herein).

In all search programs in the suite the gapped alignment routines are integral to the database search itself. Gapping can be turned off if desired. The default penalty (Q) for a gap of length one is Q=9 for proteins and BLASTP, and Q=10 for BLASTN, but may be changed to any integer. The default per-residue penalty for extending a gap (R) is R=2 for proteins and BLASTP, and R=10 for BLASTN, but may be changed to any integer. Any combination of values for Q and R can be used in order to align sequences so as to maximize overlap and identity while minimizing sequence gaps. The default amino acid comparison matrix is BLOSUM62, but other amino acid comparison matrices such as PAM can be utilized.

Alternatively or additionally, the term “homology” or “identity”, for instance, with respect to a nucleotide or amino acid sequence, can indicate a quantitative measure of homology between two sequences. The percent sequence homology can be calculated as (N_{ref}−N_{dif})*100/−N_{ref}, wherein N_{dif} is the total number of non-identical residues in the two sequences when aligned and wherein N_{ref} is the number of residues in one of the sequences. Hence, the DNA sequence AGTCAGTC will have a sequence identity of 75% with the sequence AATCAATC (N N_{ref}=8; N N_{dif}=2). “Homology” or “identity” can refer to the number of positions with identical nucleotides or amino acids divided by the number of nucleotides or amino acids in the shorter of the two sequences wherein alignment of the two sequences can be determined in accordance with the Wilbur and Lipman algorithm (Wilbur & Lipman, Proc Natl Acad Sci USA 1983; 80:726, incorporated herein by reference), for instance, using a window size of 20 nucleotides, a word length of 4 nucleotides, and a gap penalty of 4, and computer-assisted analysis and interpretation of the sequence data including alignment can be conveniently performed using commercially available programs (e.g., Intelligenetics™ Suite, Intelligenetics Inc. CA). When RNA sequences are said to be similar, or have a degree of sequence identity or homology with DNA sequences, thymidine (T) in the DNA sequence is considered equal to uracil (U) in the RNA sequence. Thus, RNA sequences are within the scope of the invention and can be derived from DNA sequences, by thymidine (T) in the DNA sequence being considered equal to uracil (U) in RNA sequences. Without undue experimentation, the skilled artisan can consult with many other programs or references for determining percent homology.

The invention further encompasses kits useful for screening nucleic acid isolated from one or more patients for allelic variation of a HLA-DR gene, or one or more gene listed in Tables A, B, C or D, and in particular for any of the SNPs described herein, wherein the kits may comprise at least one oligonucleotide selectively hybridizing to a nucleic acid comprising a HLA-DR gene, or one or more gene listed in Tables A, B, C or D described herein and instructions for using the oligonucleotide to detect variation in the nucleotide corresponding to the SNP of the isolated nucleic acid.

One embodiment of this aspect of the invention provides an oligonucleotide that specifically hybridizes to the isolated nucleic acid molecule of this aspect of the invention, and wherein the oligonucleotide hybridizes to a portion of the isolated nucleic acid molecule comprising any one of the polymorphic sites in the HLA-DR gene, or one or more gene listed in Tables A, B, C or D described herein.

Another embodiment of the invention is an oligonucleotide that specifically hybridizes under high stringency conditions to any one of the polymorphic sites of the HLA-DR gene, or one or more gene listed in Tables A, B, C or D, wherein the oligonucleotide is between about 18 nucleotides and about 50 nucleotides.

In another embodiment of the invention, the oligonucleotide comprises a central nucleotide specifically hybridizing with a gene polymorphic site of the portion of the nucleic acid molecule.

Another aspect of the invention is a method of identifying a polymorphism in a nucleic acid sample comprising isolating a nucleic acid molecule encoding HLA-DR, or one or more gene listed in Tables A, B, C or D or a fragment thereof and determining the nucleotide at the polymorphic site.

Another aspect of the invention is a method of screening patients to determine those 30 patients more likely to develop PD comprising the steps of obtaining a sample of genetic material from a patient; and assaying for the presence of a genotype in the patient which is associated with developing PD diseases, the genotype characterized by a polymorphism in a HLA-DR gene or one or more gene listed in Tables A, B, C or D.

In other embodiments of this invention, the step of assaying is selected from the group consisting of: restriction fragment length polymorphism (RFLP) analysis, minisequencing, MALD-TOF, SINE, heteroduplex analysis, single strand conformational polymorphism (SSCP), denaturing gradient gel electrophoresis (DGGE) and temperature gradient gel electrophoresis (TGGE).

In various embodiments of the invention, the method may further comprise the step of amplifying a region of a HLA-DR gene or a portion thereof that contains the polymorphism. In other embodiments of the invention, the amplification may include the step of selecting a forward and a reverse sequence primer capable of amplifying a region of a HLA-DR gene, or one or more genes listed in Tables A, B, C or D.

The present invention also encompasses a transgenic mouse which may express one or more polymorphisms of the genes listed in Tables A, B, C or D. In an exemplary embodiment, the transgenic mouse may contain one or polymorphisms of the genes listed in Table A. In another exemplary embodiment, the transgenic mouse may contain one or polymorphisms of the genes listed in Table B. In yet another exemplary embodiment, the transgenic mouse may contain one or polymorphisms of the genes listed in Table C. In still another exemplary embodiment, the transgenic mouse may contain one or polymorphisms of the genes listed in Table D. Advantageously the transgenic mouse may express a polymorphism of the HLA-DR gene, a GRIN2A gene and/or a SV2C2 gene, the methods for making a transgenic mouse are well known to one of skill in the art, see e.g., U.S. Pat. Nos. 7,709,695; 7,667,090; 7,655,700; 7,626,076; 7,566,812; 7,544,855; 7,538,258; 7,495,147; 7,479,579; 7,449,615; 7,432,414; 7,393,994; 7,371,920; 7,358,416; 7,276,644; 7,265,259; 7,220,892; 7,214,850; 7,186,882; 7,119,249; 7,112,715; 7,098,376; 7,045,678; 7,038,105; 6,750,375; 6,717,031; 6,710,226; 6,689,937; 6,657,104; 6,649,811; 6,613,958; 6,610,905; 6,593,512; 6,576,812; 6,531,645; 6,515,197; 6,452,065; 6,372,958; 6,372,957; 6,369,295; 6,323,391; 6,323,390; 6,316,693; 6,313,373; 6,300,540; 6,255,555; 6,245,963; 6,215,040; 6,211,428; 6,201,166; 6,187,992; 6,184,435; 6,175,057; 6,156,727; 6,137,029; 6,127,598; 6,037,521; 6,025,539; 6,002,067; 5,981,829; 5,936,138; 5,917,124; 5,907,078; 5,894,078; 5,850,004; 5,850,001; 5,847,257; 5,837,875; 5,824,840; 5,824,838; 5,814,716; 5,811,633; 5,723,719; 5,720,936; 5,688,692; 5,631,407; 5,620,881; 5,574,206 and 5,569,827. The transgenic mouse may be utilized to mimic PD disease conditions and may be useful to test novel treatments of PD in a mouse model.

The invention will now be further described by way of the following non-limiting examples:

### Example 1

GWAS conducted in Japan identified two novel PD loci. Five GWAS were performed in Caucasians, most confirmed the known SNCA and MAPT, but none identified any new genes that reached genome-wide significance. The inventors investigated whether the genetic component in PD in Caucasians was primarily due to the genes that have already been identified. Using NGRC data, they estimated heritability before and after excluding the known pathogenic and susceptibility loci. Heritability of PD declined from 0.6 (P<0.0001) to 0.41 (P=0.01), but was still significant, suggesting additional unidentified genes exist.

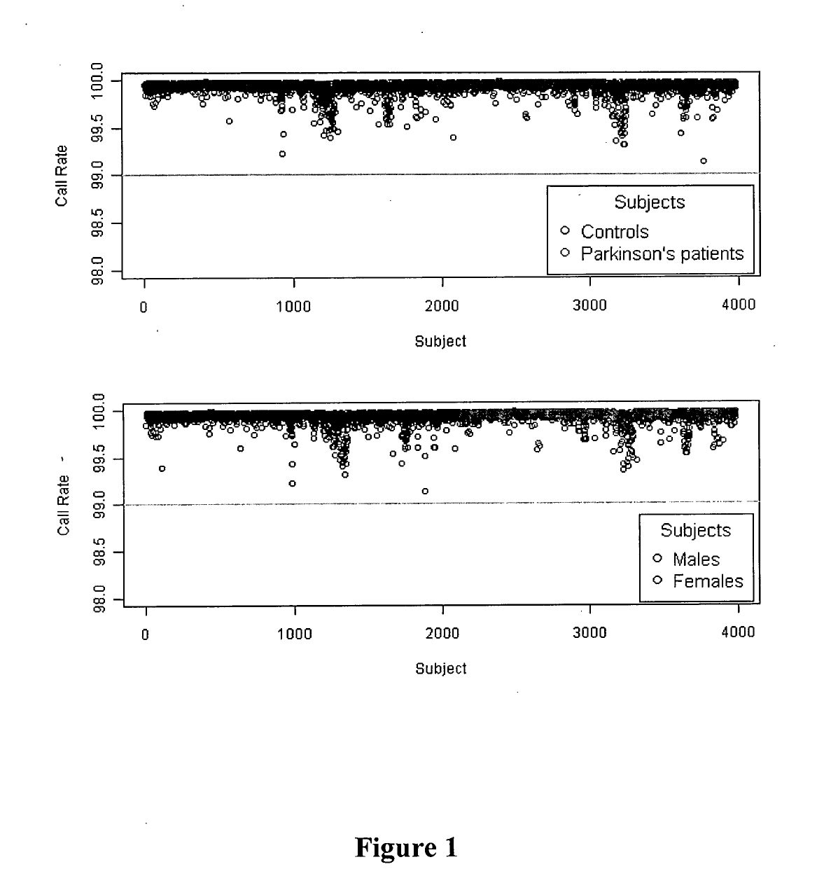

A GWAS with 2,000 PD patients, 1,986 control subjects, and 811,597 SNPs (Table 1) was performed. Subjects were recruited from NGRC clinics using uniform criteria for diagnosis, subject selection, data collection and DNA preparation. Twenty five percent of PD diagnoses change within the initial 5.4 years; therefore follow-up substantially reduces heterogeneity. Mean disease duration at enrollment was eight years, eliminating most early misdiagnoses, and with an additional mean follow-up of four years, the inventors were able to exclude another forty seven misdiagnoses before GWAS. Controls were selected by the same investigators and from the same geographic regions as patients. At enrollment, they were on average twelve years older than the patients' onset age, which increased power by reducing the likelihood that controls were at risk but too young to have developed symptoms. The HumanOmnil-Quad_v1-0_B genotyping array was used and achieved call rate of 99.92% and reproducibility rate of ≧99.99% (FIGS. 1 & 2). Association analyses were performed using PLINK V1.07. The known PD [symbol] susceptibility regions at SNCA (P=2.16×10⁻¹¹) and MAPT (P=8.71×10⁻⁷) were confirmed.

TABLE 1
Subjects.

| | Subjects | | | | | | | |
|---|---|---|---|---|---|---|---|---|
| | genotyped | | Subjects Excluded | | Subjects analyzed | | | |
| | | Call rate | Enrolled | Related | | | Onset age | Age |
| Group | N | Mean ± s.d. | twice | PI_HAT >.15 | N | % men | Mean ± s.d. | Mean ± s.d. |
| PD | 2014 | 99.92 ± 0.08 | 1 | 13 | 2000 | 67.3 | 58.34 ± 11.93 | 67.26 ± 10.67 |
| Controls | 1999 | 99.92 ± 0.08 | 3 | 10 | 1986 | 38.7 | — | 70.32 ± 14.09 |
| Total | 2013 | 99.92 ± 0.08 | 4 | 23 | 3986 | 100.0 | 58.34 ± 11.93 | 68.78 ± 12.58 |

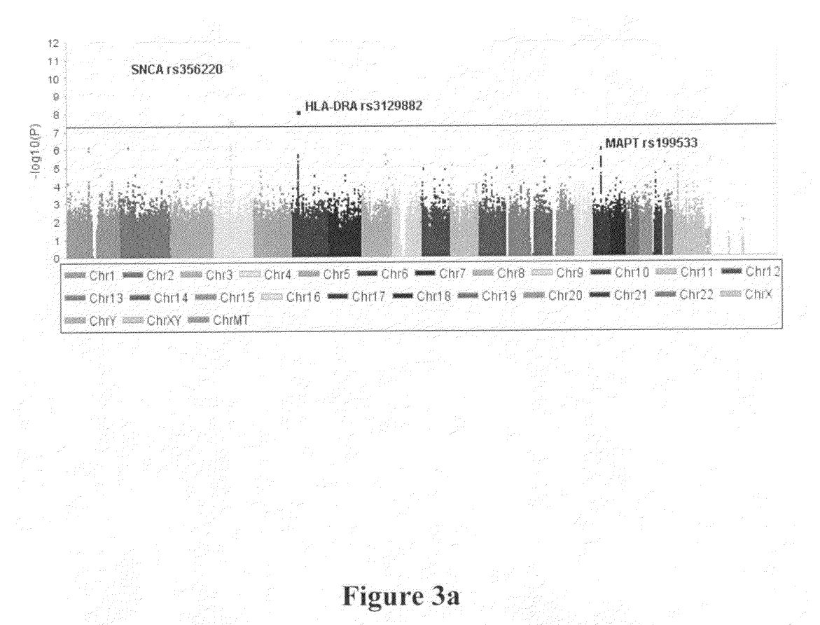

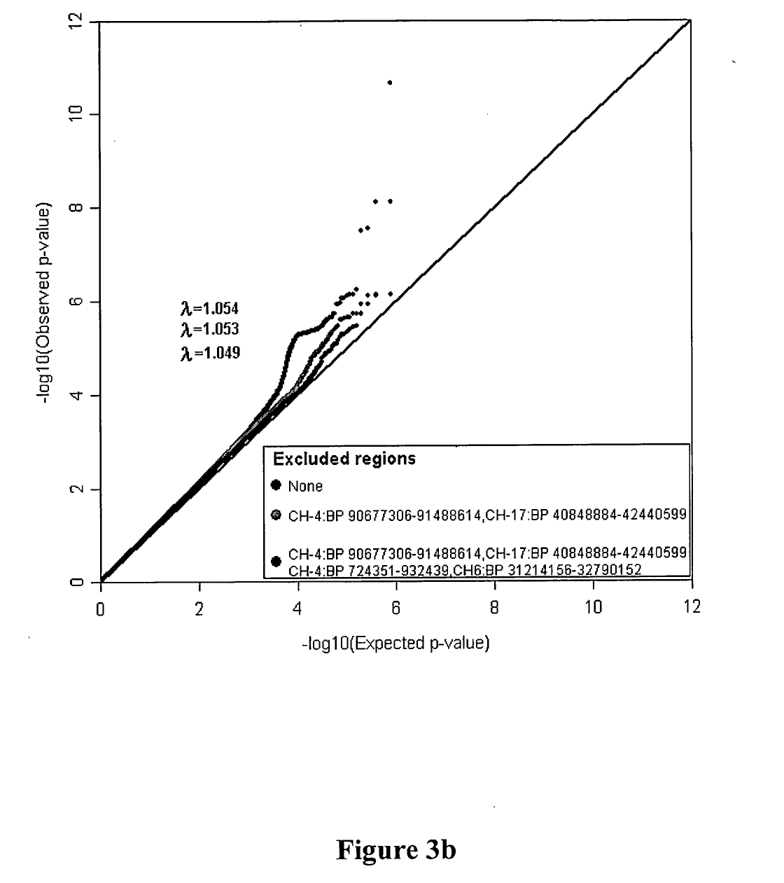

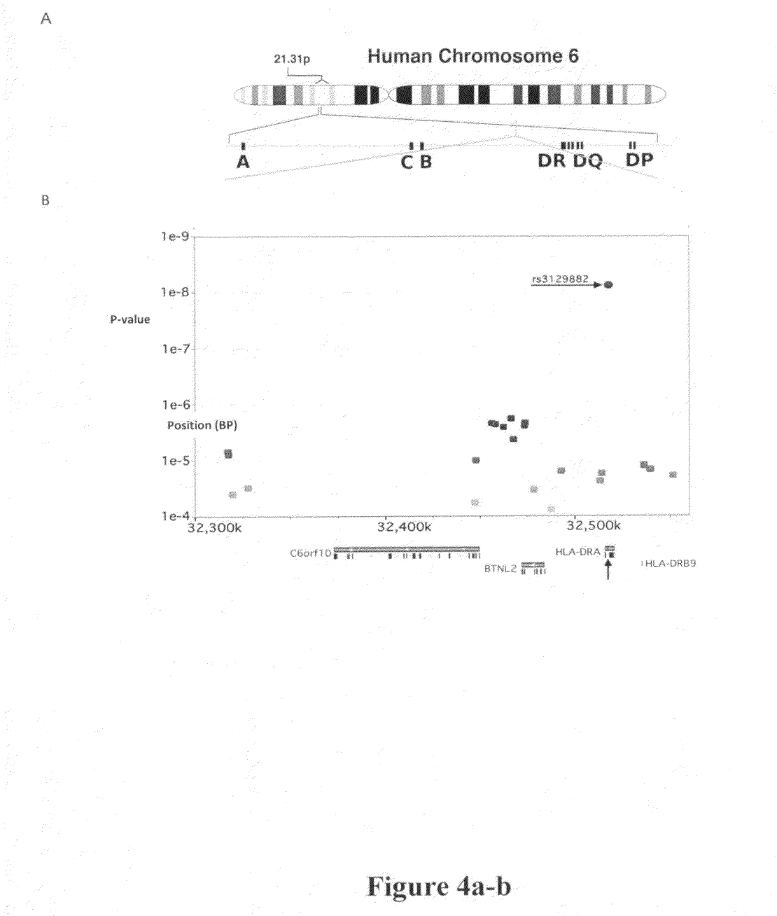

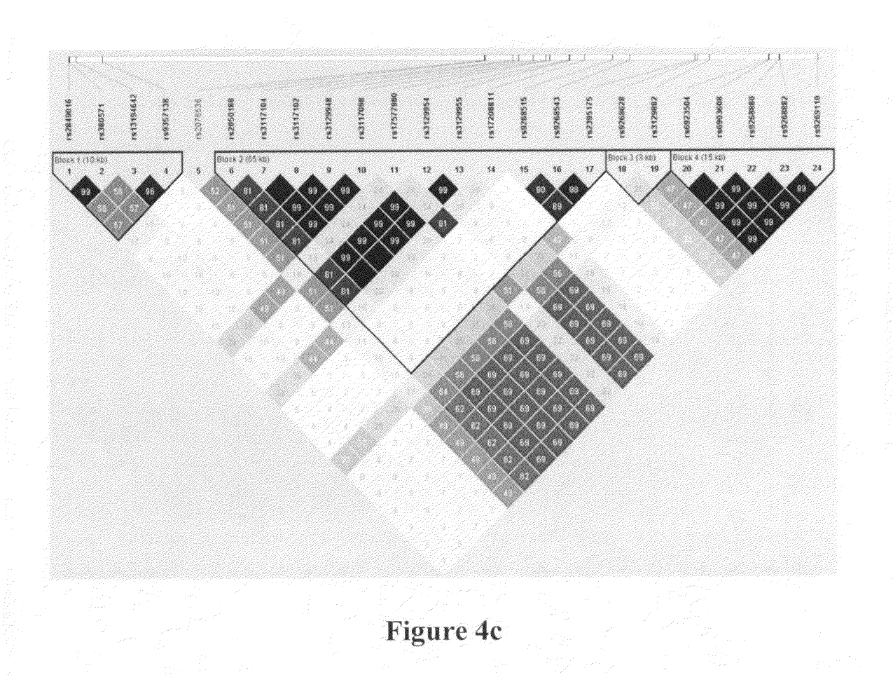

Further, a novel PD-associated SNP, rs3129882, which achieved genome-wide significance, P=7.51×10⁻⁹ (Table 2, FIG. 3) was uncovered. rs3129882 was in intron-1 of HLA-DRA gene (6p21.3). Twenty-three additional SNPs around HLA-DRA reached P=7.8×10⁻⁵-1.8×10⁻⁶ (FIG. 4). Association of rs3129882 was replicated in two independent data sets (P=5.5×10⁻³ and P=0.029). Meta-analysis yielded odds ratio (OR)=1.26 and P=2.2×10¹° for the additive model, and OR=1.38 and P=1.0×10⁻⁹ for the dominant model. The association with HLA-DRA was strongest in sporadic PD (P=1.17×10⁻¹⁰), late-onset PD (P=5.25×10⁻⁹), and men (P=4.47×10⁻⁸). The association was robust across smoking, coffee and NSAIDs strata (Table 2). There was no evidence for gene-environment interaction with smoking (P=0.42).

TABLE 2
Association results for the novel (HLA-DR) and known (SNCA, MAPT) PD
susceptibility loci

| HLA-DR (PARK17) | | N | N | MAF | MAF | | | |
|---|---|---|---|---|---|---|---|---|
| 6p21.3 | rs3129882 | Case | Control | Case | Control | HWE | P-value | OR (95% CI) |
| NGRC | | 2000 | 1986 | 0.46 | 0.40 | 1.00 | 7.5 × 10⁻⁹ | 1.32 (1.20-1.45) |
| Replication 1⁹ | | 843 | 856 | 0.45 | 0.41 | 0.57 | 5.5 × 10⁻³ | 1.21 (1.04-1.30) |
| Replication 2⁸ | | 604 | 612 | 0.44 | 0.40 | 0.06 | 0.03 | 1.17 (1.00-1.37) |
| Combined Replication 1 and 2 | | 1447 | 1468 | 0.45 | 0.40 | 0.09 | 1.1 × 10⁻³ | 1.18 (1.06-1.31) |
| Combined NGRC and Replications 1 and 2 | | 3447 | 3454 | 0.45 | 0.40 | 0.27 | 2.2 × 10⁻¹⁰ | 1.26 (1.17-1.35) |
| PD-associated risk factors | | | | | | | | |
| | Non-Familial | 1565 | 1986 | 0.47 | 0.40 | 1.00 | 1.17 × 10⁻¹⁰ | 1.39 (1.26-1.54) |
| | Familial | 435 | 1986 | 0.42 | 0.40 | 1.00 | 0.13 | 1.13 (0.97-1.32) |
| | Late onset | 1480 | 1986 | 0.46 | 0.40 | 1.00 | 5.25 × 10⁻⁹ | 1.36 (1.22-1.50) |
| | Early onset | 519 | 1986 | 0.45 | 0.40 | 1.00 | 1.29 × 10⁻³ | 1.26 (1.10-1.46) |
| | Male | 1346 | 769 | 0.46 | 0.38 | 0.25 | 4.47 × 10⁻⁸ | 1.44 (1.26-1.64) |
| | Female | 654 | 1217 | 0.45 | 0.41 | 0.37 | 8.81 × 10⁻³ | 1.20 (1.05-1.37) |
| | Smokers | 732 | 696 | 0.47 | 0.39 | 1.0 | 7.38 × 10⁻⁵ | 1.37 (1.17-1.61) |
| | Non-smokers | 866 | 810 | 0.45 | 0.40 | 0.61 | 1.62 × 10⁻³ | 1.26 (1.09-1.46) |
| | Coffee-High | 512 | 387 | 0.47 | 0.40 | 0.40 | 2.81 × 10⁻³ | 1.35 (1.11-1.64) |
| | Coffee-Low | 946 | 544 | 0.45 | 0.40 | 0.25 | 6.27 × 10⁻³ | 1.25 (1.07-1.47) |
| | NSAIDs user | 976 | 657 | 0.46 | 0.40 | 0.51 | 1.48 × 10⁻³ | 1.27 (1.11-1.48) |
| | No-NSAIDs | 594 | 326 | 0.46 | 0.40 | 0.82 | 4.61 × 10⁻³ | 1.34 (1.09-1.64) |
| Population structure | | | | | | | | |
| Entire sample adjusted for PC1 and PC2 | | 2000 | 1986 | 0.46 | 0.40 | 1.00 | 3.17 × 10⁻⁸ | 1.30 (1.19-1.43) |
| Ashkenazi Jewish | Yes | 89 | 40 | 0.45 | 0.38 | 1.00 | 0.248 | 1.40 (0.79-2.48) |
| | No/Unknown | 1911 | 1946 | 0.46 | 0.40 | 1.00 | 1.92 × 10⁻⁸ | 1.31 (1.20-1.45) |
| Recruitment site | New York | 412 | 300 | 0.52 | 0.46 | 0.35 | 0.016 | 1.32 (1.05-1.64) |
| | Oregon | 504 | 898 | 0.43 | 0.40 | 0.15 | 0.082 | 1.15 (0.98-1.35) |
| | Georgia | 231 | 113 | 0.47 | 0.37 | 1.00 | 0.013 | 1.56 (1.10-2.22) |
| | Washington | 853 | 675 | 0.44 | 0.36 | 0.21 | 4.0 × 10⁻⁵ | 1.40 (1.19-1.64) |
| Paternal or maternal | Great Britain | 537 | 356 | 0.44 | 0.37 | 0.73 | 0.002 | 1.38 (1.13-1.70) |
| ancestry | Germany/Austria | 442 | 269 | 0.45 | 0.40 | 0.90 | 0.062 | 1.24 (0.99-1.56) |
| | Ireland* | 249 | 162 | 0.41 | 0.44 | 0.42 | 0.189 | 0.81 (0.59-1.11) |
| | Scandinavia | 226 | 146 | 0.42 | 0.35 | 0.86 | 0.044 | 1.40 (1.01-1.95) |
| | Eastern Europe | 92 | 78 | 0.48 | 0.43 | 0.49 | 0.244 | 1.32 (0.83-2.09) |
| | Italy | 91 | 68 | 0.57 | 0.47 | 0.63 | 0.164 | 1.41 (0.87-2.27) |
| | France | 82 | 68 | 0.44 | 0.44 | 0.15 | 0.646 | 1.13 (0.66-1.93) |
| | Russia | 64 | 23 | 0.53 | 0.45 | 1.00 | 0.217 | 1.59 (0.76-3.33) |
| SNCA | rs356220 4q21 | 2000 | 1986 | 0.44 | 0.36 | 0.38 | 2.16 × 10⁻¹¹ | 1.38 (1.26-1.52) |
| MAPT | rs199533 17q21.1 | 2000 | 1986 | 0.17 | 0.22 | 0.05 | 8.71 × 10⁻⁷ | 0.74 (0.66-0.84) |

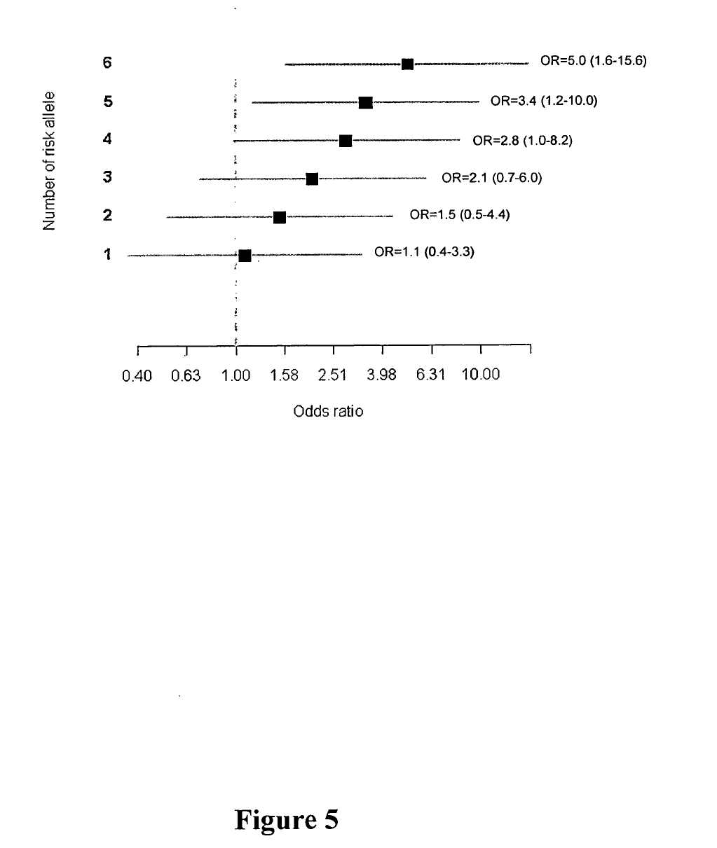

coffee (P=0.55) or NSAIDs (P=0.65), or for gene-gene interaction with SNCA (rs356220 P=0.78) or MAPT (rs199533 P=0.24). While each gene had an effect, the combined effects of SNCA, MAPT, and HLA-DR rose to OR=5.0 (95% confidence-interval=1.6-15.6) (FIG. 5).

Applicants confirmed the association of PD with cyclin G-associated kinase (GAK, 4p16), as suggested by Pankratz et al. GWAS (rsl 1248051 P_{NGRC}=5.94×10⁻⁵, OR_{NGRC+Pankratz}=1.45, P_{NGRC+Pankratz}=4.4×10⁻⁹), and found evidence for interaction with SNCA, which is on the other arm of chromosome 4 (OR_{Interaction}=1.46, P=9.41×10⁴). The Satake et al. GWAS reported PARK16 (1q32), BST1 (4p15) and LRRK2 common variants (not rare mutations that cause Mendelian-PD) as new risk factors in the Japanese, and the accompanying GWAS by Simon-Sanchez et al. provided equivocal evidence that PARK16 and LRRK2 associations may extend to Caucasians. In NGRC, PARK16 variants were not significant (P=0.053-0.468), evidence for BST1 was equivocal (P=0.01-0.09), and the reported LRRK2 variants were non-significant (P-0.3). However, several other LRRK2 SNPs reached P˜10⁻³. In NGRC, 1% of sporadic and 3% of familial PD have rare LRRK2 mutations [Kay et al. (2006) Mov Disord 21, 519-23].

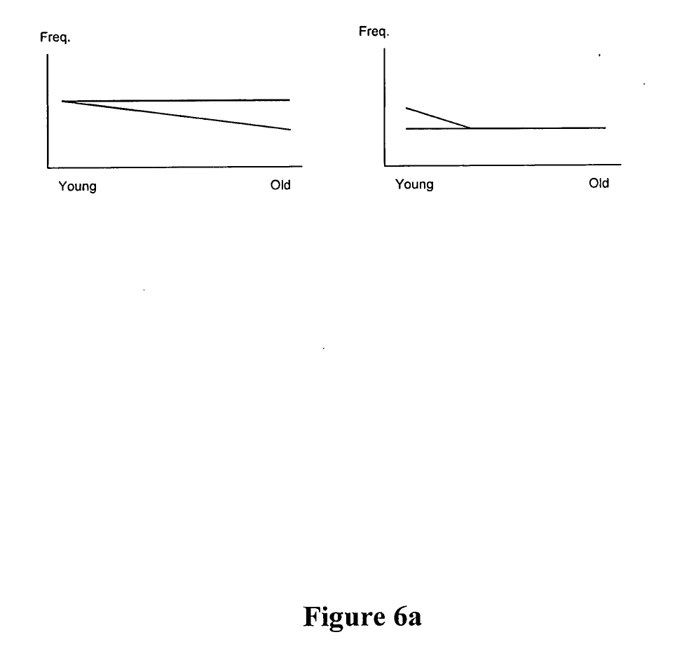

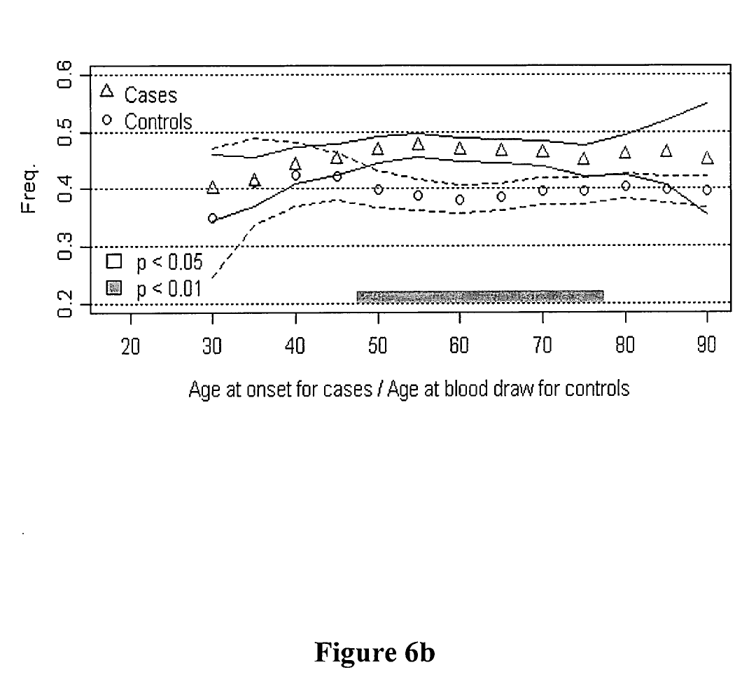

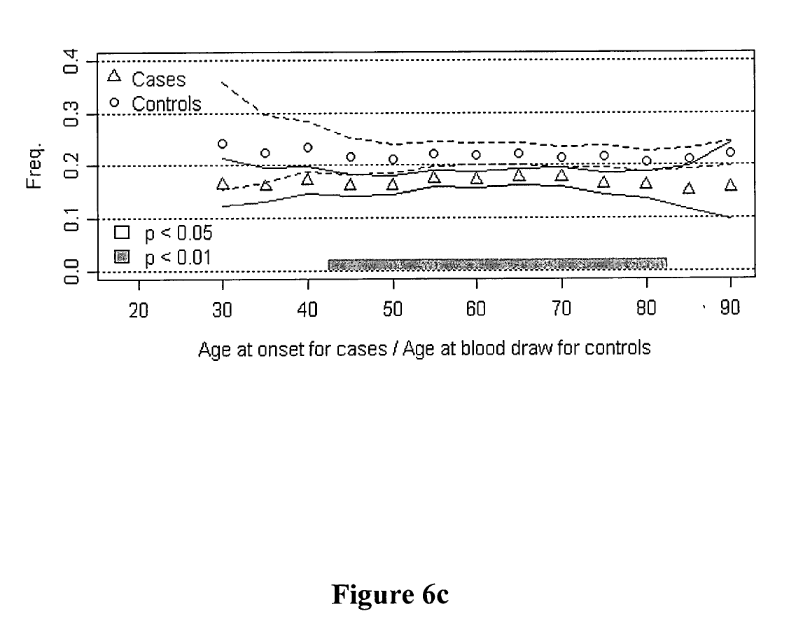

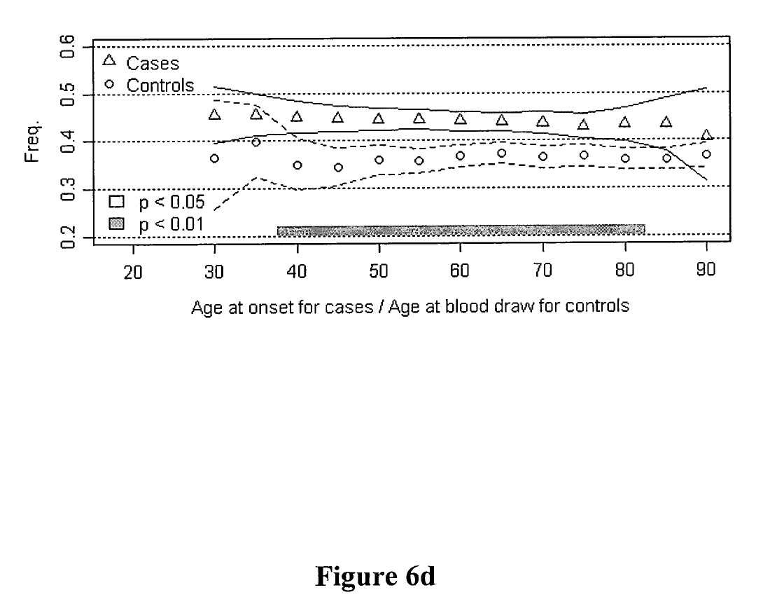

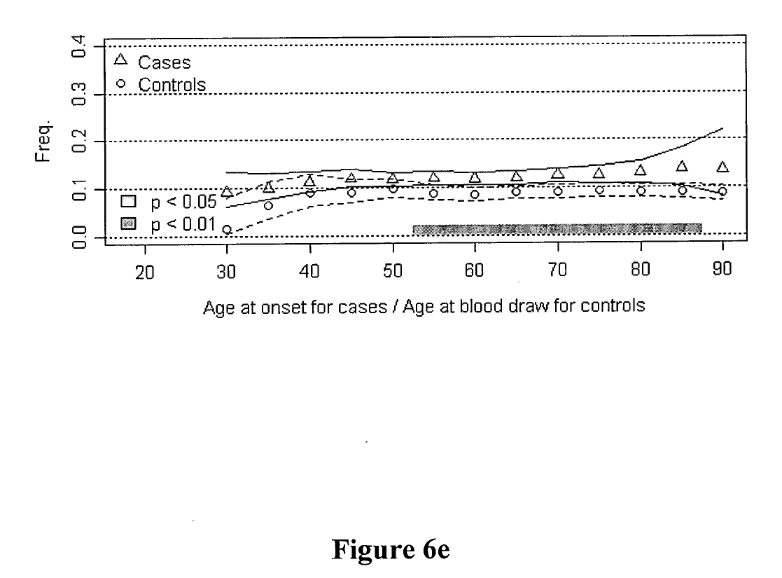

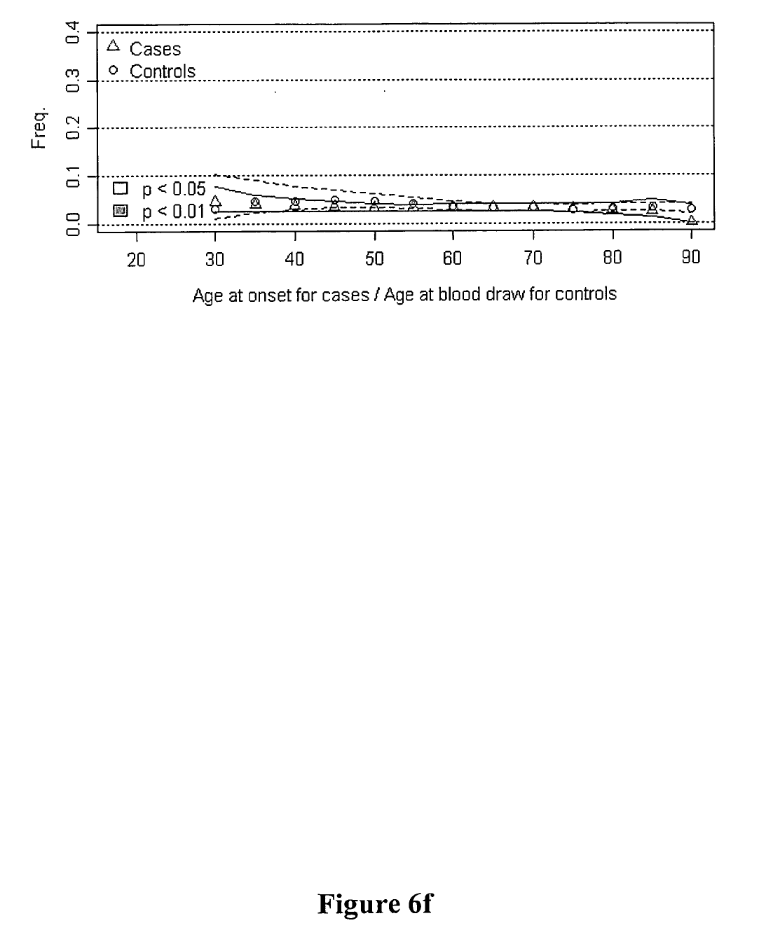

To investigate possible confounding by age, and to define the age-range where the associations are most significant, the MAP method [Payami et al. (2005) Hum Genet 118, 322-30; Payami et al. (2009) Genet Epidemiol 34, 92-9] was used. Data suggested that the association of rs3 129882 with PD is not an artifact of hidden allele frequency variation as function of age (FIG. 6). MAPs also showed that HLA-DRA, SNCA and MAPT exert their strongest effects in the 50-75 year age range.

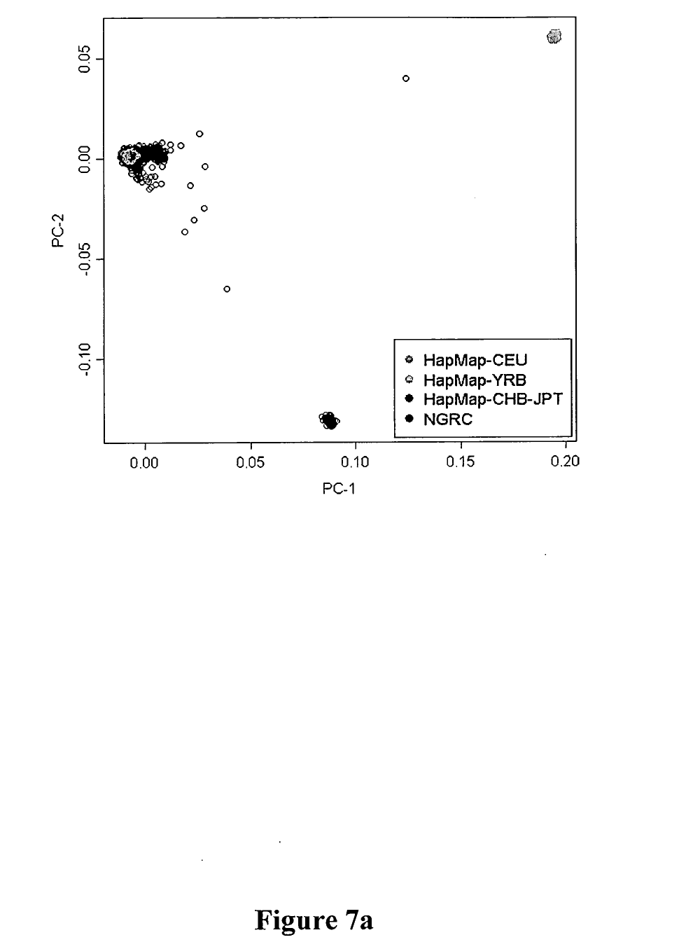

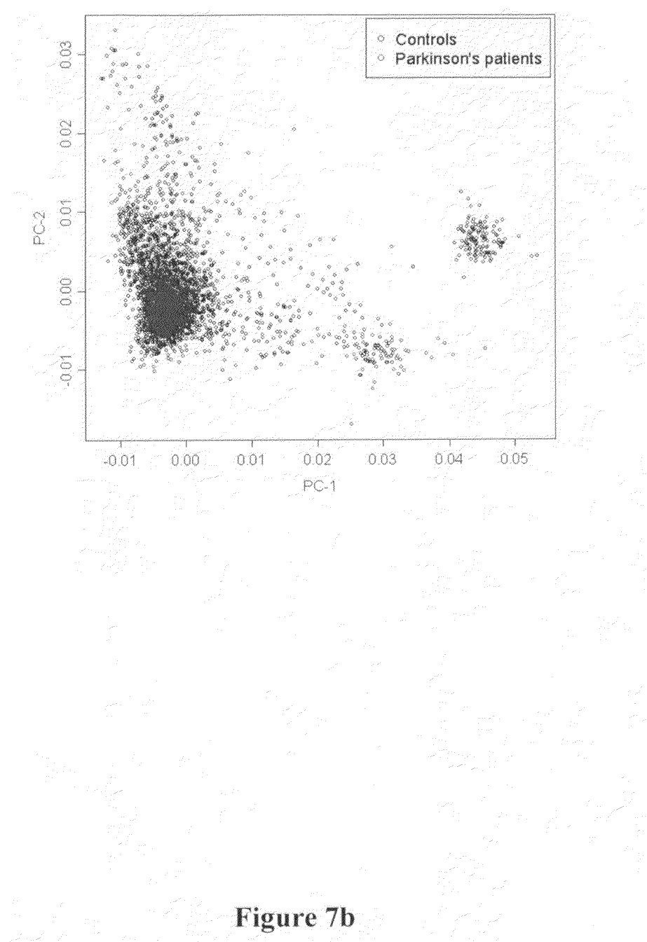

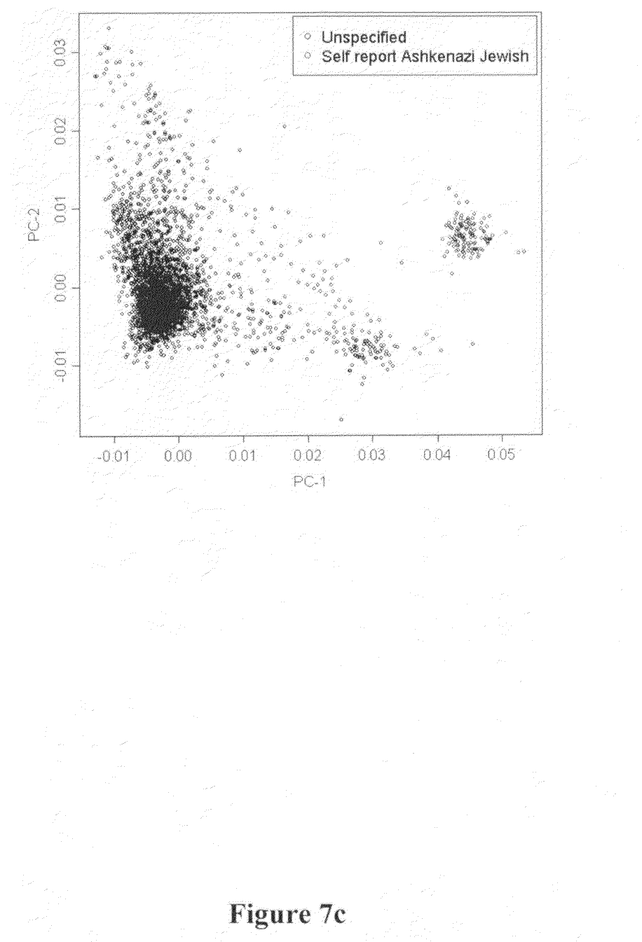

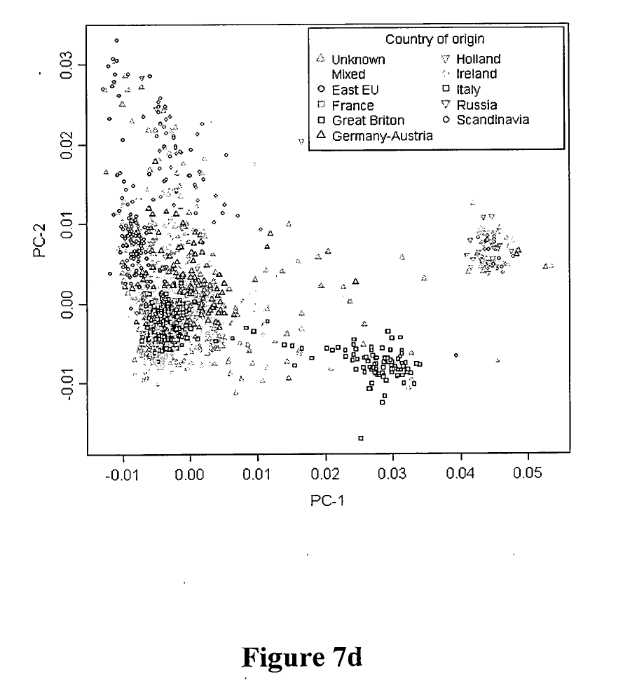

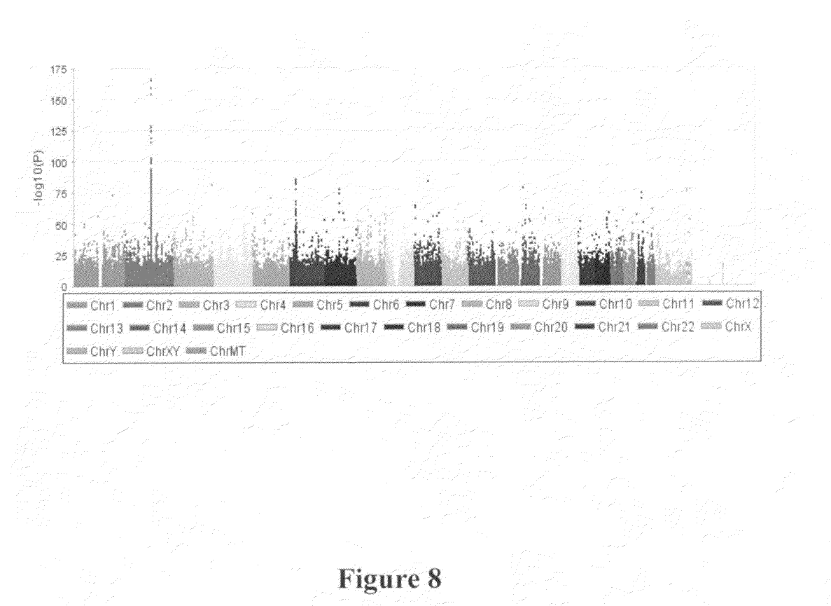

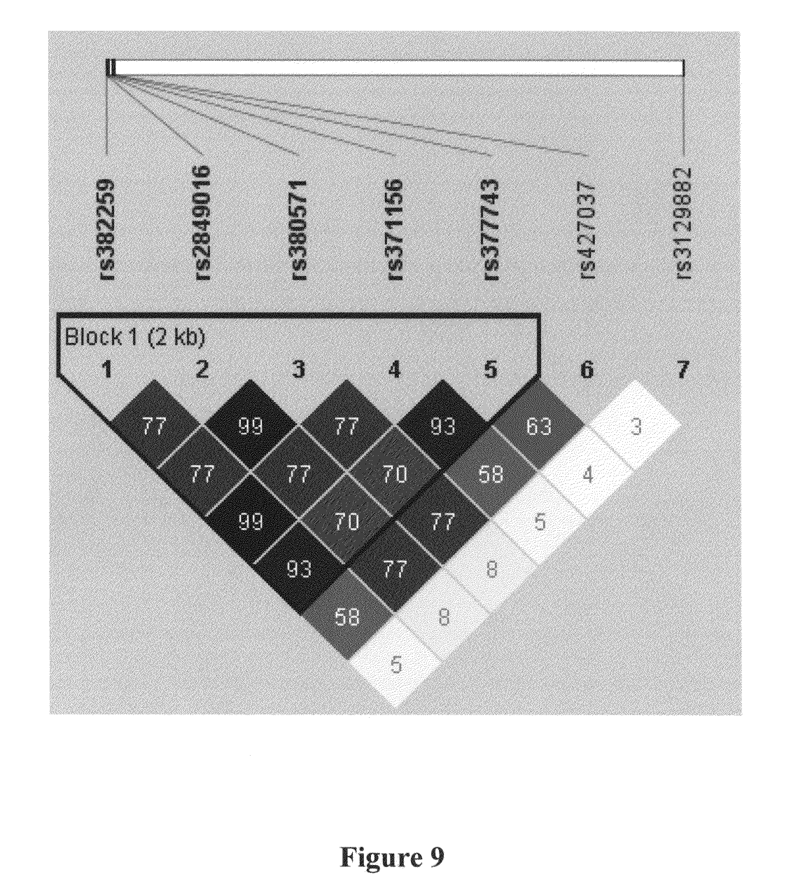

Population structure was also investigated, using Genomic Control [Devlin et al. (2001) Theor Popul Biol 60, 155-66] and principle component analysis (PCA) [Price et al. (2006) Nat Genet. 38, 904-9], augmented with self-reported ethnic and geographic origin. First, NGRC against the HapMap Caucasian, African and Asian populations was plotted. NGRC clustered with Caucasians (FIG. 7A) with only two individual falling beyond two standard deviations. The HapMap samples were then removed and population structure within NGRC explored. Genomic inflation factor was λ=1.05. The study continued with PCA, which revealed two clusters (FIG. 7B) and a significant principle component (PC 1, P=2.9×10⁻⁵, Eigen-value=3.97) suggesting membership in the clusters affects PD risk. Using self reported data, it was determined that the two clusters correlate with Ashkenazi-Jewish and non-Jewish ancestry (FIG. 3C). Next, genomic regions associated with PC 1 were identified, ruling out the concern that the association of HLA-DRA with PD was driven by PC 1-associated SNPs within HLA (FIG. 8, 9 & Table 3) excluded (P=1.92×10⁻⁸) (Table 2).

TABLE 3
HLA, PC1 and PD.

| | | | | | | Association of | | | |
|---|---|---|---|---|---|---|---|---|---|
| | | | | | | PC1- SNPs with | | Association of | |
| | | | | | | PD, adjusted | | HLA-DRA with | |
| | | | | | | for HLA-DRA | | PD, adjusted for | |
| | | | | | | rs3129882 | | PC1-associated | |
| Ch 6 | | | MAF | MAF | | and gender | | SNPs and gender | |
| VARIANTS | SNP | Position | Cases | Controls | HWE | OR | P | OR | P |
| HLA-DRA | rs3129882 | 32517508 | 0.46 | 0.40 | 1.00 | — | — | — | — |
| PC1- | rs382259 | 32317005 | 0.28 | 0.25 | 5.89 × 10⁻³ | 1.13 | 0.024 | 1.29 | 2.9 × 10⁻⁷ |
| associated | rs2849016 | 32317240 | 0.24 | 0.20 | 2.43 × 10⁻⁴ | 1.19 | 0.003 | 1.26 | 3.3 × 10⁻⁶ |
| variants in | rs380571 | 32317540 | 0.24 | 0.20 | 2.43 × 10⁻⁴ | 1.18 | 0.003 | 1.26 | 2.9 × 10⁻⁶ |
| HLA | rs371156 | 32317941 | 0.28 | 0.25 | 5.89 × 10⁻³ | 1.13 | 0.024 | 1.29 | 2.7 × 10⁻⁷ |
| | rs377743 | 32319043 | 0.27 | 0.24 | 3.79 × 10⁻³ | 1.11 | 0.046 | 1.29 | 1.4 × 10⁻⁷ |
| | rs427037 | 32320242 | 0.25 | 0.22 | 1.07 × 10⁻² | 1.16 | 0.005 | 1.28 | 3.0 × 10⁻⁷ |

PC2 was also significant (P=0.007, Eigen-value=1.26), suggesting residual genetic diversity within the larger non-Jewish cluster. When subjects' genotypes were plotted with colors representing different countries of ancestral origin, the larger cluster resembled the map of Europe: individuals originating from the same regions clustered tightly together, and the countries they represented fell in correct geographic position (FIG. 7d). Frequency of rs3129882 risk allele appeared to be low in Northern-Europeans and high in Southern-Europeans, particularly Italians (Table 2). Among the enrollment sites, New York had the highest allele frequency. According to the US census, 14.4% of New Yorkers are Italian vs. 5.6% nation-wide. Association of HLA-DRA with PD remained genome-wide significant adjusted for both PC1 and PC2 (P=3.17×10⁸). Lower-order PCs were not significant, indicating that the bulk of genetic diversity within NGRC has been identified and accounted for.

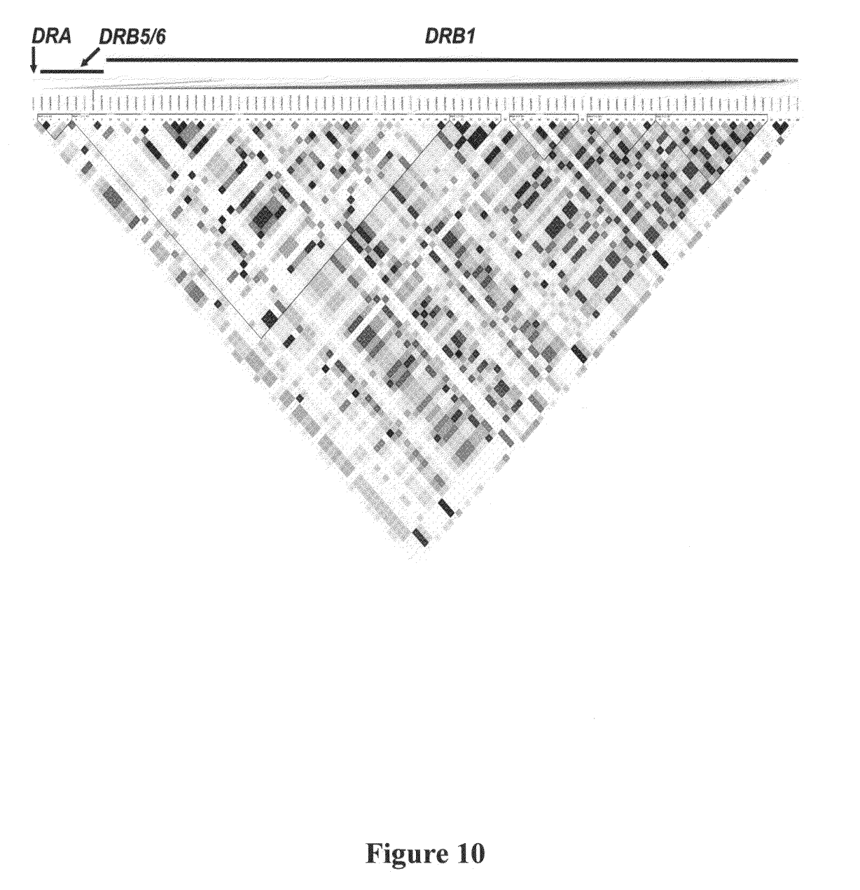

HLA-DRA is a class II major histocompatibility gene. The protein chains encoded by the closely linked HLA-DRA and HLA-DRB form the DR antigens, which are expressed by antigen presenting cells, including microglia in brain, to elicit humoral immune response. HLA-DRB chains are highly variable and associated with numerous disorders, whereas HLA-DRA is practically monomorphic. PD-association with an intronic DRA variant may represent involvement of regulatory elements, which would be in line with PD-specific over-expression of DR antigens in substantia nigra [Payami et al. (2005)]. Alternatively, PD-association may be with HLA-DRB. The inventors detected a linkage disequilibrium (LD) between rs3129882 and HLA-DRB (r²=0.00-0.26, FIG. 10), and it is possible that PD is associated with a serologically identifiable HLA-DRB antigen. It is possible that the PD-associated allele resides within the HLA class II region.

Study was approved by Human Subject Committees at participating institutions. Patients and Control subjects were recruited from eight NGRC-affiliated neurology clinics in Oregon, Washington, Georgia and New York. Methods were standardized across NGRC. Patients were diagnosed using the modified UK Brain Bank criteria. GWAS subjects met seven criteria: (1) Self-reported Caucasian, non-Hispanic, of European origin. (2) Patients with current diagnosis of PD, excluding those whose initial PD diagnosis changed during the ˜12 years of follow up. (3) DNA extracted from whole blood, unamplified, concentration≧50 ng/μl. (4) Age at blood draw≧21 years. (5) Known gender. (6) Known age at onset (one missing). (7) No blood-relation to other subjects. Data on smoking, coffee, and NSAIDs on ˜3,000 of NGRC subjects, and on country of ancestral origin for 2,080 subjects was made available. 112 subjects reported as Ashkenazi Jewish (129 were Jewish according to PCA). For replication, chosen datasets included those that (a) were published by peer review, (b) had genotyped rs3129882 (c) were Caucasian. Among four publicly available GWAS only “CIDR: Genome Wide Association Study in Familial PD (PD)” by Pankratz et al. met Applicants' criteria.

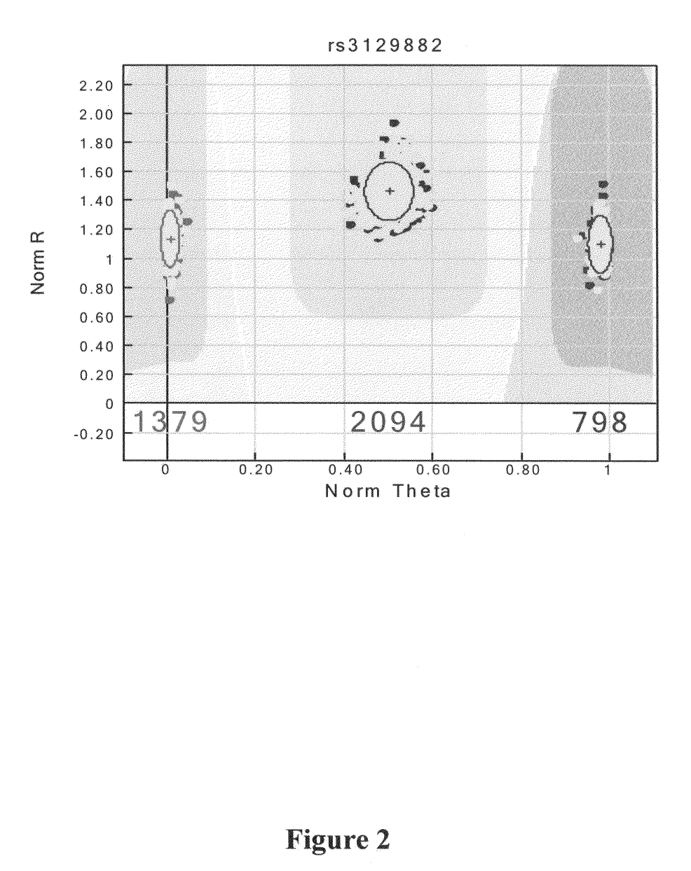

To reduce plate effects, DNA samples were randomized on genotyping plates by case-control status, recruitment site, control subjects who were healthy at age≧85 years, DNA extraction method, and DNA storage time. Samples were genotyped at the Johns Hopkins Center for Inherited Disease Research (CIDR). Data was released for 4,013 study samples (99.5% of attempted samples). Study samples, 90 duplicates, and 170 HapMap controls (151 CEU; 12 YR1, 3 JPT, 4 CHB) were genotyped using Illumina HumanOmnil-Quad_v1-0_B BeadChips (Illumina, San Diego, Calif., USA) and the Illumina Infinium II assay protocol.³² Genotype cluster definitions for each SNP were determined using Illumina BeadStudio Genotyping Module version 3.3.7 and the combined intensity data from all released samples. Genotypes were not called if the quality threshold (Gencall score) was <0.15. Genotype data was released for 1,012,895 SNPs (99.65% of attempted). SNP assay failure criteria were: call rate<85%, cluster separation<0.2, >1 HapMap replicate error, >3% (autosomal) or >4% (X) difference in call rate between genders, >0.3% male AB frequency (X), or >8.8% (autosomal) or >13.2% (XY) difference in AB frequency. Y chromosome and mitochondrial SNPs were manually reviewed and clusters adjusted or genotypes dropped as appropriate. The mean non-Y SNP call rate and mean sample call rate were both 99.9% for the released CIDR dataset. Study duplicate reproducibility was ≧99.99%. (FIGS. 1, 2 & Table 2)

Sex was determined by estimating X chromosome homozygosity and compared to self-reported gender; there was no discrepancy. The final N for was 2,000 patients and 1,986 controls. SNPs were excluded if MAF<0.01, call-rate<99%, HWE<10⁻⁶, MAF difference in males vs. females>0.15, or missing rate in PD vs. control P<10⁻⁵. 811,597 SNPs passed quality-control; with mean call rate of 99.92%. PCA was conducted with HelixTree (the website of goldenhelix) using a pruned subset of 104,064 SNPs. Pruning was carried-out using PLINK with autosomal SNPs (MAF>0.05, call rate≧95%). A 50-SNP sliding-window that shifted 5 SNPs with each move was used and SNPs with r²≧0.02 were recursively removed, followed by a second round using a 138-SNP sliding-window, resulting in 104,064 SNPs. Association was tested under an additive model using logistic regression in PLINK V 1.07. Significance was set at P≦5×10⁻⁸ for discovery (Bonferroni-corrected for one million SNP tests), and P<0.05 for replication. P-values were two-tailed for discovery and Meta-analysis; one-tailed for replication. The models were corrected for gender, PC1 and PC2. Age was not adjusted for because controls were significantly older than patients (the novel finding is genome-wide significant even when adjusted for age, see Table 2 legend). To investigate allele frequencies as a function of age and age at onset, Applicants used the MAP method to plot moving average frequency of the minor allele in patients as a function of age at onset, against that in controls as a function of age, with their 95% central posterior intervals, and tested the difference between the two distributions in a moving window. LD was assessed using Haploview V4.1. and haplotype analysis was conducted using Hapstat 3.0. Breslow-Day test-statistics was used to test heterogeneity across strata within NGRC, and across datasets for Meta-analysis. The same sample and SNP quality-control filters were applied to replication data sets, similar to what was done for NGRC. Each replication dataset was individually tested using the tests and adjustments used by authors in their original report. Individual level Meta-analysis using PLINK was performed, adjusting for gender.

SNP rs4998386 is in GRIN2A on chromosome 16 at by position 9978046. Among people who drink more than median coffee, this SNP is associated with 57% reduction in PD risk. It is also associated with risk reduction in NSAID users and smokers. This table shows the effect of coffee on PD risk. Overall, coffee reduces the risk by 24% (1-0.76). In individuals who have T+ genotype at rs4998386, neuroprotective effect of coffee/caffeine doubles to 53% (1-0.474) (i.e., the risk of developing PD is reduced by 53%).

TABLE 4
PD-Coffee/caffeine association stratified by rs4998386 genotype

| | | N | | | | | |
|---|---|---|---|---|---|---|---|
| Effect of Coffee | | Control | Case | OR | 95% CI | | P |
| Blind to | High | 387 | 512 | 0.761 | 0.643 | 0.901 | 1.5E−03 |
| genotype | Low | 544 | 946 | | | | |
| T+ | High | 104 | 71 | 0.474 | 0.322 | 0.697 | 1.5E−04 |
| genotype | Low | 111 | 160 | | | | |
| CC | High | 283 | 441 | 0.858 | 0.710 | 1.038 | 0.115 |
| genotype | Low | 433 | 786 | | | | |

rs3129882 is in HLA-DRA, which regulates immunity and inflammation. It is ubiquitously associated with all forms of PD regardless of exposures. The target drug may be anti-inflammatory drugs, other immune related drugs.

rs4855877 is in TCTA/RHOA, a T-cell leukemia translocation altered gene. This gene affects PD risk in smokers. The gene is cancer related, and Parkinson disease and cancer have been linked epidemiologically.

rs967851 is not in a gene but is near LOC650095, which is similar to superoxide dismutase gene. This SNP and its nearby SNPs interact with the protective effect of NSAIDs on PD. The SNP does not fall in a gene per se, but serves as a proxy to, for example, superoxide dismutase, which lies within the 100 KB flanking regions. Superoxide dismutase is a target for drug discovery, the most likely candidate drugs being compounds that reduce oxidative damage.

rsl 1902313 is in HECW2 which encodes a E3 ubiquitine ligase. The association is most significant in early-onset strata of PD (similar to the known gene, parkin) A drug against this subtype of PD will be one that is directed against protein degradation malfunction and oxidative damage in PD. The SNP is used as a marker for risk assessment, and due to its association with early-onset disease, it may help devise strategies to delay onset of PD.

rsl 114832 is in LRRC68/NKPD1 which encodes a Leucine-rich repeat protein. Another Leucine-rich repeat gene, LRRK2, is a well established PD gene. The discovery leads to risk assessment, drug discovery, pharmacogenomics and personalized medicine.

### Example 2

Parkinson disease (PD) is a common neurodegenerative disorder that leads to motor and cognitive disability. PD is heterogeneous; the most common forms are sporadic, late-onset, and involve gene-environment interaction. A genome-wide association study (GWAS) with 2000 PD and 1986 control Caucasian subjects from NeuroGenetics Research Consortium (NGRC) was performed. The known associations with SNCA and MAPT; independently replicated the suggested association with GAK (P_{Pankratz+NGRC}=3.2×10⁻⁹) was confirmed; and a novel association with HLA-DR (P_{NGRC}=2.9×10⁻⁸) was detected, which was replicated using two independent datasets (P_{Replication1+2}=1.1×10⁻³, P_{Replication1+2+NGRC}−1.9×10⁻¹⁰). The HLA association was uniform across genetic and environmental risk strata, and strong in sporadic (P=5.5×10⁻¹⁰) and late-onset (P=2.4×10⁻⁸) PD. The new PD-associated genes were designated PARK17 (GAK) and PARK18 (HLA). Association with HLA is in line with reported neuro-inflammation involving up-regulation of DR antigens and reactive microglia in PD pathogenesis, and with association of non-steroidal anti-inflammatory drug (NSAID) use with reduced risk of PD. Genetic association with HLA-DR may offer a novel target for PD drug development and pharmacogenetics.

Late-onset sporadic PD was long believed to be environmental with no genetic component. The initial samples that eventually coalesced to form the NGRC dataset was the basis for the first of a series of family studies that revealed the genetic component in PD. In the last decade, mutations in several genes were identified as causes of early-onset and Mendelian forms of PD, and polymorphisms in SNCA (chromosome 4q21) and MAPT (17q21.1) regions were firmly established as risk factors for common non-Mendelian PD. PD risk is also associated, inversely, with cigarette smoking, caffeinated-coffee consumption and NSAID use.

A GWAS conducted in Japan identified two novel PD loci. Five GWAS have been performed in Caucasians, most confirmed the known SNCA and MAPT, but none identified any new genes that reached genome-wide significance. The present study focused on whether the genetic component in PD in Caucasians was due to the genes that have already been identified. Using NGRC data, heritability before and after excluding the known pathogenic and susceptibility loci was estimated. Heritability of PD declined from 0.6 (P<0.0001) to 0.4 (P=0.01), but was still significant, suggesting additional unidentified genes may exist.

A GWAS was performed with 2,000 PD patients, 1,986 control subjects, and 811,597 single nucleotide polymorphisms (SNPs). Subjects were recruited from NGRC clinics using uniform criteria for diagnosis, 20 subject selection, data collection and DNA preparation. 25% of PD diagnoses change within the initial 5.4 years; therefore follow-up substantially reduced heterogeneity. Mean disease duration at enrollment was 8 years, eliminating most early misdiagnoses, and with an additional mean follow-up of 4 years, it was possible to exclude another 47 misdiagnoses before GWAS. Controls were selected by the same investigators and from the same geographic regions as patients. At enrollment, they were on average 12 years older than the patients' onset age, which increased power by reducing the likelihood that controls were at risk but too young to have developed symptoms. Illumina HumanOmnil-Quad_v1-0_B genotyping array was used and a call rate of 99.92% and reproducibility rate of ≧99.99% was achieved. Association analyses were performed using PLINK V1.07. All analyses for 4 covariates were adjusted: age to avoid survival bias, sex because PD affects more men than women, and two principal components (PC1, PC2) which marked significant genetic substructure among Caucasian Americans. The known PD-susceptibility regions at SNCA (P=3.4×10⁻¹¹) and MAPT (P=1.3×10⁻⁶) was confirmed.

A novel PD-associated SNP, rs3129882 in the HLA region (6p21.3) was uncovered, which achieved genome-wide significance, P=2.9×10⁻⁸ (Table 5).

TABLE 5
Association results for novel and known PD susceptibility genes

| | | | Minor/ | | | | | HWE | | |
|---|---|---|---|---|---|---|---|---|---|---|
| | | | Major | N | N | MAF | MAF | P- | Association | |
| Locus | CHR | SNP | allele | Case | Control | Case | Control | value | P-value | OR (95% CI) |
| HLA | 6 | rs3129882 | G/A | | | | | | | |
| NGRC^{‡} | | | | 2000 | 1986 | 0.46 | 0.40 | 1.00 | 2.9 × 10⁻⁸ | 1.31 (1.19-1.44) |
| Replication 1⁹ | | | | 843 | 856 | 0.45 | 0.41 | 0.57 | 0.01 (5 × 10⁻³)^{#} | 1.21 (1.04-1.30) |
| Replication 2⁸ | | | | 604 | 612 | 0.44 | 0.40 | 0.06 | 0.06 (0.03)^{#} | 1.17 (1.00-1.37) |
| Replication 1 + 2 | | | | 1447 | 1468 | 0.45 | 0.40 | 0.09 | 1.1 × 10⁻³ | 1.18 (1.06-1.31) |
| NGRC + Replications 1 + 2 | | | | 3447 | 3454 | 0.45 | 0.40 | 0.27 | 1.9 × 10⁻¹⁰ | 1.26 (1.17-1.35) |
| GAK | 4 | rs11248051 | T/C | | | | | | | |
| Pankratz⁹ | | | | 843 | 856 | 0.13 | 0.09 | 0.08 | 5.2 × 10⁻⁶ | 1.71 (1.36-2.16) |
| NGRC | | | | 2000 | 1986 | 0.12 | 0.09 | 1.00 | 3.1 × 10⁻⁴ | 1.32 (1.14-1.54) |
| Pankratz⁹ + NGRC | | | | 2843 | 2842 | 0.12 | 0.09 | 0.42 | 3.2 × 10⁻⁹ | 1.46 (1.29-1.65) |
| LRRK2 | 12 | rs2708453 | T/G | | | | | | | |
| Satake¹⁶ | | | | 2011 | 18381 | 0.10- 0.13^{†} | 0.08 | — | 9.7 × 10⁻⁸ | 1.38 (1.22-1.54) |
| Pankratz⁹ | | | | 843 | 856 | 0.17 | 0.15 | 0.34 | 0.05 | 1.21 (1.00-1.47) |
| NGRC | | | | 2000 | 1986 | 0.17 | 0.15 | 0.86 | 0.03 | 1.15 (1.01-1.31) |
| Pantraz + NGRC | | | | 2843 | 2842 | 0.17 | 0.15 | 0.76 | 3.9 × 10⁻³ | 1.17 (1.05-1.30) |
| BST1 | 4 | rs4698412 | A/G | | | | | | | |
| Satake | | | | 2011 | 18381 | 0.38-0.40 | 0.33-0.35 | — | 1.8 × 10⁻⁸ | 1.24 (1.15-1.33) |
| Simon-Sanchez⁷ | | | | 5074 | 8551 | 0.57* | 0.56 | — | 0.03 | 1.06 |
| NGRC | | | | 2000 | 1986 | 0.58* | 0.55 | 0.01 | 0.01 | 1.13 (1.03-1.24) |
| Simon-Sanchez + NGRC | | | | 7074 | 10537 | — | — | — | 1.5 × 10⁻³ | 1.08 |
| SNCA | 4 | rs356220 | T/C | 2000 | 1986 | 0.44 | 0.36 | 0.38 | 3.4 × 10⁻¹¹ | 1.38 (1.25-1.52) |
| MAPT | 17 | rs199533 | T/C | 2000 | 1986 | 0.17 | 0.22 | 0.05 | 1.3 × 10⁻⁶ | 0.74 (0.66-0.84) |

MAF: Minor Allele Frequency.
HWE: Hardy Weinberg Equilibrium.
OR: Odds ratio calculated for minor allele.
^{‡}NGRC data were adjusted for PC1, PC2, sex and age.
^{#}One-sided P value is given in parenthesis; the only case where a single (no-multiple testing) one-sided hypothesis test was performed was for replication of rs3129882.
Subjects were Caucasian in NGRC, Pankratz, Edwards and Simon-Sanchez studies, and Japanese in Satake study.
*The minor allele (frequency <0.5) in the Japanese was the major allele (>0.5) in Caucasians. We used the same allele as reported for the Japanese to keep the risk allele consistent.
^{†}Satake et al provided the allele frequencies in individual data sets but not the combined GWAS and two replications they used for final analysis. We therefore show the range.

107 HLA SNPs reached P<10⁻³ for association with PD. Association of rs3129882 was replicated in two independent data sets (OR_{meta-analysis}=1.26, P_{Meta-analysis}=1.9×10⁻¹⁰). The risk allele was the same in all datasets and was in Hardy-Weinberg equilibrium (HWE) in cases and in controls which in addition to the visual inspection of intensity plots supports no major problems with genotyping. Stratified analysis by family history, age at onset, gender and environmental exposures revealed ubiquitous associations across strata. Associations were particularly strong for sporadic PD (P=5.5×10⁻¹⁰), late-onset PD (P=2.4×10⁻⁸), and men (P=1.1×10⁻⁷). Tests of heterogeneity across strata were not significant. There was no evidence for gene-environment interaction with smoking (P=0.42), coffee (P=0.55) or NSAIDs (P=0.65), or for gene-gene interaction with SNCA (rs356220 P=0.78) or MAPT (rs199533 P=0.24). In silico replication and Meta-analysis of the most significant SNPs (P<10⁻⁵) in NGRC was conducted using CIDR dataset from dbGap (Pankratz et al)⁹. SNPs that replicated were in the HLA (6 SNPs in addition to rs3129882), the SNCA and the MAPT regions. One SNP (rs2046571) in the Hyaluronan synthase 2 (HAS2) gene region on chromosome 8 was marginally significant in CIDR, but did not reach genome-wide significance in Meta-analysis (3.6×10⁻⁷).

NGRC was used to replicate findings of previous PD GWAS. The association of PD with cyclin G-associated kinase (GAK, 4p16) was confirmed (rs11248051 P_{NGRC}=3.1×10⁻⁴, OR_{NGRC+Pankratz}=1.46, P_{NGRC+Pankratz}=3.2×10⁻⁹). Two of the six reported BST1 SNPs reached P<0.05. LRRK2 variants that were reported in Caucasians were not significant in NGRC (P=0.57 & 0.69), however, three LRRK2 SNPs from a Japanese study yielded P<0.05 in NGRC, and several other LRRK2 SNPs reached P˜10⁻³. In NGRC, 1% of sporadic and 3% of familial PD have rare LRRK2 mutations.

SNCA, MAPT, GAK and HLA each have a modest effect on PD risk, but when considered together, the cumulative effect can be substantial. To explore the combined effects of the four genes, the subjects were classified by the total number of risk alleles that they carry (0 to 8). Compared to subjects who had one or no risk allele, the risk of PD was doubled for individuals who had four risk alleles (OR=2.49, 95% CI=1.79-3.47, P=6.5×10⁻⁸), and was five-fold higher for individuals who had six or more risk alleles (OR=4.95, 95% CI=3.20-7.64, P=5.5×10⁻¹³). Thus the data support that PD risk may be due to cumulative effects of risk factors with modest individual effect.

A persistent problem in genetic association studies is inconsistent reproducibility which often arises from hidden genetic variation, i.e., population structure. Population structure in depth, using Genomic Control (λ) and principle component analysis (PCA), augmented with self-reported ethnic and geographic origin was investigated. Genomic inflation factor was λ=1.03. When compared to HAPMAP reference samples, NGRC clustered well with Caucasians. However, within NGRC, which is a typical mixed Caucasian European-American population, evidence was found for significant genetic diversity (PC1, P=2.9×10⁻⁵, Eigen-value=3.97; PC2, P=0.007, Eigen-value=1.26; PC3 was not significant P=0.32, Eigen-value=1.11). Using self reported data on ancestry, it was determined that the primary clusters correlate with Ashkenazi-Jewish and non-Jewish ancestry, and the residual diversity in the larger non-Ashkenazi population correlates with the European country from which the subjects' ancestors had immigrated to the US. The association was present in genetically-defined core subsamples of both Jewish (0.04≦PC1≦0.055 and 0.00≦PC2≦0.013) and non-Jewish (−0.0075≦PC1≦0.0025 & −0.005≦PC2≦0.003) clusters. The frequency of the HLA-associated risk allele varied significantly in controls (P=0.0007), from 0.36 in Washington to 0.46 in New York. Similarly, a frequency gradient was observed across Europe, low in subjects with Northern-European ancestry and high in Southern-Europeans, particularly Italians. According to the US census, 14.4% of New Yorkers are Italian vs. 5.6% nation-wide, which may explain the high allele frequency in NY.

The HLA variant that displays the strongest statistical association with PD is rs3129882, which is in intron-1 of HLA-DRA gene (6p21.3). The protein chains encoded by the closely linked HLA-DRA and HLA-DRB form the class II HLA-DR antigens, which are expressed by antigen presenting cells including microglia in brain, and interact with T cell receptors. HLA-DRB chains are highly variable and associated with numerous disorders, whereas HLA-DRA is practically monomorphic. PD-association with an intronic DRA variant may represent involvement of regulatory elements, which would be in line with PD-specific over-expression of DR antigens in substantia nigra. Alternatively; PD-association may be with a closely linked classical HLA-DRB antigen.

Human Subjects

Study was approved by Human Subject Committees at participating institutions. Patients and Control subjects were recruited from eight NGRC-affiliated neurology clinics in Oregon, Washington, Georgia and New York. Methods were standardized across NGRC. Patients were diagnosed using the modified UK Brain Bank criteria. Controls were community volunteers and patient spouses; 353 (ages 67-90; mean 86.3±4.1 years) were evaluated by neurologists in Oregon and were free of neurodegenerative disease, the remaining 1633 (ages 21-90, mean 67.0±12.0 years) self-reported as neurologically healthy. GWAS subjects met seven criteria: (1) Self-reported Caucasian, non-Hispanic, of European origin. (2) Patients with current diagnosis of PD, excluding those whose initial PD diagnosis changed during the ˜12 years of follow up. (3) DNA extracted from whole blood, unamplified, concentration≧50 ng/μl. (4) Age at blood draw≧21 years. (5) Known gender. (6) Known age at onset (one missing). (7) No blood-relation to other subjects. Applicants had data on smoking, coffee, and NSAIDs on ˜3,000 subjects and on country of ancestral origin for 2,080 subjects. 112 subjects reported as Ashkenazi Jewish (129 were Jewish according to PCA).

Genotyping

To reduce plate effects, DNA samples were randomized on genotyping plates by case-control status, recruitment site, control subjects who were healthy at age≧85 years, DNA extraction method, and DNA storage time. Samples were genotyped at the Johns Hopkins Center for Inherited Disease Research (CIDR). Data was released for 4,013 study samples (99.5% of attempted samples). Study samples, 90 duplicates, and 170 HapMap controls (151 CEU; 12 YR1, 3 JPT, 4 CHB) were genotyped using Illumina HumanOmni 1-Quad_v1-0_B BeadChips (Illumina, San Diego, Calif., USA) and the Illumina Infinium II assay protocol. Genotype cluster definitions for each SNP were determined using Illumina BeadStudio Genotyping Module version 3.3.7 and the combined intensity data from all released samples. Genotypes were not called if the quality threshold (Gencall score) was <0.15. Genotype data was released for 1,012,895 SNPs (99.65% of attempted). SNP assay failure criteria were: call rate<85%, cluster separation<0.2, >1 HapMap replicate error, >3% (autosomal) or >4% (X) difference in call rate between genders, >0.3% male AB frequency (X), or >8.8% (autosomal) or >13.2% (XY) difference in AB frequency. Y chromosome and mitochondrial SNPs were manually reviewed and clusters adjusted or genotypes dropped as appropriate. The mean non-Y SNP call rate and mean sample call rate were both 99.9% for the released dataset. Study duplicate reproducibility was ≧99.99%.

Statistical Analysis

Sex was determined by estimating X chromosome homozygosity and compared to self-reported gender; there was no discrepancy. Applicants identified and excluded 1 patient and 3 controls who were inadvertently enrolled twice, and 13 cases and 10 controls for cryptic relatedness (PI-HAT>0.15). The final N for analysis was 2,000 patients and 1,986 controls. SNPs were excluded if MAF<0.01, call-rate<99%, HWE<10⁻⁶, MAF difference in males vs. females>0.15, or missing rate in PD vs. control P<10⁻⁵. 811,597 SNPs passed quality-control; with mean call rate of 99.92%. PCA was conducted with HelixTree (The website of goldenhelix) using a pruned subset of 104,064 SNPs. Pruning was carried-out using PLINK with autosomal SNPs (MAF≧0.05, call rate≧95%). Applicants used a 50-SNP sliding-window that shifted 5 SNPs with each move and recursively removed SNPs with r²≧0.2, followed by a second round using a 138-SNP sliding-window, resulting in 104,064 SNPs.

Association was tested under an additive model using logistic regression in PLINK V1.07. The models were corrected for sex, age, PC1 and PC2. Linkage disequilibrium (LD) was assessed using Haploview V4.1. For replication of NGRC results, datasets were chosen that (a) were published by peer review, (b) had genotyped rs3129882, and (c) were Caucasian. Among four publicly available GWAS only “CIDR: Genome Wide Association Study in Familial Parkinson Disease (PD)” by Pankratz et al. met the criteria. Two other GWAS have been published; one was available to us and was used as second replication. The same sample and SNP quality-control filters was applied to replication data sets as was similarly applied for NGRC, hence the results may vary slightly from published results. Each replication dataset was individually tested using the tests and adjustments used by authors in their original report. Breslow-Day test-statistics was used to test heterogeneity across three datasets (P=0.6). Individual level Meta-analysis was performed using Cochran-Mantel-Haenszel (CMH) test statistics accounting for study and gender. Chi-square test was used to test differences in MAF across disease, ethnic, and geographic strata.

To replicate previously reported GWAS findings, genotyped data was used when available and imputed SNPs that were not present on the array. PLINK was used for imputation, and included SNPs that had call rate≧95% (information content metric value was >0.8). Meta-analysis was performed using CMH when individual level data was available, otherwise, aggregate (OR) data (PLINK was used) was used. Breslow-Day test-statistics was used to test between-study heterogeneity for individual-level data and Q-test for aggregate-level data.

Logistic regression was used to test combined effects of four loci (SNCA, MAPT, GAK, HLA). Subjects were classified by the total number of risk alleles (n) that they possessed (minimum=0, maximum=8); carriers of 0 or 1 risk allele were combined due to small numbers and set as the reference for comparison; carriers of 6 or more alleles were also combined due to small numbers at the extremes. Tests were performed comparing subjects with ‘n’ risk alleles to the reference group.

### REFERENCES

1. Payami, H., D. M. Kay, C. P. Zabetian, G. D. Schellenberg, S. A. Factor, and C. C. McCulloch (2009) Visualizing disease associations: graphic analysis of frequency distributions as a function of age using moving average plots (MAP) with application to Alzheimer's and Parkinson's disease. Genet Epidemiol [Epub ahead of print] PMID: 19582778
2. Factor, S. A., W. J. Weiner, and eds., Parkinson's Disease: Diagnosis and Clinical Management. Second ed. 2008, New York, N. Y.: Demos Medical Publishing, Inc.
3. Polymeropoulos, M. H., C. Lavedan, E. Leroy, S. E. Ide, A. Dehejia, A. Dutra, B. Pike, H. Root, J. Rubenstein, R. Boyer, E. S. Stenroos, S. Chandrasekharappa, A. Athanassiadou, T. Papapetropoulos, W. G. Johnson, A. M. Lazzarini, R. C. Duvoisin, G. Di Iorio, L. I. Golbe, and R. L. Nussbaum (1997) Mutation in the alpha-synuclein gene identified in families with Parkinson's disease. Science 276, 2045-7.
4. Kitada, T., S. Asakawa, N. Hattori, H. Matsumine, Y. Yamamura, S. Minoshima, M. Yokochi, Y. Mizuno, and N. Shimizu (1998) Mutations in the parkin gene cause autosomal recessive juvenile parkinsonism. Nature 392, 605-608.
5. Leroy, E., G. Auburger, B. Leube, G. Ulm, E. Mezey, G. Harta, M. Brownstein, S. Jonnalagada, T. Chemova, A. Dehejia, C. Lavedan, T. Gasser, P. Steinbach, K. Winkinson, and M. Polymoropoulos (1998) The ubiquitin pathway in Parkinson's disease. Nature 395, 451-452.
6. Valente, E. M., P. M. Abou-Sleiman, V. Caputo, M. M. Muqit, K. Harvey, S. Gispert, Z. Ali, D. Del Turco, A. R. Bentivoglio, D. G. Healy, A. Albanese, R. Nussbaum, R. Gonzalez-Maldonado, T. Deller, S. Salvi, P. Cortelli, W. P. Gilks, D. S. Latchman, R. J. Harvey, B. Dallapiccola, G. Auburger, and N. W. Wood (2004) Hereditary early-onset Parkinson's disease caused by mutations in PINK1. Science 304, 1158-60.
7. Bonifati, V., P. Rizzu, M. J. van Baren, O. Schaap, G. J. Breedveld, E. Krieger, M. C. Dekker, F. Squitieri, P. Ibanez, M. Joosse, J. W. van Dongen, N. Vanacore, J. C. van Swieten, A. Brice, G. Meco, C. M. van Duijn, B. A. Oostra, and P. Heutink (2003) Mutations in the DJ-1 gene associated with autosomal recessive early-onset parkinsonism. Science 299, 256-9.
8. Le, W. D., P. Xu, J. Jankovic, H. Jiang, S. H. Appel, R. G. Smith, and D. K. Vassilatis (2003) Mutations in NR4A2 associated with familial Parkinson disease. Nat Genet 33, 85-9.
9. Paisan-Ruiz, C., S. Jain, E. W. Evans, W. P. Gilks, J. Simon, M. van der Brug, A. L. de Munain, S. Aparicio, A. M. Gil, N. Khan, J. Johnson, J. R. Martinez, D. Nicholl, I. M. Carrera, A. S. Pena, R. de Silva, A. Lees, J. F. Marti-Masso, J. Perez-Tur, N. W. Wood, and A. B. Singleton (2004) Cloning of the gene containing mutations that cause PARKS-linked Parkinson's disease. Neuron 44, 595-600.
10. Zimprich, A., S. Biskup, P. Leitner, P. Lichtner, M. Farrer, S. Lincoln, J. Kachergus, M. Hulihan, R. J. Uitti, D. B. Calne, A. J. Stoessl, R. F. Pfeiffer, N. Patenge, I. C. Carbajal, P. Vieregge, F. Asmus, B. Muller-Myhsok, D. W. Dickson, T. Meitinger, T. M. Strom, Z. K. Wszolek, and T. Gasser (2004) Mutations in LRRK2 cause autosomal-dominant parkinsonism with pleomorphic pathology. Neuron 44, 601-7.
11. Kay, D. M., C. P. Zabetian, S. A. Factor, J. G. Nutt, A. Samii, A. Griffith, T. D. Bird, P. Kramer, D. S. Higgins, and H. Payami (2006) Parkinson's disease and LRRK2: frequency of a common mutation in U. S. movement disorder clinics. Mov Disord 21, 519-23.
12. Healy, D. G., M. Falchi, S. S. O'Sullivan, V. Bonifati, A. Durr, S. Bressman, A. Brice, J. Aasly, C. P. Zabetian, S. Goldwurm, J. J. Ferreira, E. Tolosa, D. M. Kay, C. Klein, D. R. Williams, C. Marras, A. E. Lang, Z. K. Wszolek, J. Berciano, A. H. Schapira, T. Lynch, K. P. Bhatia, T. Gasser, A. J. Lees, and N. W. Wood (2008) Phenotype, genotype, and worldwide genetic penetrance of LRRK2-associated Parkinson's disease: a case-control study. Lancet Neurol 7, 583-90.
13. pdgene website
14. Kay, D. M., S. A. Factor, A. Samii, D. S. Higgins, A. Griffith, J. W. Roberts, B. C. Leis, J. G. Nutt, J. S. Montimurro, R. G. Keefe, A. J. Atkins, D. Yearout, C. P. Zabetian, and H. Payami (2008) Genetic association between alpha-synuclein and idiopathic Parkinson's disease. Am J Med Genet B Neuropsychiatr Genet 147B, 1222-30.
15. Maraganore, D. M., M. de Andrade, A. Elbaz, M. J. Farrer, J. P. Ioannidis, R. Kruger, W. A. Rocca, N. K. Schneider, T. G. Lesnick, S. J. Lincoln, M. M. Hulihan, J. O. Aasly, T. Ashizawa, M. C. Chartier-Harlin, H. Checkoway, C. Ferrarese, G. Hadjigeorgiou, N. Hattori, H. Kawakami, J. C. Lambert, T. Lynch, G. D. Mellick, S. Papapetropoulos, A. Parsian, A. Quattrone, O. Riess, E. K. Tan, and C. Van Broeckhoven (2006) Collaborative analysis of alpha-synuclein gene promoter variability and Parkinson disease. JAMA 296, 661-70.
16. Healy, D. G., P. M. Abou-Sleiman, A. J. Lees, J. P. Casas, N. Quinn, K. Bhatia, A. D. Hingorani, and N. W. Wood (2004) Tau gene and Parkinson's disease: a case-control study and meta-analysis. J Neurol Neurosurg Psychiatry 75, 962-5.
17. Zabetian, C. P., C. M. Hutter, S. A. Factor, J. G. Nutt, D. S. Higgins, A. Griffith, J. W. Roberts, B. C. Leis, D. M. Kay, D. Yearout, J. S. Montimurro, K. L. Edwards, A. Samii, and H. Payami (2007) Association analysis of MAPT H1 haplotype and subhaplotypes in Parkinson's disease. Ann Neurol 62, 137-144.
18. Zareparsi, S., T. D. Taylor, E. L. Harris, and H. Payami (1998) Segregation analysis of Parkinson disease. Am J Med Genet 80, 410-417.
19. Maher, N. E., L. J. Currie, A. M. Lazzarini, J. B. Wilk, C. A. Taylor, M. H. Saint-Hilaire, R. G. Feldman, L. I. Golbe, G. F. Wooten, and R. H. Myers (2002) Segregation analysis of Parkinson disease revealing evidence for a major causative gene. Am J Med Genet 109, 191-7.
20. McDonnell, S. K., D. J. Schaid, A. Elbaz, K. J. Strain, J. H. Bower, J. E. Ahlskog, D. M. Maraganore, and W. A. Rocca (2006) Complex segregation analysis of Parkinson's disease: The Mayo Clinic Family Study. Ann Neurol 59, 788-95.
21. Moilanen, J. S., J. M. Autere, V. V. Myllyla, and K. Majamaa (2001) Complex segregation analysis of Parkinson's disease in the Finnish population. Hum Genet 108, 184-9.
22. Payami, H., K. Larsen, S. Bernard, and J. Nutt (1994) Increased risk of Parkinson's disease in parents and siblings of patients. Ann Neurol 36, 659-661.
23. Autere, J. M., J. S. Moilanen, V. V. Myllyla, and K. Majamaa (2000) Familial aggregation of Parkinson's disease in a Finnish population. J Neurol Neurosurg Psychiatry 69, 107-9.
24. Bonifati, V., E. Fabrizio, N. Vanacore, M. De Mari, and G. Meco (1995) Familial Parkinson's disease: A clinical genetic analysis. Can J Neurol Sci 22, 272-279.
25. De Michele, G., A. Filla, G. Volpe, V. De Marco, A. Gogliettino, G. Ambrosio, R. Marconi, A. E. Castellano, and G. Campanella (1996) Environmental and genetic risk factors in Parkinson's disease: a case-control study in southern Italy. Mov Disord 11, 17-23.
26. Kurz, M., G. Alves, D. Aarsland, and J. P. Larsen (2003) Familial Parkinson's disease: a community-based study. Eur J Neurol 10, 159-63.
27. Marder, K., G. Levy, E. D. Louis, H. Mejia-Santana, L. Cote, H. Andrews, J. Harris, C. Waters, B. Ford, S. Frucht, S. Fahn, and R. Ottman (2003) Familial aggregation of early- and late-onset Parkinson's disease. Ann Neurol 54, 507-13.
28. Marder, K., M.-X. Tang, H. Mejia, B. Alfaro, L. Cote, E. Louis, J. Groves, and R. Mayeux (1996) Risk of Parkinson's disease among first-degree relatives: A community-based study. Neurology 47, 155-160.
29. Payami, H., S. Zareparsi, D. James, and J. Nutt (2002) Familial aggregation of Parkinson disease: a comparative study of early-onset and late-onset disease. Arch Neurol 59, 848-50.
30. Uitti, R. J., H. Shinotoh, M. Hayward, M. Schulzer, E. Mak, and D. B. Calne (1997) “Familial Parkinson's disease”—A case-control study of families. Can J Neurol Sci 24, 127-132.
31. Vieregge, P. and I. Heberlein (1995) Increased risk of Parkinson's disease in relatives of patients. Ann Neurol 37, 685.
32. Zorzon, M., L. Capus, A. Pellegrino, G. Cazzato, and R. Zivadinov (2002) Familial and environmental risk factors in Parkinson's disease: a case-control study in north-east Italy. Acta Neurol Scand 105, 77-82.
33. Rocca, W. A., S. K. McDonnell, K. J. Strain, J. H. Bower, J. E. Ahlskog, A. Elbaz, D. J. Schaid, and D. M. Maraganore (2004) Familial aggregation of Parkinson's disease: The Mayo Clinic family study. Ann Neurol 56, 495-502.
34. Thacker, E. L. and A. Ascherio (2008) Familial aggregation of Parkinson's disease: A meta-analysis. Mov Disord 23, 1174-83.
35. Sveinbjornsdottir, S., A. A. Hicks, T. Jonsson, H. Petursson, G. Guomundsson, M. L. Frigge, A. Kong, J. R. Gulcher, and K. Stefansson (2000) Familial Aggregation of Parkinson's Disease in Iceland. N Engl J Med 343, 1765-1770.
36. Tanner, C., R. Ottman, S. Goldman, J. Ellenberg, P. Chan, R. Mayeux, and J. Langston (1999) Parkinson disease in twins: An Etiologic study. JAMA 281, 341-346.
37. Wirdefeldt, K., M. Gatz, M. Schalling, and N. L. Pedersen (2004) No evidence for heritability of Parkinson disease in Swedish twins. Neurology 63, 305-11.
38. Lin, M. T. and D. K. Simon (2005) No evidence for heritability of Parkinson disease in Swedish twins. Neurology 64, 932; author reply 932.
39. Tanner, C., Etiology: the role of environmental and genetics. In: Factor S A & Weiner W J, eds., in Parkinson's Disease: Diagnosis and Clinical Management. 2002, Demos: New York. p. 265-280.
40. Mayeux, R. (2003) Epidemiology of neurodegeneration. Annu Rev Neurosci 26, 81-104.
41. Hernan, M. A., B. Takkouche, F. Caamano-Isorna, and J. J. Gestal-Otero (2002) A meta-analysis of coffee drinking, cigarette smoking, and the risk of Parkinson's disease. Ann Neurol 52, 276-84.
42. Powers, K., D. Kay, S. Factor, C. Zabetian, D. Higgins, A. Samii, J. Nutt, A. Griffith, B. Leis, J. Roberts, E. Martinez, J. Montimurro, H. Checkoway, and H. Payami (2008) Combined effects of smoking, coffee and NSAIDs on Parkinson's disease risk. Mov Disord 23, 88-95.
43. Tanner, C. M., S. M. Goldman, D. A. Aston, R. Ottman, J. Ellenberg, R. Mayeux, and J. W. Langston (2002) Smoking and Parkinson's disease in twins. Neurology 58, 581-8.
44. Scott, W. K., F. Zhang, J. M. Stajich, B. L. Scott, M. A. Stacy, and J. M. Vance (2005) Family-based case-control study of cigarette smoking and Parkinson disease. Neurology 64, 442-7.
45. Galanaud, J. P., A. Elbaz, J. Clavel, J. S. Vidal, J. R. Correze, A. Alperovitch, and C. Tzourio (2005) Cigarette smoking and Parkinson's disease: a case-control study in a population characterized by a high prevalence of pesticide exposure. Mov Disord 20, 181-9.
46. Mellick, G. D., C. E. Gartner, P. A. Silburn, and D. Battistutta (2006) Passive smoking and Parkinson disease. Neurology 67, 179-80.
47. Can, L. A. and P. P. Rowell (1990) Attenuation of 1-methyl-4-phenyl-1,2,3,6-tetrahydropyridine-induced neurotoxicity by tobacco smoke. Neuropharmacology 29, 311-4.
48. Shahi, G. S., N. P. Das, and S. M. Moochhala (1991) 1-Methyl-4-phenyl-1,2,3,6-tetrahydropyridine-induced neurotoxicity: partial protection against striato-nigral dopamine depletion in C57BL/6J mice by cigarette smoke exposure and by beta-naphthoflavone-pretreatment. Neurosci Lett 127, 247-50.
49. Janson, A. M., K. Fuxe, and M. Goldstein (1992) Differential effects of acute and chronic nicotine treatment on MPTP-(1-methyl-4-phenyl-1,2,3,6-tetrahydropyridine) induced degeneration of nigrostriatal dopamine neurons in the black mouse. Clin Investig 70, 232-238.
50. Quik, M. (2004) Smoking, nicotine and Parkinson's disease. Trends Neurosci 27, 561-8.
51. Chen, J. F., K. Xu, J. P. Petzer, R. Staal, Y. H. Xu, M. Beilstein, P. K. Sonsalla, K. Castagnoli, N. Castagnoli, Jr., and M. A. Schwarzschild (2001) Neuroprotection by caffeine and A(2A) adenosine receptor inactivation in a model of Parkinson's disease. J Neurosci 21, RC143.
52. Joghataie, M. T., M. Roghani, F. Negandar, and L. Hashemi (2004) Protective effect of caffeine against neurodegeneration in a model of Parkinson's disease in rat: behavioral and histochemical evidence. Parkinsonism Relat Disord 10, 465-8.
53. Khwaja, M., A. McCormack, J. M. McIntosh, D. A. Di Monte, and M. Quik (2007) Nicotine partially protects against paraquat-induced nigrostriatal damage in mice; link to alpha6beta2* nAChRs. J Neurochem 100, 180-90.
54. Park, H. J., P. H. Lee, Y. W. Ahn, Y. J. Choi, G. Lee, D. Y. Lee, E. S. Chung, and B. K. Jin (2007) Neuroprotective effect of nicotine on dopaminergic neurons by anti-inflammatory action. Eur J Neurosci 26, 79-89.
55. Castagnoli, K. P., S. J. Steyn, J. P. Petzer, C. J. Van der Schyf, and N. Castagnoli, Jr. (2001) Neuroprotection in the MPTP Parkinsonian C57BL/6 mouse model by a compound isolated from tobacco. Chem Res Toxicol 14, 523-7.
56. Ikeda, K., M. Kurokawa, S. Aoyama, and Y. Kuwana (2002) Neuroprotection by adenosine A2A receptor blockade in experimental models of Parkinson's disease. J Neurochem 80, 262-70.
57. Factor, S. A. (2008) Current status of symptomatic medical therapy in Parkinson's disease. Neurotherapeutics 5, 164-80.
58. LeWitt, P. A., M. Guttman, J. W. Tetrud, P. J. Tuite, A. Mori, P. Chaikin, and N. M. Sussman (2008) Adenosine A2A receptor antagonist istradefylline (KW-6002) reduces “off” time in Parkinson's disease: a double-blind, randomized, multicenter clinical trial (6002-US-005). Ann Neurol 63, 295-302.
59. Stacy, M., D. Silver, T. Mendis, J. Sutton, A. Mori, P. Chaikin, and N. M. Sussman (2008) A 12-week, placebo-controlled study (6002-US-006) of istradefylline in Parkinson disease. Neurology 70, 2233-40.
60. McCulloch, C. C., D. M. Kay, S. A. Factor, A. Samii, J. G. Nutt, D. S. Higgins, A. Griffith, J. W. Roberts, B. C. Leis, J. S. Montimurro, C. P. Zabetian, and H. Payami (2008) Exploring gene-environment interactions in Parkinson's disease. Hum Genet 123, 257-65.
61. Hirschhorn, J. N. and M. J. Daly (2005) Genome-wide association studies for common diseases and complex traits. Nat Rev Genet 6, 95-108.
62. Estivill, X. and L. Armengol (2007) Copy Number Variants and Common Disorders: Filling the Gaps and Exploring Complexity in Genome-Wide Association Studies. PLoS Genet 3, e190.
63. Topol, E. J., S. S. Murray, and K. A. Frazer (2007) The genomics gold rush. Jama 298, 218-21.
64. Maraganore, D. M., M. de Andrade, T. G. Lesnick, K. J. Strain, M. J. Farrer, W. A. Rocca, P. V. Pant, K. A. Frazer, D. R. Cox, and D. G. Ballinger (2005) High-resolution whole-genome association study of Parkinson disease. Am J Hum Genet 77, 685-93.
65. Fung, H. C., S. Scholz, M. Matarin, J. Simon-Sanchez, D. Hernandez, A. Britton, J. R. Gibbs, C. Langefeld, M. L. Stiegert, J. Schymick, M. S. Okun, R. J. Mandel, H. H. Fernandez, K. D. Foote, R. L. Rodriguez, E. Peckham, F. W. De Vrieze, K. Gwinn-Hardy, J. A. Hardy, and A. Singleton (2006) Genome-wide genotyping in Parkinson's disease and neurologically normal controls: first stage analysis and public release of data. Lancet Neurol 5, 911-6.
66. Pankratz, N., J. B. Wilk, J. C. Latourelle, A. L. DeStefano, C. Halter, E. W. Pugh, K. F. Doheny, J. F. Gusella, W. C. Nichols, T. Foroud, and R. H. Myers (2009) Genomewide association study for susceptibility genes contributing to familial Parkinson disease. Hum Genet 124, 593-605.
67. Elbaz, A., L. M. Nelson, H. Payami, J. P. Ioannidis, B. K. Fiske, G. Annesi, A. Carmine Belin, S. A. Factor, C. Ferrarese, G. M. Hadjigeorgiou, D. S. Higgins, H. Kawakami, R. Kruger, K. S. Marder, R. P. Mayeux, G. D. Mellick, J. G. Nutt, B. Ritz, A. Samii, C. M. Tanner, C. Van Broeckhoven, S. K. Van Den Eeden, K. Wirdefeldt, C. P. Zabetian, M. Dehem, J. S. Montimurro, A. Southwick, R. M. Myers, and T. A. Trikalinos (2006) Lack of replication of thirteen single-nucleotide polymorphisms implicated in Parkinson's disease: a large-scale international study. Lancet Neurol 5, 917-23.
68. Evangelou, E., D. M. Maraganore, and J. P. Ioannidis (2007) Meta-analysis in genome-wide association datasets: strategies and application in Parkinson disease. PLoS ONE 2, e196.
69. Frayling, T. M. (2007) Genome-wide association studies provide new insights into type 2 diabetes aetiology. Nat Rev Genet 8, 657-62.
70. Ioannidis, J. P., T. A. Trikalinos, and M. J. Khoury (2006) Implications of small effect sizes of individual genetic variants on the design and interpretation of genetic association studies of complex diseases. Am J Epidemiol 164, 609-14.
71. Marchini, J., P. Donnelly, and L. R. Cardon (2005) Genome-wide strategies for detecting multiple loci that influence complex diseases. Nat Genet 37, 413-7.
72. Bilen, J. and N. M. Bonini (2005) Drosophila as a model for human neurodegenerative disease. Annu Rev Genet 39, 153-71.
73. Cauchi, R. J. and M. van den Heuvel (2006) The fly as a model for neurodegenerative diseases: is it worth the jump? Neurodegener Dis 3, 338-56.
74. Marsh, J. L. and L. M. Thompson (2006) Drosophila in the study of neurodegenerative disease. Neuron 52, 169-78.
75. Auluck, P. K., H. Y. Chan, J. Q. Trojanowski, V. M. Lee, and N. M. Bonini (2002) Chaperone suppression of alpha-synuclein toxicity in a Drosophila model for Parkinson's disease. Science 295, 865-8.
76. Feany, M. B. and W. W. Bender (2000) A Drosophila model of Parkinson's disease. Nature 404, 394-8.
77. Liu, Z., X. Wang, Y. Yu, X. Li, T. Wang, H. Jiang, Q. Ren, Y. Jiao, A. Sawa, T. Moran, C. A. Ross, C. Montell, and W. W. Smith (2008) A Drosophila model for LRRK2-linked parkinsonism. Proc Natl Acad Sci USA 105, 2693-8.
78. Sang, T. K., H. Y. Chang, G. M. Lawless, A. Ratnaparkhi, L. Mee, L. C. Ackerson, N. T. Maidment, D. E. Krantz, and G. R. Jackson (2007) A Drosophila model of mutant human parkin-induced toxicity demonstrates selective loss of dopaminergic neurons and dependence on cellular dopamine. J Neurosci 27, 981-92.
79. Trinh, K., K. Moore, P. D. Wes, P. J. Muchowski, J. Dey, L. Andrews, and L. J. Pallanck (2008) Induction of the phase II detoxification pathway suppresses neuron loss in Drosophila models of Parkinson's disease. J Neurosci 28, 465-72.
80. Greene, J. C., A. J. Whitworth, L. A. Andrews, T. J. Parker, and L. J. Pallanck (2005) Genetic and genomic studies of Drosophila parkin mutants implicate oxidative stress and innate immune responses in pathogenesis. Hum Mol Genet 14, 799-811.
81. Greene, J. C., A. J. Whitworth, I. Kuo, L. A. Andrews, M. B. Feany, and L. J. Pallanck (2003) Mitochondrial pathology and apoptotic muscle degeneration in Drosophila parkin mutants. Proc Natl Acad Sci USA 100, 4078-83.
82. Menzies, F. M., S. C. Yenisetti, and K. T. Min (2005) Roles of Drosophila DJ-1 in survival of dopaminergic neurons and oxidative stress. Curr Biol 15, 1578-82.
83. Meulener, M., A. J. Whitworth, C. E. Armstrong-Gold, P. Rizzu, P. Heutink, P. D. Wes, L. J. Pallanck, and N. M. Bonini (2005) Drosophila DJ-1 mutants are selectively sensitive to environmental toxins associated with Parkinson's disease. Curr Biol 15, 1572-7.
84. Wang, D., L. Qian, H. Xiong, J. Liu, W. S. Neckameyer, S. Oldham, K. Xia, J. Wang, R. Bodmer, and Z. Zhang (2006) Antioxidants protect PINK1-dependent dopaminergic neurons in Drosophila. Proc Natl Acad Sci USA 103, 13520-5.
85. Bier, E. (2006) Antioxidants put Parkinson flies back in the PINK. Proc Natl Acad Sci USA 103, 13269-70.
86. Pesah, Y., T. Pham, H. Burgess, B. Middlebrooks, P. Verstreken, Y. Zhou, M. Harding, H. Bellen, and G. Mardon (2004) Drosophila parkin mutants have decreased mass and cell size and increased sensitivity to oxygen radical stress. Development 131, 2183-94.
87. Whitworth, A. J., D. A. Theodore, J. C. Greene, H. Benes, P. D. Wes, and L. J. Pallanck (2005) Increased glutathione S-transferase activity rescues dopaminergic neuron loss in a Drosophila model of Parkinson's disease. Proc Natl Acad Sci USA 102, 8024-9.
88. Clark, I. E., M. W. Dodson, C. Jiang, J. H. Cao, J. R. Huh, J. H. Seol, S. J. Yoo, B. A. Hay, and M. Guo (2006) Drosophila pink1 is required for mitochondrial function and interacts genetically with parkin. Nature 441, 1162-6.
89. Yang, Y., S. Gehrke, Y. Imai, Z. Huang, Y. Ouyang, J. W. Wang, L. Yang, M. F. Beal, H. Vogel, and B. Lu (2006) Mitochondrial pathology and muscle and dopaminergic neuron degeneration caused by inactivation of Drosophila Pinkl is rescued by Parkin. Proc Natl Acad Sci USA 103, 10793-8.
90. Dinis-Oliveira, R. J., F. Remiao, H. Carmo, J. A. Duarte, A. S. Navarro, M. L. Bastos, and F. Carvalho (2006) Paraquat exposure as an etiological factor of Parkinson's disease. Neurotoxicology 27, 1110-22.
91. Costello, S., M. Cockburn, J. Bronstein, X. Zhang, and B. Ritz (2009) Parkinson's disease and residential exposure to maneb and paraquat from agricultural applications in the central valley of California. Am J Epidemiol 169, 919-26.
92. Firestone, J. A., T. Smith-Weller, G. Franklin, P. Swanson, W. T. Longstreth, Jr., and H. Checkoway (2005) Pesticides and risk of Parkinson disease: a population-based case-control study. Arch Neurol 62, 91-5.
93. Liou, H. H., M. C. Tsai, C. J. Chen, J. S. Jeng, Y. C. Chang, S. Y. Chen, and R. C. Chen (1997) Environmental risk factors and Parkinson's disease: a case-control study in Taiwan. Neurology 48, 1583-8.
94. Almasy, L. and J. Blangero (1998) Multipoint quantitative-trait linkage analysis in general pedigrees. Am J Hum Genet 62, 1198-211.
95. Khoury, M., T. Beaty, and K.-Y. Liang (1988) Can familial aggregation of disease be explained by familial aggregation of environmental risk factors? Am J Epidemiol 127, 674-683.
96. Farrer, L. A., D. M. O'Sullivan, L. A. Cupples, J. H. Growdon, and R. H. Myers (1989) Assessment of genetic risk for Alzheimer's disease among first-degree relatives. Ann Neurol 25, 485-93.
97. Payami, H., K. Montee, and J. Kaye (1994) Evidence for familial factors that protect against dementia and outweigh the effect of increasing age. Am J Hum Genet 54, 650-657.
98. Daw, E. W., H. Payami, E. J. Nemens, D. Nochlin, T. D. Bird, G. D. Schellenberg, and E. M. Wijsman (2000) The number of trait loci in late-onset Alzheimer disease. Am J Hum Genet 66, 196-204.
99. Ascherio, A., S. M. Zhang, M. A. Hernan, I. Kawachi, G. A. Colditz, F. E. Speizer, and W. C. Willett (2001) Prospective study of caffeine consumption and risk of Parkinson's disease in men and women. Ann Neurol 50, 56-63.
100. Hernan, M., G. Logroscino, and L. Garcia Rodriguez (2006) Nonsteroidal anti-inflammatory drugs and the incidence of Parkinson's disease. Neurology 11, 1097-9.
101. Ascherio, A., H. Chen, M. A. Schwarzschild, S. M. Zhang, G. A. Colditz, and F. E. Speizer (2003) Caffeine, postmenopausal estrogen, and risk of Parkinson's disease. Neurology 60, 790-5.
102. Huang, X., P. C. Chen, and C. Poole (2004) APOE-[epsilon]2 allele associated with higher prevalence of sporadic Parkinson disease. Neurology 62, 2198-202.
103. Payami, H., M. Zhu, J. Montimurro, R. Keefe, C. C. McCulloch, and L. Moses (2005) One step closer to fixing association studies: evidence for age- and gender-specific allele frequency variations and deviations from Hardy-Weinberg expectations in controls. Hum Genet 118, 322-30.
104. Spillantini, M. G., M. L. Schmidt, V. M.-Y. Lee, J. Q. Trojanowski, R. Jakes, and M. Goedert (1997)-Synuclein in Lewy bodies. Nature 388, 839-840.
105. Singleton, A. B., M. Farrer, J. Johnson, A. Singleton, S. Hague, J. Kachergus, M. Hulihan, T. Peuralinna, A. Dutra, R. Nussbaum, S. Lincoln, A. Crawley, M. Hanson, D. Maraganore, C. Adler, M. R. Cookson, M. Muenter, M. Baptista, D. Miller, J. Blancato, J. Hardy, and K. Gwinn-Hardy (2003) alpha-Synuclein locus triplication causes Parkinson's disease. Science 302, 841.
106. Scherzer, C. R., J. A. Grass, Z. Liao, I. Pepivani, B. Zheng, A. C. Eklund, P. A. Ney, J. Ng, M. McGoldrick, B. Mollenhauer, E. H. Bresnick, and M. G. Schlossmacher (2008) GATA transcription factors directly regulate the Parkinson's disease-linked gene alpha-synuclein. Proc Natl Acad Sci USA 105, 10907-12.
107. Mitchell, M. K., P. L. Gregersen, S. Johnson, R. Parsons, and D. Vlahov (2004) The New York Cancer Project: Rationale, Organization, Design, and Baseline Characteristics. Journal of Urban Health Bulletin of the New York Academy of Medicine 81, 301-310.
108. intragen.cu-genome.org website
109. Purcell, S., B. Neale, K. Todd-Brown, L. Thomas, M. A. Ferreira, D. Bender, J. Maller, P. Sklar, P. I. de Bakker, M. J. Daly, and P. C. Sham (2007) PLINK: a tool set for whole-genome association and population-based linkage analyses. Am J Hum Genet 81, 559-75.
110. Henchcliffe, C. and M. F. Beal (2008) Mitochondrial biology and oxidative stress in Parkinson disease pathogenesis. Nat Clin Pract Neurol 4, 600-9.
111. Diederich, N. J., G. Fenelon, G. Stebbins, and C. G. Goetz (2009) Hallucinations in Parkinson disease. Nat Rev Neurol 5, 331-42.
112. Buter, T. C., A. van den Hout, F. E. Matthews, J. P. Larsen, C. Brayne, and D. Aarsland (2008) Dementia and survival in Parkinson disease: a 12-year population study. Neurology 70, 1017-22.
113. Kay, D. M., T. D. Bird, C. P. Zabetian, S. A. Factor, A. Samii, D. S. Higgins, J. Nutt, J. W. Roberts, A. Griffith, B. C. Leis, J. S. Montimurro, S. Philpott, and H. Payami (2006) Validity and utility of a LRRK2 G2019S mutation test for the diagnosis of Parkinson's disease. Genet Test 10, 221-7.
114. Kay, D. M., P. Kramer, D. Higgins, C. P. Zabetian, and H. Payami (2005) Escaping Parkinson's disease: A neurologically healthy octogenarian with the LRRK2 G2019S mutation. Mov Disord 20, 1077-8.
115. Kay, D. M., D. Moran, L. Moses, P. Poorkaj, C. P. Zabetian, J. Nutt, S. A. Factor, C. E. Yu, J. S. Montimurro, R. G. Keefe, G. D. Schellenberg, and H. Payami (2007) Heterozygous parkin point mutations are as common in control subjects as in Parkinson's patients. Ann Neurol 61, 47-54.
116. Zabetian, C., A. Samii, A. Mosley, J. Roberts, B. Leis, D. Yearout, W. Raskind, and A. Griffith (2005) A Clinic-Based Study of the LRRK2 Gene in Parkinson's Disease Yields New Mutations. Neurology 65, 741-4.
117. Zabetian, C. P., C. M. Hutter, D. Yearout, A. N. Lopez, S. A. Factor, A. Griffith, B. C. Leis, T. D. Bird, J. G. Nutt, D. S. Higgins, J. W. Roberts, D. M. Kay, K. L. Edwards, A. Samii, and H. Payami (2006) LRRK2 G2019S in families with Parkinson disease who originated from Europe and the Middle East: evidence of two distinct founding events beginning two millennia ago. Am J Hum Genet 79, 752-8.
118. Zabetian, C. P., C. J. Lauricella, D. W. Tsuang, J. B. Leverenz, G. D. Schellenberg, and H. Payami (2006) Analysis of the LRRK2 G2019S mutation in Alzheimer Disease. Arch Neurol 63, 156-7.
119. Gibb, W. and A. Lees (1988) The relevance of the Lewy body to the pathogenesis of idiopathic Parkinson's disease. J Neurol Neurosurg Psychiatry 51, 745-752.
120. Hughes, A. J., S. E. Daniel, Y. Ben-Shlomo, and A. J. Lees (2002) The accuracy of diagnosis of parkinsonian syndromes in a specialist movement disorder service. Brain 125, 861-70.
121. Tan, L. C., W. P. Koh, J. M. Yuan, R. Wang, W. L. Au, J. H. Tan, E. K. Tan, and M. C. Yu (2008) Differential effects of black versus green tea on risk of Parkinson's disease in the Singapore Chinese Health Study. Am J Epidemiol 167, 553-60.
122. illumina website
123. Price, A. L., N. J. Patterson, R. M. Plenge, M. E. Weinblatt, N. A. Shadick, and D. Reich (2006) Principal components analysis corrects for stratification in genome-wide association studies. Nat Genet 38, 904-9.
124. Redon, R., S. Ishikawa, K. R. Fitch, L. Feuk, G. H. Perry, T. D. Andrews, H. Fiegler, M. H. Shapero, A. R. Carson, W. Chen, E. K. Cho, S. Dallaire, J. L. Freeman, J. R. Gonzalez, M. Gratacos, J. Huang, D. Kalaitzopoulos, D. Komura, J. R. MacDonald, C. R. Marshall, R. Mei, L. Montgomery, K. Nishimura, K. Okamura, F. Shen, M. J. Somerville, J. Tchinda, A. Valsesia, C. Woodwark, F. Yang, J. Zhang, T. Zerjal, J. Zhang, L. Armengol, D. F. Conrad, X. Estivill, C. Tyler-Smith, N. P. Carter, H. Aburatani, C. Lee, K. W. Jones, S. W. Scherer, and M. E. Hurles (2006) Global variation in copy number in the human genome. Nature 444, 444-54.
125. Lupski, J. R. (2007) Genomic rearrangements and sporadic disease. Nat Genet 39, S43-7.
126. Sebat, J., B. Lakshmi, D. Malhotra, J. Troge, C. Lese-Martin, T. Walsh, B. Yamrom, S. Yoon, A. Krasnitz, J. Kendall, A. Leotta, D. Pai, R. Zhang, Y. H. Lee, J. Hicks, S. J. Spence, A. T. Lee, K. Puura, T. Lehtimaki, D. Ledbetter, P. K. Gregersen, J. Bregman, J. S. Sutcliffe, V. Jobanputra, W. Chung, D. Warburton, M. C. King, D. Skuse, D. H. Geschwind, T. C. Gilliam, K. Ye, and M. Wigler (2007) Strong association of de novo copy number mutations with autism. Science 316, 445-9.
127. Gonzalez, E., H. Kulkarni, H. Bolivar, A. Mangano, R. Sanchez, G. Catano, R. J. Nibbs, B. I. Freedman, M. P. Quinones, M. J. Bamshad, K. K. Murthy, B. H. Rovin, W. Bradley, R. A. Clark, S. A. Anderson, J. O'Connell R, B. K. Agan, S. S. Ahuja, R. Bologna, L. Sen, M. J. Dolan, and S. K. Ahuja (2005) The influence of CCL3L1 gene-containing segmental duplications on HIV-1/AIDS susceptibility. Science 307, 1434-40.
128. Stefansson, H., D. Rujescu, S. Cichon, O. P. Pietilainen, A. Ingason, S. Steinberg, R. Fossdal, E. Sigurdsson, T. Sigmundsson, J. E. Buizer-Voskamp, T. Hansen, K. D. Jakobsen, P. Muglia, C. Francks, P. M. Matthews, A. Gylfason, B. V. Halldorsson, D. Gudbjartsson, T. E. Thorgeirsson, A. Sigurdsson, A. Jonasdottir, A. Jonasdottir, A. Bjornsson, S. Mattiasdottir, T. Blondal, M. Haraldsson, B. B. Magnusdottir, I. Giegling, H. J. Moller, A. Hartmann, K. V. Shianna, D. Ge, A. C. Need, C. Crombie, G. Fraser, N. Walker, J. Lonnqvist, J. Suvisaari, A. Tuulio-Henriksson, T. Paunio, T. Toulopoulou, E. Bramon, M. Di Forti, R. Murray, M. Ruggeri, E. Vassos, S. Tosato, M. Walshe, T. Li, C. Vasilescu, T. W. Muhleisen, A. G. Wang, H. Ullum, S. Djurovic, I. Melle, J. Olesen, L. A. Kiemeney, B. Franke, C. Sabatti, N. B. Freimer, J. R. Gulcher, U. Thorsteinsdottir, A. Kong, O. A. Andreassen, R. A. Ophoff, A. Georgi, M. Rietschel, T. Werge, H. Petursson, D. B. Goldstein, M. M. Nothen, L. Peltonen, D. A. Collier, D. St Clair, K. Stefansson, R. S. Kahn, D. H. Linszen, J. van Os, D. Wiersma, R. Bruggeman, W. Cahn, L. de Haan, L. Krabbendam, and I. Myin-Germeys (2008) Large recurrent microdeletions associated with schizophrenia. Nature 455, 232-6.
129. Zhu, M. and A. Ghodsi, Automatic dimensionality selection from the scree plot via the use of profile likelihood, in Computational Statistics and Data Analysis. 2005. p. 918-930.
130. Hawkins, D., Fitting multiple change-points to data, in Computational Statistics and Data Analysis. 2002. p. 323-341.
131. Venkatraman, E. S. and A. B. Olshen (2007) A faster circular binary segmentation algorithm for the analysis of array CGH data. Bioinformatics 23, 657-63.
132. Maere, S., K. Heymans, and M. Kuiper (2005) BiNGO: a Cytoscape plugin to assess overrepresentation of gene ontology categories in biological networks. Bioinformatics 21, 3448-9.
133. McCarroll, S. A. and D. M. Altshuler (2007) Copy-number variation and association studies of human disease. Nat Genet 39, S37-42.
134. Gatto, N. M., U. B. Campbell, A. G. Rundle, and H. Ahsan (2004) Further development of the case-only design for assessing gene-environment interaction: evaluation of and adjustment for bias. Int J Epidemiol 33, 1014-24.
135. Piegorsch, W. W., C. R. Weinberg, and J. A. Taylor (1994) Non-hierarchical logistic models and case-only designs for assessing susceptibility in population-based case-control studies. Stat Med 13, 153-62.
136. Foppa, I. and D. Spiegelman (1997) Power and sample size calculations for case-control studies of gene-environment interactions with a polytomous exposure variable. Am J Epidemiol 146, 596-604.
137. Yang, Q., M. J. Khoury, and W. D. Flanders (1997) Sample size requirements in case-only designs to detect gene-environment interaction. Am J Epidemiol 146, 713-20.
138. Albert, P. S., D. Ratnasinghe, J. Tangrea, and S. Wacholder (2001) Limitations of the case-only design for identifying gene-environment interactions. Am J Epidemiol 154, 687-93.
139. Schmidt, S. and D. J. Schaid (1999) Potential misinterpretation of the case-only study to assess gene-environment interaction. Am J Epidemiol 150, 878-85.
140. Nelson, M. R., S. L. Kardia, R. E. Ferrell, and C. F. Sing (2001) A combinatorial partitioning method to identify multilocus genotypic partitions that predict quantitative trait variation. Genome Res 11, 458-70.
141. Ritchie, M. D., L. W. Hahn, N. Roodi, L. R. Bailey, W. D. Dupont, F. F. Parl, and J. H. Moore (2001) Multifactor-dimensionality reduction reveals high-order interactions among estrogen-metabolism genes in sporadic breast cancer. Am J Hum Genet 69, 138-47.
142. Zhao, J., L. Jin, and M. Xiong (2006) Test for interaction between two unlinked loci. Am J Hum Genet 79, 831-45.
143. Kernighan, B. W. and D. M. Ritchie, The C Programming Language (2nd Edition). 2nd Edition ed. 1988: Prentice-Hall.
144. Consortium, I. H. (2005) A haplotype map of the human genome. Nature 437, 1299-320.
145. Dudbridge, F. and A. Gusnanto (2008) Estimation of significance thresholds for genomewide association scans. Genet Epidemiol 32, 227-34.
146. Storey, J. D., A direct approach to false discovery rates. 2002. p. 479-498.
147. Gauderman, W. and J. Morrison, QUANTO 1.1: A computer program for power and sample size calculations for genetic epidemiology studies, hydra.usc.edu/gx website. 2006.
148. Evans, A. H., A. D. Lawrence, J. Potts, L. MacGregor, R. Katzenschlager, K. Shaw, J. Zijlmans, and A. J. Lees (2006) Relationship between impulsive sensation seeking traits, smoking, alcohol and caffeine intake, and Parkinson's disease. J Neurol Neurosurg Psychiatry 77, 317-21.
149. Schaid, D. J., C. M. Rowland, D. E. Tines, R. M. Jacobson, and G. A. Poland (2002) Score tests for association between traits and haplotypes when linkage phase is ambiguous. Am J Hum Genet 70, 425-34.
150. Taylor, N. E. and E. A. Greene (2003) PARSESNP: A tool for the analysis of nucleotide polymorphisms. Nucleic Acids Res 31, 3808-11.
151. Ng, P. C. and S. Henikoff (2003) SIFT: Predicting amino acid changes that affect protein function. Nucleic Acids Res 31, 3812-4.
152. dcode.org website
153. genome.lbl.gov website
154. Marchini, J., B. Howie, S. Myers, G. McVean, and P. Donnelly (2007) A new multipoint method for genome-wide association studies by imputation of genotypes. Nat Genet 39, 906-13.
155. (2007) Genome-wide association study of 14,000 cases of seven common diseases and 3,000 shared controls. Nature 447, 661-78.
156. Scott, L. J., K. L. Mohlke, L. L. Bonnycastle, C. J. Willer, Y. Li, W. L. Duren, M. R. Erdos, H. M. Stringham, P. S. Chines, A. U. Jackson, L. Prokunina-Olsson, C. J. Ding, A. J. Swift, N. Narisu, T. Hu, R. Pruim, R. Xiao, X. Y. Li, K. N. Conneely, N. L. Riebow, A. G. Sprau, M. Tong, P. P. White, K. N. Hetrick, M. W. Barnhart, C. W. Bark, J. L. Goldstein, L. Watkins, F. Xiang, J. Saramies, T. A. Buchanan, R. M. Watanabe, T. T. Valle, L. Kinnunen, G. R. Abecasis, E. W. Pugh, K. F. Doheny, R. N. Bergman, J Tuomilehto, F. S. Collins, and M. Boehnke (2007) A genome-wide association study of type 2 diabetes in Finns detects multiple susceptibility variants. Science 316, 1341-5.
157. Wacholder, S., S. Chanock, M. Garcia-Closas, L. El Ghormli, and N. Rothman (2004) Assessing the probability that a positive report is false: an approach for molecular epidemiology studies. J Natl Cancer Inst 96, 434-42.
158. Hutz, J. E., A. T. Kraja, H. L. McLeod, and M. A. Province (2008) CANDID: a flexible method for prioritizing candidate genes for complex human traits. Genet Epidemiol 32, 779-90.
159. Oliveira, S. A., Y. J. Li, M. A. Noureddine, S. Zuchner, X. Qin, M. A. Pericak-Vance, and J. M. Vance (2005) Identification of risk and age-at-onset genes on chromosome 1p in Parkinson disease. Am J Hum Genet 77, 252-64.
160. Martinez, M., A. Brice, J. R. Vaughan, A. Zimprich, M. M. Breteler, G. Meco, A. Filla, M. J. Farrer, C. Betard, J. Hardy, G. De Michele, V. Bonifati, B. Oostra, T. Gasser, N. W. Wood, and A. Durr (2004) Genome-wide scan linkage analysis for Parkinson's disease: the European genetic study of Parkinson's disease. J Med Genet 41, 900-7.
161. Pankratz, N., S. K. Uniacke, C. A. Halter, A. Rudolph, C. W. Shults, P. M. Conneally, T. Foroud, and W. C. Nichols (2004) Genes influencing Parkinson disease onset: replication of PARK3 and identification of novel loci. Neurology 62, 1616-8.
162. Bertoli-Avella, A. M., M. C. Dekker, Y. S. Aulchenko, J. J. Houwing-Duistermaat, E. Simons, L. Testers, L. M. Pardo, T. A. Rademaker, P. J. Snijders, J. C. van Swieten, V. Bonifati, P. Heutink, C. M. van Duijn, and B. A. Oostra (2006) Evidence for novel loci for late-onset Parkinson's disease in a genetic isolate from the Netherlands. Hum Genet 119, 51-60.
163. Hicks, A. A., H. Petursson, T. Jonsson, H. Stefansson, H. S. Johannsdottir, J. Sainz, M. L. Frigge, A. Kong, J. R. Gulcher, K. Stefansson, and S. Sveinbjornsdottir (2002) A susceptibility gene for late-onset idiopathic Parkinson's disease. Ann Neurol 52, 549-55.
164. Scott, W. K., M. A. Nance, R. L. Watts, J. P. Hubble, W. C. Koller, K. Lyons, R. Pahwa, M. B. Stern, A. Colcher, B. C. Hiner, J. Jankovic, W. G. Ondo, F. H. Allen, Jr., C. G. Goetz, G. W. Small, D. Masterman, F. Mastaglia, N. G. Laing, J. M. Stajich, B. Slotterbeck, M. W. Booze, R. C. Ribble, E. Rampersaud, S. G. West, R. A. Gibson, L. T. Middleton, A. D. Roses, J. L. Haines, B. L. Scott, J. M. Vance, and M. A. Pericak-Vance (2001) Complete genomic screen in Parkinson disease: evidence for multiple genes. Jama 286, 2239-44.
165. Pankratz, N., W. C. Nichols, S. K. Uniacke, C. Halter, A. Rudolph, C. Shults, P. M. Conneally, and T. Foroud (2002) Genome screen to identify susceptibility genes for Parkinson disease in a sample without parkin mutations. Am J Hum Genet 71, 124-35.
166. Gasser, T., B. Muller-Myhsok, Z. K. Wszolek, R. Oehlmann, D. B. Calne, V. Bonifati, B. Bereznai, E. Fabrizio, P. Vieregge, and R. D. Horstmann (1998) A susceptibility locus for Parkinson's disease maps to chromosome 2p13. Nature Genet 18, 262-265.
167. Rosenberger, A., M. Sharma, B. Muller-Myhsok, T. Gasser, and H. Bickeboller (2007) Meta analysis of whole-genome linkage scans with data uncertainty: an application to Parkinson's disease. BMC Genet 8, 44.
168. Chaudhuri, A., K. Bowling, C. Funderburk, H. Lawal, A. Inamdar, Z. Wang, and J. M. O'Donnell (2007) Interaction of genetic and environmental factors in a Drosophila parkinsonism model. J Neurosci 27, 2457-67.
169. flystocks.bio.indiana.edu website
170. Kontopoulos, E., J. D. Parvin, and M. B. Feany (2006) Alpha-synuclein acts in the nucleus to inhibit histone acetylation and promote neurotoxicity. Hum Mol Genet 15, 3012-23.
171. Outeiro, T. F., E. Kontopoulos, S. M. Altmann, I. Kufareva, K. E. Strathearn, A. M. Amore, C. B. Volk, M. M. Maxwell, J. C. Rochet, P. J. McLean, A. B. Young, R. Abagyan, M. B. Feany, B. T. Hyman, and A. G. Kazantsev (2007) Sirtuin 2 inhibitors rescue alpha-synuclein-mediated toxicity in models of Parkinson's disease. Science 317, 516-9.
172. Lesnick, T. G., S. Papapetropoulos, D. C. Mash, J. Ffrench-Mullen, L. Shehadeh, M. de Andrade, J. R. Henley, W. A. Rocca, J. E. Ahlskog, and D. M. Maraganore (2007) A genomic pathway approach to a complex disease: axon guidance and Parkinson disease. PLoS Genet 3, e98.
173. Forsyth, J. T., R. A. Grunewald, A. Rostami-Hodjegan, M. S. Lennard, H. J. Sagar, and G. T. Tucker (2000) Parkinson's disease and CYP1A2 activity. Br J Clin Pharmacol 50, 303-9.
174. Tan, E. K., E. Chua, S. M. Fook-Chong, Y. Y. Teo, Y. Yuen, L. Tan, and Y. Zhao (2007) Association between caffeine intake and risk of Parkinson's disease among fast and slow metabolizers. Pharmacogenet Genomics 17, 1001-5.
175. Ray, R., R. F. Tyndale, and C. Lerman (2009) Nicotine Dependence Pharmacogenetics: Role of Genetic Variation in Nicotine-Metabolizing Enzymes. J Neurogenet 1-10.
176. Abou-Sleiman, P. M., M. M. Muqit, and N. W. Wood (2006) Expanding insights of mitochondrial dysfunction in Parkinson's disease. Nat Rev Neurosci 7, 207-19.
177. van der Walt, J. M., K. K. Nicodemus, E. R. Martin, W. K. Scott, M. A. Nance, R. L. Watts, J. P. Hubble, J. L. Haines, W. C. Koller, K. Lyons, R. Pahwa, M. B. Stern, A. Colcher, B. C. Hiner, J. Jankovic, W. G. Ondo, F. H. Allen, Jr., C. G. Goetz, G. W. Small, F. Mastaglia, J. M. Stajich, A. C. McLaurin, L. T. Middleton, B. L. Scott, D. E. Schmechel, M. A. Pericak-Vance, and J. M. Vance (2003) Mitochondrial polymorphisms significantly reduce the risk of Parkinson disease. Am J Hum Genet 72, 804-11.
178. Kujoth, G. C., P. C. Bradshaw, S. Haroon, and T. A. Prolla (2007) The role of mitochondrial DNA mutations in mammalian aging. PLoS Genet 3, e24.
179. Scherzer, C. R., A. C. Eklund, L. J. Morse, Z. Liao, J. J. Locascio, D. Fefer, M. A. Schwarzschild, M. G. Schlossmacher, M. A. Hauser, J. M. Vance, L. R. Sudarsky, D. G. Standaert, J. H. Growdon, R. V. Jensen, and S. R. Gullans (2007) Molecular markers of early Parkinson's disease based on gene expression in blood. Proc Natl Acad Sci USA 104, 955-60.
180. Schupf, N., M. X. Tang, H. Fukuyama, J. Manly, H. Andrews, P. Mehta, J. Ravetch, and R. Mayeux (2008) Peripheral Abeta subspecies as risk biomarkers of Alzheimer's disease. Proc Natl Acad Sci USA 105, 14052-7.
181. Wang, K., M. Li, and M. Bucan (2007) Pathway-based approaches for analysis of genome-wide association studies. Am J Hum Genet 81, [Upub ahead of print].
182. Lavara-Culebras, E. and N. Paricio (2007) Drosophila DJ-1 mutants are sensitive to oxidative stress and show reduced lifespan and motor deficits. Gene 400, 158-65.
183. Li, R. and R. S. El-Mallakh (1997) Triplet repeat gene sequences in neuropsychiatric disease. Harvard Rev Psychiatry 5, 66-74.
184. Li, Y., C. Rowland, G. Xiromerisiou, R. J. Lagier, S. J. Schrodi, E. Dradiotis, D. Ross, N. Bui, J. Catanese, K. Aggelakis, A. Grupe, and G. Hadjigeorgiou (2008) Neither replication nor simulation supports a role for the axon guidance pathway in the genetics of Parkinson's disease. PLoS ONE 3, e2707.
185. Srinivasan, B. S., J. Doostzadeh, F. Absalan, S. Mohandessi, R. Jalili, S. Bigdeli, J. Wang, J. Mahadevan, C. L. Lee, R. W. Davis, J. William Langston, and M. Ronaghi (2008) Whole genome survey of coding SNPs reveals a reproducible pathway determinant of Parkinson disease. Hum Mutat 30, 228-238.
186. Baldereschi, M., A. Di Carlo, W. Rocca, P. Vanni, S. Maggi, E. Perissinotto, F. Grigoletto, L. Amaducci, and D. Inzitari (2000) Parkinson's disease and parkinsonism in a longitudinal study. Two fold higher incidence in men. Neurology 55, 1358-1363.
187. Pollock, B. G., M. Wylie, J. A. Stack, D. A. Sorisio, D. S. Thompson, M. A. Kirshner, M. M. Folan, and K. A. Condifer (1999) Inhibition of caffeine metabolism by estrogen replacement therapy in postmenopausal women. J Clin Pharmacol 39, 936-40.
188. Xu, K., Y. Xu, D. Brown-Jermyn, J. F. Chen, A. Ascherio, D. E. Dluzen, and M. A. Schwarzschild (2006) Estrogen prevents neuroprotection by caffeine in the mouse 1-methyl-4-phenyl-1,2,3,6-tetrahydropyridine model of Parkinson's disease. J Neurosci 26, 535-41.
189. Wang, G., J. M. van der Walt, G. Mayhew, Y. J. Li, S. Zuchner, W. K. Scott, E. R. Martin, and J. M. Vance (2008) Variation in the miRNA-433 binding site of FGF20 confers risk for Parkinson disease by overexpression of alpha-synuclein. Am J Hum Genet 82, 283-9.
190. Schembri, F., S. Sridhar, C. Perdomo, A. M. Gustafson, X. Zhang, A. Ergun, J. Lu, G. Liu, X. Zhang, J. Bowers, C. Vaziri, K. Ott, K. Sensinger, J. J. Collins, J. S. Brody, R. Getts, M. E. Lenburg, and A. Spira (2009) MicroRNAs as modulators of smoking-induced gene expression changes in human airway epithelium. Proc Natl Acad Sci USA.

### Example 3

Applicants' aim was to identify genes that influence the inverse association of coffee with the risk of developing PD. Applicants used genome-wide genotype data and lifetime caffeinated-coffee-consumption data on 1458 persons with PD and 931 without PD from the NeuroGenetics Research Consortium (NGRC), and performed a genomewide association and interaction study (GWAIS), testing each SNP's main-effect plus its interaction with coffee, adjusting for sex, age and two principal-components. Applicants then stratified subjects as heavy- or light-coffeedrinkers and performed genome-wide association study (GWAS) in each group. Applicants replicated the most significant SNP. Finally, Applicants imputed the NGRC dataset, increasing genomic coverage to examine the region of interest in detail. The primary analyses (GWAIS, GWAS, Replication) were performed using genotyped data. In GWAIS, the most significant signal came from rs4998386 and neighboring SNPs in GRIN2A. GRIN2A encodes an NMDA-glutamate-receptor subunit and regulates excitatory neurotransmission in the brain. Achieving P2df=10⁻⁶, GRIN2A surpassed all known PD susceptibility genes in significance in GWAIS. In stratified GWAS, GRIN2A signal was present in heavy-coffee-drinkers (OR=0.43; P=6×10⁻⁷) but not in lightcoffee-drinkers. The a-priori Replication hypothesis that “Among heavy-coffee-drinkers, rs4998386_T carriers have lower PD risk than rs4998386_CC carriers” was confirmed: ORReplication=0.59, PReplication=10⁻³; ORPooled=0.51, PPooled=6.5×10⁻⁸. Compared to light-coffee-drinkers with rs4998386_CC genotype, heavy-coffeedrinkers with rs4998386_CC genotype had 18% lower risk (P=3×10⁻³), whereas heavy-coffee-drinkers with rs4998386_TC genotype had 59% lower risk (P=6×10⁻¹³). Imputation revealed a block of SNPs that achieved.

P_{2df}<5×10⁻⁸ in GWAIS, and OR=0.41, P=3×10⁻⁸ in heavy-coffee-drinkers. This study is proof of concept that inclusion of environmental factors can help identify genes that are missed in GWAS. Both adenosine-antagonists (caffeine-like) and glutamate-antagonists (GRIN2A-related) are being tested in clinical trials for treatment of PD. GRIN2A may be a useful pharmacogenetic marker for subdividing individuals in clinical trials to determine which medications might work best for which patients.

Parkinson's disease (PD), like most common disorders, involves interactions between genetic make-up and environmental exposures which are unique to each individual. Caffeinated-coffee consumption may protect some people from developing PD, although not all benefit equally. In a genome-wide search, Applicants discovered that variations in the glutamate-receptor gene GRIN2A modulate the risk of developing PD in coffee drinkers. The study was hypothesis-free, that is, Applicants cast a net across the entire genome allowing statistical significance to point us to a genetic variant, regardless of whether it fell in a genomic desert or an important gene. Fortuitously, the most significant finding was in a well-known gene, GRIN2A, which regulates brain signals that control movement and behavior. Applicants' finding is important for three reasons: First, it is a proof of concept that studying genes and environment on the whole-genome scale is feasible, and this approach can identify important genes that are missed when environmental exposures are ignored. Second, the knowledge of interaction between GRIN2A which is involved in neurotransmission in the brain, and caffeine which is an adenosine-A2A-receptor-antagonist will stimulate new research towards understanding the cause and progression of PD. Third, the results may lead to personalized prevention and treatment for PD.

Common disorders are thought to have both genetic and environmental components. Genome-wide association studies (GWAS) have successfully identified numerous susceptibility loci for many common disorders ranging from behavioral traits such as addiction and substance abuse, to infectious and immunerelated disorders, age-related neurodegenerative disorders like Alzheimer's, Parkinson's and macular degeneration, metabolic disorders, psychiatric disorders, and many more (for the list and results of over 800 published GWAS see genome.gov/gwastudies website). Despite the success of GWAS, the heritability of common disorders cannot be fully explained by the genes that have been discovered [1]. GWAS are built on the notion that common alleles predispose to common disorders. Rare variants, which are probably responsible for some of the missing heritability, would not have been detected by GWAS. Sequencing the genome and novel analytical methods will help identify the rare variants. Another hiding place for the missing heritability is in interactions. Genes that impact disease through interactions with other genes or environmental factors are not detected by GWAS if their main effects are small. GWAS can only identify genes that exhibit significant main effects; genes that require the interacting factor to be included in the study to show their association with disease are missed. Inclusion of key environmental factors in genome-wide studies is anticipated to be an important next step for deciphering the genetic structure of common multifactorial disorders. Amassing sufficient analytic power for gene-environment studies, however, is a challenge. Power decreases dramatically as a function of frequency of exposure, number of parameters being estimated and sample size; there are fewer datasets with both DNA and environmental exposure data than those with DNA alone, and their sample sizes are often smaller.

Parkinson's disease (PD) is a classic example of a common multifactorial disorder. PD is characterized by neurodegeneration in the substantia nigra that manifests initially as a movement disorder but often leads to cognitive and psychiatric problems as well. PD is progressive and there is no treatment currently available that could prevent or slow disease progression. PD is the second most common neurodegenerative disease after Alzheimer disease; it affects about 5 million individuals in the 10 most populous nations and is expected to double in frequency by 2030 [2]. Until 1990's PD was thought to be purely environmental with no genetic component. In the last decade, numerous genes have been identified, some of which can cause PD [3] and others that are susceptibility loci [4,5,6,7,8,9]. There are also compelling data from epidemiology that cigarette smoking and caffeinated-coffee consumption are associated with reduced risk of developing PD [10,11] and exposure to environmental neurotoxins are associated with increased risk of developing PD [12]. Thus PD is a strong candidate for studying gene-environment interactions [13].

Applicants conducted a genome-wide association and interaction study (GWAIS), using the joint test [14] of SNP's marginal association and its interaction with coffee consumption on PD risk. Applicants' aim was to identify genes that enhance or diminish the protective effect of caffeinated-coffee for use as biomarkers for pharmacogenetic prevention and treatment. Caffeine is an adenosine-receptor-antagonist. In animal models of PD, where administration of neurotoxin is used to destroy dopaminergic neurons mimicking PD, caffeine and selective A2A-antagonists have been shown to be neuroprotective and attenuate dopamine loss [15]. Selective A2A antagonists have been studied in human clinical trials and found to be safe, well tolerated and to provide symptomatic benefit for persons with PD [16,17]; however, efficacy has not been high enough in the first generation of the drugs to meet regulatory approval for use as PD drugs. Applicants posit that subsets of patients with certain genotypes may respond well to this treatment and others may not. When they are combined the average efficacy may be insufficient for regulatory approval, while a subgroup of patients with certain genotype might still benefit substantially. If Applicants' prediction is correct, incorporating genetics in clinical trials of PD could revolutionize PD drug development. By examining the interaction of caffeinated-coffee with 811,597 SNP in a hypothesis-free genome-wide study, Applicants discovered GRIN2A as a novel PD modifier gene. GRIN2A encodes a subunit of the NMDA-glutamate-receptor which is well known for regulating excitatory neurotransmission in the brain and for controlling movement and behavior.

Human Subject Committees of the participating institutions approved the study. The Discovery dataset was nested in the NeuroGenetics Research Consortium (NGRC) GWAS which successfully identified known PD genes as well as a new PD locus within HLA [4] which has been widely replicated [9,18]. Replication samples were provided by PEG [19] (Parkinson, Environment, and Gene), PAGE [20] (Parkinson's, Genes, and Environment from the prospective NIH-AARP Diet and Health Study cohort), and HIHG [8] (Hussman Institute for Human Genomics). Persons with PD had been diagnosed by neurologists using standard criteria [21,22], control subjects self-reported as not having PD. Cases and controls were all unrelated, non-Hispanic Caucasian, from United States. The NGRC cohort was clinic-based sequentially ascertained patients, PEG and PAGE were community-based incident cases, HIHG was clinic-based and self-referral cases. The numbers of cases/controls with genotype, coffee/caffeine and key clinical and demographic data were NGRC=1458/931, PEG=280/310, PAGE=525/1474, HIHG=209/133 (Table 6).

TABLE 6
Summary statistics on subject characteristics

| | | NGRC (Discovery) | | PEG (Replication) | |
|---|---|---|---|---|---|
| | | Case | Control | Case | Control |
| N | Total | 1458 | 931 | 280 | 310 |
| Sex | Male (%) | 991 (67.97) | 385 (41.35) | 156 (55.71) | 155 (50.00) |
| | Female (%) | 467 (32.03) | 546 (58.65) | 124 (44.29) | 155 (50.00) |
| GRIN2A | CC (%) | 1227 (84.16) | 716 (76.91) | 234 (83.57) | 249 (80.32) |
| rs4998386 | TC (%) | 219 (15.02) | 204 (21.91) | 42 (15.00) | 55 (17.74) |
| genotype | TT (%) | 12 (0.82) | 11 (1.18) | 4 (1.43) | 6 (1.94) |
| Caffeinated- | Light (%) | 946 (64.88) | 544 (58.43) | 184 (65.71) | 188 (60.65) |
| coffee | Heavy (%) | 512 (35.12) | 387 (41.57) | 96 (34.29) | 122 (39.35) |
| drinking | | | | | |
| Cigarette | Never (%) | 784 (54.18) | 486 (52.26) | 149 (54.18) | 121 (44.98) |
| smoking* | Ever (%) | 663 (45.82) | 444 (47.74) | 126 (45.82) | 148 (55.02) |
| Age at enrolment | Mean ± SD | 65.51 ± 10.59 | 65.82 ± 11.48 | 71.04 ± 10.17 | 67.96 ± 12.07 |
| Age at onset | Mean ± SD | 58.13 ± 11.61 | — | NA | — |
| Age at diagnosis | Mean ± SD | 60.05 ± 11.20 | — | 69.07 ± 10.20 | — |
| | | PAGE (Replication) | | HING (Replication) | |
| | | Case | Control | Case | Control |
| N | Total | 525 | 1474 | 209 | 133 |
| Sex | Male (%) | 406 (77.33) | 1164 (78.97) | 142 (67.94) | 56 (42.11) |
| | Female (%) | 119 (22.67) | 310 (21.03) | 67 (32.06) | 77 (57.89) |
| GRIN2A | CC (%) | 434 (82.67) | 1199 (81.34) | 169 (80.86) | 110 (82.71) |
| rs4998388 | TC (%) | 85 (16.19) | 267 (18.11) | 36 (17.22) | 22 (16.54) |
| genotype | TT (%) | 6 (1.14) | 8 (0.54) | 4 (1.91) | 1 (0.75) |
| Caffeinated- | Light (%) | 290 (55.24) | 738 (50.07) | 147 (70.33) | 86 (64.66) |
| coffee | Heavy (%) | 235 (44.76) | 736 (49.93) | 62 (23.67) | 47 (35.34) |
| drinking | | | | | |
| Cigarette | Never (%) | 229 (44.29) | 523 (35.82) | 113 (54.33) | 66 (50.00) |
| smoking* | Ever (%) | 289 (55.71) | 937 (64.18) | 95 (45.67) | 66 (50.00) |
| Age at enrolment | Mean ± SD | 63.98 ± 4.77 | 64.01 ± 4.84 | 65.16 ± 10.63 | 67.53 ± 10.62 |
| Age at onset | Mean ± SD | 68.24 ± 5.66 | — | 56.41 ± 11.59 | — |
| Age at diagnosis | Mean ± SD | 69.40 ± 5.34 | — | NA | — |

NA: not available
*Non-smoker: <100 cigarettes in lifetime. Smoker: ≧100 cigarettes.

NGRC, PEG and HIHG had collected lifetime caffeinated-coffee consumption data, measured as cups per day multiplied by the number of years of consumption (ccy) [11,23]. PAGE had daily mg caffeine intake from all caffeine-containing drinks and foods for 12 months prior to enrollment (1995-1996) and only incident PD cases diagnosed after 1997 were included in the analysis [24]. Despite the variation in data collection, results were consistent across studies, corroborating robustness of the interaction between coffee/caffeine and GRIN2A. Applicants could not, and did not, attempt to distinguish the bioactive ingredient in caffeinated-coffee. Although caffeine has been shown to be neuroprotective, there may be other ingredients in caffeinated coffee that may affect disease pathogenesis. To classify coffee/caffeine intake, each dataset was treated separately according to the measurements available. The median ccy or mg was determined for controls within each dataset (excluding those with zero intake) and used as the cut-off for heavy-drinkers (>median) vs. light-drinkers (0 to ≦median). The median was 67.5 ccy for NGRC, 74.0 ccy for PEG, 70.0 ccy for HIHG, and 237.8 mg/day for PAGE. For coffee dose, quartiles were defined for each dataset using the full range from zero to maximum intake in controls. Results shown for NGRC, PEG and HIHG are based on lifetime caffeinated-coffee consumption. Truncating coffee use at age-at-onset or age-at-diagnosis in patients did not affect the results. To assess the effects of caffeinated tea and soda, Applicants performed sensitivity analysis in NGRC dataset. Caffeinated soda and tea were commonly and equally consumed by heavy- and light-coffee drinkers (soda: 80% in both heavy- and light drinkers; caffeinated tea: 66% in heavy-coffee drinkers and 61% in light-coffee drinkers). Applicants repeated GWAIS and stratified GWAS with caffeinated soda and tea as covariates. Applicants also explored association of caffeinated tea and soda with PD expecting an inverse association if caffeine were the bioactive ingredient in coffee.

Genotyping.

The source of DNA was whole blood for NGRC and HIHG, saliva for PAGE, and whole blood (all PD and half of controls) or saliva (half of controls) for PEG. NGRC was genome-wide genotyped using Illumina HumanOmni1-Quad_v1-0_B array and achieved 99.92% call rate and 99.99% reproducibility. GWAS genotyping and statistical quality control (QC) have been published [4]. 811,597 SNPs (excluding Y chromosome because they are not amenable to sex adjustment) passed GWAS QC and were included in GWAIS. Replication groups genotyped GRIN2A_rs4998386. Only one SNP was genotyped for replication; Applicants have no other undisclosed replication results. PEG and HIHG used ABI TaqMan assay by-design (C_{—}28018721_{—}20), PAGE used Sequenom and all achieved call rates of 96%-99%.

Quality Control for GWAIS and Stratified GWAS in Discovery (NGRC).

The genome-wide genotypes for NGRC had been cleaned previously for GWAS using standard and rigorous measures [4]. Applicants had identified two significant principal components (PC1, PC2) marking Jewish/non-Jewish ancestry and European countries of origin [4]. Sex was a significant variable, because PD affects more men than women and Applicants' data has a significant gender disparity (Table 6). Controls were older than patients at age at onset, which was by design to minimize the chances that controls were too young to have developed the disease. Nevertheless, Applicants controlled for age at enrolment both for patients and controls to avoid confounding by age-related factors. Applicants examined coffee consumption and the most significant SNP for potential variation by disease related variables, recruitment sites, and ethnic and geographic origins of subjects (Table 7).

TABLE 7
Frequency of GRIN2A rs4998386_T and heavy coffee use in NGRC by
disease strata and population structure.

| | N | | GRIN2A rs4999386_T | | | | Heavy Coffee Use | | | |
|---|---|---|---|---|---|---|---|---|---|---|
| | Case | Cont. | MAF | | MAF | | N (%) | | N (%) | |
| | N (%) | N (%) | Case | P | Cont | P | Case | P | Cont | P |
| All subjects | 145 [symbol] | 931 | 0.0 [symbol] | | 0.12 | | 512 (35.12) | | 387 (41.57) | |
| PD-Associated | | | | | | | | | | |
| risk factors | | | | | | | | | | |
| Sporadic PD | 1142 (78. [symbol] ) | 931 (100.00) | 0.0 [symbol] | | 0.12 | | 405 (35.46) | | 387 (41.57) | |
| Famila [symbol] PD | 316 (21.67) | 931 (100.00) | 0.0 [symbol] | 0.39 | 0.12 | | 107 (33.96) | 0.60 | 387 (41.57) | |
| Late onset | 1069 (73. [symbol] 2) | 931 (100.00) | 0.0 [symbol] | | 0.12 | | 427 (39.94) | | 387 (41.57) | |
| ( [symbol] 50 yrs) | | | | | | | | | | |
| Early onset | 3 [symbol] 9 (26.68) | 931 (100.00) | 0.0 [symbol] | 0. [symbol] | 0.12 | | 85 (21.85) | 0.19 [symbol] | 387 (41.57) | |
| (≦50 yrs) | | | | | | | | | | |
| Male | 991 (67.97) | 335 (41.35) | 0.0 [symbol] | | 0.13 | | 371 (37.44) | | 187 (49.57) | |
| Female | 4 [symbol] 7 ( [symbol] ) | 54 [symbol] (5 [symbol] ) | 0.0 [symbol] | [symbol] | 0.12 | 0.35 | 141 (30.19) | 0.01 | 200 (26.63) | 0.0003 |
| Smokers | 663 (45.47) | 444 (47.69) | 0.09 | | 0.13 | | 298 (44.95) | | 233 (52.48) | |
| Non-Smokers | 784 (53.77) | 495 (52.20) | 0.0 [symbol] | 0.92 | 0.11 | 0.17 | 211 (26.91) | <0.0001 | 153 (31.48) | <0.0001 |
| Coffee—heavy | 512 (35.12) | 397 (41.57) | 0.07 | | 0.14 | | | | | |
| Coffee—light | 946 (64.88) | 544 (5 [symbol] ) | 0.09 | 0.08 | 0.11 | 0.03 | | | | |
| A [symbol] nazi Jewish | | | | | | | | | | |
| (self reported, | | | | | | | | | | |
| verified | | | | | | | | | | |
| by principal | | | | | | | | | | |
| components) | | | | | | | | | | |
| Yes | 67 (4.60) | 23 (2.47) | 0.03 | | 0.04 | | 17 (25. [symbol] 7) | | 9 (39.13) | |
| No | 1391 (95.40) | 90 [symbol] (97.53) | 0.09 | 0.02 | 0.12 | 0.10 | 485 (35.59) | 0.09 | 379 (41.63) | 0.81 |
| Re [symbol] rnent Site | | | | | | | | | | |
| New York | 346 (23.73) | 265 (28.46) | 0.0 [symbol] | | 0.11 | | 118 (34.10) | | 105 (39.62) | |
| Oregon | 221 (15.16) | 130 (13.96) | 0.0 [symbol] | | 0.11 | | 75 (33.94) | | 55 (42.31) | |
| Georgia | 220 (15.0 [symbol] ) | 107 (11.49) | 0.0 [symbol] | | 0.11 | | 82 (37.27) | | 42 (39.25) | |
| Washington | 671 (46.02) | 429 (46.08) | 0.09 | [symbol] 1 | 0.13 | 0.48 | 237 (35.32) | 0.86 | 185 (43.12) | 0.78 |
| Paternal and | | | | | | | | | | |
| Maternal ancestry | | | | | | | | | | |
| Great Britain | 100 (6. [symbol] 6) | 57 (6.12) | 0.14 | | 0.16 | | 35 (35.00) | | 26 (45.61) | |
| Germany/Austria | 76 (5.21) | 39 (4.08) | 0.07 | | 0.20 | | 31 (40.79) | | 16 (42.11) | |
| Ireland | 35 (2.40) | 14 (1.50) | 0.10 | | 0.04 | | 16 (45.71) | | 6 (42.9 [symbol] ) | |
| Scandinavia | 44 ( [symbol] .02) | 25 (2.69) | 0.14 | | 0.10 | | 19 (40. [symbol] 1) | | 12 (48.00) | |
| Eastern Europe | 27 (1. [symbol] 5) | 25 (2.69) | 0.02 | | 0.12 | | 12 (44.44) | | 11 (44.00) | |
| Italy | 43 (2.95) | 31 (3.33) | 0.05 | | 0.05 | | 22 (51.16) | | 17 (54.84) | |
| Russia | 16 (1.10) | 9 (0.97) | 0.09 | 0.03 | 0.06 | 0.19 | 4 (25.00) | 0.49 | 5 (55.55) | 0.95 |
| Paternal or | | | | | | | | | | |
| Maternal ancestry | | | | | | | | | | |
| Great Britain | 410 (2 [symbol] .12) | 262 (28.14) | 0.11 | | 0.13 | | 143 (34. [symbol] ) | | 108 (41.22) | |
| Germany/Austria | 321 (22.02) | 193 (20.73) | 0.09 | | 0.13 | | 115 (35.93) | | 71 (36.79) | |
| Irelend | 180 (12.35) | 126 (13.53) | 0.08 | | 0.13 | | 72 (40.00) | | 57 (45.24) | |
| Scandinavia | 170 (11.66) | 102 (10.96) | 0.10 | | 0.12 | | 61 (35.88) | | 46 (45.10) | |
| Eastern Europe | 72 (4.94) | 60 (6.44) | 0.02 | | 0.16 | | 25 (34.72) | | 27 (45.00) | |
| Italy | 69 (4. [symbol] ) | 59 (6.34) | 0.04 | | 0.09 | | 28 (41.18) | | 21 (35.59) | |
| France | 59 (4.05) | 45 (4.83) | 0.08 | | 0.17 | | 17 (28.81) | | 14 (31.11) | |
| Russia | 45 (3.09) | 19 (1.93) | 0.04 | 0.003 | 0.03 | 0.37 | 10 (22.22) | 0.40 | 9 (50.00) | 0.47 |

*Adjusted for age.
The higher coffee consumption in late-onset PD is because they are older than patients with early-onset PD and have had higher cumulative lifetime coffee use over the years. [symbol]
indicates data missing or illegible when filed

Family History:

Patients who had at least one first or second degree relative with PD were classified as familial. All others were classified as non-familial (sporadic) Coffee: Number of cups of caffeinated coffee drank per day multiplied by the number of years of consumption (ccy); heavy and light divided at the median in controls (67.5 ccy).

Jewish/Non-Jewish Clusters:

The core of the Jewish cluster was defined within 0.04≦PC1≦0.055 and 0.001≦PC2≦0.013. A core within non-Jewish Caucasian cluster was defined within −0.0075≦PC1≦0.0025 & −0.005≦PC2≦0.003. See Hamza et al.[4]

Recruitment Site:

US states where subjects were recruited from.

Paternal & Maternal Ancestry:

Subjects whose both paternal and maternal ancestors came from the same country.

Paternal or Maternal Ancestry:

Since having only one lineage tracing back to a country was sufficient for this classification, an individual may fall in more than one group.

Smoking was a potential confounder because it is correlated with coffee use and is an independent inverse risk factor of PD. Thus Applicants repeated all analyses with smoking included as covariate in the model (Table 8).

TABLE 8
Smoking does not alter the results.

| | | NGRC | | | | PEG (Replication 1) | | | | PAGE (Replication 2) | | | | HIHG (Replication 2) | | | |
|---|---|---|---|---|---|---|---|---|---|---|---|---|---|---|---|---|---|
| GRIN2A | Coffee | PD | Co | OR(SE) | P | PD | Co | OR(SE) | P | PD | Co | OR(SE) | P | PD | Co | OR(SE) | P |

(a) Coffee irrespective of genotype

| | | | | | | | | | | | | | | | | | |
|---|---|---|---|---|---|---|---|---|---|---|---|---|---|---|---|---|---|
| | Light | 9 [symbol] | 544 | Ref | | 181 | 159 | Ref | | 285 | 731 | Ref | | 147 | 85 | Ref | |
| [symbol] | Heavy | 509 | 386 | 0. [symbol] (0.0 [symbol] ) | 6 × 10 [symbol] | 94 | 110 | 0.69(0.1 [symbol] ) | 0.02 | 232 | 729 | 0.83(0.09) | 0.04 | 61 | 47 | 0.69(0.18) | 0.07 |

(b) GRIN2A rs [symbol] genotype [symbol] rrespective of coffee

| | | | | | | | | | | | | | | | | | |
|---|---|---|---|---|---|---|---|---|---|---|---|---|---|---|---|---|---|
| CC | [symbol] | 1216 | 716 | Ref | | 229 | 215 | Ref | | 429 | 11 [symbol] 9 | Ref | | 16 [symbol] | 109 | Ref | |
| TC | | 219 | 203 | 0. [symbol] 3(0.07) | 5 × 10 [symbol] | 42 | 4 [symbol] | 0.79(0.19) | 0.15 | 92 | 263 | 0.87(0.12) | 0.1 [symbol] | 36 | 22 | 1.00(0.31) | 0.50 |
| TT | [symbol] | 12 | 11 | 0.54(0.24) | 0.16 | 4 | 6 | 0.56(0.37) | 0.19 | 6 | 8 | 2.34(1.27) | 0.94 | 4 | 1 | 2.00(2.27) | 0.73 |

(c) GRIN2A rs [symbol] genotype [symbol] by coffee

| | | | | | | | | | | | | | | | | | |
|---|---|---|---|---|---|---|---|---|---|---|---|---|---|---|---|---|---|
| CC | Heavy | 4 [symbol] | 283 | Ref | | 78 | 84 | Ref | | 197 | 571 | Ref | | 50 | 3 [symbol] | Ref | |
| TC | Heavy | 69 | 98 | 0.43(0.08) | 3 × 10 [symbol] | 14 | 25 | 0.57(0.22) | 0.07 | 30 | 152 | 0.57(0.12) | 0.01 | 9 | 11 | 0.65(0.34) | 0.20 |
| TT | Heavy | 2 | 5 | 0.20(0.17) | 0.06 | 2 | 1 | | | 5 | 6 | | | 2 | 0 | | |
| CC | Light | 778 | 433 | Ref | | 151 | 131 | Ref | | 232 | 618 | Ref | | 118 | 73 | Ref | |
| TC | Light | 150 | 105 | 0.82(0.12) | 0.18 | 28 | 23 | 1.02(0.32) | 0.53 | 52 | 111 | 1.26(0.23) | 0. [symbol] 9 | 27 | 11 | 1.31(0.54) | 0.74 |
| TT | Light | 10 | 6 | 0.82(0.45) | 0.71 | 2 | 5 | | | 1 | 2 | | | 2 | 1 | | |

Additive

| | | | | | | | | | | | | | | | | | |
|---|---|---|---|---|---|---|---|---|---|---|---|---|---|---|---|---|---|
| T vs. C | Heavy | 509 | 38 [symbol] | 0.43(0.07) | 7 × 10 [symbol] | 94 | 110 | 0.70(0.23) | 0.14 | 232 | 729 | 0.76(0.14) | 0.07 | 61 | 47 | 0.97(0.42) | 0.47 |
| T vs. C | Light | 938 | 544 | 0.84(0.11) | 0.18 | 181 | 159 | 0.82(0.21) | 0.21 | 2 [symbol] 5 | 731 | 1.25(0.22) | 0.90 | 147 | 85 | 1.22(0.44) | 0.71 |

Dominant

| | | | | | | | | | | | | | | | | | |
|---|---|---|---|---|---|---|---|---|---|---|---|---|---|---|---|---|---|
| CC | Heavy | 438 | 283 | Ref | | 78 | 84 | Ref | | 197 | 571 | Ref | | 50 | 36 | Ref | |
| T | Heavy | 71 | 103 | 0.42(0.07) | 9 × 10 [symbol] | 16 | 25 | 0.51(0.23) | 0.09 | 35 | 158 | 0.65(0.13) | 0.02 | 11 | 11 | 0.79(0.39) | 0. [symbol] 2 |
| CC | Light | 778 | 433 | Ref | | 151 | 131 | Ref | | 232 | 61 [symbol] | Ref | | 118 | 73 | Ref | |
| T | Light | 160 | 111 | 0.82(0.12) | 0.17 | 30 | 28 | 0.89(0.27) | 0.35 | 53 | 113 | 1.25(0.23) | 0.90 | 29 | 12 | 1.29(0.51) | 0.74 |

(d) Joint effects of GRIN2A rs4998 [symbol] 86 and coffee

| | | | | | | | | | | | | | | | | | |
|---|---|---|---|---|---|---|---|---|---|---|---|---|---|---|---|---|---|
| CC | Light | 77 [symbol] | 433 | Ref | | 151 | 131 | Ref | | 232 | 618 | Ref | | 118 | 73 | Ref | |
| CC | Heavy | 438 | 283 | 0.78(0.08) | 0.02 | 78 | 84 | 0.73(0.15) | 0.07 | 197 | 571 | 0.94(0.11) | 0.29 | 50 | 36 | 0.75(0.21) | 0.16 |
| TC | Light | 150 | 16 | 0.92(0.12) | 0.18 | 28 | 23 | 0.99(0.31) | 0.49 | 52 | 111 | 1.26(0.23) | 0.99 | 27 | 11 | 1.30(0.52) | 0.74 |
| TC | Heavy | 69 | 98 | 0.32(0.45) | 7 × 10 [symbol] | 14 | 25 | 0.44(0.16) | 0.01 | 30 | 152 | 0.54(0.12) | 2 × 10 [symbol] | 9 | 11 | 0.51(0.25) | 0.09 |
| TT | Light | 10 | 6 | 0.82(0.45) | 0.71 | 2 | 5 | | | 1 | 2 | | | 2 | 1 | | |
| TT | Heavy | 2 | 5 | 0.15(0.13) | 0.02 | 2 | 1 | | | 5 | 6 | | | 2 | 0 | | |

(e) Interactionn of GRIN24 rs4998386 genotype and coffee consumption

| | | | | | | | | | | | | | | | |
|---|---|---|---|---|---|---|---|---|---|---|---|---|---|---|---|
| 1435 | 919 | 0.52(0.12) | 5 × 10 [symbol] | 271 | 263 | 0.61(0.30) | 0.16 | 511 | 1452 | 0.45(0.13) | 3 × 10 [symbol] | 208 | 131 | 0.52(0.35) | 0.16 |

(f) Genotype specific dose-dependent effect of coffee

| | | | | | | | | | | | | | | | | | |
|---|---|---|---|---|---|---|---|---|---|---|---|---|---|---|---|---|---|
| CC | <25% | 332 | 189 | Ref | | 57 | 59 | Ref | | 130 | 312 | Ref | | 49 | 32 | Ref | |
| | 25% [symbol] 50% | 342 | 179 | 1.05(0.14) | 0.74 | 63 | 55 | 1.07(0.29) | 0.51 | 102 | 206 | 0.79(0.12) | 0.05 | 55 | 24 | 1.95(0.72) | 0.96 |
| | 50% [symbol] 75% | 362 | 203 | 0.93(0.13) | 0.61 | 54 | 51 | 0.95(0.25) | 0.43 | 94 | 292 | 0.69(0.11) | 0.01 | 37 | 29 | 0.99(0.33) | 0.37 |
| | <75% | 190 | 146 | 0.60(0.09) | 1 × 10 [symbol] | 45 | 50 | 0.76(0.21) | 0.15 | 112 | 279 | 1.01(0.15) | 0.52 | 27 | 24 | 0.71(0.29) | 0.20 |
| TC | <25% | 69 | 55 | Ref | | 14 | 10 | Ref | | 32 | 53 | Ref | | 13 | 6 | Ref | |
| | 25% [symbol] 50% | 55 | 41 | 1.35(0.39) | 0.29 | 12 | 10 | 0.77(0.49) | 0.94 | 20 | 59 | 0.56(0.19) | 0.05 | 10 | 3 | 1.62(1.39) | 0.71 |
| | 50% [symbol] 75% | 59 | 55 | 0.74(0.21) | 0.30 | 9 | 14 | 0.29(0.19) | 0.03 | 14 | 67 | 0.34(01.3) | 2 × 10 [symbol] | 7 | 7 | 0.45(0.34) | 0.15 |
| | <75% | 25 | 52 | 0.32(0.11) | 9 × 10 [symbol] | 7 | 14 | 0.95(0.23) | 0.06 | 16 | 85 | 0.32(0.11) | 6 × 10 [symbol] | 6 | 6 | 0.53(0.45) | 0.29 |
| TT | <25% | 6 | 0 | | | 1 | 2 | | | 1 | 0 | | | 1 | 0 | | |
| | 25% [symbol] 50% | 2 | 3 | | | 0 | 2 | | | 0 | 2 | | | 0 | 1 | | |
| | 50% [symbol] 75% | 2 | 4 | | | 1 | 1 | | | 3 | 4 | | | 2 | 0 | | |
| | <75% | 2 | 4 | | | 2 | 1 | | | 2 | 2 | | | 1 | 0 | | |

| | | Pooled replications | | | | NGRC + Replications | | | |
|---|---|---|---|---|---|---|---|---|---|
| GRIN2A | Coffee | PD | Co | OR(SE) | P | PD | Co | OR(SE) | P |
| (a) Coffee inspective of genotype | | | | | | | | | |
| | Light | 613 | 975 | Ref | | 15 [symbol] 1 | 151 [symbol] | Ref | |
| [symbol] | Heavy | 387 | [symbol] 86 | 0.90(0.07) | 5 × 10 [symbol] | [symbol] 96 | 1272 | 0.75(0.05) | 5 × 10 [symbol] |
| (b) GRIN2A rs [symbol] genotype [symbol] rrespective of coffee | | | | | | | | | |
| CC | [symbol] | 835 | 1513 | Ref | | 2042 | 2229 | Ref | |
| TC | | 160 | 333 | 0.88(0.10) | 0.12 | 379 | 536 | 0.75(0.06) | 3 × 10 [symbol] |
| TT | [symbol] | 14 | 15 | 1.46(0. [symbol] ) | 0.83 | 26 | 26 | 0.97(0.29) | 0.91 |
| (c) GRIN2A rs [symbol] genotype [symbol] by coffee | | | | | | | | | |
| CC | Heavy | 325 | 691 | Ref | | 76 [symbol] | 974 | Ref | |
| TC | Heavy | 53 | 1 [symbol] | 0. [symbol] 8(0.10) | 10 [symbol] | 122 | 296 | 0.51(0.06) | 1 × 10 [symbol] |
| TT | Heavy | 9 | 7 | 3.06(1.64) | 0. [symbol] | 11 | l2 | 1.33(0.61) | 0.54 |
| CC | Light | 501 | 822 | Ref | | 1279 | 1255 | | |
| TC | Light | 107 | 145 | 1.24(0.18) | 0.93 | 257 | 250 | 1.00(0.10) | 0.98 |
| TT | Light | 5 | 8 | 0.63(0. [symbol] 7) | 0.22 | 15 | 14 | 0.77(0.30) | 0.51 |
| Additive | | | | | | | | | |
| T vs. C | Heavy | 387 | [symbol] 86 | 0.78(0.12) | 0.05 | 696 | 1272 | 0.60(0.07) | 9 × 10 [symbol] |
| T vs. C | Light | 613 | 975 | 1.14(0.15) | 0.83 | 1551 | 1519 | 0.98(0.09) | 0.79 |
| Dominant | | | | | | | | | |
| CC | Heavy | 325 | [symbol] 91 | Ref | | 763 | 974 | Ref | |
| T | Heavy | 61 | 195 | 0.66(0.11) | 6 × 10 [symbol] | 133 | 298 | 0.54(0.07) | 8 × 10 [symbol] |
| CC | Light | 501 | 822 | Ref | | 1279 | 1255 | Ref | |
| T | Light | 112 | 153 | 1.20(0.17) | 0.89 | 272 | 264 | 0.9 [symbol] (0.10) | 0.91 |
| (d) Joint effects of GRIN2A rs4998 [symbol] 86 and coffee | | | | | | | | | |
| CC | Light | 5 [symbol] 1 | 822 | Ref | | 1279 | 1255 | Ref | |
| CC | Heavy | [symbol] 25 | [symbol] 91 | 0.90(0.0 [symbol] ) | 0.12 | 763 | 974 | 0. [symbol] 4(0.06) | 0.01 |
| TC | Light | 107 | 145 | 1.23(0.19) | 0.92 | 257 | 250 | 1.00(0.10) | 0.99 |
| TC | Heavy | 53 | 189 | 0.52(0.09) | 7 × 10 [symbol] | 122 | 296 | 0.43(0.05) | 8 × 10 [symbol] |
| TT | Light | 5 | 8 | 0.55(0.39) | 0.24 | 15 | 14 | 0.79(0.31) | 0.54 |
| TT | Heav | 9 | 7 | 2.60(1.39) | 0.95 | 11 | 12 | 1.09(0.50) | 0.95 |
| (e) Interactive of GRIN24 rs4998386 genotype and coffee consumption | | | | | | | | | |
| | | 996 | 1845 | 0.47(0.11) | 5 × 10 [symbol] | 2421 | 2765 | 0.50(0.06) | 5 × 10 [symbol] |
| (f) Genotype specific dose-dependent effect of coffee | | | | | | | | | |
| CC | <25% | 116 | 91 | Ref | | 448 | 290 | Ref | |
| | 25% [symbol] 50% | 118 | 79 | 1.29(0.27) | 0.89 | 460 | 257 | 1.12(0.12) | 0.34 |
| | 50% [symbol] 75% | 91 | 80 | 0.93(0.20) | 0.37 | 453 | 293 | 0.94(0.11) | 0.58 |
| | <75% | 72 | 74 | 0.76(0.19) | 0.12 | 252 | 220 | 0.54(0.09) | 6 × 10 [symbol] |
| TC | <25% | 27 | 16 | Ref | | 95 | 71 | Ref | |
| | 25% [symbol] 50% | 22 | 23 | 1.04(0.51) | 0.53 | 97 | 54 | 1.33(0.32) | 0.24 |
| | 50% [symbol] 75% | 16 | 21 | 0.40(0.19) | 0.03 | 75 | 76 | 0.55(0.16) | 0.08 |
| | <75% | 13 | 20 | 0.39(0.19) | 0.03 | 39 | 72 | 0.36(0.10) | 2 × 10 [symbol] |
| TT | <25% | 2 | 2 | | | 9 | 2 | Ref | |
| | 25% [symbol] 50% | 0 | 3 | | | 2 | 6 | 0.05(0.06) | 0.02 |
| | 50% [symbol] 75% | 3 | 1 | | | 5 | 5 | 0.21(0.23) | 0.16 |
| | <75% | 3 | 1 | | | 5 | 5 | 0.32(0.96) | 0.31 |

GWAIS adjusted for smoking, sex, age, PC1, PC2 for [SNP + SNP [symbol] coffee] model yielded P [symbol] 2 × 10 [symbol] and P [symbol] 10 [symbol]
GWAS in heavy-coffee drinkers yielded OR [symbol] 0.44, P [symbol] 10 [symbol]
PD risk conditioned on GRIN2A_rs4998386 genotype and coffee use, adjusted for smoking as well as sex and age [symbol] ulated below [symbol]
indicates data missing or illegible when filed

Applicants also repeated analyses with caffeinated tea and soda in the model (Table 9).

TABLE 9
Caffeinated Soda and Tea do not alter the results. GWAIS and stratified
GWAS results were robust when caffeinated tea and soda were included as additional
covariates, along with sex, age, PC1 and PC2.
GWAIS

| | | | SNP | | | Interaction | | | 2df |
|---|---|---|---|---|---|---|---|---|---|
| Adjusted for | Case | Con. | OR | SE | P | OR | SE | P | P |

Dominant

| | | | | | | | | | |
|---|---|---|---|---|---|---|---|---|---|
| Tea | 1441 | 920 | 0.83 | 0.12 | 0.19 | 0.49 | 0.11 | 2.3 × 10⁻³ | 2.3 × 10⁻⁶ |
| Soda | 1444 | 915 | 0.86 | 0.12 | 0.30 | 0.47 | 0.11 | 1.2 × 10⁻³ | 2.3 × 10⁻⁶ |
| Tea & soda | 1427 | 908 | 0.85 | 0.12 | 0.27 | 0.48 | 0.11 | 1.8 × 10⁻³ | 3.8 × 10⁻⁶ |

Additive

| | | | | | | | | | |
|---|---|---|---|---|---|---|---|---|---|
| Tea | 1441 | 920 | 0.85 | 0.11 | 0.24 | 0.50 | 0.11 | 1.3 × 10⁻³ | 2.1 × 10⁻⁶ |
| Soda | 1444 | 915 | 0.88 | 0.12 | 0.35 | 0.48 | 0.10 | 6.4 × 10⁻⁴ | 1.7 × 10⁻⁶ |
| Tea & soda | 1427 | 908 | 0.87 | 0.12 | 0.31 | 0.49 | 0.11 | 1.0 × 10⁻³ | 3.1 × 10⁻⁶ |

Stratified GWAS

| | | | Dominant | | | Additive | | |
|---|---|---|---|---|---|---|---|---|
| Adjusted for | Case | Con. | OR | SE | P | OR | SE | P |

Heave coffee drinker

| | | | | | | | | |
|---|---|---|---|---|---|---|---|---|
| Tea | 507 | 384 | 0.42 | 0.08 | 1.6 × 10⁻⁶ | 0.43 | 0.13 | 1.2 × 10⁻⁶ |
| Soda | 508 | 383 | 0.41 | 0.07 | 1.0 × 10⁻⁶ | 0,43 | 0.07 | 7.1 × 10⁻⁷ |
| Tea & soda | 503 | 380 | 0.42 | 0.08 | 1.9 × 10⁻⁶ | 0.43 | 0.08 | 1.4 × 10⁻⁶ |

Light coffee drinker

| | | | | | | | | |
|---|---|---|---|---|---|---|---|---|
| Tea | 934 | 536 | 0.83 | 0.12 | 0.20 | 0.85 | 0.11 | 0.24 |
| Soda | 936 | 532 | 0.86 | 0.13 | 0.30 | 0.88 | 0.12 | 0.35 |
| Tea & soda | 924 | 528 | 0.85 | 0.13 | 0.27 | 0.87 | 0.12 | 0.32 |

Given the small effect that tea and soda had on the results, we questioned if soda and tea, both of which contain caffeine, had any
effect on PD risk. The amount of caffeine in an 8 oz drink is approximately 10-30 mg in soda, 40-120 mg in black tea, and 100-200 mg
in drip or brewed coffee. The following is the OR and P values for the association of tea and coffee with PD risk in (a) the full GWAIS
model, and (b) tested individually without coffee, SNP and interaction in the model but adjusted for age, sex and PC1 and PC2. Results
are consistent with an inverse association with PD in line with the caffeine content of each drink

| | (a) PD-association in GWAIS model | | | |
|---|---|---|---|---|
| | with coffee, SNP, SNP* coftee, sex, | | (b) PD-association adjusted for | |
| | age, PC1 and PC2 in the model | | sex. age, PC1 and PC2 | |
| | OR | P | OR | P |
| Tea | 0.81 | 0.03 | 0.80 | 0.02 |
| Soda | 0.89 | 0.29 | 0.87 | 0.20 |

For details on how the data on tea, soda and smoking were collected in NGRC, see [11].

GWAIS in Discovery.

811,597 SNP genotypes [4] and lifetime caffeinated-coffee-consumption data [11] from 1458 persons with PD and 931 controls from NGRC were analyzed. Applicants tested the following models: [SNP+coffee+SNP*coffee+covariate vs. coffee+covariate] henceforth referred to as [SNP+SNP*coffee] joint test [14]. Critically, the main effect of coffee on PD risk was present in both models being compared thus Applicants controlled for coffee in the test. This model conducts a 2 degrees-of-freedom (df) joint test of SNP marginal effect and its interaction with coffee with PD [14]. Sex, age, PC1 and PC2 were included as covariates. Applicants used likelihood ratio test statistics as implemented in PLINK [25], and tested the Dominant, Additive and Recessive modes of inheritance. GWAIS was repeated once with the addition of smoking as covariate, and again by addition of caffeinated tea and soda as covariates.

Stratified GWAS in Discovery.

There were 512 cases and 387 controls in the heavy-coffee-drinking group and 946 cases and 544 controls in the light-coffee-drinking group. Applicants tested association of 811,597 genotypes with PD in each group using standard GWAS with 1 df, in PLINK [25] while adjusting for age, sex, and PC1 and PC2. Stratified GWAS were repeated with smoking, caffeinated soda and tea added as covariates (Tables 8 and 9).

Replication:

Based on the main finding in Discovery, Applicants specified the replication hypothesis a-priori as “Among heavy-coffee drinkers, carriers of rs4998386_T allele have lower risk of PD than carriers of rs4998386_CC genotype”. Note that Applicants were using the GWAIS as a means to identify the genes that might enhance the inverse association of coffee with PD with the goal of carrying the discovery forward to pharmacogenetic studies. Hence, the replication hypothesis was framed as specified. Applicants used three datasets for replication PEG [19], PAGE [20], and HIHG [8]. Applicants tested between-study heterogeneity using Breslow-Day test statistics. There was no heterogeneity in coffee use, or in rs4998386_CC and rs4998386_TC genotype frequencies, but rs4998386_TT which is rare varied significantly across studies. There were a total of 26 cases and 26 controls with rs4998386_TT genotype in Discovery and Replication combined. Given the unanticipated heterogeneity in rs4998386_TT, Applicants performed genotype-specific analysis (comparing TC to CC, excluding TT) as well as Dominant and Additive models which included TT. Categorical data were analyzed using logistic regression in SAS (version 9.2) and were adjusted for age and sex and for source of data when data were pooled. Age at onset was analyzed as a continuous variable using linear regression in SAS.

Significance.

P values were two-sided for Discovery, one-sided for Replication (given the clear directional prior hypothesis), and two sided when Discovery and Replication were pooled. There is no agreed-upon significance threshold for GWAIS. Bonferroni corrected threshold for all 811,597 SNPs on the array is P<6.4×10⁻⁸. However, not all 811,597 SNPs are independent due to linkage disequilibrium (LD). SimpleM [26] provides a sound Bonferroni-based multiple testing correction method for GWAS based on the estimated number of independent tests, allowing for marker-to-marker LD. It was shown to be the best approximation for permutation, which is computationally prohibitive for GWAS. Using simpleM Applicants calculated the number of independent SNPs genome-wide for NGRC as Meff=430,151; thus the Bonferroni corrected threshold for independent tests was P<1.16×10⁻⁷.

Imputation:

Applicants used IMPUTE v2 [27] with HapMap and 1000 Genomes genotypes combined for reference data, to infer genotypes for SNPs that were not originally included on the Illumina OMNI-1 array and thus not genotyped in the NGRC data set. 2,710,971 SNPs were imputed with high reliability (information score≧0.95) and had MAF>0.01, increasing the genomic coverage to 3,522,568 SNPs total (genotyped and imputed). Applicants performed GWAIS and stratified GWAS for the GRIN2A region (Chromosome 16, 97 Mb-102 Mb) using genotype probability data (dose 2-0) in R software (r-project.org website).

Linkage disequilibrium and haplotype blocks were estimated using the Haploview software [28]. Haplotype analysis was performed using haplo.stats adjusting for sex and age [29].

Copy Number Variations (CNV):

Applicants used Golden Helix SNP Variation Suite version 7.2.3 on the goldenhelix.com website and PennCNV [30] to explore for deletions or duplications in the GRIN2A region. Golden Helix found none; PennCNV identified two controls with CNVs, which even if confirmed to be real, would not affect the results of the study.

GWAIS in Discovery.

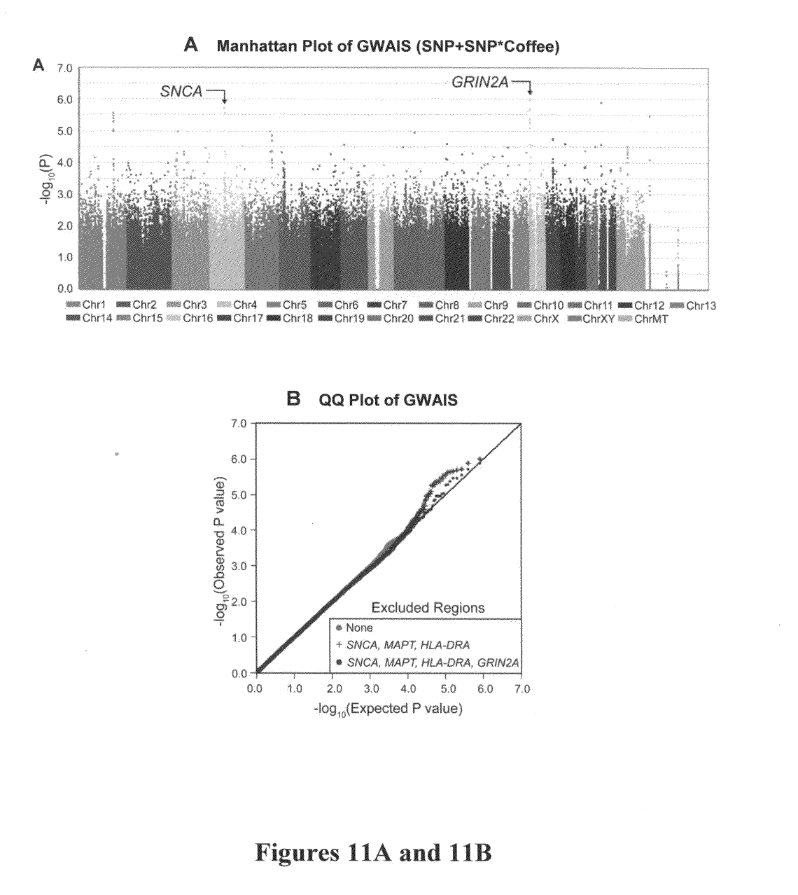

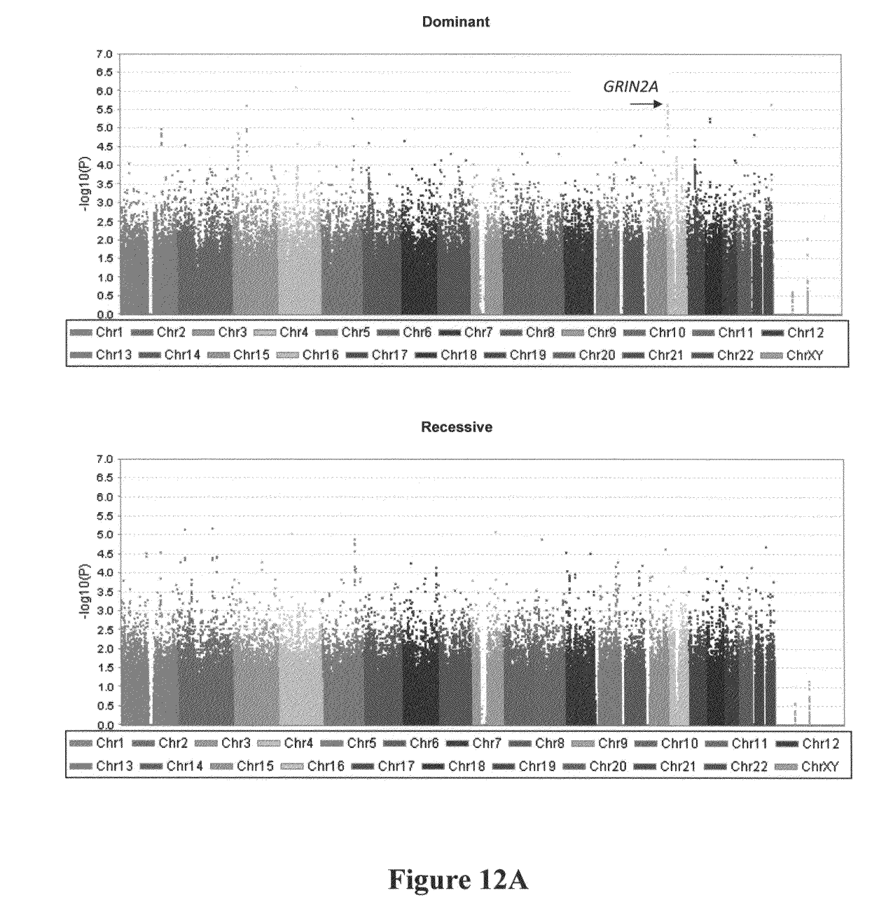

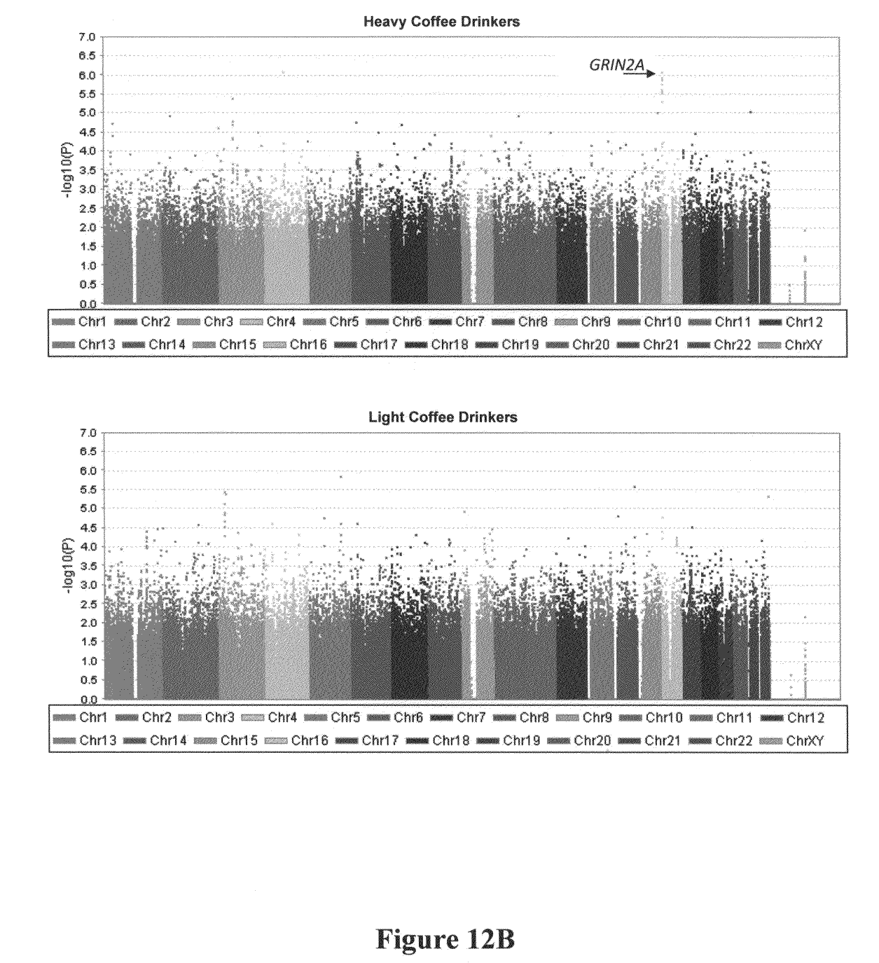

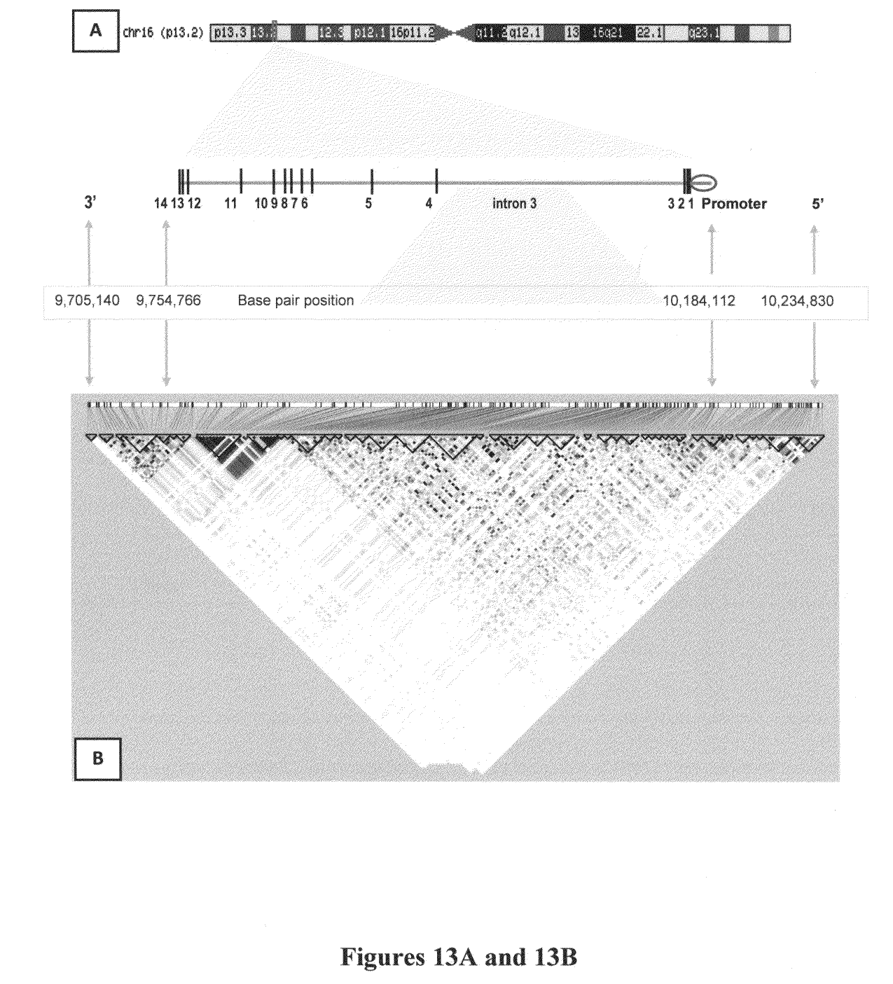

The most significant result was the novel appearance, on the Manhattan plot (FIGS. 11A and 12A), of a block of linked SNPs which map to the GRIN2A gene on chromosome 16 (FIG. 13). This locus had not been detected in GWAS previously because its main effect is modest. However, when considered in the context of interaction with coffee, GRIN2A surpassed all known PD-associated genes in significance including SNCA which has been the strongest association with PD in GWAS. As shown in Table 10, the known PD susceptibility genes were detected by GWAIS but at lower significance levels than in GWAS, because in the absence of interaction, GWAIS (2 df joint test) has substantially lower power than GWAS (1 df main effect).

TABLE 10
GRIN2A was the most significant signal in GWAIS (also see FIGS. 11
and 14). GWAIS analysis was [SNP + SNP* coffee] test with 2 df adjusting for sex, age, PC1 and
PC2. The test examines the significance of the SNP main effect and its interaction with coffee,
without introducing the significant effect of coffee on PD. Results for GRIN2A were equally
significant under Dominant and Additive models. Recessive model had no clear signal (see
FIG. 12). Also shown are the results obtained with the same dataset and under the same
analytic model for the known PD genes SNCA, MAPT, HLA. SNCA and HLA had reached P <
5 × 10⁻⁸ in Applicants' GWAS. The fall in significance in GWAIS is due in part to 1/3 reduction
in sample size due to unavailability of coffee data, and the penalty imposed by the added degree
of freedom. GRIN2A did not have a strong main effect to be noticed in GWAS, but inclusion of
coffee and interaction placed GRIN2A higher than SNCA, HLA and MAPT.

| | | | | Minor/ | | | | Dominant | | | | | | | Additive | | | | | | |
|---|---|---|---|---|---|---|---|---|---|---|---|---|---|---|---|---|---|---|---|---|---|
| | | | | Major | MAF | MAF | HWE | SNP | | | Interaction | | | 2df | SNP | | | Interaction | | | 2df |
| CHR | Gene | SNP | BP | Allele | Case | cont | P | OR | SE | P | OR | SE | P | P | OR | SE | P | OR | SE | P | P |

Novel PD-association identified via interaction with coffee

| | | | | | | | | | | | | | | | | | | | | | |
|---|---|---|---|---|---|---|---|---|---|---|---|---|---|---|---|---|---|---|---|---|---|
| 1 [symbol] | GRIN2A | rs499 [symbol] 386 | 9978046 | T/C | 0.08 | 0.12 | 0.54 | 0. [symbol] 2 | 0.12 | 0.17 | 0.49 | 0.11 | 2 × 10 [symbol] | 1 × 10 [symbol] | 0. [symbol] 4 | 0.11 | 0.19 | 0.50 | 0.11 | 1 × 10 [symbol] | 1 × 10 [symbol] |
| 1 [symbol] | GRIN2A | rs17569693 | 99 [symbol] 7686 | G/A | 0.08 | 0.12 | 0.54 | 0.79 | 0.11 | 0.10 | 0.51 | 0.12 | 4 × 10 [symbol] | 1 × 10 [symbol] | 0. [symbol] 0 | 0.10 | 0.09 | 0.54 | 0.12 | 5 × 10 [symbol] | 2 × 10 [symbol] |
| 1 [symbol] | GRIN2A | rs2043728 | 10003034 | T/C | 0.09 | 0.12 | 0.86 | 0.82 | 0.12 | 0.1 [symbol] | 0.53 | 0.12 | 5 × 10 [symbol] | 8 × 10 [symbol] | 0. [symbol] 2 | 0.11 | 0.13 | 0.57 | 0.12 | 0.01 | 5 × 10 [symbol] |
| 1 [symbol] | GRIN2A | rs8055683 | 10052710 | T/C | 0.09 | 0.13 | 1.00 | 0.82 | 0.12 | 0.17 | 0.52 | 0.12 | 3 × 10 [symbol] | 4 × 10 [symbol] | 0. [symbol] 3 | 0.11 | 0.14 | 0.55 | 0.11 | 4 × 10 [symbol] | 4 × 10 [symbol] |
| 1 [symbol] | GRIN2A | rs9927926 | 10057405 | C/T | 0.05 | 0.13 | 1.00 | 0.82 | 0.12 | 0.17 | 0.52 | 0.12 | 3 × 10 [symbol] | 4 × 10 [symbol] | 0. [symbol] 3 | 0.11 | 0.14 | 0.55 | 0.11 | 4 × 10 [symbol] | 4 × 10 [symbol] |
| 1 [symbol] | GRIN2A | rs17 [symbol] 71033 | 10065727 | A/G | 0.09 | 0.13 | 1.00 | 0.85 | 0.12 | 0.24 | 0.50 | 0.11 | 2 × 10 [symbol] | 3 × 10 [symbol] | 0. [symbol] 5 | 0.11 | 0.20 | 0.53 | 0.11 | 2 × 10 [symbol] | 4 × 10 [symbol] |
| 1 [symbol] | GRIN2A | rs9933111 | 10072100 | G/A | 0.09 | 0.13 | 0.8 [symbol] | 0.85 | 0.12 | 0.24 | 0.49 | 0.11 | 2 × 10 [symbol] | 2 × 10 [symbol] | 0. [symbol] 5 | 011 | 0.20 | 0.53 | 0.11 | 2 × 10 [symbol] | 2 × 10 [symbol] |
| 1 [symbol] | GRIN2A | rs13331465 | 100779 [symbol] | T/C | 0.02 | 0.13 | 0.8 [symbol] | 0.85 | 0.12 | 0.23 | 0.49 | 0.11 | 2 × 10 [symbol] | 2 × 10 [symbol] | 0. [symbol] 5 | 0.11 | 0.19 | 0.53 | 0.11 | 2 × 10 [symbol] | 2 × 10 [symbol] |
| 1 [symbol] | GRIN2A | rs1333 [symbol] 32 | 1007 [symbol] 155 | C/A | 0.09 | 0.13 | 0.85 | 0.85 | 0.12 | 0.23 | 0.49 | 0.11 | 2 × 10 [symbol] | 2 × 10 [symbol] | 0. [symbol] 5 | 0.11 | 0.19 | 0.52 | 0.11 | 2 × 10 [symbol] | 2 × 10 [symbol] |
| 1 [symbol] | GRIN2A | rs1445270 | 10082 [symbol] 19 | T/G | 0.09 | 0.13 | 0.77 | 0.86 | 0.12 | 0.29 | 0.49 | 0.11 | 2 × 10 [symbol] | 5 × 10 [symbol] | 0. [symbol] 5 | 0.11 | 0.24 | 0.53 | 0.11 | 2 × 10 [symbol] | 5 × 10 [symbol] |
| 1 [symbol] | GRIN2A | r1 [symbol] 570 | 10113676 | C/T | 0.11 | 0.15 | 0.15 | 0.89 | 0.12 | 0.35 | 0.51 | 0.11 | 1 × 10 [symbol] | 8 × 10 [symbol] | 0. [symbol] 2 | 0.11 | 0.46 | 0.54 | 0.11 | 2 × 10 [symbol] | 3 × 10 [symbol] |
| 1 [symbol] | GRIN2A | rs144 [symbol] 253 | 10125357 | C/T | 0.09 | 0.13 | 0.67 | 0. [symbol] 3 | 0.11 | 0.18 | 0.52 | 0.12 | 3 × 10 [symbol] | 4 × 10 [symbol] | 0. [symbol] 4 | 0.11 | 0.18 | 0.55 | 0.11 | 3 × 10 [symbol] | 5 × 10 [symbol] |

Well-established PD-associated genes identified via their main effect

| | | | | | | | | | | | | | | | | | | | | | |
|---|---|---|---|---|---|---|---|---|---|---|---|---|---|---|---|---|---|---|---|---|---|
| 4 | SNCA | rs35 [symbol] 220 | 908603 [symbol] 3 | T/C | 0.43 | 0.35 | 0.77 | 1.37 | 0.16 | 0.01 | 1.25 | 0.23 | 0.23 | 3 × 10 [symbol] | 1.3 [symbol] | 0.11 | 2 × 10 [symbol] | 1.05 | 0.14 | 0.69 | 3 × 10 [symbol] |
| 4 | SNCA | rs35816 [symbol] | 90893454 | G/A | 0.51 | 0.44 | 0.89 | 1.33 | 0.17 | 0.03 | 1.33 | 0.26 | 0.16 | 1 × 10 [symbol] | 1.2 [symbol] | 0.10 | 3 × 10 [symbol] | 1.09 | 0.14 | 0.51 | 5 × 10 [symbol] |
| 17 | MAPT | rs199533 | 421840 [symbol] | T/C | 0.17 | 0.22 | 0.15 | 0.65 | 0.05 | 2 × 10 [symbol] | 1.10 | 0.21 | 0.23 | 7 × 10 [symbol] | 0.6 [symbol] | 0.07 | 2 × 10 [symbol] | 1.10 | 0.18 | 0.57 | 7 × 10 [symbol] |
| [symbol] | HLA | rs3129882 | 32517508 | G/A | 0.46 | 0.40 | 0.78 | 1.25 | 0.15 | 0.07 | 1.26 | 0.24 | 0.22 | 2 × 10 [symbol] | 1.24 | 0.10 | 0.01 | 1.0 [symbol] | 0.14 | 0.54 | 5 × 10 [symbol] |

[symbol]

indicates data missing or illegible when filed

The signal for known PD genes were driven only by their main effects with no evidence for interaction (P_{interaction}=0.5-0.7); whereas the signals for PD-associated SNPs in GRIN2A were enhanced by SNP*coffee interaction (P_{interaction}˜10⁻³). The quantile-quantile (QQ) plot of the expected vs. observed genome-wide P values (FIG. 11B) is also clear evidence for the impact of GRIN2A on PD risk. Two points in the QQ plot are noteworthy: a deviation around P<10⁻³ and a larger deviation at the extreme P<10⁻⁵. The deviation around 10⁻³ is due to the known PD genes, SNCA, MAPT and HLA; it disappears when these genes are excluded. The extreme deviation at <10-5 is primarily due to GRIN2A; as suggested by a considerable reduction in the deviation when GRIN2A is excluded. GWAIS results described above were obtained from a test that measures the combined significance of the SNP and its interaction with coffee on risk of PD [14]. The test has 2 df; hence when interaction is absent, GWAIS is less powerful than GWAS which has only 1 df. Furthermore, the sample size was smaller in GWAIS because it required not only genotypes but also coffee data, which was available for ⅔ of NGRC. Under these conditions, GWAIS produced P_{2df}>10⁻⁶ (FIG. 11A) for the top SNP in SNCA which had reached P=3×10⁻¹¹ in NGRC GWAS [4]. This drop in significance demonstrates the dramatic loss of power in GWAIS as compared to GWAS. Under these conditions, GWAIS yielded P_{2df}=10⁻⁶ for rs4998386 in GRIN2A (as compared to P_{2df}=3×10⁻⁶ for SNCA and P_{2df}=7×10⁻⁵ for MAPT). Dominant and Additive models produced nearly identical results for GRIN2A SNPs (Table 10). Recessive model had no notable signal (FIG. 12A).

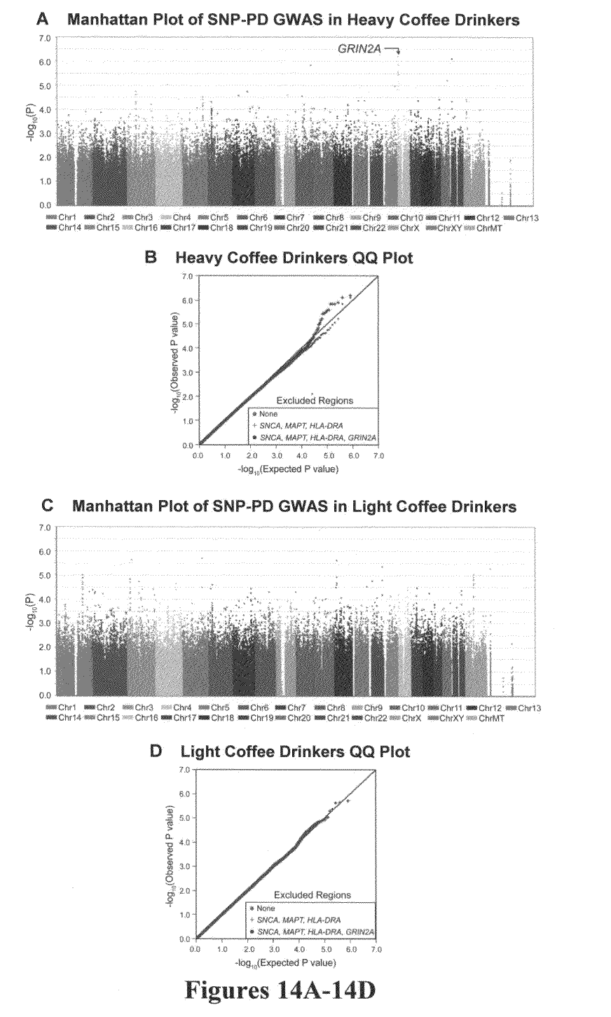

GWAS in heavy- and light-coffee drinkers in Discovery samples. (FIG. 14, Table 11). With one goal being pharmacogenetic applications, Applicants were interested in genes that modulate risk in people who consume caffeine, thus Applicants stratified the subjects as heavy-drinkers or light-drinkers (light includes nondrinkers) and performed GWAS in each group (SNP-PD test, 1 df). The sample size was now further reduced to only 512 cases and 387 controls who drank more than the median (heavy-drinker) and 946 cases and 544 control subjects who drank less than the median (light-drinkers). As expected due to interaction, which suggests different association-patterns across categories, most of the signals seen in GWAIS (FIG. 11A) appeared within either heavy-drinkers (FIG. 14A, Table 11, FIG. 12B) or light-drinkers (FIG. 14C, Table 11, FIG. 12B). In heavy-drinkers, the focus of this study, the most significant result was GRIN2A_rs4998386 (P=6×10⁻⁷) and 11 neighboring SNPs (P=10⁻⁵ to 10⁻⁶, Table 11).

TABLE 11
GRIN2A was the most significant results in GWAS for in heavy-coffee-drinkers
(see also FIG. 14). Standard GWAS (PD-SNP association, no interaction) was conducted among
heavy-and light-coffee drinkers separately. GRIN2A was most notable only among heavy coffee
drinkers. Odds ratio (OR) of 0.41-0.46 suggests that among heavy coffee drinkers, who are
known to be at reduced risk for PD, GRIN2A genotypes further modifies risk by over two-fold.
As expected due to interaction, GRIN2A did not have a significant effect in light-coffee drinkers.
This is in contrast to known PD genes which exhibited their effects regardless of coffee
consumption.

| | | | | GWAS in Heavy-Coffee Drinkers | | | | | | | | GWAS In Light Coffee Drinkers | | | | | | | |
|---|---|---|---|---|---|---|---|---|---|---|---|---|---|---|---|---|---|---|---|
| | | | | MAF | MAF | Dominant | | | Additive | | | MAF | MAF | Dominent | | | Additive | | |
| CHR | GENE | SNP | BP | Case | Cont | OR | SE | P | OR | SE | P | Case | Cont | OR | SE | P | OR | SE | P |

Nov [symbol] PD-association identified via interaction with coffee

| | | | | | | | | | | | | | | | | | | | |
|---|---|---|---|---|---|---|---|---|---|---|---|---|---|---|---|---|---|---|---|
| 15 | GRIN2A | rs [symbol] | [symbol] | 0.07 | 0.14 | 0.41 | 0.07 | 5 × 10 [symbol] | 0.43 | 0.07 | 6 × 10 [symbol] | 0.09 | 0.11 | [symbol] | 0.12 | 0.1 [symbol] | [symbol] | 0.11 | 0.19 |
| 15 | GRIN2A | rs [symbol] | [symbol] | 0.07 | 0.13 | 0.41 | 0.08 | 1 × 10 [symbol] | 0.44 | 0.08 | 3 × 10 [symbol] | 0.09 | 0.11 | [symbol] | 0.11 | 0.10 | [symbol] | 0.11 | 0.09 |
| 15 | GRIN2A | rs [symbol] | [symbol] | 0.08 | 0.14 | 0.44 | 0.08 | 5 × 10 [symbol] | 0.47 | 0.08 | 7 × 10 [symbol] | 0.09 | 0.11 | [symbol] | 0.12 | 0.17 | [symbol] | 0.11 | 0.13 |
| 15 | GRIN2A | rs [symbol] | [symbol] | 0.08 | 0.14 | 0.43 | 0.08 | 2 × 10 [symbol] | 0.47 | 0.08 | 3 × 10 [symbol] | 0.10 | 0.12 | [symbol] | 0.12 | 0.17 | [symbol] | 0.11 | [symbol] |
| 15 | GRIN2A | rs [symbol] | [symbol] | 0.08 | 0.14 | 0.43 | 0.08 | 2 × 10 [symbol] | 0.47 | 0.08 | 3 × 10 [symbol] | 0.10 | 0.12 | [symbol] | 0.12 | 0.17 | [symbol] | 0.11 | [symbol] |
| 15 | GRIN2A | rs [symbol] | [symbol] | 0.08 | 0.14 | 0.43 | 0.08 | 2 × 10 [symbol] | 0.46 | 0.08 | 3 × 10 [symbol] | 0.10 | 0.11 | [symbol] | 0.12 | 0.25 | [symbol] | 0.11 | [symbol] |
| 15 | GRIN2A | rs [symbol] | [symbol] | 0.08 | 0.15 | 0.42 | 0.08 | 1 × 10 [symbol] | 0.45 | 0.07 | 1 × 10 [symbol] | 0.10 | 0.12 | [symbol] | 0.12 | [symbol] | [symbol] | 0.11 | [symbol] |
| 15 | GRIN2A | rs [symbol] | [symbol] | 0.08 | 0.15 | 0.42 | 0.08 | 1 × 10 [symbol] | 0.45 | 0.07 | 1 × 10 [symbol] | 0.10 | 0.12 | [symbol] | 0.12 | [symbol] | [symbol] | 0.11 | [symbol] |
| 15 | GRIN2A | rs [symbol] | [symbol] | 0.08 | 0.15 | 0.42 | 0.07 | 1 × 10 [symbol] | 0.45 | 0.07 | 1 × 10 [symbol] | 0.10 | 0.12 | [symbol] | 0.12 | [symbol] | [symbol] | 0.11 | [symbol] |
| 15 | GRIN2A | rs [symbol] | [symbol] | 0.08 | 0.14 | 0.43 | 0.08 | 3 × 10 [symbol] | 0.47 | 0.08 | 3 × 10 [symbol] | 0.10 | 0.12 | [symbol] | 0.12 | 0.29 | [symbol] | 0.11 | [symbol] |
| 15 | GRIN2A | rs [symbol] | [symbol] | 0.09 | 0.1 [symbol] | 0.46 | 0.08 | 3 × 10 [symbol] | 0.51 | 0.08 | 1 × 10 [symbol] | 0.12 | 0.13 | [symbol] | 0.12 | [symbol] | [symbol] | 0.11 | [symbol] |
| 15 | GRIN2A | rs [symbol] | [symbol] | 0.09 | 0.15 | 0.44 | 0.08 | 2 × 10 [symbol] | 0.47 | 0.08 | 4 × 10 [symbol] | 0.10 | 0.12 | [symbol] | 0.11 | [symbol] | [symbol] | 0.11 | [symbol] |

Well-established PD-associated genes identified via their main effect

| | | | | | | | | | | | | | | | | | | | |
|---|---|---|---|---|---|---|---|---|---|---|---|---|---|---|---|---|---|---|---|
| 4 | SNCA | rs [symbol] | [symbol] | [symbol] .42 | 0.34 | 1.71 | 0.25 | 2 × 10 [symbol] | 1.43 | 0.15 | 6 × 10 [symbol] | 0.43 | 0.3 [symbol] | 1.37 | 0.15 | 0.01 | 1.36 | 0.11 | 2 × 10 [symbol] |
| 4 | SNCA | rs [symbol] | [symbol] | 0.50 | 0.42 | 1.75 | 0.27 | 3 × 10 [symbol] | 1.3 [symbol] | 0.14 | 1 × 10 [symbol] | 0.51 | 0.45 | 1.33 | 0.77 | [symbol] | 1. [symbol] | 0.10 | 3 × 10 [symbol] |
| 17 | MAPT | rs [symbol] | [symbol] | 0. [symbol] 5 | 0.21 | 0.71 | 0.10 | [symbol] | 0.75 | 0.10 | 0.02 | 0.17 | 0.23 | 0.55 | 0.05 | [symbol] 10 | [symbol] | 0.07 | 3 × 10 [symbol] |
| [symbol] | HLA | rs [symbol] | [symbol] | [symbol] .47 | 0.40 | 1.57 | 0.24 | 2 × 10 [symbol] | 1.34 | 0.13 | 4 × 10 [symbol] | 0.45 | 0.40 | 1 [symbol] | 0.15 | [symbol] | 1.24 | 0.10 | [symbol] |

[symbol]

indicates data missing or illegible when filed

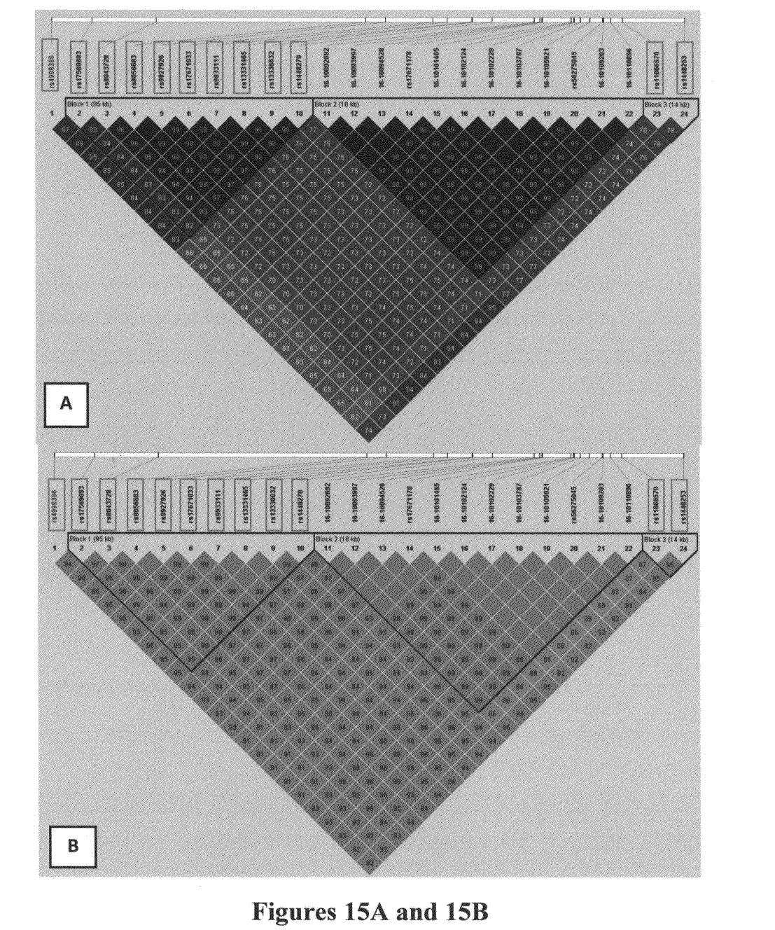

The QQ plots for stratified GWAS also demonstrate clearly that GRIN2A is the single primary PD associated locus in heavy-coffee drinkers (FIG. 14B): Exclusion of SNCA, HLA and MAPT did not have an impact in heavy drinkers, whereas exclusion of GRIN2A nearly abolished the extreme P values of 10⁻⁵-10⁻⁶. No clear signals were detected in light-coffee-drinkers (FIGS. 14C, 14D). The 12 GRIN2A SNPs that were associated with PD via heavy-coffee consumption had similar minor-allele frequencies (MAF=0.13-0.16 in controls) and odds ratios (OR=0.43-0.51) and were in strong LD (FIG. 15). Haplotype analysis did not strengthen the signal. Applicants found no evidence that CNV might have influenced the results. Applicants therefore selected only the SNP with the lowest P value for replication (GRIN2A_rs4998386).

Genotype-specific association of coffee with PD in Discovery (Table 12).

TABLE 12
PD risk conditioned on GRIN2A genotype and coffee use

| | | NGRC | | | | PEG | | | | PAGE | | | | HIHG | |
|---|---|---|---|---|---|---|---|---|---|---|---|---|---|---|---|
| | | (Discovery) | | | | (Replication 1) | | | | (Replication 2) | | | | (Replication 3) | |
| GRIN2A | Coffee | PD | CO | OR(SE) | P | PD | CO | OR(SE) | P | PD | CO | OR(SE) | P | PD | CO |

(A) Coffee Irrespective of genotype

| | | | | | | | | | | | | | | | |
|---|---|---|---|---|---|---|---|---|---|---|---|---|---|---|---|
| [symbol] | Light | [symbol] | 544 | Ref | | 1 [symbol] 4 | 1 [symbol] | Ref | | 290 | 7 [symbol] | Ref | | 147 | [symbol] 6 |
| [symbol] | Heavy | 512 | 3 [symbol] 7 | 0.56(0.05) | 5 × 10 [symbol] | [symbol] 5 | 122 | 0.59(0.12) | 0.02 | 235 | 7 [symbol] | [symbol] | 0.02 | [symbol] 2 | 47 |

(B) GRIN2A rs [symbol] genotype [symbol] of coffee

| | | | | | | | | | | | | | | | |
|---|---|---|---|---|---|---|---|---|---|---|---|---|---|---|---|
| CC | [symbol] | 1227 | 716 | Ref | | 234 | 249 | Ref | | 434 | 1199 | Ref | | 1 [symbol] 9 | 110 |
| TC | | 21 [symbol] | 204 | 0. [symbol] 2(0.07) | 2 × 10 [symbol] | 42 | 55 | [symbol] | 0.13 | [symbol] 5 | 2 [symbol] 7 | 0. [symbol] (0.12) | 0.17 | [symbol] | 22 |
| TT | | 12 | 11 | 0. [symbol] 3(0.23) | 0.15 | 4 | 6 | | | 6 | 6 | | | 4 | 1 |

(C) GRIN2A r [symbol] genotype stratified by coffee

| | | | | | | | | | | | | | | | |
|---|---|---|---|---|---|---|---|---|---|---|---|---|---|---|---|
| CC | Heavy | 441 | 253 | Ref | 22 | [symbol] | 94 | Ref | | 199 | 575 | Ref | | 51 | 36 |
| TC | Heavy | 69 | 99 | [symbol] | 2 × 10 [symbol] | 14 | 27 | 0. [symbol] 2(0.23) | 0.10 | 31 | 154 | 0.5 [symbol] (0.12) | 0.01 | 9 | 11 |
| TT | Heavy | 2 | 5 | 0.15(0.1 [symbol] ) | 0.05 | 2 | 1 | | | 5 | 6 | | | 2 | 0 |
| CC | Light | 78 [symbol] | [symbol] | Ref | | 154 | 155 | Ref | | 235 | [symbol] 23 | Ref | | 118 | 74 |
| TC | Light | 15 [symbol] | 105 | 0. [symbol] 1(0.12) | 0.16 | 2 [symbol] | 28 | 0.97(0.29) | 0.46 | 54 | 11 [symbol] | 1.27(0.23) | 0.90 | 27 | 11 |
| TT | Light | 10 | 6 | 0.81(0.44) | 0.70 | 2 | 5 | | | 1 | 2 | | | 2 | 1 |

Additive model

| | | | | | | | | | | | | | | | |
|---|---|---|---|---|---|---|---|---|---|---|---|---|---|---|---|
| T/C | Heavy | 512 | 387 | 0.42(0.07) | 3 × 10 [symbol] | 9 [symbol] | 122 | 0.78(0.25) | 0.22 | 235 | 736 | 0.75(0.14) | 0. [symbol] 6 | [symbol] 2 | 47 |
| T/C | Light | 545 | 544 | 0. [symbol] 3(0.11) | 0.16 | 1 [symbol] 4 | 1 [symbol] | 0. [symbol] 3(0.20) | 0.22 | 290 | 73 [symbol] | 1.25(0.22) | 0.90 | 147 | [symbol] |

Dominant model

| | | | | | | | | | | | | | | | |
|---|---|---|---|---|---|---|---|---|---|---|---|---|---|---|---|
| CC | Heavy | 441 | 2 [symbol] 3 | Ref | | [symbol] 0 | 94 | Ref | | 199 | 575 | Ref | | 51 | 36 |
| T [symbol] | Heavy | 71 | 104 | 0.4 [symbol] (0.07) | 5 × 10 [symbol] | 15 | 2 [symbol] | 0. [symbol] 6(0.24) | 0.14 | 36 | 160 | 0.65(0.13) | 0.02 | 11 | 11 |
| CC | Light | 7 [symbol] | 433 | Ref | | 154 | 155 | Ref | | 235 | 623 | Ref | | 118 | 74 |
| T [symbol] | Light | 160 | 111 | 0. [symbol] 1(0.12) | 0.15 | 30 | 33 | 0. [symbol] (0.25) | 0.32 | 55 | 115 | 1.27(0.23) | 0.90 | 29 | 12 |

(D) Joint effects of GRIN2A r [symbol] and coffee

| | | | | | | | | | | | | | | | |
|---|---|---|---|---|---|---|---|---|---|---|---|---|---|---|---|
| CC | Light | 7 [symbol] | 433 | Ref. | | 154 | 155 | Ref | | 235 | [symbol] | Ref | | 118 | 74 |
| CC | Heavy | 441 | 253 | 0.75(0.0 [symbol] ) | [symbol] | 50 | 94 | 0.73(0.14) | 0.05 | 199 | 576 | 0.92(0.10) | 0.22 | 51 | 36 |
| TC | Light | 150 | 105 | 0. [symbol] 1(0.12) | 0.15 | 28 | 25 | 0.99(0.29) | [symbol] | 54 | 113 | 1.27(0.23) | 0.10 | 27 | 11 |
| TC | Heavy | 69 | 99 | 0.32(0.05) | 7 × 10 [symbol] | 14 | 27 | 0.46(0.1 [symbol] ) | 0.01 | 31 | 1 [symbol] 4 | 0.54(0.11) | 2 × 10 [symbol] | 9 | 11 |
| TT | Light | 10 | [symbol] | 0. [symbol] 1(0.44) | 0.70 | 2 | 5 | | | 1 | 2 | | | 2 | 1 |
| TT | Heavy | 2 | [symbol] | 0.14(0.12) | 0.02 | 2 | 1 | | | 5 | 6 | | | 2 | 0 |

(E) Interaction of GRIN2A rs [symbol] genotypes and coffee consumption

| | | | | | | | | | | | | | |
|---|---|---|---|---|---|---|---|---|---|---|---|---|---|
| 144 [symbol] | 920 | 0.52(0.12) | 4 × 10 [symbol] | 276 | 304 | 0. [symbol] 5(0.31) | 0.13 | 519 | 14 [symbol] | 0.4 [symbol] (0.13) | 3 × 10 [symbol] | 205 | 132 |

(F) Genotype specific do [symbol] -dependent effect of coffee

| | | | | | | | | | | | | | | | |
|---|---|---|---|---|---|---|---|---|---|---|---|---|---|---|---|
| CC | [symbol] 25 | 334 | 159 | Ref | | [symbol] | 66 | Ref | | 131 | 317 | Ref | | 49 | 32 |
| | 25% [symbol] 50% | 344 | 176 | 1.03(0.14) | [symbol] .64 | [symbol] | 67 | 0.97(0.24) | 0.46 | 104 | 305 | 0. [symbol] (0.13) | 0.11 | 55 | 25 |
| | 50% [symbol] 75% | 36 [symbol] | 203 | 0.91(0.12) | [symbol] .47 | 54 | 55 | 0.88(0.23) | 0.31 | 86 | 295 | 0.71(0.11) | 0. [symbol] 2 | 37 | 29 |
| | [symbol] 75% | 183 | 146 | 0. [symbol] (0.09) | 3 × 10 [symbol] | 47 | 5 [symbol] | 0. [symbol] 8(0.15) | 0.07 | 113 | 2 [symbol] 0 | 0.99(0.15) | 0.47 | 28 | 24 |
| TC | [symbol] 25% | 69 | 55 | Ref | | 14 | 12 | Ref | | 32 | 53 | Ref | | 13 | 6 |
| | 25% [symbol] 50% | 65 | 41 | 1.31(0.37) | 0.34 | 12 | 13 | 0. [symbol] 2(0.37) | 0.21 | 22 | 60 | 0.60(0.20) | 0.07 | 10 | 3 |
| | 50% [symbol] 75% | 59 | 56 | 0.71(0.20) | 0.21 | 9 | 15 | 0.30(0.23) | 0.03 | 1 [symbol] | 68 | 0.3 [symbol] (0.13) | 3 × 10 [symbol] | 7 | 7 |
| | [symbol] 75% | 26 | 52 | 0.31(0.10) | 2 × 10 [symbol] | 7 | 15 | 0.36(0.23) | 0.05 | 16 | 56 | 0.31(0.11) | 5 × 10 [symbol] | 5 | 5 |
| TT | [symbol] 25% | [symbol] | 0 | | | 1 | 2 | | | 1 | 0 | | | 1 | 0 |
| | 25% [symbol] 50% | 2 | 3 | [symbol] | | [symbol] | 2 | | | 0 | 2 | | | 0 | 1 |
| | 50% [symbol] 75% | 2 | 4 | | | 1 | 1 | | | 3 | 4 | | | 2 | 0 |
| | [symbol] 75% | 2 | 4 | | | 2 | 1 | | | 2 | 2 | | | 1 | 0 |

| | | HIHG | | Pooled | | | | Pooled | | | |
|---|---|---|---|---|---|---|---|---|---|---|---|
| | | (Replication 3) | | Replications | | | | NGRC + Replications | | | |
| GRIN2A | Coffee | OR(SE) | P | PD | CO | OR(SE) | P | PD | CO | OR(SE) | P |
| (A) Coffee Irrespective of genotype | | | | | | | | | | | |
| [symbol] | Light | Ref | | 621 | 1012 | Ref | 15 [symbol] 7 | 621 | 1012 | Ref | |
| [symbol] | Heavy | 0.56(0.17) | 0.05 | 393 | 905 | 0.79(0.07) | 2 × 10 [symbol] | 905 | 1292 | 0.73(0.04) | 3 × 10 [symbol] |
| (B) GRIN2A rs [symbol] genotype [symbol] of coffee | | | | | | | | | | | |
| CC | [symbol] | Ref | | 837 | 1556 | Ref | | 2054 | 2274 | Ref | |
| TC | | 1.04(0.32) | 0.55 | 163 | 344 | 0.69(0.10) | 0.14 | 382 | 54 [symbol] | 0.75(0.0 [symbol] ) | 2 × 10 [symbol] |
| TT | | | | 14 | 15 | | | 2 [symbol] | 26 | [symbol] | |
| (C) GRIN2A r [symbol] genotype stratified by coffee | | | | | | | | | | | |
| CC | Heavy | Ref | | 330 | 705 | Ref | | 771 | 969 | Ref | |
| TC | Heavy | 0.54(0.33) | 0.19 | 54 | 192 | 0.59(0.10) | 1 × 10 [symbol] | 123 | 291 | 0.51(0.0 [symbol] ) | 7 × 10 [symbol] |
| TT | Heavy | | | 9 | 7 | | | 11 | 12 | [symbol] | |
| CC | Light | Ref | | 507 | 852 | Ref | | 1293 | 1285 | Ref | |
| TC | Light | 1.36(0.5) [symbol] | 0.77 | 109 | 152 | 1.24(0.1 [symbol] ) | 0.93 | 259 | 257 | 1.00(0.10) | 0.99 |
| TT | Light | | | 5 | 8 | | | 15 | 14 | | |
| Additive model | | | | | | | | | | | |
| T/C | Heavy | 0. [symbol] 3(0.40) | 0.43 | 393 | 905 | 0.77(0.11) | 0.04 | 905 | 12 [symbol] | 0. [symbol] 0(0.07) | 4 × 10 [symbol] |
| T/C | Light | 1.27(0.45) | 0.75 | 621 | 1012 | 1.15(0.15) | 0.85 | 1 [symbol] 57 | 15 [symbol] | 0.96(0.09) | 0.79 |
| Dominant model | | | | | | | | | | | |
| CC | Heavy | Ref | | 330 | 70 [symbol] | Ref | | 771 | 96 [symbol] | Ref | |
| T [symbol] | Heavy | 0.76(0.37) | 0.29 | 63 | 199 | 0.70(0.11) | 0.01 | 134 | 303 | 0.54(0.05) | 3 × 10 [symbol] |
| CC | Light | Ref | | [symbol] 07 | 852 | Ref | | 1293 | 128 [symbol] | Ref | |
| T [symbol] | Light | 1.34(0.53) | 0.77 | 114 | 160 | 1.20(0.17) | 0.90 | 771 | 271 | 0.99(0.10) | 0. [symbol] 9 |
| (D) Joint effects of GRIN2A r [symbol] and coffee | | | | | | | | | | | |
| CC | Light | Ref | | 507 | 852 | Ref | | 1293 | 1285 | Ref | |
| CC | Heavy | 0.73(0.21) | 0.13 | 330 | 705 | 0. [symbol] (0.05) | 0.0 [symbol] | 771 | 96 [symbol] | 0. [symbol] 2(0.06) | 3 × 10 [symbol] |
| TC | Light | 1.39(0.56) | 0.21 | 109 | 152 | 1.24(0.1 [symbol] ) | 0.07 | 259 | 257 | 1.00(0. [symbol] ) | 0.99 |
| TC | Heavy | 0.49(0.24) | 0.0 [symbol] | 54 | 192 | 0.52(0.09) | 5 × 10 [symbol] | 123 | 291 | 0.41(0.05) | [symbol] × 10 [symbol] |
| TT | Light | | | 5 | 8 | | | 1 [symbol] | 14 | | |
| TT | Heavy | | | 9 | 7 | | | 11 | 12 | | |
| (E) Interaction of GRIN2A rs [symbol] genotypes and coffee consumption | | | | | | | | | | | |
| | | 0. [symbol] (0.32) | [symbol] | 1000 | 19 [symbol] | 0.4 [symbol] (0.11) | 5 × 10 [symbol] | 2445 | 2 [symbol] 22 | 0.51(0.0 [symbol] ) | 3 × 10 [symbol] |
| (F) Genotype specific do [symbol] -dependent effect of coffee | | | | | | | | | | | |
| CC | [symbol] 25 | Ref | | 117 | 98 | Ref | | 451 | 27 | Ref | |
| | 25% [symbol] 50% | 1.49(0.52) | 0. [symbol] 7 | 120 | 92 | 1.11(0.12) | 0.70 | 4 [symbol] 4 | 270 | 1. [symbol] (0.12) | 0.61 |
| | 50% [symbol] 75% | 0.72(0.2 [symbol] ) | 0.18 | 91 | 87 | 0. [symbol] 3(0.17) | 0.19 | 457 | 290 | 0. [symbol] (0.10) | 0.30 |
| | [symbol] 75% | 0.58(0.23) | 0.09 | 75 | 82 | 0. [symbol] (0.15) | 0.0 [symbol] | 2 [symbol] | 22 [symbol] | 0. [symbol] (0.0 [symbol] ) | 5 × 10 [symbol] |
| TC | [symbol] 25% | Ref | | 27 | 18 | Ref | | 96 | 73 | Ref | |
| | 25% [symbol] 50% | 1.70(1.42) | 0.74 | 22 | 16 | 0. [symbol] 9(0.41) | 0.40 | [symbol] 7 | 57 | 1. [symbol] (0.12) | 0.36 |
| | 50% [symbol] 75% | 0.47(0.35) | 0.15 | 16 | 22 | 0.40(0.19) | 0.03 | 75 | 78 | 0. [symbol] 3(0.0 [symbol] ) | 0.05 |
| | [symbol] 75% | 0.57(0.46) | 0.24 | 13 | 21 | 0.37(0.1 [symbol] ) | 0.02 | 39 | 73 | 0.34(0.09) | 5 × 10 [symbol] |
| TT | [symbol] 25% | | | 2 | 2 | | | [symbol] | 2 | | |
| | 25% [symbol] 50% | | | 0 | 3 | | | 2 | 6 | | |
| | 50% [symbol] 75% | | | 3 | 1 | | | [symbol] | 5 | | |
| | [symbol] 75% | | | 3 | 1 | | | [symbol] | 5 | | |

[symbol]

indicates data missing or illegible when filed

PD: number of persons with PD. CO: number of control subjects. OR: odds ratio. P: statistical significance, two sided for NGRC and pooled analysis, one-sided for replication studies. *Heterogeneity P: Breslow-Day test statistics to assess between-study heterogeneity conducted for coffee and genotypes and found to be significant only for TT genotype. Analyses were adjusted for sex and age at interview in each dataset, and also for study in the pooled analyses. (A) Heavy coffee use was associated with 27% risk reduction (1-OR) in the pooled data. (B) GRIN2A rs4998386_TC genotype was associated with reduced risk consistently across studies. rs4998386_TT frequency varied significantly across studies. (C) The a-priori hypothesis for replication that among heavy drinkers GRIN2A_rs4998386_T carriers had a lower risk of PD than GRIN2A_rs4998386_CC was replicated under three conditions: comparing TC to CC (excluding rare and variable TT genotype), Dominant model (TT+TC vs. CC) and Additive model (TT vs. TC vs CC). As predicted from Discovery phase, genotype had no effect on risk of PD among light coffee drinkers. (D) The joint effects of genotype and coffee showed a significant 59% drop in PD risk in people who had the rs4998386_TC genotype and were heavy-drinkers, but little or no effect in other combinations. (E) A formal interaction test demonstrated that effects of coffee and genotype are dependent on each other. By definition, statistical interaction exists if the joint effect of gene (G) and exposure (E) is significantly different from the product of their individual effects. Interaction OR is the ratio of the OR of disease when G and E are present, divided by the product of the individual OR; i.e., ORinteraction=ORG+E/(ORG×ORE). (F) Dose-dependent risk reduction by coffee was clear and strong for rs4998386_TC genotype. Analyses were repeated with smoking added as covariate, results were unchanged (Table 8).

Testing the association of coffee with PD in NGRC, when calculated irrespective of genotype, showed an average of 34% lower PD risk in heavy-coffee-drinkers than light-drinkers (OR=0.66, P=6×10⁻⁶, Table 12A). GRIN2A, irrespective of coffee, had a modest main effect on PD in NGRC (Table 12B). A key question was if, and to what degree, GRIN2A_rs4998386 genotype modifies the effect of coffee on PD risk (Table 12C): Within heavy-drinkers, PD risk was 58% lower (OR=0.42, P=2×10⁻⁶) for rs4998386_TC, and 81% lower (OR=0.19, P=0.05) for rs4998386_TT genotype than rs4998386_CC; whereas in light-coffee-drinkers genotype had no effect on risk. Similar results were obtained for Additive and Dominant models (Table 12C). The joint effect comparing rs4998386_TC genotype and heavy-coffee, vs. rs4998386_CC genotype and light-coffee was most dramatic, suggesting a highly significant 68% risk reduction (OR=0.32, P=7×10⁻¹¹) in NGRC (Table 12D).

Hypothesis for Replication.

Applicants used GWAIS as a means to identify genes that might enhance the inverse association of coffee with PD with the goal of carrying the discovery forward as a genetic marker for use in pharmacogenetic studies. Hence, the replication hypothesis was specified a-priori, based on results of NGRC, as follows: “Among heavy-coffee drinkers, carriers of rs4998386_T allele have lower risk of PD than carriers of rs4998386 CC genotype”. Although this test does not reflect Applicants' most significant results, it is the test that has the clearest interpretation because it keeps the effect of coffee constant. For example, comparing TC+heavy vs. CC+light gave larger effect size and the P was 3-orders of magnitude lower than the specified hypothesis, however, unlike Applicants' hypothesis, the test included coffee, which would have made it difficult to draw firm conclusions about the effect of genotype on coffee's inverse association with PD.

Potential confounders (Table 7). Before attempting replication, the following analyses were conducted to identify potential confounders. Applicants tested the frequency of rs4998386 and coffee-use across disease-specific strata and population structure. There was no evidence for heterogeneity by presence or absence of family history of PD, age at onset, or recruitment site. rs4998386 frequency was different between Ashkenazi-Jewish and non-Jewish individuals (P=0.02) and across the European countries of ancestral origin (P=3×10⁻³) in cases, but not in controls, which, PD being heterogeneous, may indicate different ethnic-specific clusters of disease subtypes as has been noted for LRRK2-associated PD [31]. Not surprisingly, heavy-coffee use was associated with smoking (P<10⁻⁴), which itself is inversely associated with PD risk independently of coffee [11]. Adjusting for smoking, in addition to other covariates, did not change the results (Table 8). Applicants also repeated the analyses adjusting for caffeinated soda and caffeinated tea consumption and found the results to be robust (Table 9). Some reports suggest persons with PD are more likely to avoid sensation-seeking and addictive behaviors [32] and GRIN2A polymorphisms have been implicated in predisposition to heroin addiction [33] and smoking [34] raising the concern that Applicants' results could have been confounded if the GRIN2A SNPs identified here were associated with habitual coffee drinking. However, there was no evidence for association between any of the GRIN2A SNPs and heavy-vs. light-coffee consumption in cases and controls combined (OR=0.95-1.01, P=0.61-0.94).

Replication (Table 12). The a-priori hypothesis for replication that among heavy-drinkers GRIN2A_rs4998386_T carriers had a lower risk of PD than GRIN2A_rs4998386 CC was replicated under all three conditions: comparing TC to CC (excluding rare heterogeneous TT genotype): OR=0.59, P=10⁻³; Additive model (TT vs. TC vs. CC): OR=0.77, P=0.04; the Dominant model (TT+TC vs. CC): OR=0.70, P=0.01. Note that the Additive and Dominant models included the TT genotype which is rare and its frequency varied significantly across datasets. The TC vs. CC comparison is more robust for this reason; Additive and Dominant model are shown for completeness. As seen in NGRC data, genotype had no effect on risk of PD among light coffee drinkers in Replication or combined data (OR=1.0, P=0.99). In Replication, the [SNP+SNP*coffee] 2 df joint test yielded P=2.3×10⁻³ comparing TC to CC (excluding rare heterogeneous TT genotype); P=0.12 for the Additive model, P=0.02 for the Dominant model. The pooled analysis of Replication and Discovery with the [SNP+SNP*coffee] 2 df test yielded, P=1.9×10⁻⁷ comparing TC to CC (excluding rare heterogeneous TT genotype), P=1.4×10⁻⁵ for the Additive model, and P=8.6×10⁻⁷ for the Dominant model. In pooled data, compared to the light-coffee-users with GRIN2A_rs4998386_CC genotype (the group with highest risk), heavy-coffee use (with CC genotype) reduced risk by 18% (OR=0.82, P=3×10⁻³), having GRIN2A_rs4998386_T allele (light-coffee) had no effect on risk (OR=1.0, P=0.99), but the combination of heavy-coffee use and GRIN2A_rs4998386_TC genotype was associated with a highly significant 59% risk reduction (OR=0.41, P=6×10⁻¹³) (Table 12D).

Imputation (Tables 13-15). The array used in the study, Illumina OMNI-1 had nearly a million SNPs, which is a relatively dense coverage, but which could be further improved by imputing the SNPs that were not on the array using 1000 Genomes and HapMap data, a practice that has successfully aided many projects. After QC, Applicants had over 3.5 million imputed and genotyped SNPs per individual in NGRC, each with information score≧0.95 (measure of imputation certainty), and each passing standard GWAS QC. Imputation could only be applied to NGRC (Discovery) because only NGRC had genome-wide data. GWAIS and GWAS analysis of the GRIN2A region with imputed SNPs uncovered a block of densely linked SNPs embedded between genotyped GRIN2A SNPs that gave most significant signals. The information scores for imputed GRIN2A SNPs were 0.98-0.99 reflecting the highest accuracy achievable in imputation (1.0 is genotyped). The newly discovered block had stronger signals than the genotyped sequences. In GWAIS, 6 SNPs achieved P2df≦5×10⁻⁸ (Table 13). The interaction term was OR=0.44, P=4×10⁻⁵ (Table 13). In GWAS conducted in heavy-coffee drinkers, 12 SNPs achieved P=3×10⁻⁸ to 5×10⁻⁸ with OR=0.41-0.42 (Table 13).

TABLE 13
GWAIS and GWAS results on combined genotyped and imputed data. GRIN2A
was the most significant area in both a GWAIS and GWAS in heavy-coffee users. This table
shows results that achieved P ≦ 5 × 10−8. For a complete list of all SNPs that achieved P < 10−5
see Tables 14 and 15.

| | | | | IMPUTE | GWAIS in all NGRC subjects | | | | | | | | | GWAS In NGRC heavy-coffee-drinkers | | | | |
|---|---|---|---|---|---|---|---|---|---|---|---|---|---|---|---|---|---|---|
| | | | | INFO | IMPUTED | MAF | SNP | | | INTERACTION | | | 2DF | MAF | MAF | | | |
| SNP | BP | A1 | A2 | SCORE | Case | Cont | OR | SE | P | OR | SE | P | P | Case | Cont | OR | SE | P |
| 16-10105921 | 10105921 | T | C | 0.98 | 0.11 | 0.15 | 0.91 | 0.11 | 0.45 | 0.44 | 0.09 | 4 × 10 [symbol] | 5 × 10 [symbol] | 0.09 | 0.17 | 0.41 | 0.07 | 3 × 10 [symbol] |
| 16-10103787 | 10103787 | G | A | 0.98 | 0.11 | 0.15 | 0.91 | 0.11 | 0.45 | 0.44 | 0.09 | 4 × 10 [symbol] | 5 × 10 [symbol] | 0.09 | 0.17 | 0.41 | 0.07 | 3 × 10 [symbol] |
| 16-10102229 | 10102229 | T | C | 0.98 | 0.11 | 0.15 | 0.91 | 0.11 | 0.45 | 0.44 | 0.09 | 4 × 10 [symbol] | 5 × 10 [symbol] | 0.09 | 0.17 | 0.41 | 0.07 | 3 × 10 [symbol] |
| 16-10102124 | 10102124 | T | C | 0.98 | 0.11 | 0.15 | 0.91 | 0.11 | 0.45 | 0.44 | 0.09 | 4 × 10 [symbol] | 5 × 10 [symbol] | 0.09 | 0.17 | 0.41 | 0.07 | 3 × 10 [symbol] |
| rs56275045 | 10108893 | A | C | 0.99 | 0.11 | 0.15 | 0.91 | 0.11 | 0.41 | 0.45 | 0.09 | 5 × 10 [symbol] | 5 × 10 [symbol] | 0.09 | 0.17 | 0.42 | 0.07 | 3 × 10 [symbol] |
| 16-10109203 | 10109203 | A | T | 0.99 | 0.11 | 0.15 | 0.91 | 0.11 | 0.41 | 0.45 | 0.09 | 5 × 10 [symbol] | 5 × 10 [symbol] | 0.09 | 0.17 | 0.42 | 0.07 | 4 × 10 [symbol] |
| 16-10110896 | 10110396 | C | T | 0.98 | 0.11 | 0.15 | 0.91 | 0.11 | 0.41 | 0.45 | 0.09 | 6 × 10 [symbol] | 6 × 10 [symbol] | 0.09 | 0.17 | 0.42 | 0.07 | 4 × 10 [symbol] |
| 16-10101465 | 10101465 | A | G | 0.98 | 0.11 | 0.15 | 0.91 | 0.11 | 0.42 | 0.45 | 0.09 | 7 × 10 [symbol] | 7 × 10 [symbol] | 0.09 | 0.17 | 0.41 | 0.07 | 5 × 10 [symbol] |
| 16-10092692 | 10092692 | T | C | 0.98 | 0.11 | 0.15 | 0.91 | 0.11 | 0.40 | 0.46 | 0.09 | 8 × 10 [symbol] | 8 × 10 [symbol] | 0.09 | 0.17 | 0.42 | 0.07 | 5 × 10 [symbol] |
| 16-10093997 | 10093997 | T | C | 0.98 | 0.11 | 0.15 | 0.91 | 0.11 | 0.39 | 0.46 | 0.09 | 8 × 10 [symbol] | 8 × 10 [symbol] | 0.09 | 0.17 | 0.42 | 0.07 | 5 × 10 [symbol] |
| 16-10094528 | 10094528 | G | A | 0.98 | 0.11 | 0.15 | 0.91 | 0.11 | 0.39 | 0.46 | 0.09 | 8 × 10 [symbol] | 8 × 10 [symbol] | 0.09 | 0.17 | 0.42 | 0.07 | 5 × 10 [symbol] |
| rs17671178 | 10094708 | G | A | 0.98 | 0.11 | 0.15 | 0.90 | 0.11 | 0.39 | 0.46 | 0.09 | 8 × 10 [symbol] | 8 × 10 [symbol] | 0.09 | 0.17 | 0.42 | 0.07 | 5 × 10 [symbol] |

[symbol]

indicates data missing or illegible when filed

TABLE 14
GWAIS on NGRC genotyped and imputed data uncovered 248 SNPs in GRIN2A
that achieved P ≦ 10−5. SNPs are ordered according to their base-pair position (BP). Information
score is measure of accuracy of imputed data. SNPs with information score 1 were genotyped.

| | | | | MAF | MAF | INFO | SNP | | | Interaction | | | P |
|---|---|---|---|---|---|---|---|---|---|---|---|---|---|
| SNP | BP | A1 | A2 | Case | Cont | SCORE | OR | SE | P | OR | SE | P | 2DF |
| 16-9942563 | 9942563 | C | G | 0.09 | 0.12 | 0.96 | 0.85 | 0.11 | 0.21 | 0.53 | 0.11 | 3 × 10 [symbol] | 6 × 10 [symbol] |
| rs57576479 | 9945823 | A | C | 0.09 | 0.12 | 0.97 | 0.85 | 0.11 | 0.21 | 0.53 | 0.11 | 3 × 10 [symbol] | 5 × 10 [symbol] |
| rs59775432 | 9954194 | T | G | 0.09 | 0.12 | 0.98 | 0.85 | 0.11 | 0.21 | 0.52 | 0.11 | 2 × 10 [symbol] | 3 × 10 [symbol] |
| rs7192121 | 9956084 | C | T | 0.09 | 0.12 | 0.98 | 0.85 | 0.11 | 0.23 | 0.51 | 0.11 | 2 × 10 [symbol] | 3 × 10 [symbol] |
| rs17670318 | 9957403 | G | T | 0.09 | 0.12 | 0.98 | 0.86 | 0.11 | 0.23 | 0.51 | 0.11 | 2 × 10 [symbol] | 2 × 10 [symbol] |
| rs9925439 | 9962962 | G | T | 0.09 | 0.12 | 0.99 | 0.86 | 0.11 | 0.23 | 0.51 | 0.11 | 1 × 10 [symbol] | 2 × 10 [symbol] |
| rs17670396 | 9966823 | C | T | 0.09 | 0.12 | 0.99 | 0.85 | 0.11 | 0.23 | 0.51 | 0.11 | 2 × 10 [symbol] | 2 × 10 [symbol] |
| rs7197048 | 9970116 | T | C | 0.09 | 0.12 | 0.98 | 0.84 | 0.11 | 0.18 | 0.52 | 0.11 | 2 × 10 [symbol] | 2 × 10 [symbol] |
| 16-9970628 | 9970628 | T | A | 0.09 | 0.12 | 0.98 | 0.83 | 0.11 | 0.17 | 0.53 | 0.11 | 3 × 10 [symbol] | 3 × 10 [symbol] |
| 16-9971030 | 9971030 | G | T | 0.09 | 0.12 | 0.98 | 0.83 | 0.11 | 0.16 | 0.54 | 0.11 | 3 × 10 [symbol] | 3 × 10 [symbol] |
| rs8045558 | 9972307 | A | G | 0.09 | 0.12 | 0.98 | 0.83 | 0.11 | 0.16 | 0.53 | 0.11 | 3 × 10 [symbol] | 2 × 10 [symbol] |
| 16-9973459 | 9973459 | G | A | 0.09 | 0.12 | 0.99 | 0.83 | 0.11 | 0.16 | 0.52 | 0.11 | 3 × 10 [symbol] | 2 × 10 [symbol] |
| rs57497843 | 9973912 | C | G | 0.09 | 0.12 | 0.99 | 0.83 | 0.11 | 0.16 | 0.52 | 0.11 | 2 × 10 [symbol] | 2 × 10 [symbol] |
| 16-9975154 | 9975154 | G | A | 0.09 | 0.12 | 0.99 | 0.83 | 0.11 | 0.17 | 0.52 | 0.11 | 2 × 10 [symbol] | 1 × 10 [symbol] |
| rs4998386 | 9978046 | T | C | 0.08 | 0.12 | 1.00 | 0.84 | 0.11 | 0.19 | 0.50 | 0.11 | 1 × 10 [symbol] | 6 × 10 [symbol] |
| rs7191736 | 9978079 | T | C | 0.08 | 0.12 | 0.99 | 0.83 | 0.11 | 0.17 | 0.51 | 0.11 | 2 × 10 [symbol] | 9 × 10 [symbol] |
| rs7190716 | 9978240 | T | G | 0.08 | 0.12 | 0.99 | 0.83 | 0.11 | 0.17 | 0.51 | 0.11 | 2 × 10 [symbol] | 9 × 10 [symbol] |
| rs4254328 | 9978571 | A | G | 0.08 | 0.12 | 0.99 | 0.83 | 0.11 | 0.17 | 0.51 | 0.11 | 2 × 10 [symbol] | 9 × 10 [symbol] |
| 16-9979028 | 9979028 | C | T | 0.08 | 0.12 | 0.99 | 0.83 | 0.11 | 0.16 | 0.51 | 0.11 | 2 × 10 [symbol] | 8 × 10 [symbol] |
| rs17569609 | 9979290 | A | G | 0.08 | 0.12 | 0.99 | 0.83 | 0.11 | 0.16 | 0.51 | 0.11 | 2 × 10 [symbol] | 8 × 10 [symbol] |
| rs3897996 | 9980130 | T | C | 0.08 | 0.12 | 0.99 | 0.83 | 0.11 | 0.15 | 0.51 | 0.11 | 2 × 10 [symbol] | 7 × 10 [symbol] |
| 16-9980602 | 9980602 | C | T | 0.08 | 0.12 | 0.99 | 0.83 | 0.11 | 0.15 | 0.51 | 0.11 | 2 × 10 [symbol] | 6 × 10 [symbol] |
| 16-9981345 | 9981345 | C | T | 0.09 | 0.13 | 0.98 | 0.82 | 0.11 | 0.13 | 0.53 | 0.11 | 2 × 10 [symbol] | 7 × 10 [symbol] |
| rs9929020 | 9981643 | A | T | 0.09 | 0.13 | 0.99 | 0.82 | 0.11 | 0.13 | 0.53 | 0.11 | 2 × 10 [symbol] | 7 × 10 [symbol] |
| 16-9982616 | 9982616 | T | C | 0.09 | 0.13 | 0.99 | 0.82 | 0.11 | 0.14 | 0.53 | 0.11 | 2 × 10 [symbol] | 7 × 10 [symbol] |
| 16-9982918 | 9982918 | G | C | 0.09 | 0.13 | 0.99 | 0.83 | 0.11 | 0.14 | 0.52 | 0.11 | 2 × 10 [symbol] | 7 × 10 [symbol] |
| rs17670509 | 9983495 | C | T | 0.09 | 0.13 | 0.99 | 0.83 | 0.11 | 0.14 | 0.52 | 0.11 | 2 × 10 [symbol] | 7 × 10 [symbol] |
| 16-9984703 | 9984703 | A | G | 0.09 | 0.13 | 0.97 | 0.82 | 0.11 | 0.12 | 0.52 | 0.11 | 2 × 10 [symbol] | 6 × 10 [symbol] |
| rs8044626 | 9986196 | T | G | 0.09 | 0.13 | 0.99 | 0.83 | 0.11 | 0.14 | 0.52 | 0.11 | 2 × 10 [symbol] | 7 × 10 [symbol] |
| rs7190619 | 9986625 | A | G | 0.09 | 0.13 | 0.99 | 0.83 | 0.11 | 0.14 | 0.62 | 0.11 | 2 × 10 [symbol] | 7 × 10 [symbol] |
| rs17670544 | 9987533 | T | A | 0.08 | 0.12 | 0.99 | 0.79 | 0.10 | 0.08 | 0.55 | 0.12 | 6 × 10 [symbol] | 2 × 10 [symbol] |
| rs17569693 | 9987686 | G | A | 0.08 | 0.12 | 1.00 | 0.81 | 0.11 | 0.11 | 0.55 | 0.12 | 6 × 10 [symbol] | 4 × 10 [symbol] |
| rs61628643 | 9988786 | G | A | 0.09 | 0.13 | 0.99 | 0.83 | 0.11 | 0.14 | 0.52 | 0.11 | 2 × 10 [symbol] | 7 × 10 [symbol] |
| rs59931374 | 9988788 | A | G | 0.09 | 0.13 | 0.99 | 0.83 | 0.11 | 0.14 | 0.52 | 0.11 | 2 × 10 [symbol] | 7 × 10 [symbol] |
| 16-9991621 | 9991621 | G | A | 0.09 | 0.13 | 0.99 | 0.83 | 0.11 | 0.14 | 0.53 | 0.11 | 2 × 10 [symbol] | 8 × 10 [symbol] |
| 16-9994644 | 9994644 | G | A | 0.09 | 0.13 | 0.99 | 0.83 | 0.11 | 0.14 | 0.53 | 0.11 | 2 × 10 [symbol] | 9 × 10 [symbol] |
| rs60547225 | 9996064 | G | A | 0.09 | 0.13 | 0.99 | 0.83 | 0.11 | 0.13 | 0.53 | 0.11 | 2 × 10 [symbol] | 9 × 10 [symbol] |
| 16-9999095 | 9999095 | T | G | 0.09 | 0.13 | 0.98 | 0.82 | 0.11 | 0.13 | 0.53 | 0.11 | 3 × 10 [symbol] | 1 × 10 [symbol] |
| 16-9999864 | 9999864 | C | T | 0.09 | 0.13 | 0.98 | 0.82 | 0.11 | 0.13 | 0.55 | 0.11 | 4 × 10 [symbol] | 2 × 10 [symbol] |
| 16-10000299 | 10000299 | A | G | 0.09 | 0.13 | 0.99 | 0.82 | 0.11 | 0.13 | 0.55 | 0.12 | 4 × 10 [symbol] | 3 × 10 [symbol] |
| 16-10000942 | 10000942 | T | C | 0.09 | 0.13 | 0.99 | 0.82 | 0.11 | 0.13 | 0.56 | 0.12 | 5 × 10 [symbol] | 3 × 10 [symbol] |
| 16-10001082 | 10001082 | C | G | 0.09 | 0.13 | 0.99 | 0.82 | 0.11 | 0.13 | 0.56 | 0.12 | 5 × 10 [symbol] | 3 × 10 [symbol] |
| 16-10001459 | 10001459 | A | C | 0.09 | 0.13 | 0.99 | 0.82 | 0.11 | 0.13 | 0.56 | 0.12 | 5 × 10 [symbol] | 4 × 10 [symbol] |
| rs7200719 | 10002047 | T | C | 0.09 | 0.13 | 0.99 | 0.82 | 0.11 | 0.13 | 0.56 | 0.12 | 5 × 10 [symbol] | 4 × 10 [symbol] |
| rs4107019 | 10002700 | C | T | 0.09 | 0.13 | 0.99 | 0.82 | 0.11 | 0.13 | 0.56 | 0.12 | 6 × 10 [symbol] | 5 × 10 [symbol] |
| rs8043728 | 10003004 | T | C | 0.09 | 0.12 | 1.00 | 0.82 | 0.11 | 0.13 | 0.57 | 0.12 | 7 × 10 [symbol] | 6 × 10 [symbol] |
| 16-10005429 | 10005429 | C | G | 0.09 | 0.13 | 0.99 | 0.82 | 0.11 | 0.13 | 0.56 | 0.12 | 6 × 10 [symbol] | 5 × 10 [symbol] |
| rs7203512 | 10006587 | C | T | 0.09 | 0.13 | 0.99 | 0.82 | 0.11 | 0.13 | 0.56 | 0.12 | 5 × 10 [symbol] | 4 × 10 [symbol] |
| rs7198528 | 10006596 | T | A | 0.09 | 0.13 | 0.99 | 0.82 | 0.11 | 0.13 | 0.56 | 0.12 | 5 × 10 [symbol] | 4 × 10 [symbol] |
| 16-10008653 | 10008653 | A | C | 0.09 | 0.13 | 0.99 | 0.83 | 0.11 | 0.13 | 0.56 | 0.12 | 5 × 10 [symbol] | 4 × 10 [symbol] |
| rs17670766 | 10020926 | C | T | 0.09 | 0.13 | 0.98 | 0.83 | 0.11 | 0.14 | 0.55 | 0.12 | 5 × 10 [symbol] | 5 × 10 [symbol] |
| rs17569940 | 10021840 | G | C | 0.09 | 0.13 | 0.98 | 0.83 | 0.11 | 0.13 | 0.56 | 0.12 | 5 × 10 [symbol] | 4 × 10 [symbol] |
| rs1827197 | 10022865 | A | C | 0.09 | 0.13 | 0.98 | 0.82 | 0.11 | 0.13 | 0.56 | 0.12 | 5 × 10 [symbol] | 4 × 10 [symbol] |
| 16-10025128 | 10026128 | G | C | 0.09 | 0.13 | 0.99 | 0.82 | 0.10 | 0.12 | 0.56 | 0.12 | 6 × 10 [symbol] | 4 × 10 [symbol] |
| rs7193123 | 10030548 | T | C | 0.09 | 0.13 | 0.99 | 0.82 | 0.10 | 0.11 | 0.57 | 0.12 | 7 × 10 [symbol] | 5 × 10 [symbol] |
| 16-10037753 | 100 [symbol] 7753 | C | G | 0.09 | 0.13 | 0.99 | 0.82 | 0.10 | 0.12 | 0.56 | 0.12 | 6 × 10 [symbol] | 4 × 10 [symbol] |
| 16-10040481 | 10040481 | G | C | 0.09 | 0.13 | 0.99 | 0.82 | 0.10 | 0.12 | 0.56 | 0.12 | 6 × 10 [symbol] | 4 × 10 [symbol] |
| 16-10041339 | 10041339 | T | C | 0.09 | 0.13 | 0.99 | 0.82 | 0.10 | 0.12 | 0.56 | 0.12 | 6 × 10 [symbol] | 4 × 10 [symbol] |
| 16-10043792 | 10043792 | T | C | 0.09 | 0.13 | 0.98 | 0.82 | 0.10 | 0.12 | 0.56 | 0.12 | 6 × 10 [symbol] | 4 × 10 [symbol] |
| 16-10044321 | 10044321 | G | T | 0.09 | 0.13 | 0.98 | 0.82 | 0.10 | 0.12 | 0.56 | 0.12 | 6 × 10 [symbol] | 4 × 10 [symbol] |
| 16-10044670 | 10044670 | A | G | 0.09 | 0.13 | 0.98 | 0.82 | 0.10 | 0.12 | 0.56 | 0.12 | 6 × 10 [symbol] | 4 × 10 [symbol] |
| rs7193471 | 10045134 | T | C | 0.09 | 0.13 | 0.98 | 0.82 | 0.10 | 0.12 | 0.56 | 0.12 | 6 × 10 [symbol] | 4 × 10 [symbol] |
| 16-10048254 | 10048254 | C | T | 0.09 | 0.13 | 0.98 | 0.82 | 0.10 | 0.12 | 0.56 | 0.12 | 5 × 10 [symbol] | 3 × 10 [symbol] |
| rs13332501 | 10048640 | A | G | 0.09 | 0.13 | 0.98 | 0.82 | 0.10 | 0.13 | 0.56 | 0.12 | 5 × 10 [symbol] | 3 × 10 [symbol] |
| 16-10048787 | 10048787 | T | C | 0.10 | 0.14 | 0.98 | 0.87 | 0.11 | 0.25 | 0.53 | 0.11 | 2 × 10 [symbol] | 3 × 10 [symbol] |
| 16-10049656 | 10049656 | C | T | 0.09 | 0.13 | 0.98 | 0.82 | 0.10 | 0.13 | 0.56 | 0.12 | 5 × 10 [symbol] | 3 × 10 [symbol] |
| rs28528871 | 10049884 | A | G | 0.09 | 0.13 | 0.98 | 0.82 | 0.10 | 0.13 | 0.55 | 0.12 | 5 × 10 [symbol] | 3 × 10 [symbol] |
| rs7190321 | 10051520 | C | G | 0.09 | 0.13 | 0.99 | 0.83 | 0.11 | 0.14 | 0.55 | 0.11 | 4 × 10 [symbol] | 3 × 10 [symbol] |
| 16-10052589 | 10052589 | A | G | 0.09 | 0.13 | 0.99 | 0.83 | 0.11 | 0.15 | 0.55 | 0.11 | 4 × 10 [symbol] | 3 × 10 [symbol] |
| rs28491487 | 10052697 | C | T | 0.09 | 0.12 | 0.97 | 0.83 | 0.11 | 0.16 | 0.55 | 0.12 | 5 × 10 [symbol] | 6 × 10 [symbol] |
| rs8056683 | 10052710 | T | C | 0.09 | 0.13 | 1.00 | 0.83 | 0.11 | 0.14 | 0.55 | 0.11 | 4 × 10 [symbol] | 3 × 10 [symbol] |
| rs8061963 | 10052788 | G | T | 0.09 | 0.13 | 0.99 | 0.83 | 0.11 | 0.14 | 0.55 | 0.11 | 4 × 10 [symbol] | 3 × 10 [symbol] |
| rs8062320 | 10052970 | C | T | 0.09 | 0.13 | 0.99 | 0.83 | 0.11 | 0.14 | 0.55 | 0.11 | 4 × 10 [symbol] | 3 × 10 [symbol] |
| rs7203194 | 10053433 | C | G | 0.09 | 0.13 | 0.99 | 0.83 | 0.11 | 0.14 | 0.55 | 0.11 | 4 × 10 [symbol] | 3 × 10 [symbol] |
| rs8056514 | 10054493 | A | G | 0.09 | 0.13 | 0.99 | 0.83 | 0.11 | 0.14 | 0.55 | 0.11 | 4 × 10 [symbol] | 3 × 10 [symbol] |
| rs7200649 | 10054977 | T | C | 0.09 | 0.13 | 0.99 | 0.83 | 0.11 | 0.14 | 0.55 | 0.11 | 4 × 10 [symbol] | 3 × 10 [symbol] |
| rs7206296 | 10054979 | G | T | 0.09 | 0.13 | 0.99 | 0.83 | 0.11 | 0.14 | 0.55 | 0.11 | 4 × 10 [symbol] | 3 × 10 [symbol] |
| 16-10055961 | 10055961 | G | A | 0.09 | 0.13 | 0.99 | 0.83 | 0.11 | 0.14 | 0.55 | 0.11 | 4 × 10 [symbol] | 3 × 10 [symbol] |
| rs13335102 | 10056129 | C | G | 0.09 | 0.13 | 0.99 | 0.83 | 0.11 | 0.14 | 0.55 | 0.11 | 4 × 10 [symbol] | 3 × 10 [symbol] |
| 16-10056132 | 10056132 | A | G | 0.09 | 0.13 | 0.99 | 0.83 | 0.11 | 0.14 | 0.55 | 0.11 | 4 × 10 [symbol] | 3 × 10 [symbol] |
| rs58834898 | 10056278 | A | G | 0.09 | 0.13 | 0.99 | 0.83 | 0.11 | 0.14 | 0.55 | 0.11 | 4 × 10 [symbol] | 3 × 10 [symbol] |
| rs12102500 | 10056680 | G | A | 0.09 | 0.13 | 0.99 | 0.83 | 0.11 | 0.14 | 0.55 | 0.11 | 4 × 10 [symbol] | 3 × 10 [symbol] |
| rs12102636 | 10056729 | G | T | 0.09 | 0.13 | 0.99 | 0.83 | 0.11 | 0.14 | 0.55 | 0.11 | 4 × 10 [symbol] | 3 × 10 [symbol] |
| rs9925707 | 10057268 | T | A | 0.09 | 0.13 | 0.99 | 0.83 | 0.11 | 0.14 | 0.55 | 0.11 | 4 × 10 [symbol] | 3 × 10 [symbol] |
| rs9927914 | 10057354 | C | T | 0.09 | 0.13 | 0.99 | 0.83 | 0.11 | 0.14 | 0.55 | 0.11 | 4 × 10 [symbol] | 3 × 10 [symbol] |
| rs9925801 | 10057393 | C | A | 0.09 | 0.13 | 0.99 | 0.83 | 0.11 | 0.14 | 0.55 | 0.11 | 4 × 10 [symbol] | 3 × 10 [symbol] |
| rs9927926 | 10057405 | C | T | 0.09 | 0.13 | 1.00 | 0.83 | 0.11 | 0.14 | 0.55 | 0.11 | 4 × 10 [symbol] | 3 × 10 [symbol] |
| rs1463128 | 10057535 | C | T | 0.09 | 0.13 | 0.99 | 0.83 | 0.11 | 0.14 | 0.55 | 0.11 | 4 × 10 [symbol] | 3 × 10 [symbol] |
| rs9937970 | 10057671 | T | C | 0.09 | 0.13 | 0.99 | 0.83 | 0.11 | 0.15 | 0.55 | 0.11 | 4 × 10 [symbol] | 3 × 10 [symbol] |
| 16-10061092 | 10061092 | C | A | 0.09 | 0.13 | 0.99 | 0.84 | 0.11 | 0.17 | 0.54 | 0.11 | 3 × 10 [symbol] | 2 × 10 [symbol] |
| rs7194796 | 10061139 | T | A | 0.09 | 0.13 | 0.99 | 0.84 | 0.11 | 0.17 | 0.54 | 0.11 | 3 × 10 [symbol] | 2 × 10 [symbol] |
| rs7198175 | 10061667 | A | G | 0.09 | 0.13 | 0.99 | 0.84 | 0.11 | 0.17 | 0.54 | 0.11 | 3 × 10 [symbol] | 2 × 10 [symbol] |
| rs17670977 | 10062772 | C | T | 0.09 | 0.13 | 0.99 | 0.84 | 0.11 | 0.17 | 0.54 | 0.11 | 3 × 10 [symbol] | 2 × 10 [symbol] |
| 16-10063369 | 10063369 | T | C | 0.09 | 0.13 | 0.99 | 0.84 | 0.11 | 0.17 | 0.54 | 0.11 | 2 × 10 [symbol] | 2 × 10 [symbol] |
| rs7189360 | 10063493 | T | C | 0.09 | 0.13 | 0.99 | 0.84 | 0.11 | 0.17 | 0.54 | 0.11 | 2 × 10 [symbol] | 2 × 10 [symbol] |
| re7188122 | 10063546 | A | G | 0.09 | 0.13 | 0.99 | 0.84 | 0.11 | 0.17 | 0.54 | 0.11 | 2 × 10 [symbol] | 2 × 10 [symbol] |
| rs9935354 | 10064292 | T | C | 0.09 | 0.13 | 0.99 | 0.84 | 0.11 | 0.17 | 0.53 | 0.11 | 2 × 10 [symbol] | 2 × 10 [symbol] |
| rs8043864 | 10064430 | A | C | 0.09 | 0.13 | 0.99 | 0.84 | 0.11 | 0.17 | 0.53 | 0.11 | 2 × 10 [symbol] | 2 × 10 [symbol] |
| rs9922383 | 10065179 | A | G | 0.09 | 0.13 | 0.98 | 0.84 | 0.11 | 0.18 | 0.53 | 0.11 | 2 × 10 [symbol] | 2 × 10 [symbol] |
| rs9922460 | 10065294 | T | G | 0.09 | 0.13 | 0.99 | 0.84 | 0.11 | 0.17 | 0.53 | 0.11 | 2 × 10 [symbol] | 2 × 10 [symbol] |
| rs9935774 | 10065592 | G | A | 0.09 | 0.13 | 0.98 | 0.85 | 0.11 | 0.19 | 0.53 | 0.11 | 2 × 10 [symbol] | 2 × 10 [symbol] |
| rs9938061 | 10065671 | C | T | 0.09 | 0.13 | 0.99 | 0.84 | 0.11 | 0.17 | 0.53 | 0.11 | 2 × 10 [symbol] | 2 × 10 [symbol] |
| rs7197200 | 10065921 | C | T | 0.09 | 0.13 | 0.99 | 0.84 | 0.11 | 0.17 | 0.53 | 0.11 | 2 × 10 [symbol] | 2 × 10 [symbol] |
| 16-10066267 | 10066267 | A | G | 0.09 | 0.13 | 0.99 | 0.84 | 0.11 | 0.17 | 0.53 | 0.11 | 2 × 10 [symbol] | 2 × 10 [symbol] |
| rs7192622 | 10066343 | G | C | 0.09 | 0.13 | 0.99 | 0.84 | 0.11 | 0.17 | 0.53 | 0.11 | 2 × 10 [symbol] | 2 × 10 [symbol] |
| 16-10066691 | 10066691 | C | A | 0.09 | 0.13 | 0.99 | 0.84 | 0.11 | 0.17 | 0.53 | 0.11 | 2 × 10 [symbol] | 2 × 10 [symbol] |
| rs1448261 | 10068325 | T | G | 0.09 | 0.13 | 0.99 | 0.84 | 0.11 | 0.17 | 0.53 | 0.11 | 2 × 10 [symbol] | 2 × 10 [symbol] |
| 16-10068351 | 10068351 | T | C | 0.09 | 0.13 | 0.99 | 0.84 | 0.11 | 0.17 | 0.54 | 0.11 | 2 × 10 [symbol] | 2 × 10 [symbol] |
| rs1448262 | 10068370 | T | G | 0.09 | 0.13 | 0.99 | 0.84 | 0.11 | 0.17 | 0.54 | 0.11 | 2 × 10 [symbol] | 2 × 10 [symbol] |
| rs7188081 | 10068435 | G | A | 0.09 | 0.13 | 0.99 | 0.84 | 0.11 | 0.17 | 0.54 | 0.11 | 2 × 10 [symbol] | 2 × 10 [symbol] |
| rs7188291 | 10068537 | G | A | 0.09 | 0.13 | 0.99 | 0.84 | 0.11 | 0.17 | 0.54 | 0.11 | 2 × 10 [symbol] | 2 × 10 [symbol] |
| rs17671033 | 10068727 | A | G | 0.09 | 0.13 | 1.00 | 0.85 | 0.11 | 0.20 | 0.53 | 0.11 | 2 × 10 [symbol] | 3 × 10 [symbol] |
| rs9933478 | 10068753 | A | G | 0.09 | 0.13 | 0.99 | 0.84 | 0.11 | 0.17 | 0.54 | 0.11 | 2 × 10 [symbol] | 2 × 10 [symbol] |
| rs13331457 | 10069136 | C | A | 0.09 | 0.13 | 0.99 | 0.84 | 0.11 | 0.18 | 0.53 | 0.11 | 2 × 10 [symbol] | 2 × 10 [symbol] |
| rs28581562 | 10069342 | A | G | 0.09 | 0.13 | 0.99 | 0.84 | 0.11 | 0.18 | 0.53 | 0.11 | 2 × 10 [symbol] | 2 × 10 [symbol] |
| rs28465345 | 10069439 | A | G | 0.09 | 0.13 | 0.99 | 0.84 | 0.11 | 0.18 | 0.53 | 0.11 | 2 × 10 [symbol] | 2 × 10 [symbol] |
| rs28375797 | 10069497 | T | C | 0.09 | 0.13 | 0.99 | 0.84 | 0.11 | 0.18 | 0.53 | 0.11 | 2 × 10 [symbol] | 2 × 10 [symbol] |
| 16-10070145 | 10070145 | C | T | 0.09 | 0.13 | 0.99 | 0.84 | 0.11 | 0.18 | 0.53 | 0.11 | 2 × 10 [symbol] | 2 × 10 [symbol] |
| rs80570 [symbol] 4 | 10070452 | A | G | 0.09 | 0.13 | 0.99 | 0.84 | 0.11 | 0.18 | 0.53 | 0.11 | 2 × 10 [symbol] | 2 × 10 [symbol] |
| rs8059513 | 10070729 | G | A | 0.09 | 0.13 | 0.97 | 0.85 | 0.11 | 0.20 | 0.53 | 0.11 | 3 × 10 [symbol] | 3 × 10 [symbol] |
| rs8059547 | 10070795 | G | A | 0.09 | 0.13 | 0.99 | 0.84 | 0.11 | 0.18 | 0.53 | 0.11 | 2 × 10 [symbol] | 2 × 10 [symbol] |
| 16-10070978 | 10070978 | C | T | 0.09 | 0.13 | 0.99 | 0.85 | 0.11 | 0.18 | 0.53 | 0.11 | 2 × 10 [symbol] | 2 × 10 [symbol] |
| 16-10071089 | 10071089 | A | T | 0.09 | 0.13 | 0.99 | 0.85 | 0.11 | 0.18 | 0.53 | 0.11 | 2 × 10 [symbol] | 2 × 10 [symbol] |
| rs9930396 | 10071278 | G | A | 0.09 | 0.13 | 0.99 | 0.85 | 0.11 | 0.10 | 0.53 | 0.11 | 2 × 10 [symbol] | 2 × 10 [symbol] |
| 16-10071761 | 10071764 | T | C | 0.09 | 0.13 | 0.99 | 0.85 | 0.11 | 0.19 | 0.53 | 0.11 | 2 × 10 [symbol] | 2 × 10 [symbol] |
| 16-10071991 | 10071991 | C | T | 0.09 | 0.13 | 0.99 | 0.85 | 0.11 | 0.19 | 0.53 | 0.11 | 2 × 10 [symbol] | 1 × 10 [symbol] |
| 16-10071996 | 10071996 | C | G | 0.09 | 0.13 | 0.99 | 0.85 | 0.11 | 0.19 | 0.53 | 0.11 | 2 × 10 [symbol] | 1 × 10 [symbol] |
| rs9933111 | 10072100 | G | A | 0.09 | 0.13 | 1.00 | 0.86 | 0.11 | 0.24 | 0.52 | 0.11 | 1 × 10 [symbol] | 2 × 10 [symbol] |
| rs9922871 | 10072317 | A | C | 0.09 | 0.13 | 0.99 | 0.85 | 0.11 | 0.19 | 0.53 | 0.11 | 2 × 10 [symbol] | 1 × 10 [symbol] |
| rs9922338 | 10072329 | A | G | 0.09 | 0.13 | 0.99 | 0.85 | 0.11 | 0.19 | 0.52 | 0.11 | 2 × 10 [symbol] | 1 × 10 [symbol] |
| rs9302409 | 10072531 | C | T | 0.09 | 0.13 | 0.99 | 0.85 | 0.11 | 0.19 | 0.53 | 0.11 | 2 × 10 [symbol] | 1 × 10 [symbol] |
| 16-10072565 | 10072565 | T | C | 0.09 | 0.13 | 0.99 | 0.85 | 0.11 | 0.19 | 0.53 | 0.11 | 2 × 10 [symbol] | 1 × 10 [symbol] |
| rs17570290 | 10072647 | G | T | 0.09 | 0.13 | 0.99 | 0.85 | 0.11 | 0.19 | 0.53 | 0.11 | 2 × 10 [symbol] | 1 × 10 [symbol] |
| rs10518152 | 10072788 | G | A | 0.09 | 0.13 | 0.99 | 0.85 | 0.11 | 0.19 | 0.53 | 0.11 | 2 × 10 [symbol] | 1 × 10 [symbol] |
| 16-10073124 | 10073124 | C | A | 0.09 | 0.13 | 0.99 | 0.85 | 0.11 | 0.19 | 0.53 | 0.11 | 2 × 10 [symbol] | 1 × 10 [symbol] |
| rs13332018 | 10073171 | T | C | 0.09 | 0.13 | 0.99 | 0.85 | 0.11 | 0.19 | 0.53 | 0.11 | 2 × 10 [symbol] | 1 × 10 [symbol] |
| rs9938467 | 10073412 | C | T | 0.09 | 0.13 | 0.99 | 0.85 | 0.11 | 0.19 | 0.53 | 0.11 | 2 × 10 [symbol] | 1 × 10 [symbol] |
| rs8061018 | 100739 [symbol] 3 | C | T | 0.09 | 0.13 | 0.99 | 0.85 | 0.11 | 0.19 | 0.53 | 0.11 | 2 × 10 [symbol] | 1 × 10 [symbol] |
| rs9927648 | 10074002 | A | G | 0.09 | 0.13 | 0.99 | 0.85 | 0.11 | 0.19 | 0.53 | 0.11 | 2 × 10 [symbol] | 1 × 10 [symbol] |
| 16-10074078 | 10074078 | A | C | 0.09 | 0.13 | 0.99 | 0.85 | 0.11 | 0.19 | 0.53 | 0.11 | 2 × 10 [symbol] | 1 × 10 [symbol] |
| rs58797003 | 10074188 | C | A | 0.09 | 0.13 | 0.99 | 0.85 | 0.11 | 0.19 | 0.53 | 0.11 | 2 × 10 [symbol] | 1 × 10 [symbol] |
| rs59568026 | 10074278 | C | A | 0.09 | 0.13 | 0.99 | 0.85 | 0.11 | 0.19 | 0.53 | 0.11 | 2 × 10 [symbol] | 1 × 10 [symbol] |
| 16-10074282 | 10074282 | A | G | 0.09 | 0.13 | 0.99 | 0.85 | 0.11 | 0.19 | 0.53 | 0.11 | 2 × 10 [symbol] | 1 × 10 [symbol] |
| 16-10074631 | 10074631 | C | G | 0.09 | 0.13 | 0.99 | 0.85 | 0.11 | 0.19 | 0.53 | 0.11 | 2 × 10 [symbol] | 1 × 10 [symbol] |
| 16-10074643 | 10074643 | T | A | 0.09 | 0.13 | 0.99 | 0.85 | 0.11 | 0.19 | 0.53 | 0.11 | 2 × 10 [symbol] | 1 × 10 [symbol] |
| rs61224814 | 10074756 | T | C | 0.09 | 0.13 | 0.99 | 0.85 | 0.11 | 0.19 | 0.53 | 0.11 | 2 × 10 [symbol] | 1 × 10 [symbol] |
| rs60950183 | 10074799 | G | A | 0.09 | 0.13 | 0.99 | 0.85 | 0.11 | 0.19 | 0.53 | 0.11 | 2 × 10 [symbol] | 1 × 10 [symbol] |
| 16-10074810 | 10074810 | A | C | 0.09 | 0.13 | 0.99 | 0.85 | 0.11 | 0.19 | 0.53 | 0.11 | 2 × 10 [symbol] | 1 × 10 [symbol] |
| 16-10074932 | 10074932 | C | T | 0.09 | 0.13 | 0.99 | 0.85 | 0.11 | 0.19 | 0.53 | 0.11 | 2 × 10 [symbol] | 1 × 10 [symbol] |
| 16-10074942 | 10074942 | C | T | 0.09 | 0.13 | 0.99 | 0.85 | 0.11 | 0.19 | 0.53 | 0.11 | 2 × 10 [symbol] | 1 × 10 [symbol] |
| 16-10075021 | 10075021 | G | A | 0.09 | 0.13 | 0.99 | 0.85 | 0.11 | 0.19 | 0.53 | 0.11 | 2 × 10 [symbol] | 1 × 10 [symbol] |
| rs5 [symbol] 6352 | 10075139 | C | T | 0.09 | 0.13 | 0.99 | 0.85 | 0.11 | 0.19 | 0.53 | 0.11 | 2 × 10 [symbol] | 1 × 10 [symbol] |
| 16-10075411 | 10075411 | T | A | 0.09 | 0.13 | 0.99 | 0.85 | 0.11 | 0.19 | 0.53 | 0.11 | 2 × 10 [symbol] | 1 × 10 [symbol] |
| 16-10075461 | 10075461 | T | C | 0.09 | 0.13 | 0.99 | 0.85 | 0.11 | 0.19 | 0.53 | 0.11 | 2 × 10 [symbol] | 1 × 10 [symbol] |
| 16-10075477 | 10075477 | G | A | 0.09 | 0.13 | 0.99 | 0.85 | 0.11 | 0.19 | 0.53 | 0.11 | 2 × 10 [symbol] | 1 × 10 [symbol] |
| rs12102832 | 10075633 | C | T | 0.09 | 0.13 | 0.99 | 0.65 | 0.11 | 0.19 | 0.53 | 0.11 | 2 × 10 [symbol] | 1 × 10 [symbol] |
| 16-10075722 | 10075722 | C | T | 0.09 | 0.13 | 0.99 | 0.85 | 0.11 | 0.19 | 0.53 | 0.11 | 2 × 10 [symbol] | 1 × 10 [symbol] |
| rs8062241 | 1007809 | C | T | 0.09 | 0.13 | 0.99 | 0.85 | 0.11 | 0.19 | 0.53 | 0.11 | 2 × 10 [symbol] | 1 × 10 [symbol] |
| rs80626 [symbol] | 10075998 | C | T | 0.09 | 0.13 | 0.99 | 0.85 | 0.11 | 0.19 | 0.53 | 0.11 | 2 × 10 [symbol] | 1 × 10 [symbol] |
| rs9931653 | 10076193 | C | T | 0.09 | 0.13 | 0.99 | 0.85 | 0.11 | 0.19 | 0.53 | 0.11 | 2 × 10 [symbol] | 1 × 10 [symbol] |
| rs10518150 | 10076257 | G | T | 0.09 | 0.13 | 0.99 | 0.85 | 0.11 | 0.19 | 0.53 | 0.11 | 2 × 10 [symbol] | 2 × 10 [symbol] |
| rs9933945 | 10076487 | A | T | 0.09 | 0.13 | 0.99 | 0.65 | 0.11 | 0.19 | 0.53 | 0.11 | 2 × 10 [symbol] | 2 × 10 [symbol] |
| rs10518149 | 10076563 | C | A | 0.09 | 0.13 | 0.99 | 0.85 | 0.11 | 0.19 | 0.53 | 0.11 | 2 × 10 [symbol] | 2 × 10 [symbol] |
| rs28634372 | 10076780 | G | A | 0.09 | 0.13 | 0.99 | 0.85 | 0.11 | 0.19 | 0.53 | 0.11 | 2 × 10 [symbol] | 2 × 10 [symbol] |
| rs60276136 | 10077091 | G | A | 0.09 | 0.13 | 0.99 | 0.85 | 0.11 | 0.19 | 0.53 | 0.11 | 2 × 10 [symbol] | 2 × 10 [symbol] |
| rs28540790 | 10077145 | G | T | 0.09 | 0.13 | 0.99 | 0.85 | 0.11 | 0.19 | 0.53 | 0.11 | 2 × 10 [symbol] | 2 × 10 [symbol] |
| rs28574279 | 10077172 | A | G | 0.03 | 0.13 | 0.99 | 0.85 | 0.11 | 0.19 | 0.53 | 0.11 | 2 × 10 [symbol] | 2 × 10 [symbol] |
| 16-10077221 | 10077221 | T | A | 0.09 | 0.13 | 0.99 | 0.85 | 0.11 | 0.19 | 0.53 | 0.11 | 2 × 10 [symbol] | 2 × 10 [symbol] |
| rs57587803 | 10077247 | T | C | 0.09 | 0.13 | 0.99 | 0.85 | 0.11 | 0.19 | 0.53 | 0.11 | 2 × 10 [symbol] | 2 × 10 [symbol] |
| rs60864732 | 10077257 | G | T | 0.09 | 0.13 | 0.99 | 0.85 | 0.11 | 0.19 | 0.53 | 0.11 | 2 × 10 [symbol] | 2 × 10 [symbol] |
| rs58861142 | 10077314 | G | A | 0.09 | 0.13 | 0.99 | 0.85 | 0.11 | 0.19 | 0.53 | 0.11 | 2 × 10 [symbol] | 2 × 10 [symbol] |
| rs16956843 | 10077466 | A | G | 0.09 | 0.13 | 0.99 | 0.85 | 0.11 | 0.19 | 0.53 | 0.11 | 2 × 10 [symbol] | 2 × 10 [symbol] |
| 16-10077488 | 10077488 | A | G | 0.09 | 0.13 | 0.99 | 0.85 | 0.11 | 0.19 | 0.53 | 0.11 | 2 × 10 [symbol] | 2 × 10 [symbol] |
| rs61686802 | 10077552 | C | T | 0.09 | 0.13 | 0.99 | 0.85 | 0.11 | 0.19 | 0.53 | 0.11 | 2 × 10 [symbol] | 2 × 10 [symbol] |
| 16-10077618 | 10077618 | T | A | 0.09 | 0.13 | 0.99 | 0.85 | 0.11 | 0.19 | 0.53 | 0.11 | 2 × 10 [symbol] | 2 × 10 [symbol] |
| 16-10077656 | 10077656 | A | G | 0.09 | 0.13 | 0.99 | 0.85 | 0.11 | 0.19 | 0.53 | 0.11 | 2 × 10 [symbol] | 2 × 10 [symbol] |
| rs13331030 | 10077692 | C | G | 0.09 | 0.13 | 0.99 | 0.85 | 0.11 | 0.19 | 0.53 | 0.11 | 2 × 10 [symbol] | 2 × 10 [symbol] |
| 16-10077704 | 10077704 | C | A | 0.09 | 0.13 | 0.99 | 0.85 | 0.11 | 0.19 | 0.53 | 0.11 | 2 × 10 [symbol] | 2 × 10 [symbol] |
| rs13331085 | 10077809 | T | G | 0.09 | 0.13 | 0.99 | 0.85 | 0.11 | 0.19 | 0.53 | 0.11 | 2 × 10 [symbol] | 2 × 10 [symbol] |
| rs13331088 | 10077819 | A | G | 0.09 | 0.13 | 0.99 | 0.85 | 0.11 | 0.19 | 0.53 | 0.11 | 2 × 10 [symbol] | 2 × 10 [symbol] |
| rs13331428 | 10077870 | A | C | 0.09 | 0.13 | 0.99 | 0.85 | 0.11 | 0.19 | 0.53 | 0.11 | 2 × 10 [symbol] | 2 × 10 [symbol] |
| rs13331461 | 10077948 | T | C | 0.09 | 0.13 | 0.99 | 0.85 | 0.11 | 0.19 | 0.53 | 0.11 | 2 × 10 [symbol] | 2 × 10 [symbol] |
| rs13331168 | 10077998 | C | G | 0.09 | 0.13 | 0.99 | 0.85 | 0.11 | 0.19 | 0.53 | 0.11 | 2 × 10 [symbol] | 2 × 10 [symbol] |
| 16-10078003 | 10078003 | C | G | 0.09 | 0.13 | 0.99 | 0.85 | 0.11 | 0.19 | 0.53 | 0.11 | 2 × 10 [symbol] | 2 × 10 [symbol] |
| rs13331514 | 10078110 | A | C | 0.09 | 0.13 | 0.99 | 0.85 | 0.11 | 0.19 | 0.53 | 0.11 | 2 × 10 [symbol] | 2 × 10 [symbol] |
| rs13336632 | 10078155 | C | A | 0.09 | 0.13 | 1.00 | 0.85 | 0.11 | 0.19 | 0.52 | 0.11 | 1 × 10 [symbol] | 6 × 10 [symbol] |
| rs57737700 | 10078197 | T | G | 0.09 | 0.13 | 0.99 | 0.85 | 0.11 | 0.19 | 0.53 | 0.11 | 2 × 10 [symbol] | 2 × 10 [symbol] |
| rs13336710 | 10078329 | T | A | 0.09 | 0.13 | 0.99 | 0.85 | 0.11 | 0.19 | 0.53 | 0.11 | 2 × 10 [symbol] | 2 × 10 [symbol] |
| rs13332665 | 10078506 | G | C | 0.09 | 0.13 | 0.99 | 0.85 | 0.11 | 0.19 | 0.53 | 0.11 | 2 × 10 [symbol] | 2 × 10 [symbol] |
| rs13332699 | 10078588 | A | C | 0.09 | 0.13 | 0.99 | 0.85 | 0.11 | 0.19 | 0.53 | 0.11 | 2 × 10 [symbol] | 2 × 10 [symbol] |
| 16-10078887 | 10078837 | T | C | 0.09 | 0.13 | 0.99 | 0.85 | 0.11 | 0.20 | 0.53 | 0.11 | 2 × 10 [symbol] | 2 × 10 [symbol] |
| 16-10079158 | 10079158 | A | G | 0.09 | 0.13 | 0.99 | 0.85 | 0.11 | 0.20 | 0.53 | 0.11 | 2 × 10 [symbol] | 2 × 10 [symbol] |
| 16-10079160 | 10079160 | A | G | 0.09 | 0.13 | 0.09 | 0.85 | 0.11 | 0.20 | 0.53 | 0.11 | 2 × 10 [symbol] | 2 × 10 [symbol] |
| rs9940458 | 10079364 | G | T | 0.09 | 0.13 | 0.99 | 0.85 | 0.11 | 0.21 | 0.53 | 0.11 | 2 × 10 [symbol] | 2 × 10 [symbol] |
| rs2352741 | 1008020 [symbol] | T | C | 0.09 | 0.13 | 0.99 | 0.86 | 0.11 | 0.22 | 0.53 | 0.11 | 2 × 10 [symbol] | 2 × 10 [symbol] |
| rs1375072 | 10080576 | T | C | 0.09 | 0.13 | 0.99 | 0.86 | 0.11 | 0.22 | 0.53 | 0.11 | 2 × 10 [symbol] | 3 × 10 [symbol] |
| rs7191991 | 10081191 | T | C | 0.09 | 0.13 | 0.98 | 0.86 | 0.11 | 0.22 | 0.53 | 0.11 | 2 × 10 [symbol] | 3 × 10 [symbol] |
| rs1448268 | 10082178 | C | T | 0.09 | 0.13 | 0.98 | 0.86 | 0.11 | 0.22 | 0.53 | 0.11 | 2 × 10 [symbol] | 3 × 10 [symbol] |
| rs1448270 | 10082819 | T | G | 0.09 | 0.13 | 1.00 | 0.86 | 0.11 | 0.24 | 0.53 | 0.11 | 2 × 10 [symbol] | 4 × 10 [symbol] |
| rs1448271 | 10082972 | T | C | 0.09 | 0.13 | 0.98 | 0.86 | 0.11 | 0.22 | 0.53 | 0.11 | 2 × 10 [symbol] | 3 × 10 [symbol] |
| rs1375075 | 10083230 | G | A | 0.09 | 0.13 | 0.98 | 0.86 | 0.11 | 0.22 | 0.53 | 0.11 | 2 × 10 [symbol] | 3 × 10 [symbol] |
| rs1375076 | 10083232 | A | C | 0.09 | 0.13 | 0.98 | 0.86 | 0.11 | 0.22 | 0.53 | 0.11 | 2 × 10 [symbol] | 3 × 10 [symbol] |
| rs4780784 | 10083589 | G | A | 0.09 | 0.13 | 0.98 | 0.85 | 0.11 | 0.22 | 0.53 | 0.11 | 2 × 10 [symbol] | 2 × 10 [symbol] |
| rs1448272 | 10083636 | G | C | 0.09 | 0.13 | 0.98 | 0.85 | 0.11 | 0.22 | 0.53 | 0.11 | 2 × 10 [symbol] | 2 × 10 [symbol] |
| 16-10084653 | 10084653 | A | G | 0.09 | 0.13 | 0.97 | 0.85 | 0.11 | 0.21 | 0.52 | 0.11 | 2 × 10 [symbol] | 2 × 10 [symbol] |
| rs7199096 | 10085009 | G | C | 0.10 | 0.14 | 0.96 | 0.87 | 0.11 | 0.28 | 0.49 | 0.10 | 5 × 10 [symbol] | 6 × 10 [symbol] |
| rs7206094 | 10085011 | T | C | 0.10 | 0.14 | 0.96 | 0.87 | 0.11 | 0.28 | 0.49 | 0.10 | 5 × 10 [symbol] | 6 × 10 [symbol] |
| 16-10086160 | 10086160 | A | G | 0.11 | 0.15 | 0.96 | 0.91 | 0.11 | 0.43 | 0.47 | 0.09 | 1 × 10 [symbol] | 2 × 10 [symbol] |
| 16-10086580 | 10086580 | C | T | 0.11 | 0.15 | 0.96 | 0.91 | 0.11 | 0.43 | 0.47 | 0.09 | 1 × 10 [symbol] | 2 × 10 [symbol] |
| rs7201627 | 10087459 | C | T | 0.10 | 0.14 | 0.96 | 0.87 | 0.11 | 0.28 | 0.49 | 0.10 | 4 × 10 [symbol] | 4 × 10 [symbol] |
| 16-10090107 | 10090107 | T | C | 0.11 | 0.15 | 0.97 | 0.91 | 0.11 | 0.43 | 0.46 | 0.09 | 8 × 10 [symbol] | 1 × 10 [symbol] |
| 16-10092692 | 10092692 | T | C | 0.11 | 0.15 | 0.98 | 0.91 | 0.11 | 0.40 | 0.46 | 0.09 | 7 × 10 [symbol] | 8 × 10 [symbol] |
| 16-10093997 | 10093997 | T | C | 0.11 | 0.15 | 0.98 | 0.91 | 0.11 | 0.39 | 0.46 | 0.09 | 7 × 10 [symbol] | 8 × 10 [symbol] |
| 16-10094528 | 10094528 | G | A | 0.11 | 0.15 | 0.98 | 0.91 | 0.11 | 0.39 | 0.46 | 0.09 | 7 × 10 [symbol] | 8 × 10 [symbol] |
| rs17671178 | 10094708 | G | A | 0.11 | 0.15 | 0.98 | 0.90 | 0.11 | 0.39 | 0.46 | 0.09 | 7 × 10 [symbol] | 8 × 10 [symbol] |
| 16-10098086 | 10098086 | C | T | 0.11 | 0.14 | 0.9 [symbol] | 0.92 | 0.11 | 0.49 | 0.47 | 0.10 | 2 × 10 [symbol] | 1 × 10 [symbol] |
| 16-10098493 | 10098493 | C | T | 0.10 | 0.13 | 0.95 | 0.92 | 0.11 | 0.52 | 0.46 | 0.10 | 2 × 10 [symbol] | 1 × 10 [symbol] |
| 16-10101465 | 10101465 | A | G | 0.11 | 0.15 | 0.98 | 0.91 | 0.11 | 0.42 | 0.45 | 0.09 | 6 × 10 [symbol] | 7 × 10 [symbol] |
| 16-10102124 | 10102124 | T | C | 0.11 | 0.15 | 0.98 | 0.91 | 0.11 | 0.45 | 0.44 | 0.09 | 4 × 10 [symbol] | 5 × 10 [symbol] |
| 16-10102229 | 10102229 | T | C | 0.11 | 0.15 | 0.98 | 0.91 | 0.11 | 0.45 | 0.44 | 0.09 | 4 × 10 [symbol] | 5 × 10 [symbol] |
| 16-10103787 | 10103787 | G | A | 0.11 | 0.15 | 0.98 | 0.91 | 0.11 | 0.45 | 0.44 | 0.09 | 4 × 10 [symbol] | 5 × 10 [symbol] |
| 16-10105921 | 10105921 | T | C | 0.11 | 0.15 | 0.98 | 0.91 | 0.11 | 0.45 | 0.44 | 0.09 | 4 × 10 [symbol] | 5 × 10 [symbol] |
| rs56275045 | 10108893 | A | C | 0.11 | 0.15 | 0.99 | 0.91 | 0.11 | 0.41 | 0.45 | 0.09 | 5 × 10 [symbol] | 5 × 10 [symbol] |
| 16-10109203 | 10109203 | A | T | 0.11 | 0.15 | 0.99 | 0.91 | 0.11 | 0.41 | 0.45 | 0.09 | 5 × 10 [symbol] | 5 × 10 [symbol] |
| 16-10109483 | 10109483 | G | A | 0.10 | 0.14 | 0.98 | 0.86 | 0.11 | 0.23 | 0.47 | 0.10 | 3 × 10 [symbol] | 1 × 10 [symbol] |
| 16-10110896 | 10110896 | C | T | 0.11 | 0.15 | 0.98 | 0.91 | 0.11 | 0.41 | 0.45 | 0.09 | 6 × 10 [symbol] | 6 × 10 [symbol] |
| 16-10116251 | 10116251 | G | A | 0.09 | 0.13 | 0.99 | 0.84 | 0.11 | 0.18 | 0.50 | 0.11 | 1 × 10 [symbol] | 9 × 10 [symbol] |
| 16-10116566 | 10116566 | T | C | 0.09 | 0.13 | 0.99 | 0.84 | 0.11 | 0.18 | 0.50 | 0.11 | 1 × 10 [symbol] | 8 × 10 [symbol] |
| 16-10118795 | 10118795 | C | T | 0.09 | 0.13 | 0.99 | 0.83 | 0.10 | 0.14 | 0.52 | 0.11 | 2 × 10 [symbol] | 6 × 10 [symbol] |
| 16-10120657 | 10120657 | C | A | 0.09 | 0.13 | 0.99 | 0.83 | 0.10 | 0.14 | 0.51 | 0.11 | 1 × 10 [symbol] | 5 × 10 [symbol] |
| 16-10121144 | 10121144 | T | C | 0.09 | 0.13 | 0.99 | 0.83 | 0.10 | 0.14 | 0.51 | 0.11 | 1 × 10 [symbol] | 5 × 10 [symbol] |
| rs7196139 | 10121899 | C | T | 0.09 | 0.13 | 0.99 | 0.83 | 0.10 | 0.14 | 0.51 | 0.11 | 1 × 10 [symbol] | 5 × 10 [symbol] |
| 16-10122294 | 10122294 | C | T | 0.09 | 0.13 | 0.99 | 0.82 | 0.10 | 0.13 | 0.52 | 0.11 | 2 × 10 [symbol] | 4 × 10 [symbol] |
| 16-10123191 | 10123191 | G | T | 0.09 | 0.13 | 0.98 | 0.83 | 0.11 | 0.15 | 0.51 | 0.11 | 1 × 10 [symbol] | 5 × 10 [symbol] |
| 16-10123460 | 10123460 | C | A | 0.08 | 0.12 | 0.95 | 0.80 | 0.11 | 0.09 | 0.53 | 0.12 | 4 × 10 [symbol] | 1 × 10 [symbol] |
| rs1375068 | 10123958 | A | C | 0.09 | 0.13 | 0.99 | 0.83 | 0.11 | 0.15 | 0.53 | 0.11 | 2 × 10 [symbol] | 1 × 10 [symbol] |
| 16-10125149 | 10125149 | C | T | 0.09 | 0.13 | 0.99 | 0.83 | 0.10 | 0.14 | 0.54 | 0.11 | 3 × 10 [symbol] | 2 × 10 [symbol] |
| rs4782266 | 10125491 | A | C | 0.09 | 0.13 | 0.99 | 0.84 | 0.11 | 0.17 | 0.53 | 0.11 | 2 × 10 [symbol] | 2 × 10 [symbol] |
| rs56135508 | 10125997 | G | A | 0.09 | 0.13 | 0.98 | 0.84 | 0.11 | 0.17 | 0.53 | 0.11 | 2 × 10 [symbol] | 2 × 10 [symbol] |
| rs1448253 | 10128367 | C | T | 0.09 | 0.13 | 1.00 | 0.84 | 0.11 | 0.18 | 0.55 | 0.11 | 3 × 10 [symbol] | 4 × 10 [symbol] |
| 16-10128920 | 10128920 | C | T | 0.09 | 0.13 | 0.98 | 0.84 | 0.11 | 0.16 | 0.55 | 0.11 | 3 × 10 [symbol] | 3 × 10 [symbol] |
| rs9930364 | 10128984 | T | A | 0.09 | 0.13 | 0.98 | 0.84 | 0.11 | 0.16 | 0.55 | 0.11 | 3 × 10 [symbol] | 3 × 10 [symbol] |
| 16-10129181 | 10129181 | T | C | 0.10 | 0.13 | 0.98 | 0.84 | 0.11 | 0.16 | 0.55 | 0.11 | 4 × 10 [symbol] | 4 × 10 [symbol] |
| rs11645379 | 10129471 | A | C | 0.09 | 0.13 | 0.97 | 0.84 | 0.11 | 0.18 | 0.54 | 0.11 | 3 × 10 [symbol] | 3 × 10 [symbol] |
| rs6497716 | 10130309 | C | T | 0.10 | 0.14 | 0.97 | 0.84 | 0.10 | 0.16 | 0.56 | 0.11 | 4 × 10 [symbol] | 5 × 10 [symbol] |
| rs2197778 | 10130956 | A | C | 0.10 | 0.14 | 0.97 | 0.84 | 0.10 | 0.15 | 0.56 | 0.11 | 5 × 10 [symbol] | 5 × 10 [symbol] |
| 16-10132126 | 10132126 | C | T | 0.10 | 0.14 | 0.97 | 0.84 | 0.10 | 0.15 | 0.56 | 0.11 | 5 × 10 [symbol] | 6 × 10 [symbol] |
| rs1070479 | 10136873 | G | A | 0.10 | 0.14 | 0.9 [symbol] | 0.84 | 0.10 | 0.16 | 0.57 | 0.12 | 5 × 10 [symbol] | 9 × 10 [symbol] |

[symbol]

indicates data missing or illegible when filed

TABLE 15
GWAS in heavy-coffee-drinkers using genotyped and imputed data uncovered
249 GRIN2A SNPs that achieved P ≦ 10−5. SNPs are ordered according to their base-pair
position (BP). Information score is measure of accuracy of imputed data. SNPs with information
score 1 were genotyped.

| | | | | | EST | EST | | | |
|---|---|---|---|---|---|---|---|---|---|
| | | INFO | | | MAF | MAF | | | |
| SNP | BP | SCORE | A1 | A2 | CASE | CONT | OR | SE | P |
| 16-9942563 | 9942563 | 0.96 | C | G | 0.08 | 0.14 | 0.45 | 0.08 | 4 × 10−6 |
| rs57576479 | 9945823 | 0.97 | A | C | 0.08 | 0.14 | 0.46 | 0.08 | 4 × 10−6 |
| rs59775432 | 9954194 | 0.98 | T | G | 0.08 | 0.14 | 0.45 | 0.08 | 3 × 10−6 |
| rs7192121 | 9956084 | 0.98 | C | T | 0.08 | 0.14 | 0.44 | 0.08 | 2 × 10 [symbol] |
| rs17670318 | 9957403 | 0.98 | G | T | 0.07 | 0.14 | 0.44 | 0.08 | 2 × 10 [symbol] |
| rs9925439 | 9962962 | 0.99 | G | T | 0.07 | 0.14 | 0.44 | 0.08 | 2 × 10 [symbol] |
| rs17670396 | 9966823 | 0.99 | C | T | 0.07 | 0.14 | 0.44 | 0.08 | 2 × 10 [symbol] |
| rs7197048 | 9970116 | 0.98 | T | C | 0.08 | 0.14 | 0.45 | 0.08 | 2 × 10 [symbol] |
| 16-9970628 | 9970628 | 0.98 | T | A | 0.08 | 0.14 | 0.45 | 0.08 | 3 × 10 [symbol] |
| 16-9971030 | 9971030 | 0.98 | G | T | 0.08 | 0.14 | 0.45 | 0.08 | 3 × 10 [symbol] |
| rs8045558 | 9972307 | 0.98 | A | G | 0.08 | 0.14 | 0.45 | 0.08 | 2 × 10 [symbol] |
| 16-9973459 | 9973459 | 0.99 | G | A | 0.07 | 0.14 | 0.44 | 0.08 | 2 × 10 [symbol] |
| rs57497843 | 9973912 | 0.99 | C | G | 0.07 | 0.14 | 0.44 | 0.08 | 2 × 10 [symbol] |
| 16-9975154 | 9975154 | 0.99 | G | A | 0.07 | 0.14 | 0.44 | 0.07 | 1 × 10 [symbol] |
| rs4998386 | 9978046 | 1.00 | T | C | 0.07 | 0.14 | 0.43 | 0.07 | 6 × 10 [symbol] |
| rs7191736 | 9978079 | 0.99 | T | C | 0.07 | 0.14 | 0.43 | 0.07 | 9 × 10 [symbol] |
| rs7190716 | 9978240 | 0.99 | T | G | 0.07 | 0.14 | 0.43 | 0.07 | 9 × 10 [symbol] |
| rs4254328 | 9978571 | 0.99 | A | G | 0.07 | 0.14 | 0.43 | 0.07 | 9 × 10 [symbol] |
| 16-9979028 | 9979028 | 0.99 | C | T | 0.07 | 0.14 | 0.43 | 0.07 | 8 × 10 [symbol] |
| rs17569609 | 9979290 | 0.99 | A | G | 0.07 | 0.14 | 0.43 | 0.07 | 8 × 10 [symbol] |
| re3897996 | 9980130 | 0.99 | T | C | 0.07 | 0.14 | 0.43 | 0.07 | 7 × 10 [symbol] |
| 16-9980602 | 9980602 | 0.99 | C | T | 0.07 | 0.14 | 0.43 | 0.07 | 7 × 10 [symbol] |
| 16-9981346 | 9981345 | 0.98 | C | T | 0.08 | 0.16 | 0.44 | 0.07 | 9 × 10 [symbol] |
| rs9929020 | 9981643 | 0.99 | A | T | 0.08 | 0.15 | 0.44 | 0.07 | 9 × 10 [symbol] |
| 16-9982616 | 9982616 | 0.99 | T | C | 0.08 | 0.15 | 0.44 | 0.07 | 9 × 10 [symbol] |
| 16-9982918 | 9982918 | 0.99 | G | C | 0.08 | 0.15 | 0.44 | 0.07 | 9 × 10 [symbol] |
| rs17670609 | 9983495 | 0.99 | C | T | 0.08 | 0.15 | 0.44 | 0.07 | 9 × 10 [symbol] |
| 16-9984703 | 9984703 | 0.97 | A | G | 0.07 | 0.14 | 0.43 | 0.07 | 9 × 10 [symbol] |
| rs8044626 | 9986196 | 0.99 | T | G | 0.08 | 0.15 | 0.44 | 0.07 | 9 × 10 [symbol] |
| rs7190619 | 9986625 | 0.99 | A | G | 0.08 | 0.15 | 0.44 | 0.07 | 9 × 10 [symbol] |
| rs17670544 | 9987533 | 0.99 | T | A | 0.07 | 0.13 | 0.44 | 0.08 | 3 × 10 [symbol] |
| rs17569693 | 9987686 | 1.00 | G | A | 0.07 | 0.13 | 0.46 | 0.08 | 5 × 10 [symbol] |
| rs61628643 | 9988786 | 0.99 | G | A | 0.08 | 0.15 | 0.44 | 0.07 | 9 × 10 [symbol] |
| rs59931374 | 9988788 | 0.99 | A | G | 0.08 | 0.15 | 0.44 | 0.07 | 9 × 10 [symbol] |
| 16-9991621 | 9991621 | 0.99 | G | A | 0.08 | 0.15 | 0.44 | 0.07 | 9 × 10 [symbol] |
| 16-9994644 | 9994644 | 0.99 | G | A | 0.08 | 0.15 | 0.44 | 0.07 | 1 × 10 [symbol] |
| rs60547225 | 9996064 | 0.99 | G | A | 0.08 | 0.15 | 0.44 | 0.07 | 1 × 10 [symbol] |
| 16-9999095 | 9999095 | 0.98 | T | G | 0.08 | 0.14 | 0.45 | 0.07 | 2 × 10 [symbol] |
| 16-9999864 | 9999864 | 0.98 | C | T | 0.08 | 0.14 | 0.46 | 0.08 | 3 × 10 [symbol] |
| 16-10000299 | 10000299 | 0.99 | A | G | 0.08 | 0.14 | 0.46 | 0.08 | 3 × 10 [symbol] |
| 16-10000942 | 10000942 | 0.99 | T | C | 0.08 | 0.14 | 0.46 | 0.08 | 4 × 10 [symbol] |
| 16-10001082 | 10001082 | 0.99 | C | G | 0.08 | 0.14 | 0.46 | 0.08 | 4 × 10 [symbol] |
| 16-10001459 | 10001459 | 0.99 | A | G | 0.08 | 0.14 | 0.47 | 0.08 | 4 × 10 [symbol] |
| rs7200719 | 10002047 | 0.99 | T | C | 0.08 | 0.14 | 0.47 | 0.08 | 5 × 10 [symbol] |
| rs4107019 | 10002700 | 0.99 | C | T | 0.08 | 0.14 | 0.47 | 0.08 | 6 × 10 [symbol] |
| rs8043728 | 10003004 | 1.00 | T | C | 0.08 | 0.14 | 0.47 | 0.08 | 7 × 10 [symbol] |
| 16-10005429 | 10005429 | 0.99 | C | G | 0.08 | 0.14 | 0.47 | 0.08 | 5 × 10 [symbol] |
| rs7203512 | 10006587 | 0.99 | C | T | 0.08 | 0.14 | 0.47 | 0.08 | 5 × 10 [symbol] |
| rs7198528 | 10006596 | 0.99 | T | A | 0.08 | 0.14 | 0.47 | 0.08 | 5 × 10 [symbol] |
| 16-10008653 | 10008653 | 0.99 | A | C | 0.08 | 0.14 | 0.47 | 0.08 | 5 × 10 [symbol] |
| rs17670766 | 10020926 | 0.98 | C | T | 0.08 | 0.14 | 0.47 | 0.08 | 5 × 10 [symbol] |
| rs17669940 | 10021840 | 0.99 | G | C | 0.08 | 0.14 | 0.47 | 0.08 | 5 × 10 [symbol] |
| rs1827197 | 10022865 | 0.98 | A | C | 0.08 | 0.14 | 0.47 | 0.08 | 5 × 10 [symbol] |
| 16-10025128 | 10025128 | 0.99 | G | C | 0.08 | 0.14 | 0.47 | 0.08 | 5 × 10 [symbol] |
| rs7193123 | 10030548 | 0.99 | T | G | 0.08 | 0.14 | 0.47 | 0.08 | 6 × 10 [symbol] |
| 16-10037753 | 10037753 | 0.99 | C | G | 0.08 | 0.14 | 0.47 | 0.08 | 5 × 10 [symbol] |
| 16-10040481 | 10040481 | 0.99 | G | G | 0.08 | 0.14 | 0.47 | 0.08 | 5 × 10 [symbol] |
| 16-10041339 | 10041339 | 0.99 | T | C | 0.08 | 0.14 | 0.47 | 0.08 | 5 × 10 [symbol] |
| 16-10043792 | 10043792 | 0.98 | T | C | 0.08 | 0.14 | 0.47 | 0.08 | 5 × 10 [symbol] |
| 16-10044321 | 10044321 | 0.98 | G | T | 0.08 | 0.14 | 0.47 | 0.08 | 5 × 10 [symbol] |
| 16-10044670 | 10044670 | 0.98 | A | G | 0.08 | 0.14 | 0.47 | 0.08 | 5 × 10 [symbol] |
| rs7193471 | 10045134 | 0.98 | T | G | 0.08 | 0.14 | 0.47 | 0.08 | 5 × 10 [symbol] |
| 16-10048254 | 10048254 | 0.98 | C | T | 0.08 | 0.14 | 0.47 | 0.08 | 4 × 10 [symbol] |
| rs13332501 | 10048640 | 0.98 | A | G | 0.08 | 0.14 | 0.46 | 0.08 | 4 × 10 [symbol] |
| 16-10048787 | 10048787 | 0.98 | T | C | 0.09 | 0.16 | 0.47 | 0.07 | 2 × 10 [symbol] |
| 16-10049656 | 10049656 | 0.98 | C | T | 0.08 | 0.14 | 0.46 | 0.08 | 4 × 10 [symbol] |
| rs28528871 | 10049884 | 0.98 | A | G | 0.08 | 0.14 | 0.46 | 0.08 | 4 × 10 [symbol] |
| rs7190321 | 10051520 | 0.99 | C | G | 0.08 | 0.14 | 0.46 | 0.08 | 4 × 10 [symbol] |
| 16-10052589 | 10052589 | 0.99 | A | G | 0.08 | 0.14 | 0.47 | 0.08 | 3 × 10 [symbol] |
| rs28491487 | 10052697 | 0.97 | C | T | 0.08 | 0.14 | 0.47 | 0.08 | 6 × 10 [symbol] |
| rs8056683 | 10062710 | 1.00 | T | C | 0.08 | 0.14 | 0.47 | 0.08 | 3 × 10 [symbol] |
| rs8061963 | 10052788 | 0.99 | G | T | 0.08 | 0.14 | 0.47 | 0.08 | 3 × 10 [symbol] |
| rs8062320 | 10052970 | 0.99 | C | T | 0.08 | 0.14 | 0.47 | 0.08 | 3 × 10 [symbol] |
| rs7203194 | 10053433 | 0.99 | C | G | 0.08 | 0.14 | 0.47 | 0.08 | 3 × 10 [symbol] |
| rs8056514 | 10054493 | 0.99 | A | G | 0.08 | 0.14 | 0.46 | 0.08 | 3 × 10 [symbol] |
| rs7200649 | 10054977 | 0.99 | T | C | 0.08 | 0.14 | 0.46 | 0.08 | 3 × 10 [symbol] |
| rs7206296 | 10054979 | 0.99 | G | T | 0.08 | 0.14 | 0.46 | 0.08 | 3 × 10 [symbol] |
| 16-10055961 | 10055961 | 0.99 | G | A | 0.08 | 0.14 | 0.46 | 0.08 | 3 × 10 [symbol] |
| rs13335102 | 10056129 | 0.99 | C | G | 0.08 | 0.14 | 0.46 | 0.08 | 3 × 10 [symbol] |
| 16-10056132 | 10056132 | 0.99 | A | G | 0.08 | 0.14 | 0.46 | 0.08 | 3 × 10 [symbol] |
| rs58834898 | 10056278 | 0.99 | A | G | 0.08 | 0.14 | 0.46 | 0.08 | 3 × 10 [symbol] |
| rs12102500 | 10056680 | 0.99 | G | A | 0.08 | 0.14 | 0.46 | 0.08 | 3 × 10 [symbol] |
| rs12102636 | 10056729 | 0.99 | G | T | 0.08 | 0.14 | 0.46 | 0.08 | 3 × 10 [symbol] |
| rs9925707 | 10057268 | 0.99 | T | A | 0.08 | 0.14 | 0.46 | 0.08 | 3 × 10 [symbol] |
| rs9927914 | 10057354 | 0.99 | C | T | 0.08 | 0.14 | 0.46 | 0.08 | 3 × 10 [symbol] |
| rs9925801 | 10057393 | 0.99 | C | A | 0.08 | 0.14 | 0.46 | 0.08 | 3 × 10 [symbol] |
| rs9927926 | 10057405 | 1.00 | C | T | 0.08 | 0.14 | 0.47 | 0.08 | 3 × 10 [symbol] |
| rs1463128 | 10057535 | 0.99 | C | T | 0.08 | 0.14 | 0.46 | 0.08 | 3 × 10 [symbol] |
| rs9937970 | 10057671 | 0.99 | T | C | 0.08 | 0.14 | 0.46 | 0.08 | 3 × 10 [symbol] |
| 16-10061092 | 10061092 | 0.99 | C | A | 0.08 | 0.15 | 0.48 | 0.06 | 2 × 10 [symbol] |
| rs7194796 | 10061139 | 0.99 | T | A | 0.08 | 0.15 | 0.46 | 0.08 | 2 × 10 [symbol] |
| rs7198175 | 10061667 | 0.99 | A | G | 0.08 | 0.15 | 0.46 | 0.08 | 2 × 10 [symbol] |
| rs17670977 | 10062772 | 0.99 | C | T | 0.08 | 0.15 | 0.46 | 0.08 | 2 × 10 [symbol] |
| 16-10063369 | 10063369 | 0.99 | T | C | 0.08 | 0.15 | 0.46 | 0.08 | 2 × 10 [symbol] |
| rs7189360 | 10063496 | 0.99 | T | C | 0.08 | 0.15 | 0.46 | 0.08 | 2 × 10 [symbol] |
| rs7188122 | 10063546 | 0.99 | A | G | 0.08 | 0.15 | 0.46 | 0.08 | 2 × 10 [symbol] |
| rs9335354 | 10064292 | 0.99 | T | C | 0.08 | 0.15 | 0.46 | 0.08 | 2 × 10 [symbol] |
| rs8043864 | 10064430 | 0.99 | A | G | 0.08 | 0.15 | 0.46 | 0.08 | 2 × 10 [symbol] |
| rs9922383 | 10065179 | 0.98 | A | G | 0.08 | 0.15 | 0.45 | 0.08 | 2 × 10 [symbol] |
| rs9922460 | 10065294 | 0.99 | T | G | 0.08 | 0.15 | 0.46 | 0.08 | 2 × 10 [symbol] |
| rs9935774 | 10065592 | 0.98 | G | A | 0.08 | 0.15 | 0.46 | 0.07 | 2 × 10 [symbol] |
| rs9938061 | 10065671 | 0.99 | C | T | 0.08 | 0.15 | 0.46 | 0.08 | 2 × 10 [symbol] |
| rs7197200 | 10065921 | 0.99 | C | T | 0.08 | 0.15 | 0.46 | 0.08 | 2 × 10 [symbol] |
| 16-10066267 | 10066267 | 0.99 | A | G | 0.08 | 0.15 | 0.46 | 0.08 | 2 × 10 [symbol] |
| rs7192622 | 10066343 | 0.99 | G | C | 0.08 | 0.15 | 0.46 | 0.08 | 2 × 10 [symbol] |
| 16-10066691 | 10066691 | 0.99 | C | A | 0.08 | 0.15 | 0.46 | 0.08 | 2 × 10 [symbol] |
| rs1448261 | 10068325 | 0.99 | T | G | 0.08 | 0.15 | 0.46 | 0.08 | 2 × 10 [symbol] |
| 16-10068351 | 10068351 | 0.99 | T | C | 0.08 | 0.15 | 0.46 | 0.08 | 2 × 10 [symbol] |
| rs1448262 | 10068370 | 0.99 | T | G | 0.08 | 0.15 | 0.46 | 0.08 | 2 × 10 [symbol] |
| rs7188081 | 10068435 | 0.99 | G | A | 0.08 | 0.15 | 0.46 | 0.08 | 2 × 10 [symbol] |
| rs7188291 | 10068537 | 0.99 | G | A | 0.08 | 0.15 | 0.46 | 0.08 | 2 × 10 [symbol] |
| rs17671033 | 10068727 | 1.00 | A | G | 0.08 | 0.14 | 0.46 | 0.08 | 3 × 10 [symbol] |
| rs9933478 | 10068753 | 0.99 | A | G | 0.08 | 0.15 | 0.46 | 0.08 | 2 × 10 [symbol] |
| rs13331457 | 10069136 | 0.99 | C | A | 0.08 | 0.15 | 0.46 | 0.07 | 2 × 10 [symbol] |
| rs28581562 | 10069342 | 0.99 | A | G | 0.08 | 0.15 | 0.46 | 0.07 | 2 × 10 [symbol] |
| rs28465345 | 10069439 | 0.99 | A | G | 0.08 | 0.15 | 0.46 | 0.07 | 2 × 10 [symbol] |
| rs28375797 | 10069497 | 0.99 | T | C | 0.08 | 0.15 | 0.46 | 0.07 | 2 × 10 [symbol] |
| 16-10070145 | 10070145 | 0.99 | C | T | 0.08 | 0.15 | 0.46 | 0.07 | 2 × 10 [symbol] |
| rs8057034 | 10070452 | 0.99 | A | G | 0.08 | 0.15 | 0.46 | 0.07 | 2 × 10 [symbol] |
| rs8059513 | 10070729 | 0.97 | G | A | 0.08 | 0.14 | 0.46 | 0.08 | 3 × 10 [symbol] |
| rs8059547 | 10070795 | 0.99 | G | A | 0.08 | 0.15 | 0.45 | 0.07 | 2 × 10 [symbol] |
| 16-10070978 | 10070978 | 0.99 | C | T | 0.08 | 0.15 | 0.45 | 0.07 | 2 × 10 [symbol] |
| 16-10071089 | 10071089 | 0.99 | A | T | 0.08 | 0.15 | 0.45 | 0.07 | 2 × 10 [symbol] |
| rs9930396 | 10071278 | 0.99 | G | A | 0.08 | 0.15 | 0.45 | 0.07 | 2 × 10 [symbol] |
| 16-10071764 | 10071764 | 0.99 | T | C | 0.08 | 0.15 | 0.45 | 0.07 | 1 × 10 [symbol] |
| 16-10071991 | 10071991 | 0.99 | C | T | 0.08 | 0.15 | 0.45 | 0.07 | 1 × 10 [symbol] |
| 16-10071996 | 10071996 | 0.99 | C | G | 0.08 | 0.15 | 0.45 | 0.07 | 1 × 10 [symbol] |
| rs9933111 | 10072100 | 1.00 | G | A | 0.08 | 0.15 | 0.45 | 0.07 | 1 × 10 [symbol] |
| rs9922871 | 10072317 | 0.99 | A | C | 0.08 | 0.15 | 0.45 | 0.07 | 1 × 10 [symbol] |
| rs9922338 | 10072329 | 0.98 | A | G | 0.08 | 0.14 | 0.44 | 0.07 | 1 × 10 [symbol] |
| rs9302409 | 10072531 | 0.99 | C | T | 0.08 | 0.15 | 0.45 | 0.07 | 1 × 10 [symbol] |
| 16-10072565 | 10072565 | 0.99 | T | C | 0.08 | 0.15 | 0.45 | 0.07 | 1 × 10 [symbol] |
| rs17570290 | 10072647 | 0.99 | G | T | 0.08 | 0.15 | 0.45 | 0.07 | 1 × 10 [symbol] |
| rs10518152 | 10072788 | 0.99 | G | A | 0.08 | 0.15 | 0.45 | 0.07 | 1 × 10 [symbol] |
| 16-10073124 | 10073124 | 0.99 | C | A | 0.08 | 0.15 | 0.45 | 0.07 | 1 × 10 [symbol] |
| rs13332018 | 10073171 | 0.99 | T | C | 0.08 | 0.15 | 0.45 | 0.07 | 1 × 10 [symbol] |
| rs9938467 | 10073412 | 0.99 | C | T | 0.08 | 0.15 | 0.45 | 0.07 | 1 × 10 [symbol] |
| rs8061018 | 10073903 | 0.99 | C | T | 0.08 | 0.15 | 0.45 | 0.07 | 1 × 10 [symbol] |
| rs9927648 | 10074002 | 0.99 | A | G | 0.08 | 0.15 | 0.45 | 0.07 | 1 × 10 [symbol] |
| 16-10074078 | 10074078 | 0.99 | A | C | 0.08 | 0.15 | 0.45 | 0.07 | 1 × 10 [symbol] |
| rs58797003 | 10074188 | 0.99 | C | A | 0.08 | 0.15 | 0.45 | 0.07 | 1 × 10 [symbol] |
| rs59568026 | 10074278 | 0.99 | C | A | 0.08 | 0.15 | 0.45 | 0.07 | 1 × 10 [symbol] |
| 16-10074282 | 10074282 | 0.99 | A | G | 0.08 | 0.15 | 0.45 | 0.07 | 1 × 10 [symbol] |
| 16-10074631 | 10074631 | 0.99 | C | G | 0.08 | 0.15 | 0.45 | 0.07 | 1 × 10 [symbol] |
| 16-10074643 | 10074643 | 0.99 | T | A | 0.08 | 0.15 | 0.45 | 0.07 | 1 × 10 [symbol] |
| rs61224814 | 10074756 | 0.99 | T | C | 0.08 | 0.15 | 0.45 | 0.07 | 1 × 10 [symbol] |
| rs60950183 | 10074799 | 0.99 | G | A | 0.08 | 0.15 | 0.45 | 0.07 | 1 × 10 [symbol] |
| 16-10074810 | 10074810 | 0.99 | A | C | 0.08 | 0.15 | 0.45 | 0.07 | 1 × 10 [symbol] |
| 16-10074932 | 10074932 | 0.99 | C | T | 0.08 | 0.15 | 0.45 | 0.07 | 1 × 10 [symbol] |
| 16-10074942 | 10074942 | 0.99 | C | T | 0.08 | 0.15 | 0.45 | 0.07 | 1 × 10 [symbol] |
| 16-10075021 | 10075021 | 0.99 | G | A | 0.08 | 0.15 | 0.45 | 0.07 | 1 × 10 [symbol] |
| rs56666352 | 10075139 | 0.99 | C | T | 0.08 | 0.15 | 0.45 | 0.07 | 1 × 10 [symbol] |
| 16-10075411 | 10075411 | 0.99 | T | A | 0.08 | 0.15 | 0.45 | 0.07 | 1 × 10 [symbol] |
| 16-10075461 | 10075461 | 0.99 | T | C | 0.08 | 0.15 | 0.45 | 0.07 | 1 × 10 [symbol] |
| 16-10075477 | 10075477 | 0.99 | G | A | 0.08 | 0.15 | 0.45 | 0.07 | 1 × 10 [symbol] |
| rs12102832 | 10075633 | 0.99 | C | T | 0.08 | 0.15 | 0.45 | 0.07 | 1 × 10 [symbol] |
| 16-10075722 | 10075722 | 0.99 | C | T | 0.08 | 0.15 | 0.45 | 0.07 | 1 × 10 [symbol] |
| rs8062241 | 10075809 | 0.99 | C | T | 0.08 | 0.15 | 0.45 | 0.07 | 1 × 10 [symbol] |
| rs8062624 | 10075998 | 0.99 | C | T | 0.08 | 0.15 | 0.45 | 0.07 | 1 × 10 [symbol] |
| rs9931653 | 10076193 | 0.99 | C | T | 0.08 | 0.15 | 0.45 | 0.07 | 1 × 10 [symbol] |
| rs10518150 | 10076257 | 0.99 | G | T | 0.08 | 0.15 | 0.45 | 0.07 | 1 × 10 [symbol] |
| rs9933945 | 10076487 | 0.99 | A | T | 0.08 | 0.15 | 0.45 | 0.07 | 1 × 10 [symbol] |
| rs10518149 | 10076563 | 0.99 | C | A | 0.08 | 0.15 | 0.45 | 0.07 | 1 × 10 [symbol] |
| rs28634372 | 10076780 | 0.99 | G | A | 0.08 | 0.15 | 0.45 | 0.07 | 1 × 10 [symbol] |
| rs60276136 | 10077091 | 0.99 | G | A | 0.08 | 0.15 | 0.45 | 0.07 | 1 × 10 [symbol] |
| rs28540790 | 10077145 | 0.99 | G | T | 0.08 | 0.15 | 0.45 | 0.07 | 1 × 10 [symbol] |
| rs28574279 | 10077172 | 0.99 | A | G | 0.08 | 0.15 | 0.45 | 0.07 | 1 × 10 [symbol] |
| 16-10077221 | 10077221 | 0.99 | T | A | 0.08 | 0.15 | 0.45 | 0.07 | 1 × 10 [symbol] |
| rs57587803 | 10077247 | 0.99 | T | C | 0.08 | 0.15 | 0.45 | 0.07 | 1 × 10 [symbol] |
| rs60864732 | 10077257 | 0.99 | G | T | 0.08 | 0.15 | 0.45 | 0.07 | 1 × 10 [symbol] |
| rs58861142 | 10077314 | 0.99 | G | A | 0.08 | 0.15 | 0.45 | 0.07 | 1 × 10 [symbol] |
| rs16956843 | 10077466 | 0.99 | A | G | 0.08 | 0.15 | 0.45 | 0.07 | 1 × 10 [symbol] |
| 16-10077488 | 10077488 | 0.99 | A | G | 0.08 | 0.15 | 0.45 | 0.07 | 1 × 10 [symbol] |
| rs61686802 | 10077552 | 0.99 | C | T | 0.08 | 0.15 | 0.45 | 0.07 | 1 × 10 [symbol] |
| 16-10077618 | 10077618 | 0.99 | T | A | 0.08 | 0.15 | 0.4.5 | 0.07 | 1 × 10 [symbol] |
| 16-10077656 | 10077656 | 0.99 | A | G | 0.08 | 0.15 | 0.45 | 0.07 | 1 × 10 [symbol] |
| rs13331030 | 10077692 | 0.99 | C | G | 0.08 | 0.15 | 0.45 | 0.07 | 1 × 10 [symbol] |
| 16-10077704 | 10077704 | 0.99 | C | A | 0.08 | 0.15 | 0.45 | 0.07 | 1 × 10 [symbol] |

[symbol]

indicates data missing or illegible when filed

In a genome-wide gene-environment study Applicants identified GRIN2A as a genetic modifier of the inverse association of coffee with the risk of developing PD. The discovery was made in NGRC, and replicated in independent data. Risk reduction by heavy-coffee use, which was estimated to be 27% on average, was genotype-specific and varied according to GRIN2A genotype from 18% (P=3×10⁻³) for individuals with rs4998386_CC genotype to 59% (P=6×10⁻¹³) for those with rs4998386_TC genotype. When coffee intake was categorized in four doses, the dose trend was more prominent in individuals with rs4998386_T allele than those with rs4998386_CC genotype, with the 3rd and 4th quartiles exhibiting only 11% and 39% risk reduction for rs4998386_CC carriers, vs. 37% and 66% for rs4998386_T carriers. With imputation, Applicants uncovered a block of GRIN2A SNPs not included on the genotyping array, which achieved P=3×10⁻⁸ to 5×10⁻⁸. Applicants propose GRIN2A as a new modifier gene for PD, and posit that if coffee-consumption is considered, GRIN2A may prove to be one of the most important PD-associated genes to have emerged from genome-wide studies. Applicants base this suggestion on statistics, biology and the potential for immediate translation to clinical medicine, as Applicants discuss below. GRIN2A had not previously been tested as a candidate gene for PD, and was not detected in PD GWAS which have all been examining gene main effects without considering interactions with relevant environmental exposures. The most significant and consistently replicated main effects detected to date are for SNCA, MAPT and HLA. Here Applicants added, for the first time, a common and relevant environmental exposure (coffee) to a genome-wide study. Inclusion of coffee allowed GRIN2A to rise to the top. In the gene-environment (GWAIS) model, GRIN2A surpassed SNCA, MAPT and HLA in statistical significance. Among heavy-coffee-drinkers, the impact of GRIN2A on PD risk (measured as OR) was 50% greater, and 2 to 5 orders of magnitude more significant (measured as P value) than the strongest associations reported for SNCA, MAPT or HLA. This study is proof of concept that inclusion of environmental factors can help identify disease-associated genes that are missed in SNP-only GWAS.

GRIN2A is an important gene for the central nervous system. Accelerated evolution of GRIN2A in primates is said to have contributed to the dramatic increase in the size and complexity of the human brain which defines human evolution [35]. GRIN2A encodes a subunit of the N-methyl-D-aspartate-2A (NMDA) glutamate receptor. It is central to excitatory neurotransmission and the control of movement and behavior [36,37,38]. The literature suggest imbalances in NMDA-dependent neurotransmission contribute to neurodegeneration in PD, possibly through massive influx of calcium and impaired mitochondrial function leading to apoptosis; and/or disruption of glutamate-mediated autophagy which is implicated in degradation and removal of proteins like α-synuclein (see [39] for review). The portion of intron 3 containing SNPs with the most significant associations (from 9978046 basepair to 10128367 basepair, Tables 10, 11, 12 and 13) includes numerous transcription factor binding sites, and two peaks of enhanced histone H3K4 mono-methylation (genome.ucsc.edu website) [40]. Polymorphisms throughout this region could therefore disrupt regulatory elements, potentially leading to variation in levels of GRIN2A transcript. GRIN2A is expressed at high levels in the brain, most notably in the subthalamic nucleus (STN) [41]. Pharmacologic inhibition of STN with an NMDA-antagonist reduces nigral neuron loss in a rodent model of PD [42]. Deep-brain-stimulation, which also targets STN, is an effective surgical symptomatic therapy for PD.

The other piece of this finding is coffee/caffeine. Applicants' study was not designed to distinguish the active ingredients in coffee. However, Applicants note that the largest replication study (PAGE) measured specifically the caffeine intake in mg from all food sources (drink, food, and chocolate) and replicated Applicants' hypothesis and interaction robustly. Applicants also found trends for inverse association of tea and soda with PD, and interestingly, the varied effect size and strength of association was consistent with the relative amount of caffeine in each drink (Table 9). Thus, Applicants' data is consistent with experimental observations that caffeine is neuroprotective.

Caffeine is an adenosine A2A-receptor-antagonist. A2A-receptor enhances calcium influx via NMDA [38] and A2A-receptor-antagonists are neuroprotective in animal models of PD; they attenuate excitotoxicity by reducing extracellular glutamate levels in the striatum [43,44]. Thus interaction between coffee/caffeine and GRIN2A is biologically plausible, and can help formulate testable hypotheses towards a better understanding of the disease pathogenesis.

GRIN2A genotyping may be useful for pharmacogenetic studies. Genetics has not yet entered drug development for PD but the time is here. Applicants now have several susceptibility loci (SNCA, MAPT, HLA, BST1, PARK16, GAK [4,5,6,7,8,9]) that can help identify individuals who are at moderately increased risk of developing PD. Applicants also have at least one neuroprotective compound (coffee/caffeine) which can be pharmacologically modified to alleviate its undesirable side effects. GRIN2A genotyping might also inform treatments for people who already have PD. L-DOPA, the primary PD drug for 40 years, does not slow disease progression and has serious side effects. Clinical trials for new PD drugs have not found drugs that surpass the symptomatic benefits of L-DOPA. There have been numerous drug trials for glutamate-receptor blockers as well as for selective A2A-receptor-antagonists. Most were shown to be safe, well tolerated and beneficial [16,17,45]; however, the majority did not reach the regulatory threshold for efficacy to be approved as PD drugs. Applicants wonder if the clinical trials will succeed if patients are subdivided by GRIN2A genotype. Applicants acknowledge the distinction that the present study examined risk of developing PD; whereas clinical trials have thus far aimed for symptomatic improvements in patients. Nonetheless, there are sufficient parallels to suggest that GRIN2A genotype might also influence efficacy of glutamate-receptor-antagonists and A_{2A}-receptor-antagonists. This is a simple and inexpensive hypothesis that can be tested in future, ongoing and even closed clinical trials that have banked DNA.

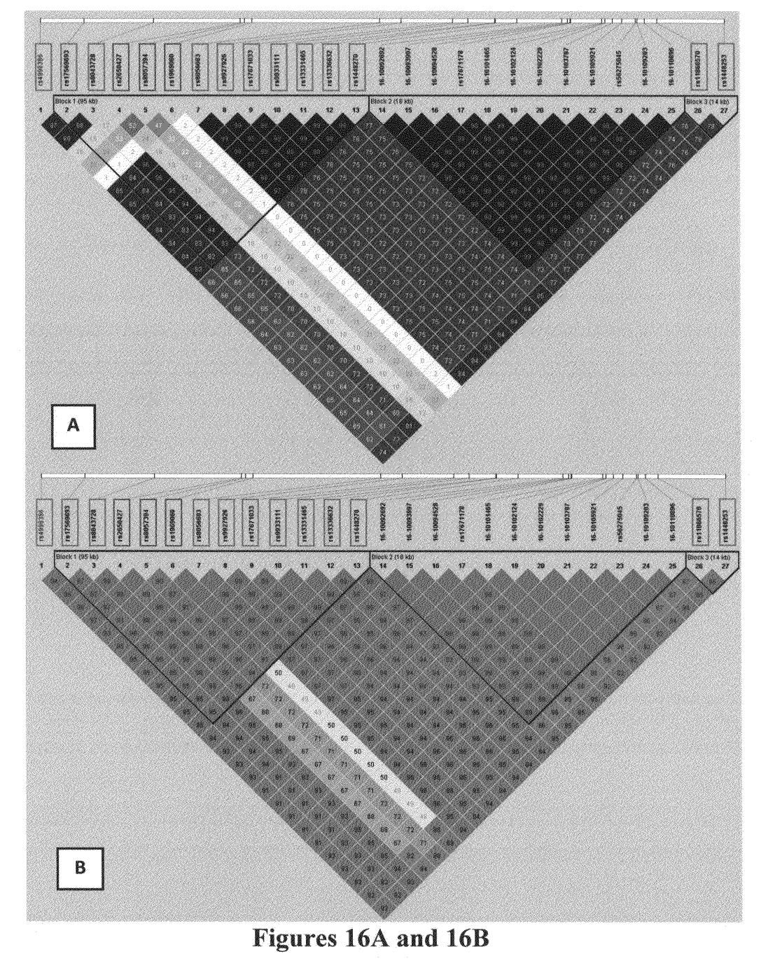

Common non-coding variants in GRIN2A have been associated with Huntington disease (HD) [46,47] and schizophrenia [48], and rare mutations have been described in patients with neurodevelopmental phenotypes [49]. Schizophrenia is associated with a (GT)n repeat in GRIN2A promoter that may increase disease risk by suppressing gene expression [48]. Three GRIN2A SNPs have been associated with onset-age of HD; they are conserved and reportedly tag a binding site for CCAAT/enhancer-binding protein [46,47]. HD and PD are both neurodegenerative movement disorders, thus the possibility of a common genetic element was of interest. The reported HD-associated GRIN2A SNPs, rs1969060, rs8057394 and rs2650427, were not on the genotyping array but were imputed with high fidelity (information score>0.99). They map within the 150 kb region identified here for PD, they are in strong LD with PD-associated SNPs defined by D′ (0.48-1.0) but not by r2 (0-0.33) (FIG. 16). Applicants tested HD-SNPs for association with onset age and risk of PD in NGRC while conditioning on the neighboring top PD-SNP (rs4998386). One HD-SNP, rs8057394, yielded OR=0.85, P=0.02 for PD overall; OR=0.79, P=0.04 for heavy-coffee drinkers; and OR=0.90, P=0.24 for light-coffee drinkers. Applicants found no other evidence for association of HD-SNPs with PD, including when Applicants jointly tested HD SNPs and possible interaction with coffee [SNP+SNP*coffee] on risk or onset of PD. Conversely, Applicants retested, in NGRC, the association of top genotyped PD SNP (rs4998386) with PD, conditioning on HD-SNP (rs8057394) and found it to be robust (P_{2df}=8×10⁻⁶).

Amassing a large enough sample size for GWAIS is challenging. GWAIS requires larger sample sizes than GWAS yet there exist fewer samples that have data on relevant environmental exposures in addition to DNA and phenotype. To Applicants' knowledge, NGRC is the largest genetic study of PD that has collected exposure data. No other publically available PD GWAS has coffee data, eliminating the possibility of in-silico replication. Applicants were able to identify and get access to only 3 datasets that had DNA and coffee, giving us a total sample size of 393 cases and 905 controls to replicate the GRIN2A effect in heavy-coffee-drinkers. In contrast, replications and meta-analyses for gene-only GWAS now have over 17,000 PD cases and controls [9]. Applicants detected the known and confirmed PD-associated genes (SNCA, MAPT and HLA) in GWAIS but at much lower significance levels than in GWAS because of the smaller sample size with coffee data and the added degree of freedom in GWAIS. It is noteworthy, however, that at P_{2df}=10⁻⁶, GRIN2A surpassed all known PD loci in significance. With the aid of imputation, Applicants achieved P=3×10⁻⁸ for a 2.4-fold difference in genotype specific effect of coffee on risk of PD. Importantly, Applicants were able to replicate the hypothesis that Applicants set out a-priori based on discovery and approved it.

NGRC genome-wide genotypes, phenotype and environmental data are available at the website of the national center of biotechnology information under gap study accession number phs000196.v1.p1.

### REFERENCES

1. Manolio T A, Collins F S, Cox N J, Goldstein D B, Hindorff L A, et al. (2009) Finding the missing heritability of complex diseases. Nature 461: 747-753.
2. Dorsey E R, Constantinescu R, Thompson J P, Biglan K M, Holloway R G, et al. (2007) Projected number of people with Parkinson disease in the most populous nations, 2005 through 2030. Neurology 68: 384-386.
3. Hardy J (2010) Genetic analysis of pathways to Parkinson disease. Neuron 68: 201-206.
4. Hamza T H, Zabetian C P, Tenesa A, Laederach A, Montimurro J, et al. (2010) Common genetic variation in the HLA region is associated with late-onset sporadic Parkinson's disease. Nat Genet 42: 781-785.
5. Satake W, Nakabayashi Y, Mizuta I, Hirota Y, Ito C, et al. (2009) Genome-wide association study identifies common variants at four loci as genetic risk factors for Parkinson's disease. Nat Genet 41: 1303-1307.
6. Simon-Sanchez J, Schulte C, Bras J M, Sharma M, Gibbs J R, et al. (2009) Genome-wide association study reveals genetic risk underlying Parkinson's disease. Nat Genet 41: 1308-1312.
7. Pankratz N, Wilk J B, Latourelle J C, DeStefano A L, Halter C, et al. (2009) Genomewide association study for susceptibility genes contributing to familial Parkinson disease. Hum Genet 124: 593-605.
8. Edwards T L, Scott W K, Almonte C, Burt A, Powell E H, et al. (2010) Genome-Wide Association Study Confirms SNPs in SNCA and the MAPT Region as Common Risk Factors for Parkinson Disease. Ann Hum Genet 74: 97-109.
9. Nails M A, Plagnol V, Hernandez D G, Sharma M, Sheerin U M, et al. (2011) Imputation of sequence variants for identification of genetic risks for Parkinson's disease: a meta-analysis of genome-wide association studies. Lancet 377: 641-649.
10. Hernan M A, Takkouche B, Caamano-Isorna F, Gestal-Otero J J (2002) A meta-analysis of coffee drinking, cigarette smoking, and the risk of Parkinson's disease. Ann Neurol 52: 276-284.
11. Powers K, Kay D, Factor S, Zabetian C, Higgins D, et al. (2008) Combined effects of smoking, coffee and NSAIDs on Parkinson's disease risk. Mov Disord 23: 88-95.
12. Costello S, Cockburn M, Bronstein J, Zhang X, Ritz B (2009) Parkinson's disease and residential exposure to maneb and paraquat from agricultural applications in the central valley of California. Am J Epidemiol 169:919-926.
13. McCulloch C C, Kay D M, Factor S A, Samii A, Nutt J G, et al. (2008) Exploring gene-environment interactions in Parkinson's disease. Hum Genet 123: 257-265.
14. Kraft P, Yen Y C, Stram D O, Morrison J, Gauderman W J (2007) Exploiting gene-environment interaction to detect genetic associations. Hum Hered 63: 111-119.
15. Chen J F, Xu K, Petzer J P, Staal R, Xu Y H, et al. (2001) Neuroprotection by caffeine and A(2A) adenosine receptor inactivation in a model of Parkinson's disease. J Neurosci 21: RC143.
16. LeWitt P A, Guttman M, Tetrud J W, Tuite P J, Mori A, et al. (2008) Adenosine A2A receptor antagonist istradefylline (KW-6002) reduces “off” time in Parkinson's disease: a double-blind, randomized, multicenter clinical trial (6002-US-005). Ann Neurol 63: 295-302.
17. Fernandez H H, Greeley D R, Zweig R M, Wojcieszek J, Mori A, et al. (2010) Istradefylline as monotherapy for Parkinson disease: results of the 6002-US-051 trial. Parkinsonism Relat Disord 16: 16-20.
18. Simon-Sanchez J, van Hilten J J, van de Warrenburg B, Post B, Berendse H W, et al. (2010) Genome-wide association study confirms extant PD risk loci among the Dutch. Eur J Hum Genet.
19. Ritz B R, Manthripragada A D, Costello S, Lincoln S J, Farrer M J, et al. (2009) Dopamine transporter genetic variants and pesticides in Parkinson's disease. Environ Health Perspect 117: 964-969.
20. Chen H, Huang X, Guo X, Mailman R B, Park Y, et al. Smoking duration, intensity, and risk of Parkinson disease. Neurology 74: 878-884.
21. Hughes A J, Daniel S E, Ben-Shlomo Y, Lees A J (2002) The accuracy of diagnosis of parkinsonian syndromes in a specialist movement disorder service. Brain 125: 861-870.
22. Gao J, Xu H, Weinberg C, Huang X, Park Y, et al. (2011) An Exploratory Study on the CHRNA3-CHRNAS-CHRNB4 Cluster, Smoking, and Parkinson's Disease. Neurodegener Dis.
23. Hancock D B, Martin E R, Stajich J M, Jewett R, Stacy M A, et al. (2007) Smoking, caffeine, and nonsteroidal anti-inflammatory drugs in families with Parkinson disease. Arch Neurol 64: 576-580.
24. Thompson F E, Kipnis V, Midthune D, Freedman L S, Carroll R J, et al. (2008) Performance of a foodfrequency questionnaire in the US NIH-AARP (National Institutes of Health-American Association of Retired Persons) Diet and Health Study. Public Health Nutr 11: 183-195.
25. Purcell S, Neale B, Todd-Brown K, Thomas L, Ferreira M A, et al. (2007) PLINK: a tool set for wholegenome association and population-based linkage analyses. Am J Hum Genet 81: 559-575.
26. Gao X, Becker L C, Becker D M, Starmer J D, Province M A (2010) Avoiding the high Bonferroni penalty in genome-wide association studies. Genet Epidemiol 34: 100-105.
27. Howie B N, Donnelly P, Marchini J (2009) A flexible and accurate genotype imputation method for the next generation of genome-wide association studies. PLoS Genet 5: e1000529.
28. Barrett J C, Fry B, Maller J, Daly M J (2005) Haploview: analysis and visualization of LD and haplotype maps. Bioinformatics 21: 263-265.
29. Schaid D J, Rowland C M, Tines D E, Jacobson R M, Poland G A (2002) Score tests for association between traits and haplotypes when linkage phase is ambiguous. Am J Hum Genet 70: 425-434.
30. Wang K, Li M, Hadley D, Liu R, Glessner J, et al. (2007) PennCNV: an integrated hidden Markov model designed for high-resolution copy number variation detection in whole-genome SNP genotyping data. Genome Res 17: 1665-1674.
31. Zabetian C P, Hutter C M, Yearout D, Lopez A N, Factor S A, et al. (2006) LRRK2 G2019S in families with Parkinson disease who originated from Europe and the Middle East: evidence of two distinct founding events beginning two millennia ago. Am J Hum Genet 79: 752-758.
32. Evans A H, Lawrence A D, Potts J, MacGregor L, Katzenschlager R, et al. (2006) Relationship between impulsive sensation seeking traits, smoking, alcohol and caffeine intake, and Parkinson's disease. J Neurol Neurosurg Psychiatry 77: 317-321.
33. Levran O, Londono D, O'Hara K, Randesi M, Rotrosen J, et al. (2009) Heroin addiction in African Americans: a hypothesis-driven association study. Genes Brain Behav 8: 531-540.
34. Vink J M, Smit A B, de Geus E J, Sullivan P, Willemsen G, et al. (2009) Genome-wide association study of smoking initiation and current smoking. Am J Hum Genet 84: 367-379.
35. Dorus S, Vallender E J, Evans P D, Anderson J R, Gilbert S L, et al. (2004) Accelerated evolution of nervous system genes in the origin of Homo sapiens. Cell 119: 1027-1040.
36. Shen W, Flajolet M, Greengard P, Surmeier D J (2008) Dichotomous dopaminergic control of striatal synaptic plasticity. Science 321: 848-851.
37. Jin X, Costa R M (2010) Start/stop signals emerge in nigrostriatal circuits during sequence learning. Nature 466: 457-462.
38. Higley M J, Sabatini B L (2010) Competitive regulation of synaptic Ca2+ influx by D2 dopamine and A2A adenosine receptors. Nat Neurosci 13: 958-966.
39. Caudle W M, Zhang J (2009) Glutamate, excitotoxicity, and programmed cell death in Parkinson disease. Exp Neurol 220: 230-233.
40. Birney E, Stamatoyannopoulos J A, Dutta A, Guigo R, Gingeras T R, et al. (2007) Identification and analysis of functional elements in 1% of the human genome by the ENCODE pilot project. Nature 447: 799-816.
41. Su A I, Wiltshire T, Batalov S, Lapp H, Ching K A, et al. (2004) A gene atlas of the mouse and human protein-encoding transcriptomes. Proc Natl Acad Sci USA 101: 6062-6067.
42. Blandini F, Nappi G, Greenamyre J T (2001) Subthalamic infusion of an NMDA antagonist prevents basal ganglia metabolic changes and nigral degeneration in a rodent model of Parkinson's disease. Aim Neurol 49:525-529.
43. Schwarzschild M A, Xu K, Oztas E, Petzer J P, Castagnoli K, et al. (2003) Neuroprotection by caffeine and more specific A2A receptor antagonists in animal models of Parkinson's disease. Neurology 61: S55-61.
44. Tebano M T, Pintor A, Frank C, Domenici M R, Martire A, et al. (2004) Adenosine A2A receptor blockade differentially influences excitotoxic mechanisms at pre- and postsynaptic sites in the rat striatum. J Neurosci Res 77: 100-107.
45. Shoulson I, Penney J, McDermott M, Schwid S, Kayson E, et al. (2001) A randomized, controlled trial of remacemide for motor fluctuations in Parkinson's disease. Neurology 56: 455-462.
46. Arning L, Saft C, Wieczorek S, Andrich J, Kraus P H, et al. (2007) NR2A and NR2B receptor gene variations modify age at onset in Huntington disease in a sex-specific manner. Hum Genet 122: 175-182.
47. Andresen J M, Gayan J, Cherny S S, Brocklebank D, Alkorta-Aranburu G, et al. (2007) Replication of twelve association studies for Huntington's disease residual age of onset in large Venezuelan kindreds. J Med Genet 44: 44-50.
48. Itokawa M, Yamada K, Yoshitsugu K, Toyota T, Suga T, et al. (2003) A microsatellite repeat in the promoter of the N-methyl-D-aspartate receptor 2A subunit (GRIN2A) gene suppresses transcriptional activity and correlates with chronic outcome in schizophrenia. Pharmacogenetics 13: 271-278.
49. Endele S, Rosenberger G, Geider K, Popp B, Tamer C, et al. (2010) Mutations in GRIN2A and GRIN2B encoding regulatory subunits of NMDA receptors cause variable neurodevelopmental phenotypes. Nat Genet.

### Example 4

### A Genetic Basis for the Variable Effect of Smoking/Nicotine on Parkinson's Disease

Prior studies have established an inverse association between cigarette-smoking and the risk of developing Parkinson's disease (PD), and currently, disease-modifying potential of the nicotine-patch is being tested in clinical trials. To identify genes that interact with the effect of smoking/nicotine, Applicants conducted genome-wide interaction studies in humans and in Drosophila. Applicants identified SV2C which encodes a synaptic-vesicle protein in PD-vulnerable substantia-nigra (P=9×10⁻⁸ for gene-smoking interaction on PD risk), and CG14691 which is predicted to encode a synaptic-vesicle protein in Drosophila (P=2×10⁻¹¹ for nicotine-paraquat interaction on gene-expression). SV2C is biologically plausible because nicotine enhances release of dopamine through synaptic vesicles, and PD is caused by depletion of dopamine. Effect of smoking on PD varied by SV2C genotype from protective to neutral to harmful)(P=4×10⁻¹⁰. Taken together, cross-validating evidence from humans and Drosophila suggest SV2C is involved in PD pathogenesis and it might be a useful marker for pharmacogenomics studies involving nicotine.

PD is a progressive degenerative disorder of the central nervous system. Dopamine-producing neurons in the substantia nigra selectively degenerate, resulting in a drastic reduction in the brain dopamine levels. Dopamine is a neurotransmitter and can impact many functions including voluntary movement, cognition, mood, behavior and sleep, all of which are altered in PD.Current treatments are directed towards dopamine replacement. While they help with early motor difficulties, they do not slow the progression of the disease and are associated with several late complications. To date, none of the clinical trials for neuroprotective treatments of PD have succeeded. Applicants suspect that the inability so far to account for genetic differences that affect drug response has been a hindrance to treatment trials.

Epidemiological studies have shown that caffeinated-coffee and cigarette-smoking are inversely associated with risk of developing PD^{1, 2}. Although neuroprotective effects of caffeine and nicotine have been demonstrated in animal models of PD^{3, 4}, there is controversy as to whether the inverse associations in humans signify true protective effects or a personality trait that renders those predisposed to PD to avoid habit forming behaviors⁵. Previously, Applicants identified GRIN2A as a novel gene for PD through a genome-wide interaction study with caffeine, and showed that the genetic association was specific to the risk of PD and not to the tendency for caffeine use⁶. In the present study, Applicants sought to identify genes that influence the effect of smoking on PD.

Applicants conducted genome-wide studies in humans, searching for genes that interact with the effect of smoking, and in Drosophila, searching for genes whose expression was affected by the interaction between paraquat, which Applicants used to induce parkinsonism in the fly, and nicotine, which Applicants used to rescue the flies from paraquat toxicity. Several genetic and toxin animal models of parkinsonism are available. Applicants chose the paraquat model in Drosophila because paraquat is an environmental risk factor for PD⁷⁻⁹, and Drosophila is amenable to powerful genetic analyses. The paraquat fly model of parkinsonism is created by feeding paraquat to flies, which results in selective and progressive loss of dopaminergic neurons, motor abnormalities and shortened lifespan¹⁰. Thus, in this model many of the phenotypic hallmarks of the human parkinsonism are recapitulated, and the significantly shortened life span provides a parkinsonism-associated phenotype that is amenable to rapid screening. The two experiments, conducted in humans and Drosophila, converged on a pair of homologous genes, CG14691 and SV2C, which encode a synaptic vesicle protein involved in release of dopamine.

Human Study.

All research participants gave informed consent as approved by the Institutional Review Boards of the participating institutions. Subjects were from NeuroGenetics Research Consortium (NGRC)¹¹. PD was diagnosed using standard criteria¹². Controls were self-reported as not having any neurologic disease. Subjects were classified as being an ever- or never-smoker, using the common definition of having smoked ≧100 cigarettes in the lifetime². All subjects were unrelated, Caucasian Americans of European ancestry, by self-report and confirmed by principal component (PC) analysis¹¹. DNA was extracted from whole blood and umamplified. The Illumina HumanOmni 1-Quad_v1-0_B array was used for genotyping, achieving a call rate of 99.92% and 99.99% reproducibility. Details of genotyping procedures and quality control have been published¹¹. Every NGRC subject for whom genome-wide genotype and smoking data were available was included in the present study (1 600 persons with PD and 1 506 without PD; see Table 19).

TABLE 19
Human: Subjects characteristics

| | | | | [00328] Subjects Analyzed | | | | | | |
|---|---|---|---|---|---|---|---|---|---|---|
| | [00326] | [symbol] [00327] | | | | | [00332] | | | |
| [00325] | Geno- | Excluded | | | | | Age at | | [00333] | |
| ase | typed | smoking | N | | [00330 [symbol] | [00331] | onset | | Age Mean ± | |
| status | for GWAS | data | No | [0032 [symbol] | Men | Smoked | Mean ± S.E | | S.E | |
| [00334] | [00335] | 2 [00336] | 4 | [0033 [symbol] | [00338 [symbol] | [00339] | [00340] | 5 | [00341] | 6 |
| D | 000 | 00 | | 600 | 7.6 | 5.9 | 8.3 ± | | 6.8 ± | |
| | | | | | | | 0.3 | | 0.3 | |
| [00342] | [00343] | 1 [00344] | 4 | [0034 [symbol] | [00346 [symbol] | [00347] | [00348] | — | [00349] | 7 |
| ontrols | 986 | 80 | | 506 | 0.2 | 6.1 | | | 2.5 ± | |
| | | | | | | | | | 0.4 | |

[symbol]

indicates data missing or illegible when filed

Genotypes in the NGRC dataset had been previously cleaned for GWAS¹¹, but since Applicants used only subjects who had smoking data, Applicants rechecked all single nucleotide polymorphisms (SNPs) and included only those that had a minor allele frequency (MAF)≧0.01 in the subset of data used here. This yielded 810 234 genotyped SNPs. For each SNP, genotype was defined in dosage as 0, 1 or 2 copies of the minor allele (additive model). Smoking was treated as a binary variable as ever- or never-smoked at least 100 cigarettes in the life-time. Applicants tested SNP*smoking interaction using logistic regression and adjusting for four covariates that associate with PD risk in the NGRC dataset;¹¹ namely, PC1 and PC2, sex, and age at blood draw. Applicants used ProbABEL v1.0 software¹³ to test SNP*smoking interaction and to estimate standard errors using the Huber-White¹⁴ sandwich robust covariance estimator, and R software to obtain Wald test asymptotic P-values.

Haplotype blocks were constructed using Haploview¹⁵. Conditional analysis was performed in R.Association of SV2C with smoking was tested in R using logistic regression and adjusting for sex. A copy number variation (CNV) exists in SV2C. Using the PennCNV software¹⁶, Applicants detected 4 cases and 4 controls with a CNV in SV2C (all were duplications). The detected CNV ranged from 6 kb to 20 kb, and were all contained in intron 2 which is downstream to and does not overlap with the region that exhibits evidence for interaction with smoking.

Drosophila Study.

All experiments were conducted with D. melanogaster w¹¹¹⁸ females (Bloomington Drosophila Stock Center) kept at consistent conditions at 25° C., 55% humidity, and ambient light. Flies were collected within 24 hrs after eclosion, placed on media with or without nicotine for eight or ten days, and then placed on food with or without paraquat while continuing on nicotine at the dose they were pretreated on. Flies were transferred to new vials every other day. Nicotine ([−]-Nicotine in PESTANAL, Sigma-Aldrich) and paraquat (Methyl viologen dichloride hydrate, Sigma-Aldrich) were dissolved in dH₂O at stock concentrations of 50 mg/ml and 1M, respectively. These solutions were prepared fresh before each batch of media preparation and were added to warmed, liquefied standard fly food (prepared with agar, cornmeal, sucrose and yeast) and mixed thoroughly prior to pouring into vials. Food was prepared fresh at least monthly.

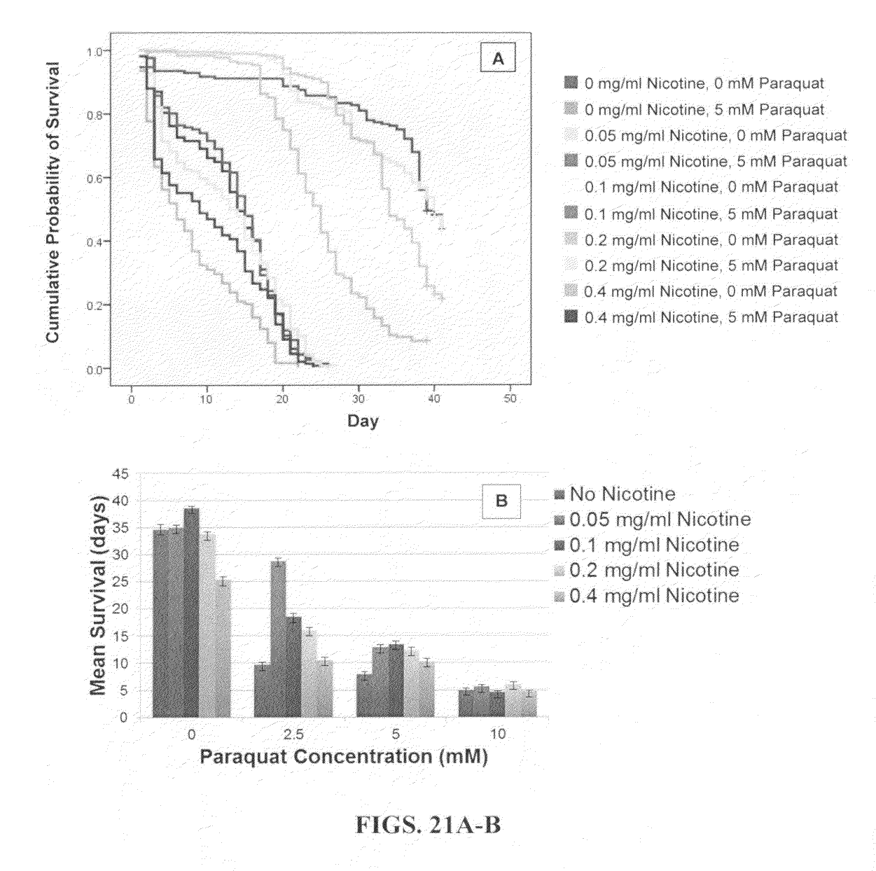

Three sets of experiments were conducted: a pilot study, a large-scale survival experiment, and a gene expression study. For the pilot study, flies were pretreated with 0, 0.05, 0.1, 0.2, or 0.4 mg/ml of nicotine for eight days and then transferred to food containing 0, 2.5, 5, or 10 mM paraquat while continuing on nicotine at the dose they were pretreated on. For each nicotine-paraquat dose combination, Applicants had six vials each with 30 flies for a total of 180 flies. The number of dead flies was counted daily until nearly all flies were dead (FIG. 21).

For the full experiment Applicants set up 420 flies (14 vials each containing 30 flies) for each dose combination of 0, 0.01, 0.05, 0.1, 0.2, 0.4 mg/ml for nicotine pretreatment for eight days followed with addition of 0 or 5 mM paraquat. Applicants did not pursue paraquat at 2.5 mM or 10 mM. 2.5 mM paraquat was associated with high vial-to-vial variability in survival times. At 10 mM, nicotine did not have a notable effect on survival. Flies were followed according to the same protocol, counting dead flies daily until all were dead. Survival data were plotted using Kaplan Meier survival analysis¹⁷, mean and median survival times were calculated, and the differences between the survival curves were tested using log rank statistics in SPSS.

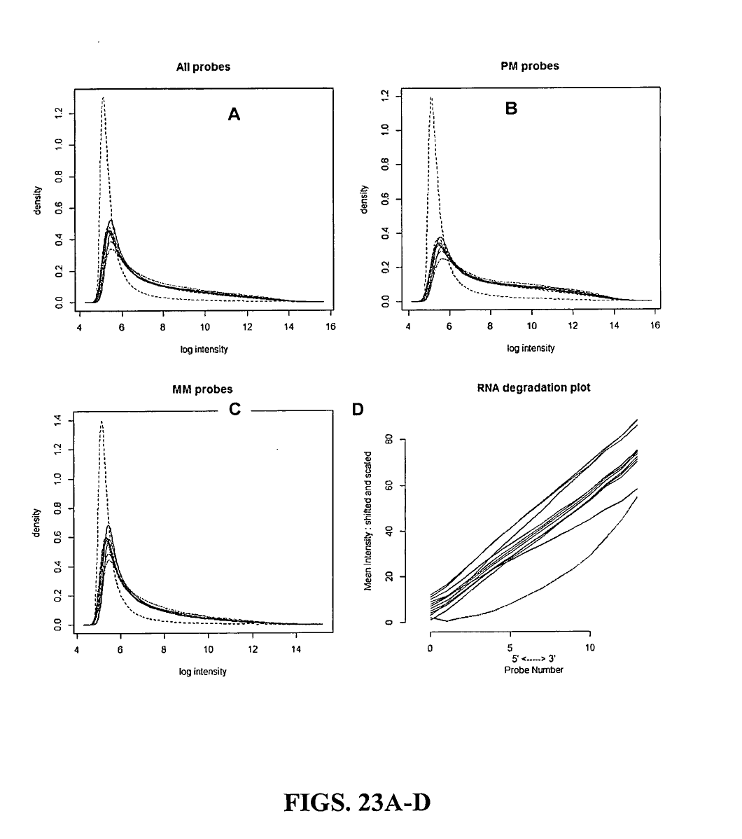

For gene expression study, Applicants had four dose combinations, each conducted in triplicate, at the same time and under the same conditions. 30 flies per vial were pretreated with 0 or 0.1 mg/ml nicotine for ten days and then co-treated with 0 or 5 mM of paraquat for six days. At the end of the treatment period, heads were dissected from 20 flies per vial and frozen at −80° C. for up to a month. RNA was extracted from each sample using TriReagent and its provided protocol, and cleaned using Qiagen RNeasy Cleanup kit and the associated protocol. RNA was stored at −80° C. for approximately two months. Affymetrix GeneChip Drosophila Genome 2.0 arrays were used for genome-wide quantification of transcript abundance. Expression data were analyzed using Bioconductor version 2.9¹⁸ packages in R (version 2.14). Raw signal data were examined for signs of RNA degradation using AffyRNAdeg implemented in Bioconductor, and for inconsistencies in overall probe intensity by visually inspecting log(intensity) density plots. One replicate of the paraquat-only treatment was found to be an outlier and excluded from data analysis (FIG. 23). Data were normalized using the GCRMA¹⁹ algorithm including quantile normalization, pmonly correction, and median polish summarization. Statistical interaction between nicotine and paraquat on gene expression (specified as log₂(signal)) was tested using the linear model implemented in limma²⁰. Expression differences were tested for 18 954 transcripts. Microarray data P-values were corrected using multiple testing adjustment²¹ included in the limma package.

Drosophila and Human studies, independently, revealed a pair of homologous genes as the most significant signal for interaction with nicotine and smoking, respectively. Applicants present the results of the Drosophila study first because the signal passed the genome-wide significance threshold for discovery. The human study was highly significant as corroborating evidence for validation.

Drosophila Paraquat-Nicotine Model.

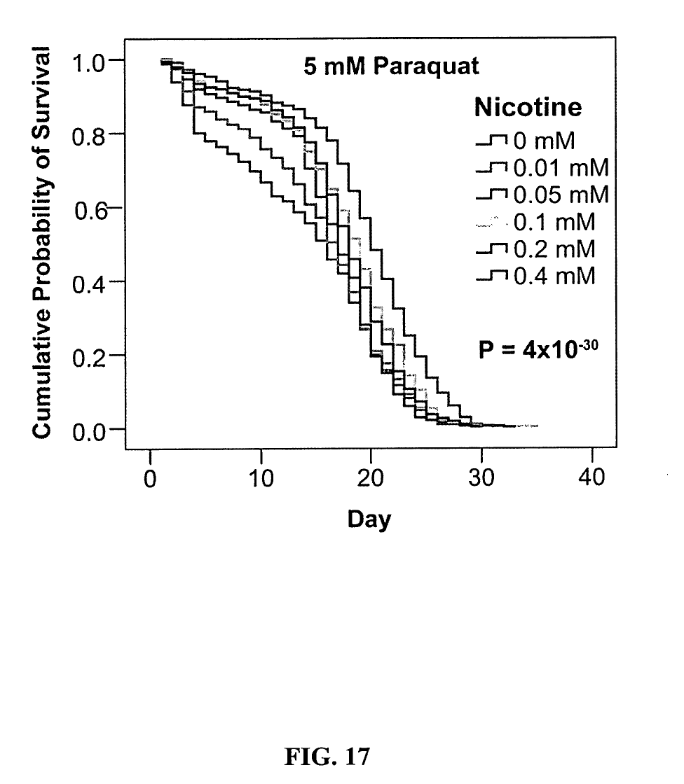

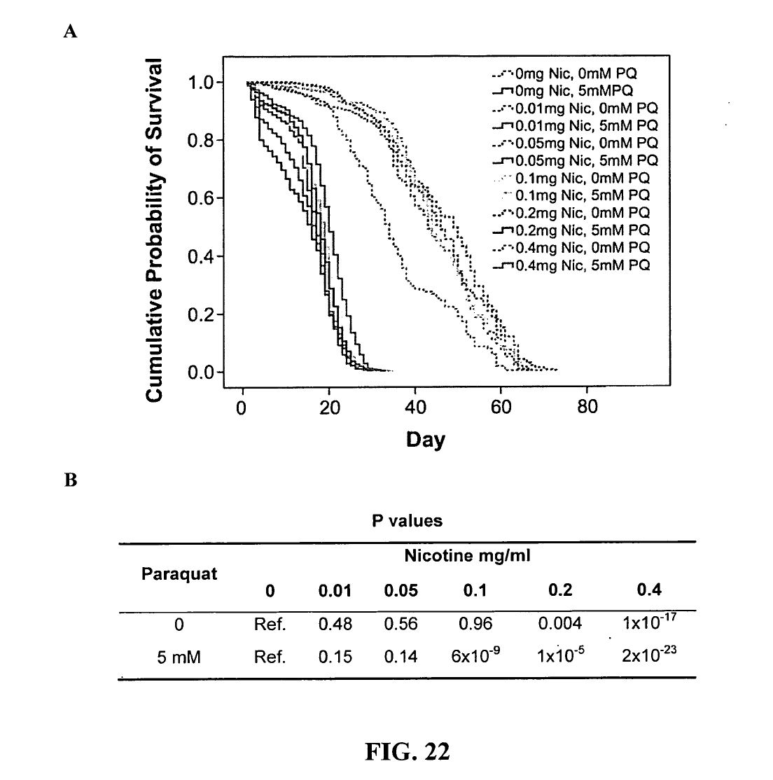

It has previously been established that reduced lifespan is a part of the paraquat-induced parkinsonism phenotype¹⁰. Consistent with this notion, Applicants found that paraquat shortened flies' lifespan by 63% (P=9×10⁻¹⁶⁸). When co-treated with nicotine, Applicants found that nicotine improved survival for paraquat-treated flies in a dose-dependent manner by up to 25% (P=2×10⁻²³). A beneficial effect of nicotine on survival was evident in both paraquat-treated (P=1×10⁻⁵) and untreated (P=4×10⁻³) flies, up to 0.2 mg/ml nicotine. However, at high dose (0.4 mg/ml), nicotine became toxic for flies that were not exposed to paraquat causing a 21% decline in median survival (P=1×10⁻¹⁷), though it continued to extend the lifespan of paraquat-treated flies up to 25% (P=2×10⁻²³). These results, detailed in Table 16, FIG. 17, and FIG. 22, demonstrate successful construction of a nicotine-paraquat model in Drosophila. Furthermore, they reaffirm that nicotine can be protective against paraquat toxicity.

TABLE 16
Drosophila: Effects of paraquat and nicotine on survival.
Paraquat reduced median survival by 63%
(43 ± 1.0 vs. 16 ± 0.5, P = 9 × 10−168). Nicotine restored
survival of paraquat-treated flies by up to 25% (P = 2 × 10−23).

| | No paraquat | | | 5 mM paraquat | | |
|---|---|---|---|---|---|---|
| | Mean | Median | | Mean | Median | |
| Nicotine | survival | survival | | survival | survival | |
| mg/ml | Days ± SE | Days ± SE | P | Days ± SE | Days ± SE | P |
| 0 | 43.3 ± 0.7 | 43 ± 1.0 | Ref | 14.0 ± 0.4 | 16 ± 0.5 | Ref |
| 0.01 | 44.4 ± 0.6 | 46 ± 0.8 | 0.48 | 15.2 ± 0.3 | 17 ± 0.4 | 0.15 |
| 0.05 | 45.1 ± 0.6 | 46 ± 0.8 | 0.56 | 16.1 ± 0.3 | 17 ± 0.3 | 0.14 |
| 0.1 | 44.4 ± 0.6 | 44 ± 0.6 | 0.96 | 17.6 ± 0.3 | 19 ± 0.3 | 6 × 10−9 |
| 0.2 | 46.4 ± 0.7 | 49 ± 1.2 | 4 × 10⁻³ | 17.3 ± 0.3 | 18 ± 0.3 | 1 × 10⁻⁵ |
| 0.4 | 35.3 ± 0.7 | 34 ± 0.6 | 1 × 10⁻¹⁷ | 19.4 ± 0.3 | 20 ± 0.3 | 2 × 10⁻²³ |

Gene-Expression Study in Drosophila.

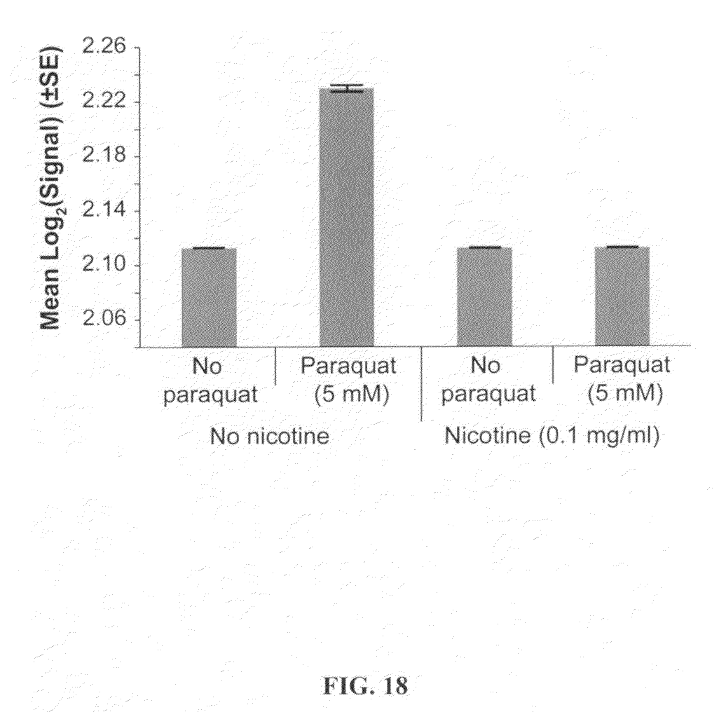

Test of statistical interaction between paraquat and nicotine on 18 954 transcripts gave a very strong signal for CG14691 (P_{Interaction}=2×10⁻¹¹, adjusted for multiple testing P_{Interaction}=4×10⁻⁷), followed by marginal signals for skpB (P_{Interaction}=1×10⁻⁶, adjusted P_{Interaction}=0.01) and CG1885 (P_{Interaction}=5×10⁻⁶, adjusted P_{Interaction}=0.03). FIG. 18 shows that compared to untreated flies, paraquat-treated flies had a modest (8%) but highly significant (P=5×10⁻⁸) rise in CG14691 expression, whereas flies that were treated with nicotine+paraquat or nicotine alone were similar to untreated flies (P=1.0).

CG14691 is predicted to encode a synaptic vesicle membrane protein (Flybase website). Genome comparison analysis revealed that the Drosophila CG14691 is orthologous to the SV2A/SV2B/SV2C family of synaptic vesicle proteins in humans²². The next step was to look for evidence of interaction between SV2 genes and the protective effect of smoking on PD in humans.

Genome-Wide Gene-Environment Interaction in Human.

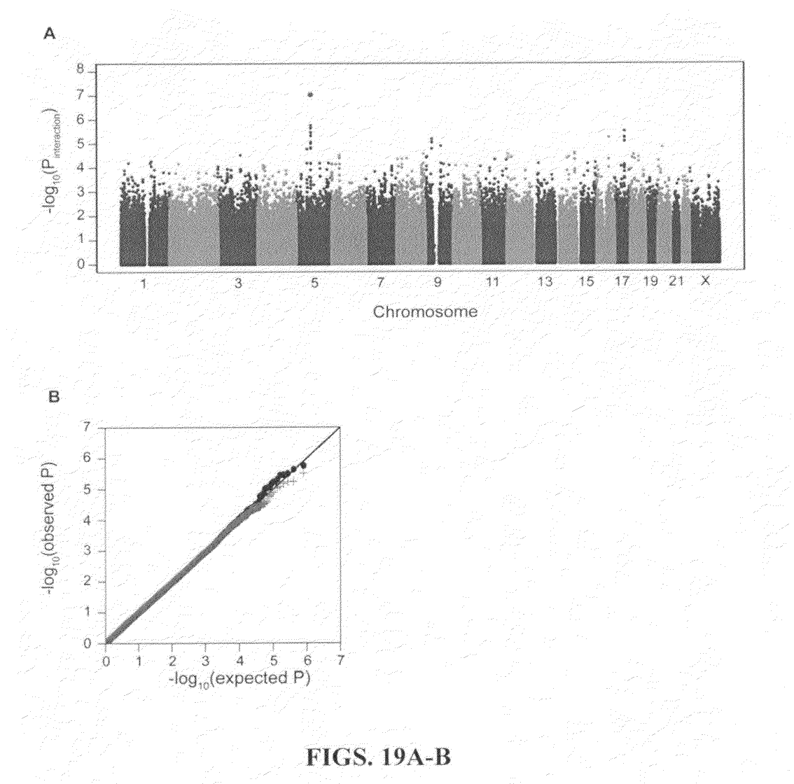

As depicted in the Manhattan plot of genome-wide interaction with smoking (FIG. 19), none of the SNPs achieved the genome-wide significance threshold of P<5×10⁻⁸. However, the highest peak was on chromosome 5 and mapped to the 5′ of the synaptic vesicle protein SV2C gene (FIG. 19, Table 20). Although the significance of SV2C (P=2×10⁻⁶ for single-SNP and P=9×10⁻⁸ for the joint effect of two independent SNPs) did not meet genome-wide significance criteria for discovery, it surpassed the significance threshold for validation of a candidate gene that was discovered, at genome-wide significance, in Drosophila.

TABLE 20
Human: SNPs that achieved P_{Interaction} < 5 × 10⁻⁶ in SNP*
smoking interaction test. The SNPs' main effects on PD were
negligible (OR = 0.88-1.02, P = 0.02-0.77) but they rose in significance
when tested for interaction with smoking (P = 4.8 × 10⁻⁶-1.7 × 10⁻⁶)

| | | | | | | | SNP | | SNP* | |
|---|---|---|---|---|---|---|---|---|---|---|
| | | | | | | | main | | smoking | |
| | | Base- | Minor | | | | effect | | Interaction | |
| Ch | SNP | pair | allele | Gene | Location | MAF | OR | P | OR | P |
| 5 | rs30196 | 754133 | A | SV2C | 5 prime | 0.46 | 0.89 | 0.03 | 1.68 | 1.7 × 10⁻⁶ |
| | | 07 | | | | | | | | |
| 5 | rs28621 | 754175 | T | SV2C | Intron 1 | 0.45 | 0.89 | 0.04 | 1.67 | 2.2 × 10⁻⁶ |
| | | 59 | | | | | | | | |
| 17 | rs22872 | 507478 | A | HLF | Intron 3 | 0.38 | 1.02 | 0.77 | 1.58 | 3.1 × 10⁻⁶ |
| | 23 | 51 | | | | | | | | |
| 5 | rs29377 | 754518 | C | SV2C | Intron 1 | 0.46 | 0.89 | 0.04 | 1.66 | 3.4 × 10⁻⁶ |
| | 05 | 10 | | | | | | | | |
| 5 | rs27317 | 754319 | C | SV2C | Intron 1 | 0.46 | 0.90 | 0.04 | 1.66 | 3.5 × 10⁻⁶ |
| | 35 | 92 | | | | | | | | |
| 5 | rs26526 | 753782 | C | SV2C | 5 prime | 0.45 | 0.88 | 0.02 | 1.64 | 4.8 × 10⁻⁶ |
| | 01 | 65 | | | | | | | | |

Only genotyped SNPs are shown.

SNP main effect was calculated using logistic regression using robust sandwich variance estimation in R adjusted for age, sex and PC1 and 2, without smoking in the model.

MAF=Minor allele frequency in cases and controls combined.

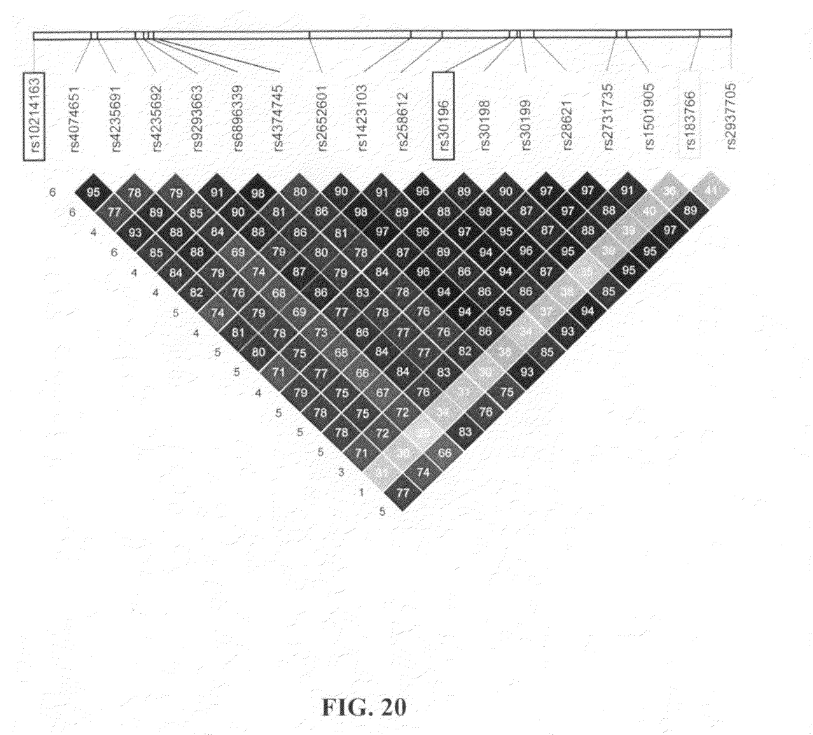

Eighteen SNPs spanning from ˜90 kb upstream to intron 1 of SV2C with 10⁻³>P_{Interaction}≧2×10⁻⁶ formed three haplotype blocks (FIG. 20) tagged by rs10214163 (minor allele frequency (MAF)=0.21, P_{Interaction}=4×10⁻⁴), rs30196 (MAF=0.46, P_{Interaction}=2×10⁻⁶), and rs183766 (MAF=0.33, P_{Interaction}=4×10⁻⁴). When Applicants conditioned on rs30196*smoking interaction and tested for independent effects of the other two signals, the rs183766*smoking signal was abolished (P_{Interaction}=0.51), but rs10214163*smoking remained significant (P_{Interaction}=0.01). In an additive two-SNP model, where each individual was assigned a genotype with 0, 1, 2, 3 or 4 minor alleles for rs30196 and rs10214163, SV2C*smoking interaction test yielded P_{Interaction}=9×10⁻⁸. These data suggest rs30196 and rs10214163 mark two distinct disease associated variants with additive effects.

Applicants tested whether SV2C was associated with smoking per se. Using the combined genotypes at rs30196 and rs10214163, Applicants found no evidence for association with smoking in cases and controls combined (OR=1.03, P=0.36). Therefore, the signal for SV2C-smoking interaction on PD risk (P=9×10⁻⁸) cannot be attributed to an association between the gene and the smoking habit. Furthermore, Applicants found the evidence for this interaction to be robust across all disease-related strata including familial and sporadic PD, early and late onset, males and females, Jewish and non-Jewish ancestry, and there was no evidence for significant heterogeneity across European countries of ancestral origin (Table 17).

TABLE 17
Human: SV2C*smoking interaction in strata
defined by disease related variables.
Evidence for interaction was present in all strata and there was no
significant heterogeneity across European countries of origin as reported
by participants and verified by principal component analysis ¹¹.

| Strata | N case | N control | OR_{Interaction} | SE (OR) | P_{Interaction} | P_{Heterogeneity} |
|---|---|---|---|---|---|---|
| All | 1600 | 1506 | 1.50 | 0.11 | 9.35 × 10⁻⁸ | |
| PD-associated risk factors | | | | | | |
| Familial PD | 346 | 1506 | 1.57 | 0.19 | 2.25 × 10⁻⁴ | |
| Sporadic PD | 1254 | 1506 | 1.48 | 0.12 | 1.51 × 10⁻⁶ | NC |
| Male | 1082 | 606 | 1.44 | 0.15 | 4.33 × 10⁻⁴ | |
| Female | 518 | 900 | 1.51 | 0.17 | 3.66 × 10⁻⁴ | 0.78 |
| Early onset (≦50 years) | 416 | 1506 | 1.44 | 0.18 | 3.70 × 10⁻³ | |
| Late onset (>50 years) | 1184 | 1506 | 1.53 | 0.12 | 1.49 × 10⁻⁷ | NC |
| Coffee—heavy | 510 | 385 | 1.34 | 0.19 | 0.04 | |
| Coffee—light | 940 | 545 | 1.65 | 0.19 | 2.34 × 10⁻⁵ | 0.26 |
| OTC NSAIDs—ever | 969 | 657 | 1.33 | 0.14 | 6.56 × 10⁻³ | |
| OTC NSAIDs—never | 588 | 326 | 1.82 | 0.27 | 4.29 × 10⁻⁵ | 0.08 |
| Recruitment site | | | | | | |
| New York | 376 | 294 | 1.55 | 0.25 | 7.08 × 10⁻³ | |
| Oregon | 241 | 633 | 1.53 | 0.29 | 0.03 | |
| Georgia | 230 | 113 | 1.16 | 0.27 | 0.53 | |
| Washington | 753 | 466 | 1.54 | 0.19 | 4.61 × 10⁻⁴ | 0.80 |
| Ashkenazi Jewish | | | | | | |
| (genetically defined | | | | | | |
| by principal components) | | | | | | |
| Yes | 73 | 36 | 2.18 | 0.87 | 0.05 | |
| No | 1527 | 1470 | 1.48 | 0.12 | 4.51 × 10⁻⁷ | 0.34 |
| Paternal and Maternal ancestry | | | | | | |
| Great Britain | 105 | 61 | 1.14 | 0.43 | 0.73 | |
| Germany/Austria | 87 | 44 | 1.30 | 0.57 | 0.55 | |
| Ireland | 37 | 16 | 3.13 | 2.61 | 0.17 | |
| Scandinavia | 48 | 29 | 0.99 | 0.51 | 0.99 | |
| Eastern Europe | 31 | 28 | 0.76 | 0.43 | 0.63 | |
| Italy | 47 | 34 | 5.68 | 3.76 | 8.58 × 10⁻³ | |
| Russia | 17 | 10 | 3.08 | 3.37 | 0.30 | 0.13 |
| Paternal or Maternal ancestry | | | | | | |
| Great Britain | 445 | 284 | 1.62 | 0.26 | 3.13 × 10⁻³ | |
| Germany/Austria | 357 | 213 | 1.25 | 0.23 | 0.22 | |
| Ireland | 196 | 138 | 1.67 | 0.42 | 0.04 | |
| Scandinavia | 187 | 112 | 1.36 | 0.36 | 0.26 | |
| Eastern Europe | 78 | 66 | 1.93 | 0.62 | 0.04 | |
| Italy | 76 | 62 | 4.23 | 1.97 | 2.04 × 10⁻³ | |
| France | 68 | 50 | 1.60 | 0.62 | 0.23 | |
| Russia | 48 | 20 | 1.73 | 0.98 | 0.34 | 0.39 |

All tests were performed using robust sandwich variance estimation in R, adjusted for age, sex (except for male and female strata), PC1, and PC2. Genotype is defined as the number of minor alleles at the two loci (rs30196+rs10214163) an individual possesses: 0, 1, 2, 3 or 4. Genotype effect was treated in an additive model for the number of minor alleles. Tests were conducted for interaction of genotype*smoking. OTC NSAIDs=over-the-counter nonsteroidal anti-inflammatory drug use. NC: Not calculated because the same controls were used for both strata.

The effect of smoking on PD risk was significantly different across genotypes (P_{Heterogeneity}=4×10⁻¹⁰, Table 18). When classified by the genotypes at the two independent SNPs, the effect of smoking on PD risk changed incrementally by the number of minor alleles from 56% risk reduction (P=1×10⁻⁶) for individuals homozygous for the common (major) alleles at rs30196 and rs10214163, to 223% risk increase (P=0.04) for individuals homozygous for the minor alleles. Applicants' data suggest that only a fraction of the population benefits from the protective effect of nicotine, and that this group can be identified by genotyping SV2C.

TABLE 18
Human: Variation in the effect of smoking on PD risk according to SV2C
genotype. The effect of smoking on PD risk varied significantly, changing incrementally
and consistently with the number of minor alleles from highly beneficial to harmful
(odds ratio OR = 0.44, 56% risk reduction to OR = 3.23, 223% risk increase).

| No. | SV2C | | | | | | | | |
|---|---|---|---|---|---|---|---|---|---|
| of | genotype | | | | | PD risk smoker vs. | | | |
| minor | rs30196- | Genotype | | N | N | non-smoker | | | Heterogeneity * |
| alleles | rs10214163 | frequency | Smoking | case | control | OR | SE | P | P |
| Irrespective of | | 1.00 | No | 866 | 811 | 0.81 | 0.06 | 8 × 10⁻³ | |
| genotype | | | Yes | 734 | 695 | | | | |
| 0 | CC-TT | 0.23 | No | 242 | 168 | 0.44 | 0.08 | 1 × 10⁻⁶ | Ref |
| | | | Yes | 145 | 162 | | | | |
| 1 | CA-TT | 0.37 | No | 321 | 278 | 0.81 | 0.10 | 0.10 | 4 × 10⁻³ |
| | CC-CT | | Yes | 281 | 264 | | | | |
| | AA-TT | | No | 204 | 239 | | | | |
| 2 | CA-CT | 0.27 | Yes | 199 | 190 | 0.98 | 0.15 | 0.88 | 5 × 10⁻⁴ |
| | CC-CC | | | | | | | | |
| 3 | AA-CT | 0.11 | No | 87 | 97 | 1.42 | 0.35 | 0.16 | 9 × 10⁻⁵ |
| | CA-CC | | Yes | 90 | 66 | | | | |
| 4 | AA-CC | 0.02 | No | 12 | 28 | 3.23 | 1.85 | 0.04 | 8 × 10⁻⁴ |
| | | | Yes | 19 | 13 | | | | |

| | |
|---|---|
| Heterogeneity across all five genotypic groups | 4 × 10⁻¹⁰ |

* Testing the difference between genotypes for the effect of smoking on PD risk

Applicants set out to find genes that modulate the effect of smoking on PD risk reduction, with the goal of carrying them forward as markers into upcoming clinical trials of nicotine for PD. Applicants used an integrated approach where Applicants conducted genome-wide studies in Drosophila and in humans, in parallel. The human genome-wide interaction study did not achieve the genome-wide significance threshold of 5×10⁻⁸; which was not surprising considering that detecting interactions requires much larger sample sizes than the standard genome-wide association studies²³. The Drosophila study, however, revealed a gene with genome-wide significance. Applicants had planned that if the Drosophila study were successful in identifying a gene, Applicants would test the association of its human homologue with PD to establish its relevance to disease. Surprisingly, the gene that emerged from the Drosophila study (CG14691) was a homologue of the gene that displayed the most significant evidence for interaction with smoking in the human study (SV2C). Thus, while SV2C did not achieve the genome-wide significance level of 5×10⁻⁸ required to qualify as a discovery, it did reach a highly significant level (P<2×10⁻⁶, or 9×10⁻⁸) to qualify as a validation of the discovery made in the fly. Applicants also noted that SV2C genotype was not associated with the smoking habit. Thus, Applicants' data point to an interactive effect of SV2C genotype and nicotine on protection against parkinsonism.

The Drosophila and the human experiments were set up with the same goal of identifying genes that are involved in protection by smoking, although the study designs were inherently quite different. The fly experiment was done on a uniform genetic background, parkinsonism was induced with a single neurotoxin, and rescue was with controlled doses of pure nicotine. In contrast, humans were genetically diverse, represented an unknown level of heterogeneity in the causes of PD, and were exposed to all ˜600 toxins in cigarettes. Furthermore, in flies, paraquat and nicotine were the predictors and gene expression the outcome; whereas in humans, genes and smoking were the predictors and PD the outcome. That two distinct, hypothesis-free experiments conducted in two species converge on a pair of homologues (CG14691 and SV2C) as the most significant genes argues for an important role for SV2C in the pathogenesis of parkinsonism and protection by nicotine.

In the brain, nicotine binds to nicotinic acetylcholine receptors with high affinity and enhances vesicular release of dopamine²⁴. Dopamine depletion is a hallmark of PD. A growing body of work has implicated altered synaptic transmitter release in the pathogenesis of PD²⁵. Synaptic vesicle proteins SV2A/SV2B/SV2C are integral membrane components of synaptic vesicles and have been implicated in storage and release of neurotransmitters^{26,27}. A recent study has shown that modest changes in SV2 expression, in either direction, can have a significant impact on synaptic function²⁸. SV2C is densely expressed in dopaminergic neurons in substantia nigra²⁹. The findings of Applicants' study may therefore reflect a connection between nicotinic enhancement of vesicular dopamine release and altered neurotransmission due to changes in expression of SV2C.

The association of smoking with PD was genotype-specific and varied from highly protective to neutral and even harmful depending on SV2C genotype. This suggests that efficacy of nicotine as a neuroprotective treatment will not be uniform for all individuals and that clinical trials may benefit from pre-classification of subjects as high and low responders based on genotype. Nicotine has long been considered as a possible therapeutic agent for PD³⁰. Clinical studies of the symptomatic efficacy of transdermal nicotine treatment for PD have been relatively small, and most, but not all, have shown a beneficial effect on motor and cognitive functions³¹⁻³⁴. A randomized, placebo-controlled, double-blind multi-center trial was recently launched, with a larger sample size than attempted previously, to examine for the first time disease modifying potential of a nicotine patch for early PD (The Michael J. Fox foundation website). This study provides an opportunity to evaluate utility of SV2C genotype for improving power and precision in assessing the efficacy of transdermal nicotine treatment. Applicants acknowledge the distinction that Applicants' study examined risk of developing PD, whereas the trials are aimed at disease modification after onset of symptoms. It is however possible that nicotine plays a similar role in disease process before and after the onset of symptoms, a simple hypothesis that can be tested easily in any clinical trial setting.

In summary, Applicants have identified a novel PD-associated gene, SV2C, via interaction with protective effect of smoking/nicotine. Taken together, the cross-validating evidence from human and Drosophila studies, and the biological plausibility of the pathway that has emerged, suggest that SV2C plays a role in the pathogenesis of PD and that SV2C genotype might be a useful marker for pharmacogenomics prevention and treatment studies of PD involving nicotine.

NGRC genome-wide genotype, phenotype, and environmental data are available from dbGaP (The NCBI website, accession number phs000196.v2.p1). Microarray data on Drosophila paraquat-nicotine model are available from Gene Expression Omnibus (The NCBI website).

The Example was funded by grants from the National Institute of Neurological Disorders And Stroke (R01NS36960 and R01NS067469). Additional support was provided by a Merit Review Award from the Department of Veterans Affairs (1101BX000531), National Institutes of Aging (P30AG08017), Office of Research & Development, Clinical Sciences Research & Development Service, Department of Veteran Affairs, and the Close to the Cure Foundation. Genotyping services were provided by the Center for Inherited Disease Research which is funded by the National Institutes of Health (HHSN268200782096C).

### REFERENCES

1. Hernan M A, Takkouche B, Caamano-Isorna F, Gestal-Otero J J. A meta-analysis of coffee drinking, cigarette smoking, and the risk of Parkinson's disease. Ann Neurol 2002; 52(3): 276-284.
2. Powers K, Kay D, Factor S, Zabetian C, Higgins D, Samii A, et al. Combined effects of smoking, coffee and NSAIDs on Parkinson's disease risk. Mov Disord 2008; 23(1): 88-95.
3. Quik M, Parameswaran N, McCallum S E, Bordia T, Bao S, McCormack A, et al. Chronic oral nicotine treatment protects against striatal degeneration in MPTP-treated primates. J Neurochem 2006; 98(6): 1866-1875.
4. Chen J F, Xu K, Petzer J P, Staal R, Xu Y H, Beilstein M, et al. Neuroprotection by caffeine and A(2A) adenosine receptor inactivation in a model of Parkinson's disease. J Neurosci 2001; 21(10): RC143.
5. Evans A H, Lawrence A D, Potts J, MacGregor L, Katzenschlager R, Shaw K, et al. Relationship between impulsive sensation seeking traits, smoking, alcohol and caffeine intake, and Parkinson's disease. J Neurol Neurosurg Psychiatry 2006; 77(3): 317-321.
6. Hamza T H, Chen H, Hill-Burns E M, Rhodes S L, Montimurro J, Kay D M, et al. Genome-Wide Gene-Environment Study Identifies Glutamate Receptor Gene GRIN2A as a Parkinson's Disease Modifier Gene via Interaction with Coffee. PLoS Genet 2011; 7(8): e1002237.
7. Costello S, Cockburn M, Bronstein J, Zhang X, Ritz B. Parkinson's disease and residential exposure to maneb and paraquat from agricultural applications in the central valley of California. Am J Epidemiol 2009; 169(8): 919-926.
8. Wang A, Costello S, Cockburn M, Zhang X, Bronstein J, Ritz B. Parkinson's disease risk from ambient exposure to pesticides. European journal of epidemiology 2011; 26(7): 547-555.
9. Tanner C M, Kamel F, Ross G W, Hoppin J A, Goldman S M, Korell M, et al. Rotenone, paraquat, and Parkinson's disease. Environ Health Perspect 2011; 119(6): 866-872.
10. Chaudhuri A, Bowling K, Funderburk C, Lawal H, Inamdar A, Wang Z, et al. Interaction of genetic and environmental factors in a Drosophila parkinsonism model. J Neurosci 2007; 27(10): 2457-2467.
11. Hamza T H, Zabetian C P, Tenesa A, Laederach A, Montimurro J, Yearout D, et al. Common genetic variation in the HLA region is associated with late-onset sporadic Parkinson's disease. Nat Genet 2010; 42(9): 781-785.
12. Hughes A J, Daniel S E, Ben-Shlomo Y, Lees A J. The accuracy of diagnosis of parkinsonian syndromes in a specialist movement disorder service. Brain 2002; 125(Pt 4): 861-870.
13. Aulchenko Y, Struchalin M, van Duijn C. ProbABEL package for genome-wide association analysis of imputed data. BMC Bioinformatics 2010; 11(1): 134.
14. White H. A Heteroskedasticity-Consistent Covariance Matrix Estimator and a Direct Test for Heteroskedasticity. Econometrica 1980; 48(4): 817-838.
15. Barrett J C, Fry B, Mailer J, Daly M J. Haploview: analysis and visualization of LD and haplotype maps. Bioinformatics 2005; 21(2): 263-265.
16. Wang K, Li M, Hadley D, Liu R, Glessner J, Grant S F, et al. PennCNV: an integrated hidden Markov model designed for high-resolution copy number variation detection in whole-genome SNP genotyping data. Genome Res 2007; 17(11): 1665-1674.
17. Kaplan E, Meier E L. Nonparametric estimation from incomplete observations. J Am Statist Assoc 1958; 53: 457-481.
18. Gentleman R, Carey V, Bates D, Bolstad B, Dettling M, Dudoit S, et al. Bioconductor: open software development for computational biology and bioinformatics. Genome Biology 2004; 5(10): R80.
19. Wu Z, Irizarry R A. Preprocessing of oligonucleotide array data. 2004; 22(6): 656-658.
20. Smyth G. limma: Linear Models for Microarray Data. In: Gentleman R, Carey V J, Huber W, Irizarry R A, Dudoit S (eds). Bioinformatics and Computational Biology Solutions Using R and Bioconductor. Springer: New York, 2005, pp 397-420.
21. Benjamini Y, Hochberg Y. Controlling the false discovery rate: A practical and powerful approach to multiple testing. J Roy Statist Soc Ser B 1995; 57: 289-300.
22. Hu Y, Flockhart I, Vinayagam A, Bergwitz C, Berger B, Perrimon N, et al. An integrative approach to ortholog prediction for disease-focused and other functional studies. BMC Bioinformatics 2011; 12(1): 357.
23. Thomas D. Gene-environment-wide association studies: emerging approaches. Nat Rev Genet 2010; 11(4): 259-272.
24. Turner T J. Nicotine enhancement of dopamine release by a calcium-dependent increase in the size of the readily releasable pool of synaptic vesicles. J Neurosci 2004; 24(50): 11328-11336.
25. Esposito G, Ana Clara F, Verstreken P. Synaptic vesicle trafficking and Parkinson's disease. Dev Neurobiol 2012; 72(1): 134-144.
26. Feany M B, Lee S, Edwards R H, Buckley K M. The synaptic vesicle protein SV2 is a novel type of transmembrane transporter. Cell 1992; 70(5): 861-867.
27. Dardou D, Dassesse D, Cuvelier L, Deprez T, De Ryck M, Schiffmann S N. Distribution of SV2C mRNA and protein expression in the mouse brain with a particular emphasis on the basal ganglia system. Brain Res 2011; 1367: 130-145.
28. Nowack A, Malarkey E B, Yao J, Bleckert A, Hill J, Bajjalieh S M. Levetiracetam reverses synaptic deficits produced by overexpression of SV2A. PLoS One 2011; 6(12): e29560.
29. Janz R, Sudhof T C. SV2C is a synaptic vesicle protein with an unusually restricted localization: anatomy of a synaptic vesicle protein family. Neuroscience 1999; 94(4): 1279-1290.
30. Quik M, O'Leary K, Tanner C M. Nicotine and Parkinson's disease: implications for therapy. Mov Disord 2008; 23(12): 1641-1652.
31. Itti E, Villafane G, Malek Z, Brugieres P, Capacchione D, Itti L, et al. Dopamine transporter imaging under high-dose transdermal nicotine therapy in Parkinson's disease: an observational study. Nuclear medicine communications 2009; 30(7): 513-518.
32. Villafane G, Cesaro P, Rialland A, Baloul S, Azimi S, Bourdet C, et al. Chronic high dose transdermal nicotine in Parkinson's disease: an open trial. Eur J Neurol 2007; 14(12): 1313-1316.
33. Lemay S, Chouinard S, Blanchet P, Masson H, Soland V, Beuter A, et al. Lack of efficacy of a nicotine transdermal treatment on motor and cognitive deficits in Parkinson's disease. Progress in neuro psychopharmacology & biological psychiatry 2004; 28(1): 31-39.
34. Vieregge A, Sieberer M, Jacobs H, Hagenah J M, Vieregge P. Transdermal nicotine in PD: a randomized, double-blind, placebo-controlled study. Neurology 2001; 57(6): 1032-1035.

The invention is further described by the following numbered paragraphs:
1. A method of identifying in a patient or person a genetic predisposition to Parkinson disease (PD), wherein said method comprises:
   (a) obtaining a blood, saliva or tissue sample from the patient or person;
   (b) analyzing DNA from the blood, saliva or tissue sample for the presence or absence of a polymorphic site in a gene listed in Tables A, B, C or D, whereby the presence of the polymorphic site identifies a gene variant;
      wherein the presence of the gene variant indicates a higher predisposition to PD, and the absence of the gene variant indicates a lower predisposition to PD, compared to a control sample.
2. The method of paragraph 1, wherein PD is sporadic PD or late-onset PD.
3. The method of paragraph 1, wherein the polymorphic site is a single nucleotide polymorphism (SNP).
4. The method of paragraph 3, wherein the SNP is at a position listed in Tables A, B, C or D.
5. The method of paragraph 4, wherein the SNP is at a position about 200 kb upstream or about 200 kb downstream from a position listed in Tables A, B, C or D.
6. The method of paragraph 5, wherein the SNP is at a position about 50 kb upstream or about 50 kb downstream from a position listed in Tables A, B, C or D.
7. The method of paragraph 6, wherein the SNP is at a position about 1 kb upstream or about 1 kb downstream from a position listed in Tables A, B, C or D.
8. The method of paragraph 1, wherein the gene is on chromosome 6.
9. The method of paragraph 8, wherein the gene is HLA.
10. The method of paragraph 9, wherein the polymorphic site is a SNP on intron-1.
11. The method of paragraph 10, wherein the polymorphic site is a SNP at position 32517508.
12. The method of paragraph 1, further comprising determining whether the patient smokes cigarettes, consumes caffeinated coffee, and/or uses NSAIDs, wherein a predisposition to developing PD is associated, inversely, with cigarette smoking, caffeinated coffee consumption and NSAID use.
13. The method of paragraph 12, wherein the patient is identified via a questionnaire, personal interview or medical history analysis.
14. A method of testing a blood, saliva or tissue sample of a patient for the presence of a polymorphic site, wherein said method comprises:
   (a) obtaining a blood, saliva or tissue sample from the patient;
   (b) analyzing the DNA from the blood, saliva or tissue sample for the presence or absence of a polymorphic site in a gene listed in Tables A, B, C or D, whereby the presence of the polymorphic site identifies a gene variant;
      wherein the presence of the gene variant indicates a higher predisposition to PD, and the absence of the gene variant indicates a lower predisposition to PD, compared to a control sample.
15. The method of paragraph 14, wherein PD is sporadic PD or late-onset PD.
16. The method of paragraph 14, wherein the polymorphic site is a single nucleotide polymorphism (SNP).
17. The method of paragraph 16, wherein the SNP is at a position listed in Tables A, B, C or D.
18. The method of paragraph 17, wherein the SNP is at a position about 200 kb upstream or about 200 kb downstream from a position listed in Tables A, B, C or D.
19. The method of paragraph 18, wherein the SNP is at a position about 50 kb upstream or about 50 kb downstream from a position listed in Tables A, B, C or D.
20. The method of paragraph 19, wherein the SNP is at a position about 1 kb upstream or about 1 kb downstream from a position listed in Tables A, B, C or D.
21. The method of paragraph 14, wherein the gene is on chromosome 6.
22. The method of paragraph 21, wherein the gene is HLA.
23. The method of paragraph 22, wherein the polymorphic site is a SNP on intron-1.
23. The method of paragraph 23, wherein the polymorphic site is a SNP at position 32517508.
24. The method of paragraph 14, further comprising determining whether the patient smokes cigarettes, consumes caffeinated coffee, and/or uses NSAIDs, wherein a predisposition to developing PD is associated, inversely, with cigarette smoking, caffeinated coffee consumption and NSAID use.
25. The method of paragraph 24, wherein the patient is identified via a questionnaire, personal interview or medical history analysis.
26. A method of treating, or inhibiting the development of, PD in a patient or person, comprising:
   (a) determining in a patient the presence or absence of a polymorphic site in a gene listed in Tables A, B, C or D, whereby the presence of the polymorphic site identifies a gene variant,
      wherein the presence of the gene variant indicates a higher predisposition to PD, and the absence of the gene variant indicates a lower predisposition to PD, compared to a control sample; and
   (b) administering to the patient or person having the gene variant a therapeutically effective amount of a medicament comprising one or more non-steroidal anti-inflammatory drugs (NSAIDs), caffeine or caffeine analogs, nicotine or nicotine analogs, or combinations thereof, wherein the medicament treats, or inhibits the development of, PD.
27. The method of paragraph 26, wherein component (a) comprises:
   (i) obtaining a blood, saliva or tissue sample from the patient or person,
   (ii) isolating DNA from the blood, saliva or tissue sample, and
   (iii) genotyping the DNA.
28. The method of paragraph 26, wherein the one or more NSAID is selected from the group consisting of: ibuprofen, aspirin, naproxen, indomethacin, nabumetone, nimesulide, selective cyclooxygenase (COX) inhibitors, and licofelone.
29. The method of paragraph 26, wherein the medicament may further comprise levodopa, a dopamine agonist, a catechol 0-methyltransferase (COMT) inhibitor, a monoamine oxidase B (MAO B) inhibitor, an anticholinergic agent, an antiviral agent or a combination thereof.
30. The method of paragraph 26, wherein the medicament is administered orally, intravenously or topically.
31. The method of paragraph 26, wherein PD is sporadic PD or late-onset PD.
32. The method of paragraph 26, wherein the polymorphic site is a single nucleotide polymorphism (SNP).
33. The method of paragraph 32, wherein the SNP is at a position listed in Tables A, B, C or D.
34. The method of paragraph 33, wherein the SNP is at a position about 200 kb upstream or about 200 kb downstream from a position listed in Tables A, B, C or D.
35. The method of paragraph 34, wherein the SNP is at a position about 50 kb upstream or about 50 kb downstream from a position listed in Tables A, B, C or D.
36. The method of paragraph 35, wherein the SNP is at a position about 1 kb upstream or about 1 kb downstream from a position listed in Tables A, B, C or D.
37. The method of paragraph 26, wherein the gene is on chromosome 6.
38. The method of paragraph 37, wherein the gene is HLA.
39. The method of paragraph 38, wherein the polymorphic site is a SNP on intron-1.
40. The method of paragraph 39, wherein the polymorphic site is a SNP at position 32517508.
41. The method of paragraph 26, further comprising determining whether the patient smokes cigarettes, consumes caffeinated coffee, and/or uses NSAIDs, wherein a predisposition to developing PD is associated, inversely, with cigarette smoking, caffeinated coffee consumption and NSAID use.
42. The method of paragraph 41, wherein the patient is identified via a questionnaire, personal interview or medical history analysis.
43. A kit comprising:
   (a) a medicament comprising one or more NSAIDs, caffeine or caffeine analogs, nicotine or nicotine analogs, or combinations thereof; and
   (b) optionally instructions for administering the medicament to a patient having a genetic predisposition to PD, identified by the method of paragraph 1.
44. The kit of paragraph 43, further comprising levodopa, a dopamine agonist, a catechol O-methyltransferase (COMT) inhibitor, a monoamine oxidase B (MAO B) inhibitor, an anticholinergic agent, an antiviral agent or combinations thereof
45. The kit of paragraph 43, wherein the medicament comprises a pharmaceutically acceptable excipient or carrier.
46. A method for identifying the heritage of an individual, comprising:
   (a) obtaining a blood, saliva or tissue sample from the individual;
   (b) analyzing the DNA from the blood, saliva or tissue sample for the presence or absence of one or more genetic markers;
   (c) assigning a genetic profile to the individual based on the presence or absence of the one or more genetic markers; and
   (d) correlating the genetic profile with a geographic location and ethnic heritage; and
   (e) identifying the heritage of an individual based on the correlation.
47. The method of paragraph 46, wherein the individual is of Jewish heritage.
48. The method of paragraph 46, wherein the geographic location is Eastern Europe, France, Great Britain, Germany-Austria, Holland, Ireland, Italy, Russia, Scandinavia.
49. A method of treating, or inhibiting the development of, PD in a patient, comprising:
   (a) determining in a patient the presence or absence of a polymorphic site at or within about 100 kb of one or more positions listed in Tables A, B, C or D, whereby the presence of the polymorphic site indicates a higher predisposition to PD, and the absence of the polymorphic site indicates a lower predisposition to PD, compared to a control sample; and
   (b) identifying what gene has the polymorphic site, thereby identifying a gene variant;
   (c) optionally determining if the gene variant increases or decreases the neuroprotective effect of smoking/nicotine, coffee/caffeine, or NSAID consumption;
   (d) administering to the patient having the polymorphic site a therapeutically effective amount of a medicament comprising one or more NSAIDs, caffeine or caffeine analogs, nicotine or nicotine analogs, or combinations thereof,
   (e) wherein the medicament treats, or inhibits the development of, PD.
50. A method of identifying in a patient a genetic predisposition to an HLA-associated disease, wherein said method comprises:
   (a) obtaining a blood, saliva or tissue sample from the patient;
   (b) analyzing DNA from the blood, saliva or tissue sample for the presence or absence of a polymorphic site in a HLA gene, whereby the presence of the polymorphic site identifies a gene variant;
      wherein the presence of the gene variant indicates a higher predisposition to the HLA-associated disease, and the absence of the gene variant indicates a lower predisposition to the HLA-associated disease, compared to a control sample.
51. The method of paragraph 50, wherein the HLA-associated disease is a cancer, an autoimmune disease, or an infectious disease.
52. The method of paragraph 50, wherein the polymorphic site is a single nucleotide polymorphism (SNP).
53. The method of paragraph 52, wherein the SNP is at position 32517508 on chromosome 6.
54. The method of paragraph 53, wherein the SNP is at a position about 200 kb upstream or about 200 kb downstream from position 32517508.
55. The method of paragraph 54, wherein the SNP is at a position about 50 kb upstream or about 50 kb downstream from position 32517508.
56. The method of paragraph 55, wherein the SNP is at a position about 1 kb upstream or about 1 kb downstream from position 32517508.
57. The method of paragraph 51, wherein the HLA-associated disease is primary hemochromatosis, ankylosing spondylitis, inflammatory bowel, reiter's disease, psoriatic arthritis, system lupus erythematosus, rheumatoid arthritis, Graves' disease, celiac sprue, multiple sclerosis, hay fever, Goodpasture's syndrome, Sjögren's syndrome, pernicious anemia, Hashimoto's thyroiditis, narcolepsy, lyme disease, pemphigus vulgaris, Type 1 diabetes mellitus, acute uveitis, or psoriasis.
101. A method of identifying in a person a genetic predisposition to Parkinson disease (PD), wherein said method comprises: analyzing DNA from a blood, saliva or tissue sample obtained from the person for the presence or absence of a polymorphic site in a gene listed in Tables A, B, C or D, whereby the presence of the polymorphic site identifies a gene variant; wherein the presence of the gene variant indicates a higher predisposition to PD, and the absence of the gene variant indicates a lower predisposition to PD, as compared to an individual with PD and the presence of a polymorphic site in a gene listed in Tables A, B, C or D or an individual without PD and the absence of a polymorphic site in a gene listed in Tables A, B, C or D.
102. The method of paragraph 101, wherein PD is sporadic PD or late-onset PD.
103. The method of paragraph 101, wherein the polymorphic site is a single nucleotide polymorphism (SNP).
104. The method of paragraph 101, wherein the gene is listed in Table A.
105. The method of paragraph 101, wherein the gene is HLA.
106. The method of paragraph 101, wherein the gene is GRIN2A.
107. The method of paragraph 101, further comprising determining whether the person smokes cigarettes, consumes caffeinated coffee, and/or uses NSAIDs, wherein a predisposition to developing PD is associated, inversely, with cigarette smoking, caffeinated coffee consumption and NSAID use.
108. A method of treating or inhibiting the development of PD in a person, comprising:
(a) determining in a person the presence or absence of a polymorphic site in a gene listed in Tables A, B, C or D, whereby the presence of the polymorphic site identifies a gene variant,
wherein the presence of the gene variant indicates a higher predisposition to PD, and the absence of the gene variant indicates a lower predisposition to PD, as compared to an individual with PD and the presence of a polymorphic site in a gene listed in Tables A, B, C or D or an individual without PD and the absence of a polymorphic site in a gene listed in Tables A, B, C or D;
(b) optionally determining if the gene variant increases or decreases the neuroprotective effect of smoking/nicotine, coffee/caffeine, or NSAID consumption and
(c) administering to the person having the gene variant a therapeutically effective amount of a medicament comprising one or more non-steroidal anti-inflammatory drugs (NSAIDs), caffeine or caffeine analogs, nicotine or nicotine analogs, or a combination thereof,
wherein the medicament treats, or inhibits the development of, PD.
109. The method of paragraph 108, wherein step (a) comprises:
(i) obtaining a blood, saliva or tissue sample from the person,
(ii) isolating DNA from the blood, saliva or tissue sample, and
(iii) genotyping the DNA.
110. The method of paragraph 108, wherein the one or more NSAID is selected from the group consisting of: ibuprofen, aspirin, naproxen, indomethacin, nabumetone, nimesulide, selective cyclooxygenase (COX) inhibitors, and licofelone.
111. The method of paragraph 108, wherein the medicament further comprises levodopa, a dopamine agonist, a catechol O-methyltransferase (COMT) inhibitor, a monoamine oxidase B (MAO B) inhibitor, an anticholinergic agent, an antiviral agent or a combination thereof.
112. The method of paragraph 108, further comprising determining whether the person smokes cigarettes, consumes caffeinated coffee, and/or uses NSAIDs, wherein a predisposition to developing PD is associated, inversely, with cigarette smoking, caffeinated coffee consumption and NSAID use.
113. A kit comprising:
(a) a medicament comprising one or more NSAIDs, caffeine or caffeine analogs, nicotine or nicotine analogs, or combinations thereof; and
(b) instructions for administering the medicament to a person having a genetic predisposition to PD, identified by the method of paragraph 101.
114. The kit of paragraph 113, further comprising levodopa, a dopamine agonist, a catechol O-methyltransferase (COMT) inhibitor, a monoamine oxidase B (MAO B) inhibitor, an anticholinergic agent, an antiviral agent or combinations thereof
115. The kit of paragraph 113, wherein the gene is listed in Table A.
116. A method for identifying the heritage of an individual, comprising:
(a) analyzing the DNA from a blood, saliva or tissue sample obtained from the individual for the presence or absence of one or more genetic markers;
(b) assigning a genetic profile to the individual based on the presence or absence of the one or more genetic markers; and
(c) correlating the genetic profile with a geographic location and ethnic heritage; and
(d) identifying the heritage of an individual based on the correlation.
117. The method of paragraph 116, wherein the individual is of Jewish heritage.
118. The method of paragraph 116, wherein the geographic location is Eastern Europe, France, Great Britain, Germany-Austria, Holland, Ireland, Italy, Russia, Scandinavia.
119. A method of identifying in a person a genetic predisposition to an HLA-associated disease, wherein said method comprises:
(a) obtaining a blood, saliva or tissue sample from the person;
(b) analyzing DNA from the blood, saliva or tissue sample for the presence or absence of a polymorphic site in a HLA gene, whereby the presence of the polymorphic site identifies a gene variant;
   wherein the presence of the gene variant indicates a higher predisposition to the HLA-associated disease, and the absence of the gene variant indicates a lower predisposition to the HLA-associated disease,
   as compared to an individual with the HLA-associated disease and the presence of a polymorphic site in a HLA gene or an individual without the HLA-associated disease and the absence of a polymorphic site in a HLA gene.
120. The method of paragraph 119, wherein the HLA-associated disease is a cancer, an autoimmune disease, or an infectious disease.
121. The method of paragraph 119, wherein the HLA-associated disease is primary hemochromatosis, ankylosing spondylitis, inflammatory bowel, reiter's disease, psoriatic arthritis, system lupus erythematosus, rheumatoid arthritis, Graves' disease, celiac sprue, multiple sclerosis, hay fever, Goodpasture's syndrome, Sjögren's syndrome, pernicious anemia, Hashimoto's thyroiditis, narcolepsy, lyme disease, pemphigus vulgaris, Type 1 diabetes mellitus, acute uveitis, or psoriasis.

### Lengthy Table

The patent application contains lengthy table sections. Tables A, B, C and D are filed in electronic form with the RO/US under PCT AI §801(a).

Having thus described in detail embodiments of the present invention, it is to be understood that the invention defined by the above paragraphs is not to be limited to particular details set forth in the above description as many apparent variations thereof are possible without departing from the spirit or scope of the present invention.

Each patent, patent application, and publication cited or described in the present application is hereby incorporated by reference in its entirety as if each individual patent, patent application, or publication was specifically and individually indicated to be incorporated by reference.

What is claimed is:

1. A method of treating or inhibiting the development of Parkinson's Disease in a person in need thereof, comprising providing individualized or personalized treatment comprising:
(a) analyzing DNA from a blood, saliva or tissue sample obtained from the person;
(b) determining from said analyzing the presence of a polymorphic site in a genetic locus, whereby the presence of the polymorphic site identifies a gene variant that allows or increases the neuroprotective effect of (i) nicotine (ii) caffeine or (iii) NSAID consumption; and
(c) if the polymorphic site is present and (i) the presence of the polymorphic site identifies a gene variant that allows or increases the neuroprotective effect of nicotine, then administering to the person a medicament comprising nicotine or a nicotine analog, or (ii) the presence of the polymorphic site identifies a gene variant that allows or increases the neuroprotective effect of caffeine, then administering to the person a medicament comprising caffeine or a caffeine analog, or (iii) the presence of the polymorphic site identifies a gene variant that allows or increases the neuroprotective effect of NSAID consumption, then administering to the person a medicament comprising one or more NSAID or NSAID analogs;
wherein the nicotine or nicotine analog, the caffeine or caffeine analog or NSAID or NSAID analog is administered in an effective Parkinson's disease treating amount or an amount effective for inhibiting the development of Parkinson's Disease.

2. The method of claim 1, wherein step (a) comprises:
(i) obtaining a blood, saliva or tissue sample from the person,
(ii) isolating DNA from the blood, saliva or tissue sample, and
(iii) genotyping the DNA.

3. The method of claim 1, wherein the NSAID is selected from the group consisting of: ibuprofen, aspirin, indomethacin, nabumetone, nimesulide, selective cyclooxygenase (COX) inhibitors, licofelone, Anaprox (naproxen), Arthrotec (diclofenac sodium), Bextra (valdecoxib), Cataflam (diclofenac potassium), Celebrex (celecoxib), Clinoril (sulindac), Dolobid (diflunisal), EC-naprosyn (naproxen), Feldene (piroxicam), Indocin (indomethacin), Mobic (meloxicam), Motrin (ibuprofen), Naprelan (naproxen controlled release), Naprosyn (naproxen), Ponstel (mefenamic acid), Relafen (nabumetone), Toradol (ketorolac tromethamine), Trilisate (choline magnesium salicylate), Vioxx (rofecoxib) and Voltaren (diclofenac sodium).

4. The method of claim 1, wherein the caffeine analog is selected from the group consisting of Theophylline, 1-Proparagyl 3,7-Dimethyl Xanthine, 7-Proparagyl 1,3-Dimethyl Xanthine, 3-Proparagyl 1,7-Dimethyl Xanthine, 1,3,7-Triproparagyl Xanthine, IBMX, 1,3,7-Tripropyl Xanthine, 7-Benzyl-IBMX, 1-Propyl 3,7-Dimethyl Xanthine, 1,3-Dipropyl 7-Methyl Xanthine, 1,3-Dipropyl 7-Proparagyl Xanthine, 3,7-Dimethyl 1-Propyl Xanthine and 7-allyl 1,3-dimethyl Xanthine.

5. The method of claim 1, wherein the caffeine analog is a adenosine A2A receptor antagonist.

6. The method of claim 1, wherein the nicotine analog is selected from the group consisting of N-succinyl-6-amino-(+/−)-nicotine, 6-(sigma-aminocapramido)-(+/−)-nicotine, O-succinyl-3′-hydroxymethyl-nicotine and 3′-(hydroxymethyl)-nicotine hemisuccinate.

7. The method of claim 1, wherein the polymorphic site comprises a single nucleotide polymorphism (SNP).

8. The method of claim 7, wherein the genetic locus is GRIN2A locus, the SNP comprises rs4998386 having a thymine (T) in heterozygous or homozygous state and the administering is of caffeine or a caffeine analog.

9. The method of claim 7, wherein the genetic locus is SV2C locus, the SNP comprises rs30196 having a cytosine (C) in heterozygous or homozygous state or rs10214163 having a thymine (T) in heterozygous or homozygous state and the administering is of nicotine or a nicotine analog.

10. The method of claim 7, wherein the SNP comprises rs2338971 having a cytosine (C) in homozygous state or rs2072029 having a thymine (T) in heterozygous or homozygous state and the administering is of a NSAID.

11. The method of claim 10, wherein the genetic locus is LAMA4 locus, the SNP comprises rs2072029 having a thymine (T) in heterozygous or homozygous state and the administering is of ibuprofen.

12. The method of claim 1, wherein the polymorphic site comprises a proxy SNP.

13. The method of claim 12, wherein the genetic locus is GRIN2A locus, the pry, SNP has at least 50% correlation with rs4998386 having a thymine (T) in heterozygous or homozygous state and the administering is of caffeine or a caffeine analog.

14. The method of claim 12, wherein the genetic locus is SV2C locus, the proxy SNP has at least 50% correlation with rs30196 having a cytosine (C) in heterozygous or homozygous state or at least 50% correlation with rs10214163 having a thymine (T) in heterozygous or homozygous state and the administering is of nicotine or a nicotine analog.

15. The method of claim 12, wherein the proxy SNP has at least 50% correlation with rs2338971 having a cytosine (C) in homozygous state or has at least 50% correlation with rs2072029 having a thymine (T) in heterozygous or homozygous state and the administering is of a NSAID.

16. The method of claim 15, wherein the genetic locus is LAMA4 locus, the proxy SNP has at least 50% correlation with rs2072029 having a thymine (T) in heterozygous or homozygous state and the administering is of ibuprofen.

17. The method of any one of claims 1 to 16, wherein the medicament further comprises levodopa, a dopamine agonist, a catechol O-methyltransferase (COMT) inhibitor, a monoamine oxidase B (MAO B) inhibitor, an anticholinergic agent, an antiviral agent or a combination thereof.

18. A kit comprising:
(a) a medicament comprising one or more NSAIDs, caffeine or caffeine analogs, nicotine or nicotine analogs, or combinations thereof; and
(b) instructions for performing the method of any one of claims 1-16.

19. The kit of claim 18 further comprising levodopa, a dopamine agonist, a catechol O-methyltransferase (COMT) inhibitor, a monoamine oxidase B (MAO B) inhibitor, an anticholinergic agent, an antiviral agent or a combination thereof and instructions for administration thereof.
